(19) United States
(12) Patent Application Publication
(10) Pub. No.: US 2025/0129087 A1
(43) Pub. Date: Apr. 24, 2025

(54) SALT CRYSTALS

(71) Applicant: INTRA-CELLULAR THERAPIES, INC., New York, NY (US)

(72) Inventor: Peng LI, New Milford, NJ (US)

(73) Assignee: INTRA-CELLULAR THERAPIES, INC., New York, NY (US)

(21) Appl. No.: 18/833,086

(22) PCT Filed: Jan. 31, 2023
(86) PCT No.: PCT/US2023/061696

§ 371 Date: Jul. 25, 2024

Related U.S. Application Data
(60) Provisional application No. 63/267,347, filed on Jan. 31, 2022.

(51) Int. Cl. C07D 487/14; C07C 51/43; C07C 51/64; C07C 53/00; C07C 53/122; C07C 53/126; C07C 53/18; C07C 55/02; C07C 55/07; C07C 55/10; C07C 55/14; C07C 57/15; C07C 59/245; C07C 59/255; C07C 62/18; C07C 63/08; C07C 65/01; C07C 65/10; C07C 65/11; C07C 227/42; C07C 229/64; C07C 231/24; C07C 235/38; C07C 303/44; C07C 309/04; C07C 309/05; C07C 309/25; C07C 309/29; C07C 309/30; C07C 309/35; C07D 213/80; C07D 213/803; C07D 239/557

(52) U.S. Cl. CPC C07D 487/14; C07C 51/43; C07C 51/64; C07C 53/00; C07C 53/122; C07C 53/126; C07C 53/18; C07C 55/02; C07C 55/07; C07C 55/10; C07C 55/14; C07C 57/15; C07C 59/245; C07C 59/255; C07C 62/18; C07C 63/08; C07C 65/01; C07C 65/10; C07C 65/11; C07C 227/42; C07C 229/64; C07C 231/24; C07C 235/38; C07C 303/44; C07C 309/04; C07C 309/05; C07C 309/25; C07C 309/29; C07C 309/30; C07C 309/35; C07D 213/80; C07D 213/803; C07D 239/557; C07B 2200/13; C07C 2601/16; C07C 2602/10; C07C 2602/42

(57) ABSTRACT

Disclosed herein are acid addition salt and salt crystals of (6aR, 9aS)-5,6a,7,8,9,9a-hexahydro-5-methyl-3-(phenylamino)-2-((4(6-fluoropyridin-2-yl)phenyl)methyl)-cyclopent[4,5]imidazo[1,2-a]pyrazolo[4,3-e]pyrimidin-4(2H)-one, compositions comprising the same as well as methods of making and using such salts and crystals.

### CROSS-REFERENCE TO RELATED APPLICATIONS

This application is an international application which claims priority to and the benefit of U.S. Provisional Application Ser. No. 63/267,347, filed on Jan. 31, 2022, the contents of which is hereby incorporated by reference in its entirety.

### FIELD OF THE DISCLOSURE

The present invention relates to acid addition salts and salt crystals of (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one, composition comprising the same and the method of making and using such salts and salt crystals.

### BACKGROUND OF THE DISCLOSURE

The compound (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one is disclosed in WO 2009/075784 (U.S. Pub. No. 2010/0273754). This compound has been found to be a potent and selective phosphodiesterase 1 (PDE 1) inhibitor useful for the treatment or prophylaxis of disorders characterized by low levels of cAMP and/or cGMP in cells expressing PDE1, and/or reduced dopamine D1 receptor signalling activity (e.g., Parkinson's disease, Tourette's Syndrome, Autism, fragile X syndrome, ADHD, restless leg syndrome, depression, cognitive impairment of schizophrenia, narcolepsy); and/or any disease or condition that may be ameliorated by the enhancement of progesterone signalling. This list of disorders is exemplary and not intended to be exhaustive.

The publication WO 2009/075784 discloses (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one in free base form and generally in pharmaceutically acceptable salt form, but no specific salt was shown to have particular stability or desired properties. Because many pharmaceutical compounds can exist in different physical forms (e.g., liquid or solid in different crystalline, amorphous, polymorphous, hydrate or solvate forms) which can vary the stability, solubility, bioavailability or pharmacokinetics (absorption, distribution, metabolism, excretion or the like) and/or bioequivalence of a drug, it is of critical importance in the pharmaceutical development to identify a pharmaceutical compound of optimal physical form (e.g., free base or salt in solid, liquid, crystalline, hydrate, solvate, amorphous or polymorphous forms).

### SUMMARY OF THE DISCLOSURE

In a first aspect, the present disclosure is directed to salt forms, e.g., crystalline salt forms, of (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one (Compound A) acid addition salts. These salts and salt crystals are especially advantageous in the preparation of galenic formulations of various and diverse kind. In various embodiments, the Salt Crystals of the Disclosure are selected from the group consisting of hydrochloride, malate, fumarate, sulfate, esylate, galactarate, adipate, lactate, oxalate, palmitate, 2-oxo-glutarate, xinafoate, tosylate, tartrate, succinate, mesylate, napadisylate, edisylate, propionate, caprylate, besylate, benzoate, nicotinate, isonicotinate, orotate, camsylate, salicylate, aminosalicylate, mandelate, acetamido-benzoate, trifluoroacetate, dichloroacetate, caproate, or laurate salts. The various salt crystals according to the present disclosure may be in anhydrous or solvate form.

In a second aspect, the present disclosure further provides a method for the production of stable acid addition salts of (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one (“Compound A”), e.g., crystallinic acid addition salts with particular acids comprising the steps of reacting Compound A in free base form with an acid in a solvent and isolating the salt obtained. In various embodiments, the method further comprises the step of forming a slurry of Compound A with the acid in the solvent at a temperature between about 30° C. to 70° C., e.g., for a period of at least one hour. In various embodiments, the method further comprises the step of cooling the solution to a temperature of about −10° C. to about 20° C. In various embodiments, the method further comprises the step of drying the solution by evaporation. In various embodiments, the obtained salt is crystalline, and are dissolved in a second solvent and are subjected to one or more cooling cycles.

In a third aspect, the present disclosure provides a method for the prophylaxis or treatment of a patient, e.g., a human, suffering from a disorder selected from one or more of neurodegenerative diseases; mental disorders; circulatory and cardiovascular disorders; respiratory and inflammatory disorders; diseases which may be alleviated by the enhancement of progesterone signalling; a disease or disorder such as psychosis, glaucoma, or elevated intraocular pressure; a traumatic brain injury; a cancer or tumor; a renal disorder; any disease or condition characterized by low levels of cAMP and/or cGMP; and any disease or condition characterized by reduced dopamine D1 receptor signalling activity, wherein the method comprises the step of administering to a patient in need thereof a therapeutically effective amount of the compound (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one (Compound A) in acid addition salt form, e.g., according to any of the Salt Crystals of the Disclosure.

### BRIEF DESCRIPTION OF THE FIGURES

FIG. 1A illustrates an x-ray powder diffraction pattern of Hydrochloride Salt 1.

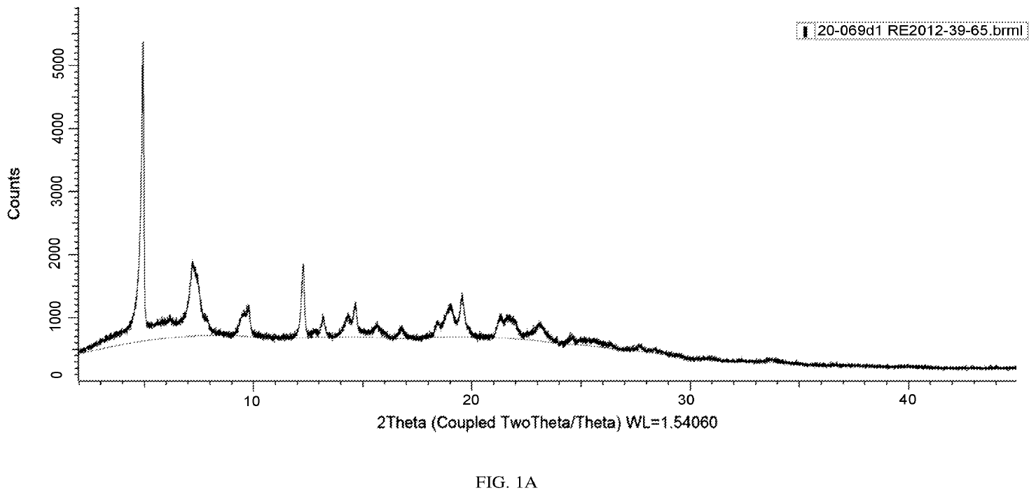

FIG. 1B illustrates a combination differential scanning calorimetry (DSC)/thermogravimetric analysis (TGA) thermograph of Hydrochloride Salt 1.

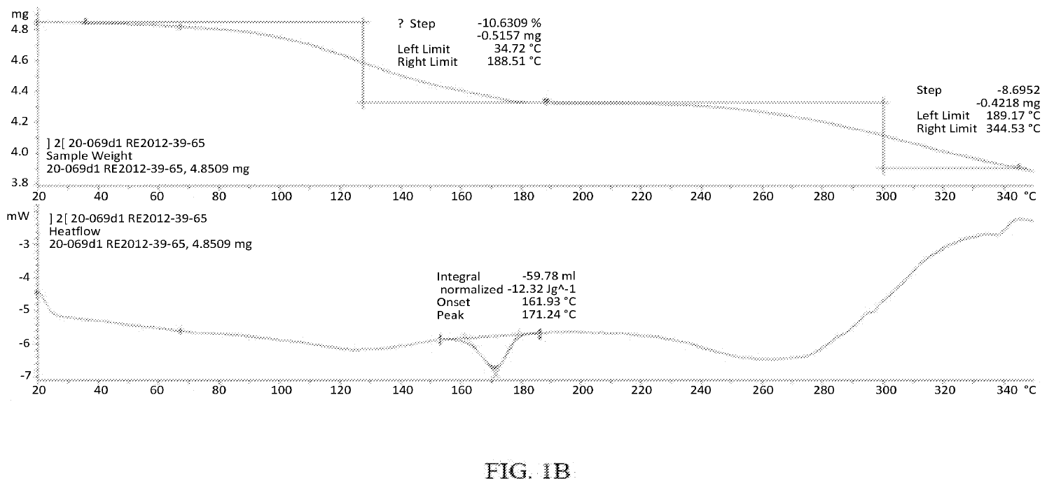

FIG. 2A illustrates an x-ray powder diffraction pattern of Hydrochloride Salt 2.

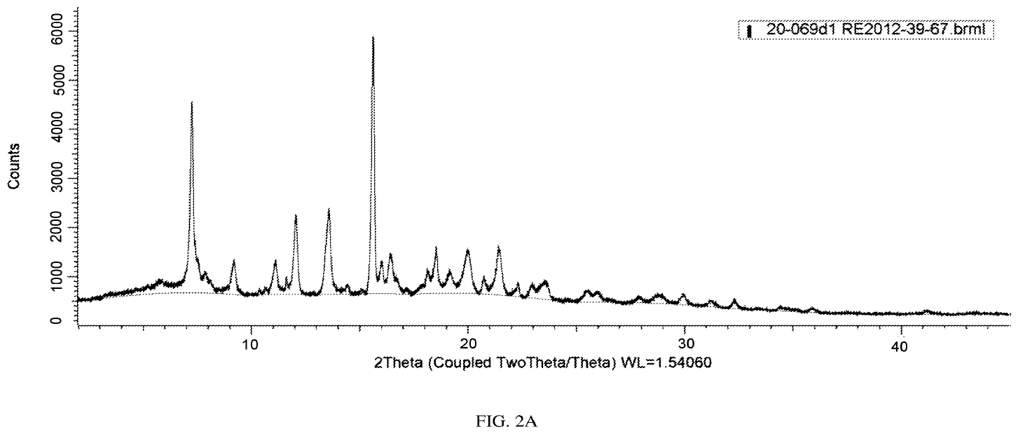

FIG. 2B illustrates a combination differential scanning calorimetry (DSC)/thermogravimetric analysis (TGA) thermograph of Hydrochloride Salt 2.

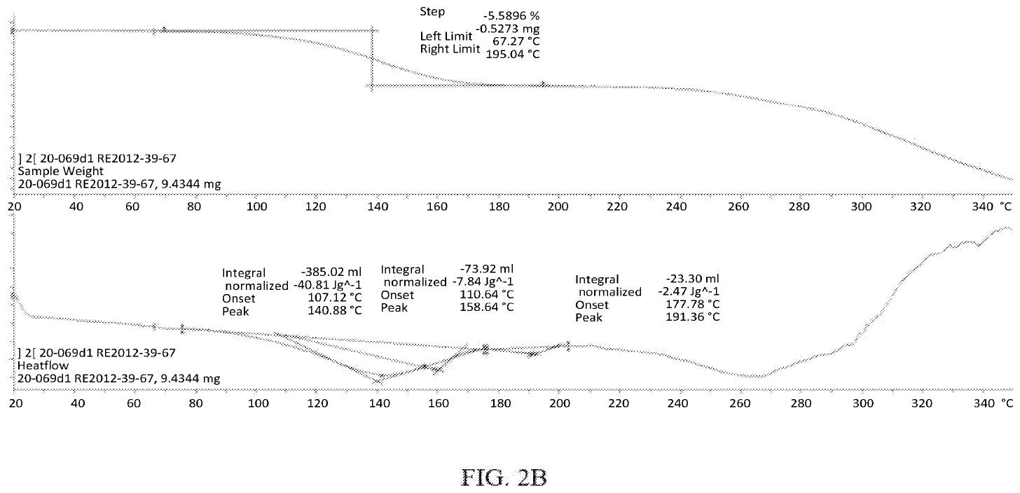

FIG. 3A illustrates an x-ray powder diffraction pattern of Hydrochloride Salt 3.

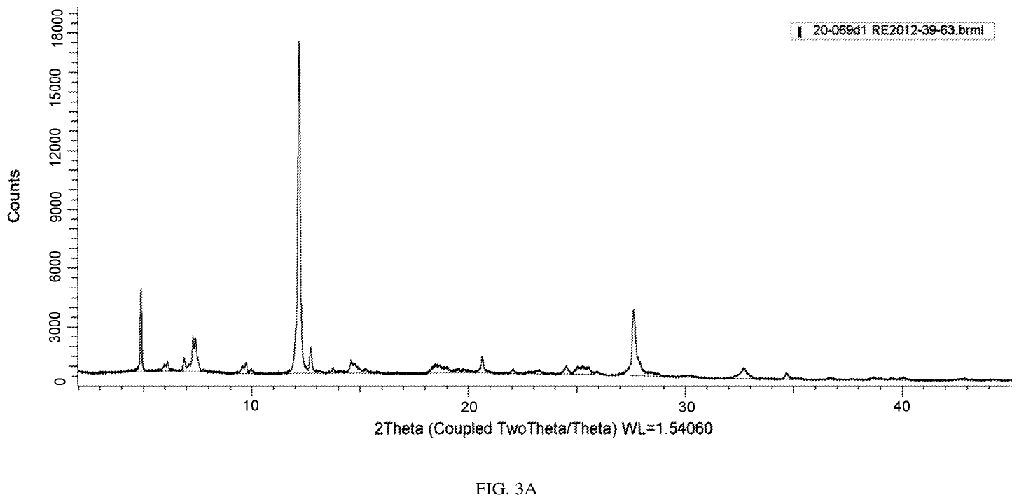

FIG. 3B illustrates a combination differential scanning calorimetry (DSC)/thermogravimetric analysis (TGA) thermograph of Hydrochloride Salt 3.

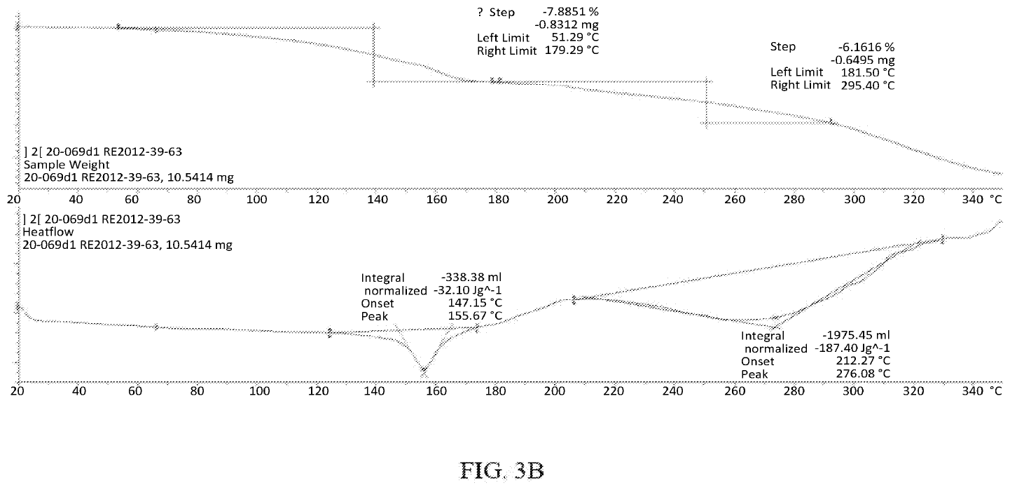

FIG. 4A illustrates an x-ray powder diffraction pattern of Hydrochloride Salt 4.

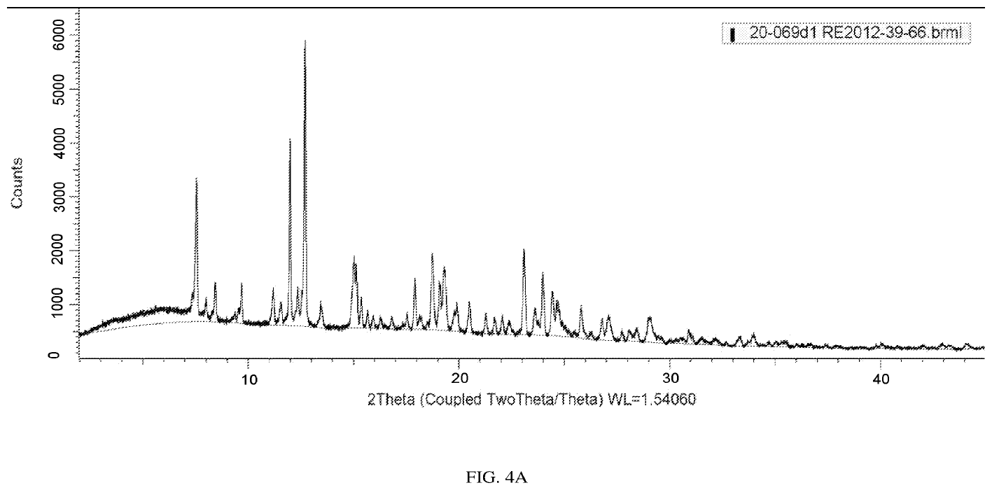

FIG. 4B illustrates a combination differential scanning calorimetry (DSC)/thermogravimetric analysis (TGA) thermograph of Hydrochloride Salt 4.

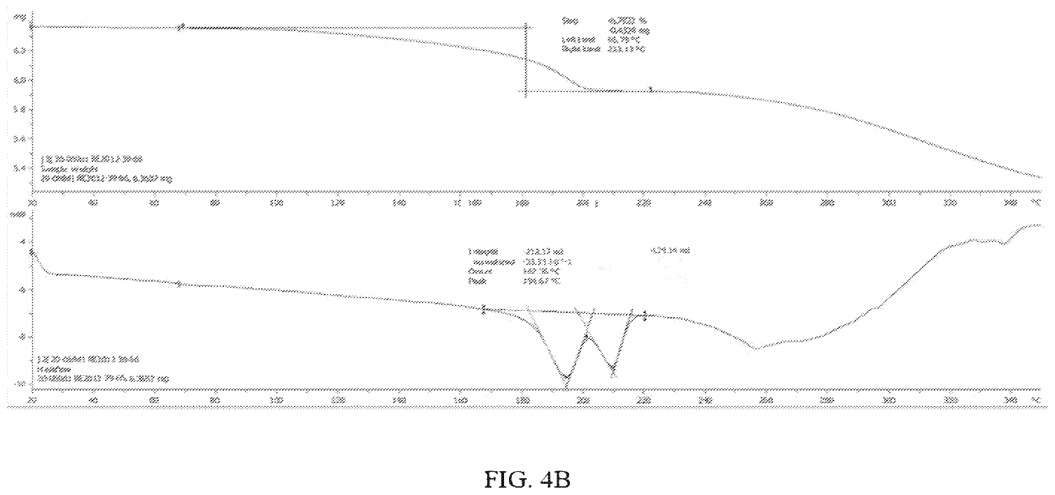

FIG. 5A illustrates an x-ray powder diffraction pattern of Hydrochloride Salt 5.

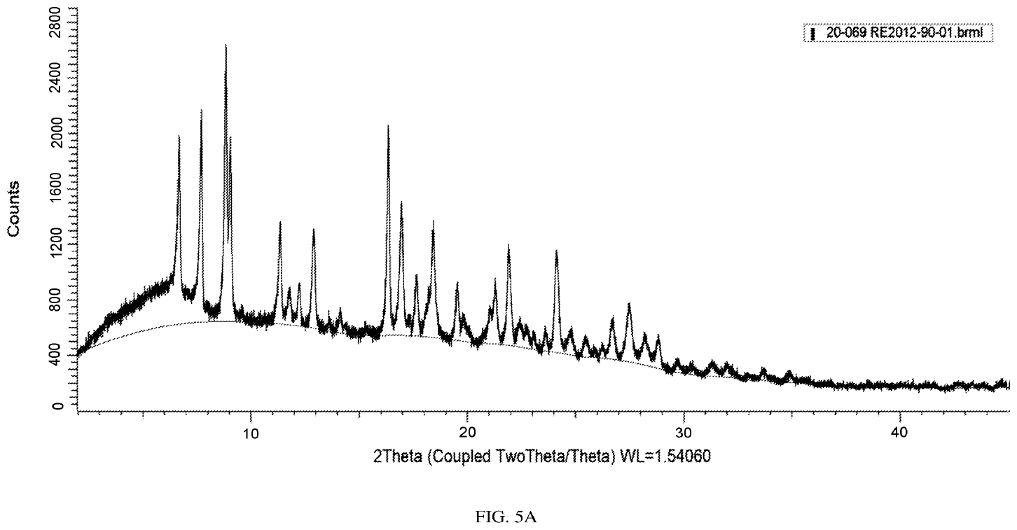

FIG. 5B illustrates a combination differential scanning calorimetry (DSC)/thermogravimetric analysis (TGA) thermograph of Hydrochloride Salt 5.

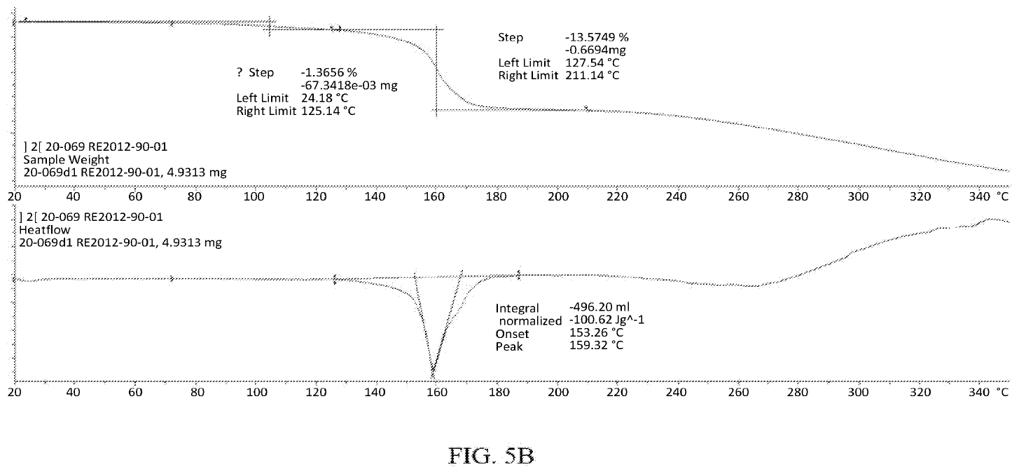

FIG. 6A illustrates an x-ray powder diffraction pattern of Hydrochloride Salt 6.

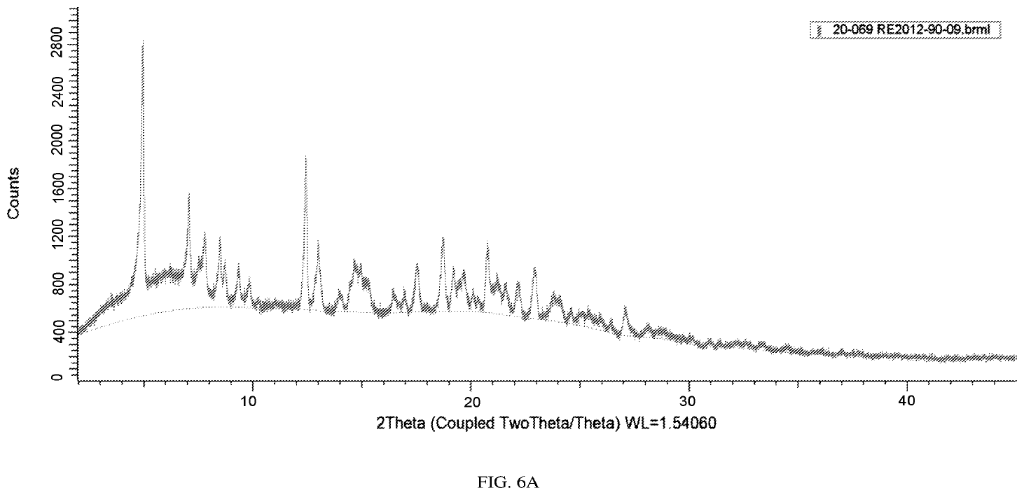

FIG. 6B illustrates a combination differential scanning calorimetry (DSC)/thermogravimetric analysis (TGA) thermograph of Hydrochloride Salt 6.

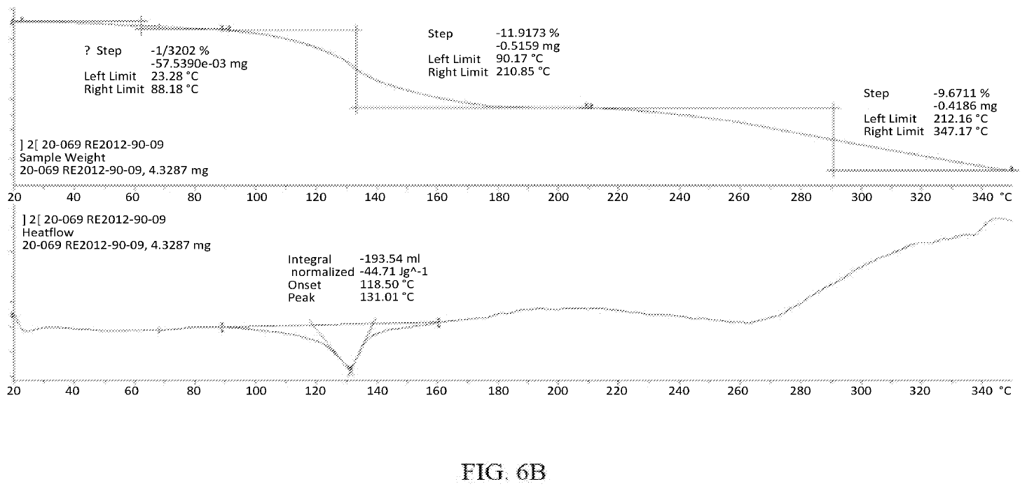

FIG. 7A illustrates an x-ray powder diffraction pattern of Hydrochloride Salt 7.

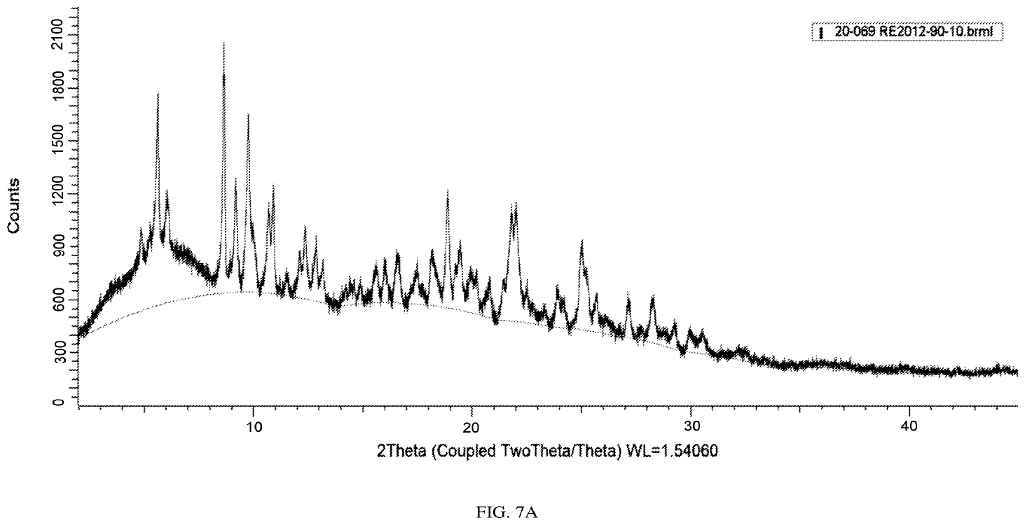

FIG. 7B illustrates a combination differential scanning calorimetry (DSC)/thermogravimetric analysis (TGA) thermograph of Hydrochloride Salt 7.

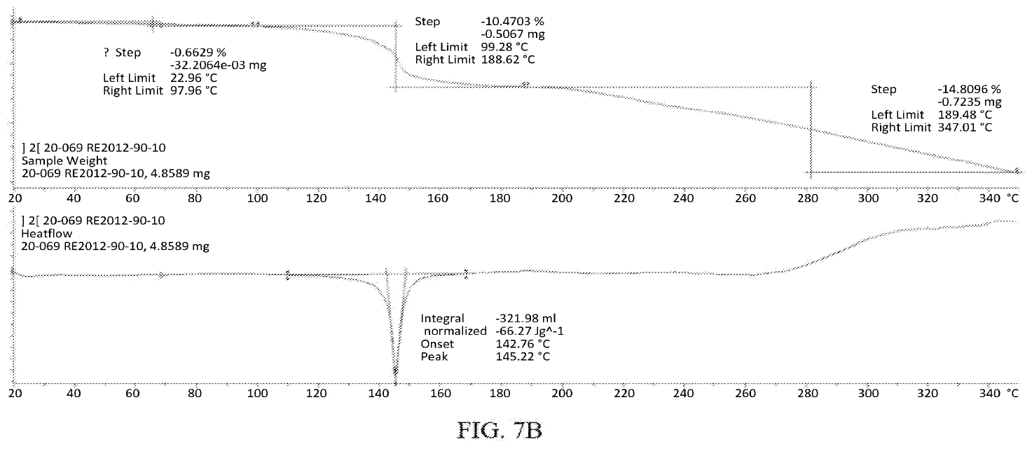

FIG. 8A illustrates an x-ray powder diffraction pattern of Hydrochloride Salt 8.

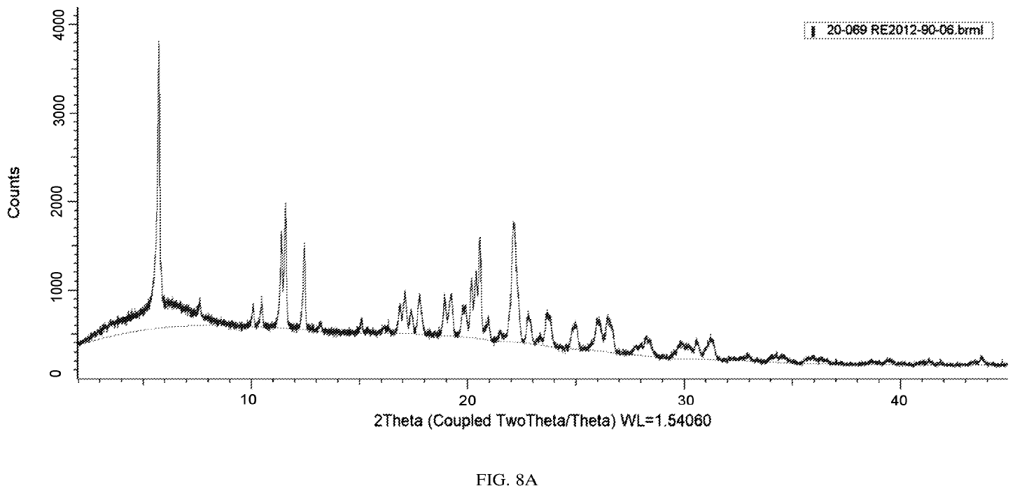

FIG. 8B illustrates a combination differential scanning calorimetry (DSC)/thermogravimetric analysis (TGA) thermograph of Hydrochloride Salt 8.

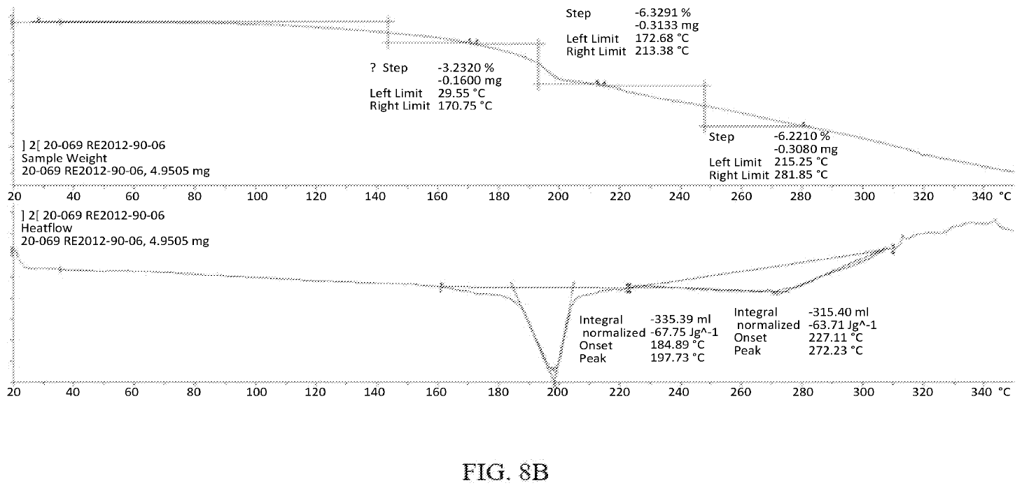

FIG. 9A illustrates an x-ray powder diffraction pattern of Malate Salt 1.

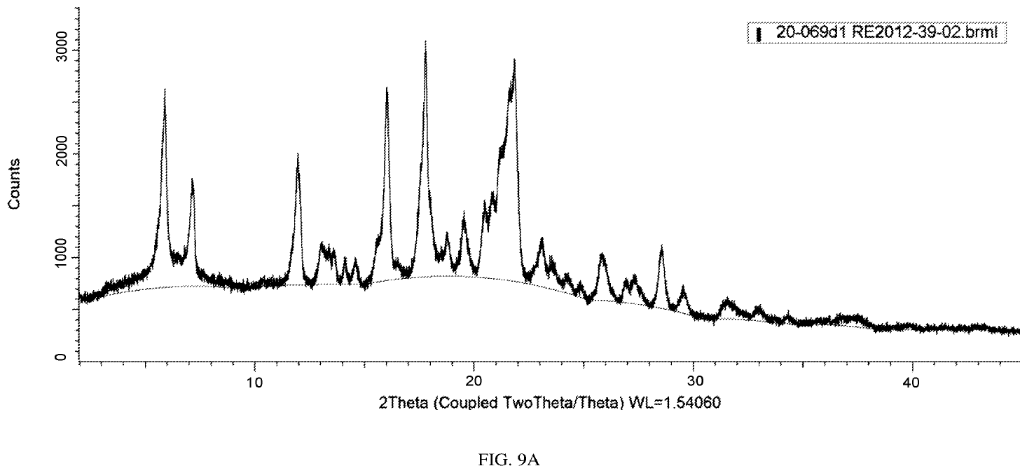

FIG. 9B illustrates a combination differential scanning calorimetry (DSC)/thermogravimetric analysis (TGA) thermograph of Malate Salt 1.

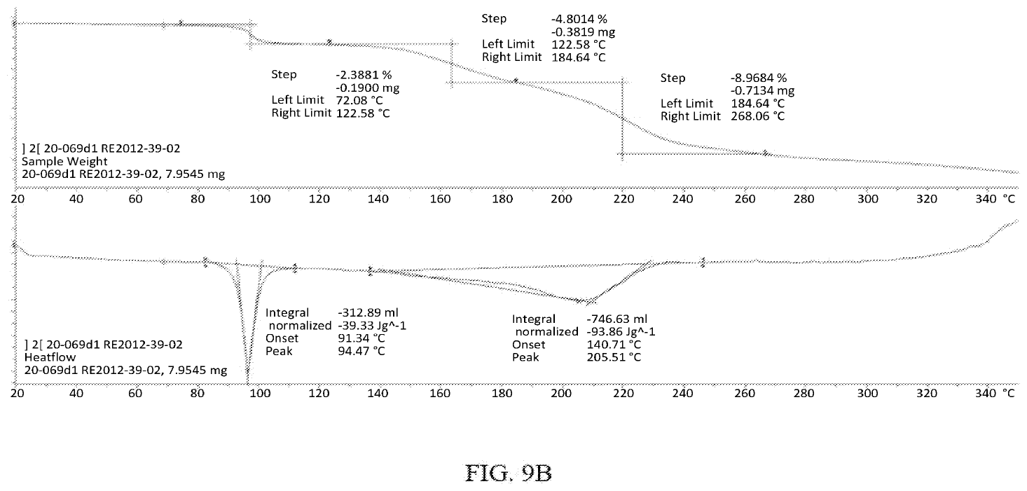

FIG. 10A illustrates an x-ray powder diffraction pattern of Tartrate Salt 1.

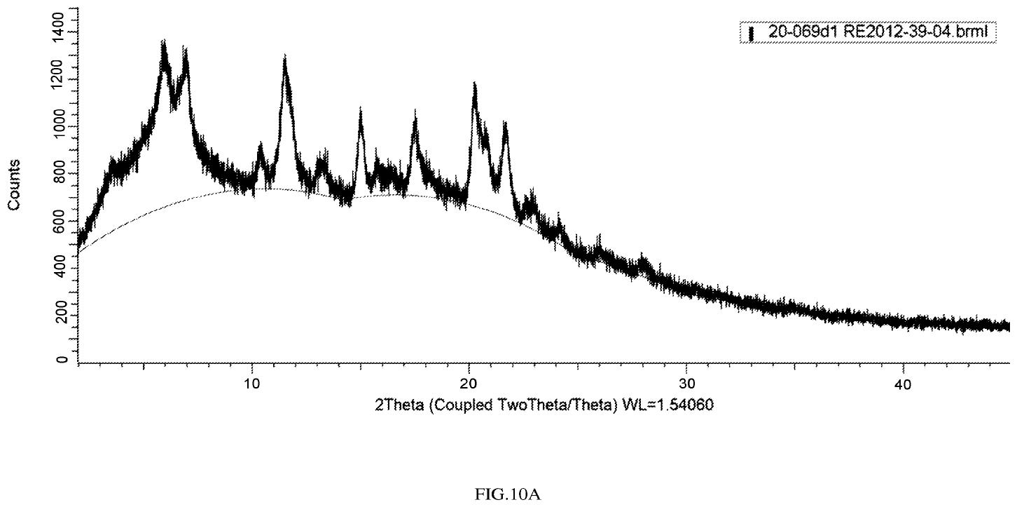

FIG. 10B illustrates a combination differential scanning calorimetry (DSC)/thermogravimetric analysis (TGA) thermograph of Tartrate Salt 1.

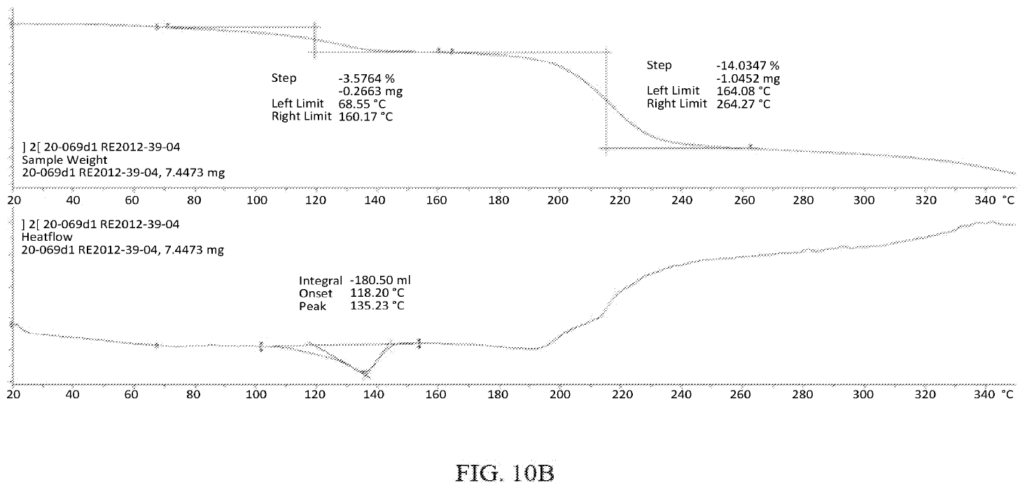

FIG. 11A illustrates an x-ray powder diffraction pattern of Tartrate Salt 2.

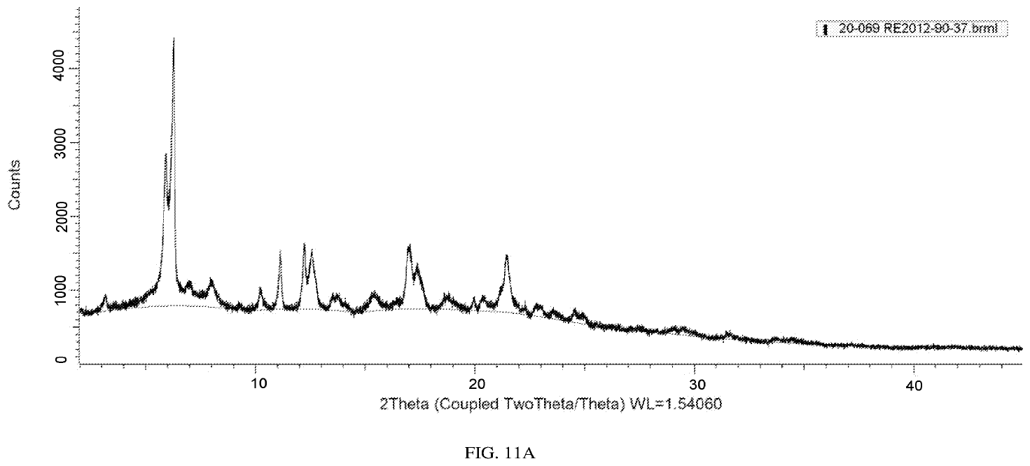

FIG. 11B illustrates a combination differential scanning calorimetry (DSC)/thermogravimetric analysis (TGA) thermograph of Tartrate Salt 2.

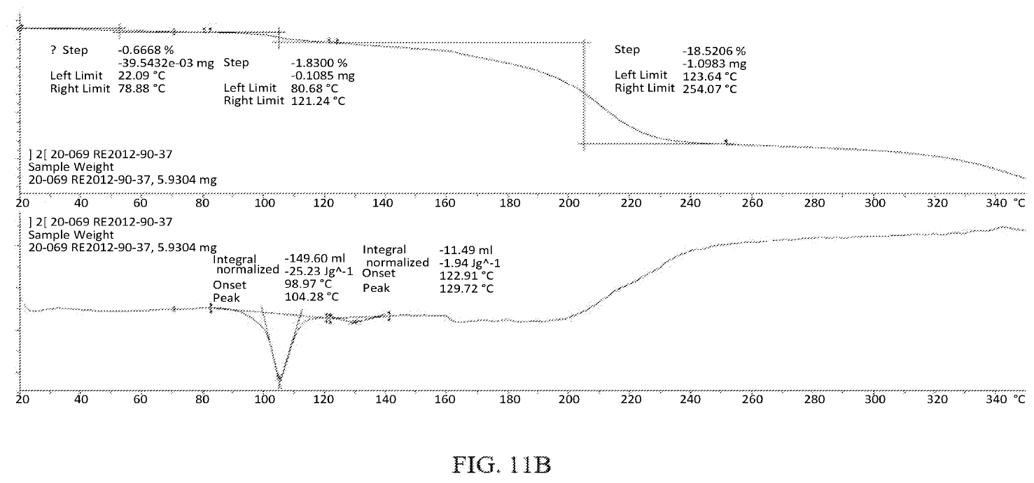

FIG. 12A illustrates an x-ray powder diffraction pattern of Oxalate Salt 1.

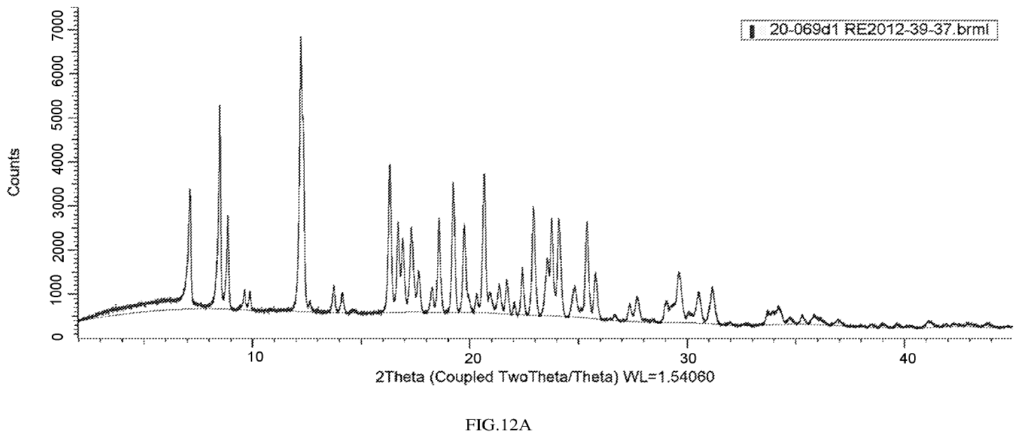

FIG. 12B illustrates a combination differential scanning calorimetry (DSC)/thermogravimetric analysis (TGA) thermograph of Oxalate Salt 1.

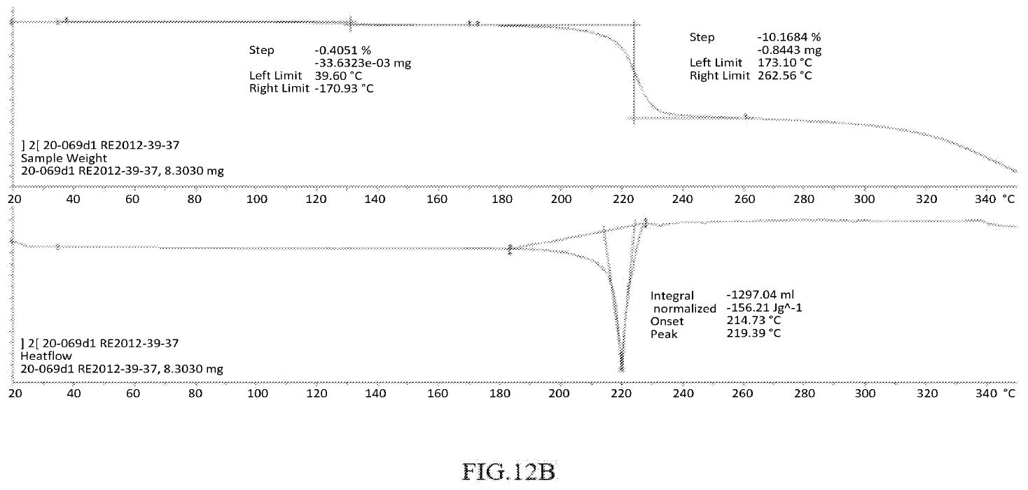

FIG. 13A illustrates an x-ray powder diffraction pattern of Oxalate Salt 2.

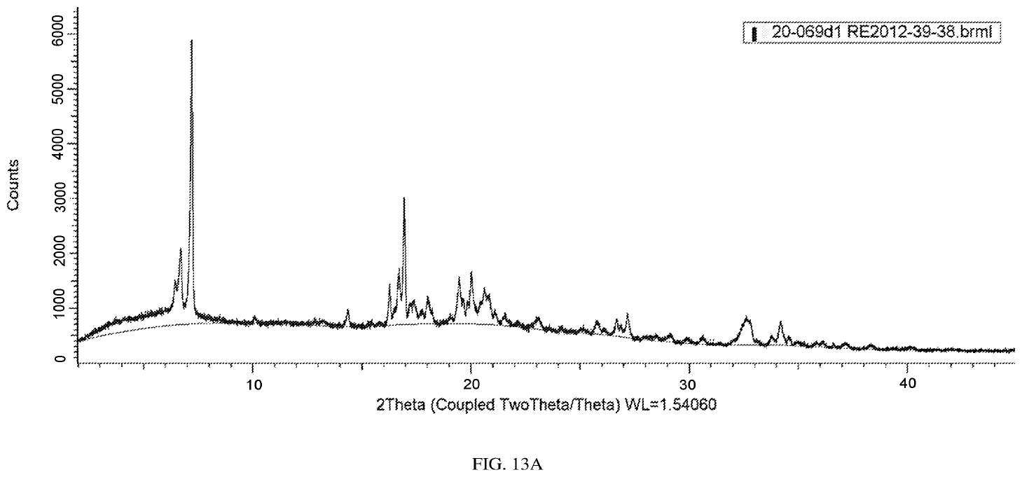

FIG. 13B illustrates a combination differential scanning calorimetry (DSC)/thermogravimetric analysis (TGA) thermograph of Oxalate Salt 2.

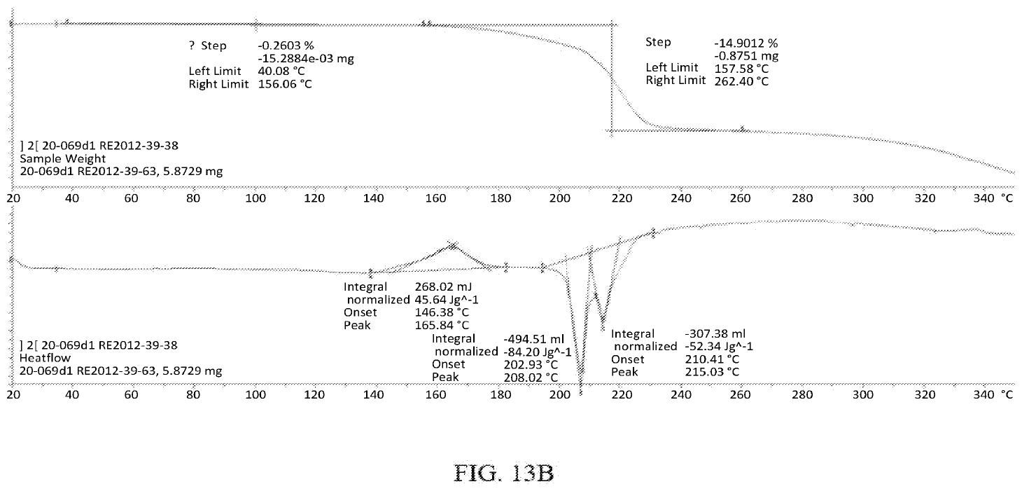

FIG. 14A illustrates an x-ray powder diffraction pattern of Oxalate Salt 3.

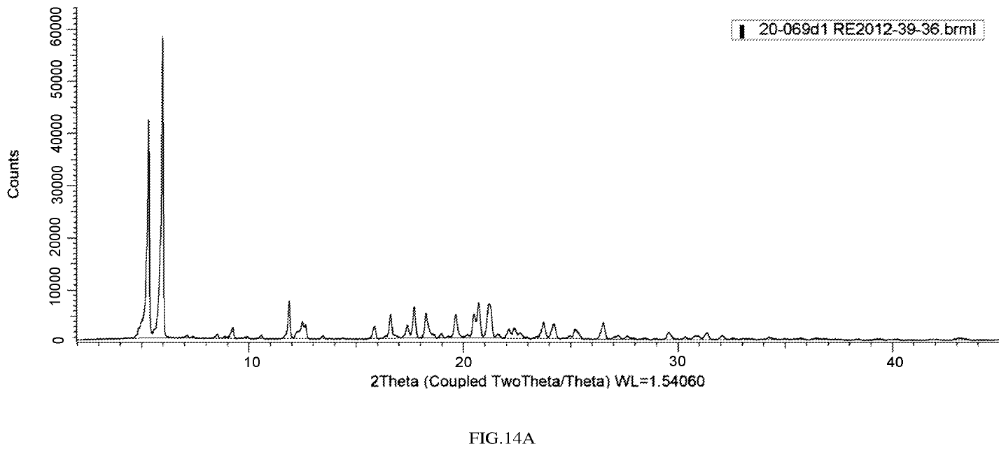

FIG. 14B illustrates a combination differential scanning calorimetry (DSC)/thermogravimetric analysis (TGA) thermograph of Oxalate Salt 3.

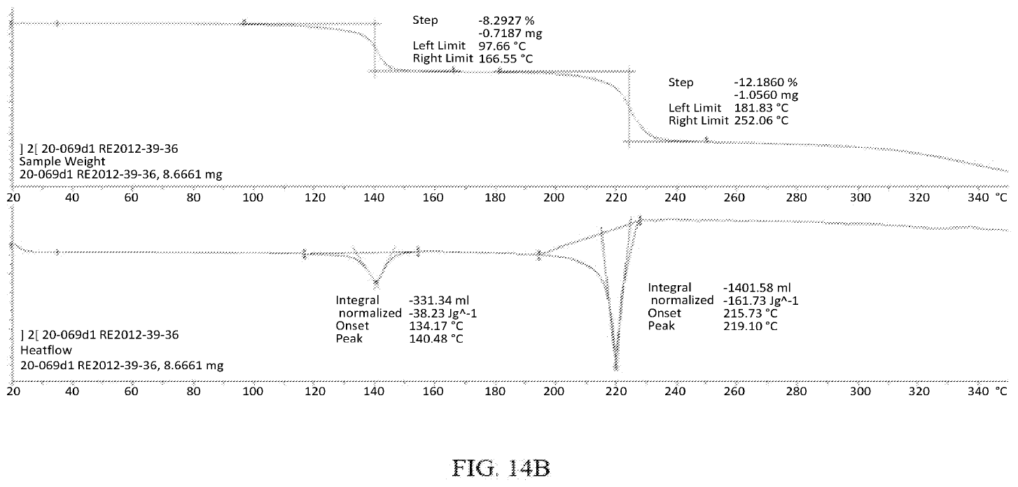

FIG. 15A illustrates an x-ray powder diffraction pattern of Oxalate Salt 4.

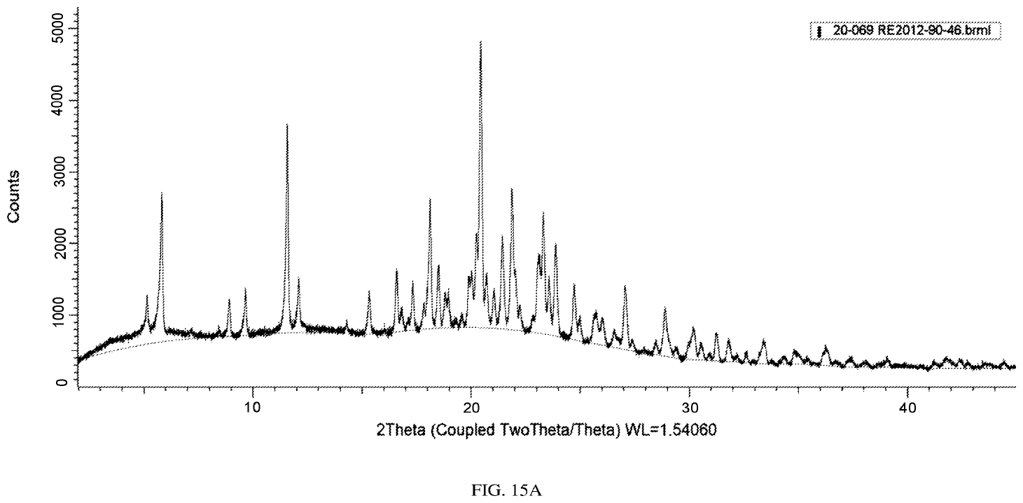

FIG. 15B illustrates a combination differential scanning calorimetry (DSC)/thermogravimetric analysis (TGA) thermograph of Oxalate Salt 4.

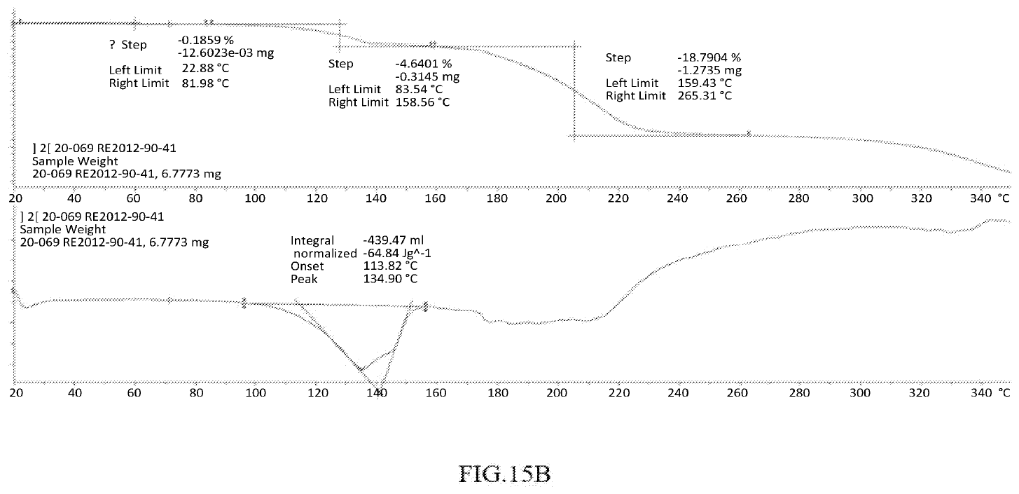

### DETAILED DESCRIPTION

As use herein, the term “crystal” or “crystals” or “crystalline” or “crystallinic” refers to any solid that has a short- or long-range order of the molecules, atoms or ions in a fixed lattice arrangement. Salt Crystals of the Disclosure may be in a single crystal form. Therefore, the Salt Crystals of the Disclosure may be in a triclinic, monoclinic, orthorhombic, tetragonal, rhombohedral, hexagonal or cubic crystal form or mixtures thereof. In particular, the Salt Crystals of the Disclosure are in dry crystalline form. In a particular embodiment, the Salt Crystals of the Disclosure are substantially free of other forms, e.g., free of amorphous or other crystal forms.

The term “substantially free” of other crystal forms refer to less than about 10 wt. %, preferably less than about 5 wt. %, more preferably less than about 2 wt. %, still preferably less than about 1 wt. %, still preferably less than about 0.1%, most preferably less than about 0.01 wt. % of other forms or other crystal forms, e.g., amorphous or other crystal forms.

The term “predominantly” or “substantially entirely in a single form” refers to less than about 10 wt. %, preferably less than about 5 wt. %, more preferably less than about 2 wt. %, still preferably less than about 1 wt. %, still preferably less than about 0.1%, most preferably less than about 0.01 wt. % of other solid forms, e.g., amorphous or other crystal forms.

In particular embodiment, the Salt Crystals of the Disclosure may contain trace amounts of solvent, e.g., in solvate form, or trace amounts of water, e.g., in hydrate form. Preferably, the Salt Crystals of the disclosure are in non-solvate form. Still preferably, the crystals of the disclosure are in non-solvate and non-hydrate form.

The Salt Crystals of the Disclosure may have a free base to acid ratio of 1 to 1, 1 to 0.5 or 1 to >1, e.g., 1 to 1.3 or 1 to 2, etc.

The term “solvate” refers to crystalline solid adducts containing either stoichiometric or nonstoichiometric amounts of a solvent incorporated within the crystal structure. Therefore, the term “non-solvate” form herein refers to salt crystals that are free or substantially free of solvent molecules within the crystal structures of the disclosure.

The term “amorphous” form refers to solids of disordered arrangements of molecules and do not possess a distinguishable crystal lattice.

Unless further modified, the term “Compound A” refers to (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one in free base form, having the following structure:

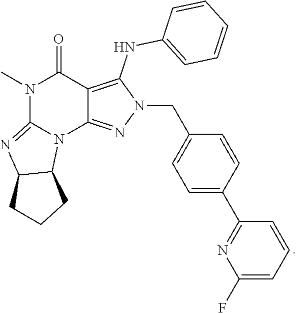 [SMILES: CN1C(=O)c2c(nn(Cc3ccc(-c4cccc(F)n4)cc3)c2Nc2ccccc2)N2C1=N[C@@H]1CCC[C@@H]12]

The crystallinity or the morphology of the crystals of the Present Disclosure may be determined by a number of methods, including, but not limited to single crystal X-ray diffraction, X-ray powder diffraction, polarizing optical microscopy, thermal microscopy, differential scanning calorimetry (DSC), thermogravimetric analysis (TGA), infrared adsorption spectroscopy and Raman spectroscopy. Characterization of solvates or hydrates or lack thereof may also be determined by DSC and/or TGA.

It is to be understood that X-ray powder diffraction pattern or the differential scanning calorimetry pattern of a given sample may vary a little (standard deviation) depending on the instrument used, the time and temperature of the sample when measured and standard experimental errors. Therefore, the temperature or the 2-theta values, d-spacing values, heights and relative intensity of the peaks as set forth herein in the Tables or in the Figures will have an acceptable level of deviation. For example, the values may have an acceptable deviation of e.g., about 20%, 15%, 10%, 5%, 3%, 2% or 1%. In particular embodiment, the 2-theta values or the d-spacing values of the XRPD pattern of the crystals of the current disclosure may have an acceptable deviation of ±0.2 degrees and/or ±0.2 Å. Further, the XRPD pattern of the crystals of the disclosure may be identified by the characteristic peaks as recognized by one skilled in the art. For example, the crystals of the disclosure may be identified by e.g., at least five characteristic peaks, e.g., at least three or at least five peaks, e.g., at least three or at least five 2-theta values and/or at least three or at least five d-spacing values as set forth in the XRPD patterns set forth herein. Therefore, the term “corresponding with or substantially as” set forth in any of the Tables or depicted in any of the Figures refers to any crystals which has an XRPD having the major or characteristic peaks as set forth in the tables/figures.

The term “about” in front of a numerical value refers to the numerical value itself ±20%, ±15%, ±10%, preferably ±5%, preferably ±3%, preferably ±2%, preferably ±1% of that value. When referencing temperature, the term about refers to the temperature value itself ±10° C., preferably ±5° C., preferably ±3° C. of the reference temperature. In another example, when referencing 2-theta angle values, the term “about” refers to the numerical 2-theta angle value itself ±0.2 degrees of the reference 2-theta angle value. In still another example, when referencing d-spacing values, the term “about” refers to the numerical 2-theta angle value itself ±0.2 Å of the reference d-spacing value.

The crystals of the disclosure are selective PDE1 inhibitors. Therefore, the crystals of the disclosure are useful for the treatment of PDE1 related disorders as set forth in e.g., WO 2014/151409, WO 2018/049417, WO 2019/227004, WO 2019/152697, WO 2009/075784, WO 2010/132127, WO 2006/133261 and WO 2011/153129, the contents of each of which are incorporated by reference in their entireties.

The term “patient” includes human and non-human. In one embodiment, the patient is a human. In another embodiment, the patient is a non-human.

### Salts and Salt Crystals of the Disclosure

In a first aspect, the present disclosure is directed to a salt of the compound (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one (Compound A) in acid addition salt form [Salt 1]. These salts may be in the form of salt crystals and are especially advantageous in the preparation of galenic formulations of various and diverse kind. Therefore, in the first aspect, the invention provides the following:
1.1 Salt 1, wherein compound (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one (Compound A) in an acid addition salt form, e.g., selected from the group consisting of hydrochloride, malate, fumarate, sulfate, esylate, galactarate, adipate, lactate, oxalate, palmitate, 2-oxo-glutarate, xinafoate, tosylate, tartrate, succinate, mesylate, napadisylate, edisylate, propionate, caprylate, besylate, benzoate, nicotinate, isonicotinate, orotate, camsylate, salicylate, aminosalicylate, mandelate, acetamido-benzoate, trifluoroacetate, dichloroacetate, caproate, or laurate salts.
1.2 Salt 1 or 1.1, wherein the salt is crystalline.
1.3 Salt 1 or 1.1, wherein the salt is in anhydrous crystalline form.
1.4 Any of Salts 1-1.2, wherein the salt is in solvate crystalline form.
1.5 Any of the preceding Salts, wherein the salt is a hydrochloride salt.
1.6 Any of the Salts 1.1-1.4, wherein the salt is a malate salt.
1.7 Any of the Salts 1.1-1.4, wherein the salt is a fumarate salt.
1.8 Any of the Salts 1.1-1.4, wherein the salt is a sulfate salt.
1.9 Any of the Salts 1.1-1.4, wherein the salt is an esylate salt.
1.10 Any of the Salts 1.1-1.4, wherein the salt is a tosylate salt.
1.11 Any of the Salts 1.1-1.4, wherein the salt is a tartrate salt.
1.12 Any of the Salts 1.1-1.4, wherein the salt is a succinate salt.
1.13 Any of the Salts 1.1-1.4, wherein the salt is a mesylate salt.
1.14 Any of the Salts 1.1-1.4, wherein the salt is a napadisylate salt.
1.15 Any of the Salts 1.1-1.4, wherein the salt is an edisylate salt.
1.16 Any of the Salts 1.1-1.4, wherein the salt is a propionate salt.
1.17 Any of the Salts 1.1-1.4, wherein the salt is a caprylate salt.
1.18 Any of the Salts 1.1-1.4, wherein the salt is a besylate salt.
1.19 Any of the Salts 1.1-1.4, wherein the salt is a benzoate salt.
1.20 Any of the Salts 1.1-1.4, wherein the salt is a nicotinate salt.
1.21 Any of the Salts 1.1-1.4, wherein the salt is an isonicotinate salt.
1.22 Any of the Salts 1.1-1.4, wherein the salt is an orotate salt.
1.23 Any of the Salts 1.1-1.4, wherein the salt is a camsylate salt.
1.24 Any of the Salts 1.1-1.4, wherein the salt is a salicylate salt.
1.25 Any of the Salts 1.1-1.4, wherein the salt is an aminosalicylate salt.
1.26 Any of the Salts 1.1-1.4, wherein the salt is a mandelate salt.
1.27 Any of the Salts 1.1-1.4, wherein the salt is an acetamido-benzoate salt.
1.28 Any of the Salts 1.1-1.4, wherein the salt is a trifluoroacetate salt.
1.29 Any of the Salts 1.1-1.4, wherein the salt is a dichloroacetate salt.
1.30 Any of the Salts 1.1-1.4, wherein the salt is a caproate salt.
1.31 Any of the Salts 1.1-1.4, wherein the salt is a laurate salt.
1.32 Any of the preceding Salts, wherein the Salt is crystalline and is in the form of a solvate selected from an acetonitrile, ethyl acetate, acetone, 2-butanone, 2-ethyl-1-butanol, ethyl salicylate, ethyl butyl ketone, acetone, 3-heptanone, toluene, methanol, ethanol, propanol (e.g., isopropanol, 2-propanol), butanol (e.g., 2-ethyl-1-butanol), dimethyl sulfoxide (DMSO), anisole, or ethyl butyl ketone solvate.
1.33 Any of the preceding Salts, wherein the Salt is crystalline and is in the form of a solvate selected from an acetonitrile, ethyl acetate, 2-butanone, acetone, 3-heptanone, or 2-butanone solvate.
1.34 Any of the preceding Salts, wherein the Salt is crystalline and the Compound A free base and hydrochloride counterion are present in a molar ratio of about 2:1 to about 1:2, e.g., about 1:1, e.g., about 1:0.3, about 1:0.4, about 1:0.4, about 1:0.5, about 1:0.6, about 1:0.7, about 1:0.8, about 1:0.9.

It has also been surprisingly found that particular Salts of the Present Invention are in crystalline form, and therefore are preferred for galenic and/or therapeutic use. Therefore, in a further embodiment, the invention provides a hydrochloride salt [Hydrochloride Salt 1] of Compound A.
1.1 Hydrochloride Salt 1, which is in crystalline form.
1.2 Hydrochloride Salt 1 or 1.1, which is in solvate form.
1.3 Any of Hydrochloride Salts 1-1.2, wherein the salt crystal is in the form of a solvate with one or more of acetonitrile, ethyl acetate, acetone, 2-butanone, 2-ethyl-1-butanol, ethyl salicylate, ethyl butyl ketone, acetone, or combinations thereof.
1.4 Any of Hydrochloride Salts 1-1.3, wherein the salt crystal is an ethyl acetate solvate.
1.5 Any of Hydrochloride Salts 1-1.4, wherein said salt crystal exhibits an X-ray powder diffraction pattern comprising at least five peaks having 2-theta angle values selected from the group consisting of: 4.9, 7.3, 9.5, 9.7, 12.3, 14.4, 14.6, 19.0, 19.6, and 21.4 degrees, wherein the XRPD pattern is measured in a diffractometer using copper anode, e.g., at wavelength alpha1 of 1.5406 Å and wavelength alpha2 of 1.5444 Å.
1.6 Any of Hydrochloride Salts 1-1.5, wherein said salt crystal exhibits an X-ray powder diffraction pattern comprising at least five peaks having 2-theta angle values selected from the group consisting of: 4.9, 7.3, 12.3, 19.0, and 19.6 degrees, wherein the XRPD pattern is measured in a diffractometer using copper anode, e.g., at wavelength alpha1 of 1.5406 Å and wavelength alpha2 of 1.5444 Å.
1.7 Any of Hydrochloride Salts 1-1.6, wherein said salt crystal exhibits an X-ray powder diffraction pattern comprising at least five peaks having d-spacing values selected from the group consisting of 17.9, 12.1, 9.3, 9.1, 7.2, 6.2, 6.0, 4.7, 4.5, and 4.2 Å.
1.8 Any of Hydrochloride Salts 1-1.7, wherein said salt crystal exhibits an X-ray powder diffraction pattern comprising peaks having d-spacing values selected from the group consisting of 17.9, 12.1, 7.2, 4.7, and 4.5 Å.
1.9 Any of Hydrochloride Salts 1-1.8, wherein said salt crystal exhibits an X-ray powder diffraction pattern comprising at least five peaks having 2-theta angle values or comprising at least five peaks having d-spacing values selected from those set forth in Table 1 as defined herein, wherein the XRPD pattern is measured in a diffractometer using copper anode, at wavelength alpha1 of 1.5406 Å and wavelength alpha2 of 1.5444 Å.
1.10 Any of Hydrochloride Salts 1-1.9, wherein said salt crystal exhibits a Differential Scanning Calorimetry (DSC) pattern comprising an endothermic peak at about 169° C.-172° C., e.g., at about 170° C.
1.11 Any of Hydrochloride Salts 1-1.10, wherein the Compound A free base and hydrochloride counterion are present in a molar ratio of about 2:1 to about 1:2, e.g., about 1:1, e.g., about 1:0.9.

In a further embodiment, the present disclosure provides for a second hydrochloride salt [Hydrochloride Salt 2] of Compound A.
2.1 Hydrochloride Salt 2, which is in crystalline form.
2.2 Hydrochloride Salt 2 or 2.1, which is in solvate form.
2.3 Any of Hydrochloride Salts 2-2.2, wherein the salt crystal is in the form of a solvate with one or more of acetonitrile, ethyl acetate, acetone, 2-butanone, 2-ethyl-1-butanol, ethyl salicylate, ethyl butyl ketone, acetone, or combinations thereof.
2.4 Any of Hydrochloride Salts 2-2.3, wherein the salt crystal is an ethyl butyl ketone solvate.
2.5 Any of Hydrochloride Salts 2-2.4, wherein said salt crystal exhibits an X-ray powder diffraction pattern comprising at least five peaks having 2-theta angle values selected from the group consisting of: 7.3, 12.1, 13.6, 15.6, 16.4, 18.5, 20.0, 21.3, 21.4, and 21.5 degrees, wherein the XRPD pattern is measured in a diffractometer using copper anode, e.g., at wavelength alpha1 of 1.5406 Å and wavelength alpha2 of 1.5444 Å.
2.6 Any of Hydrochloride Salts 2-2.5, wherein said salt crystal exhibits an X-ray powder diffraction pattern comprising at least five peaks having 2-theta angle values selected from the group consisting of: 7.3, 12.1, 13.6, 15.6, and 18.5 degrees, wherein the XRPD pattern is measured in a diffractometer using copper anode, e.g., at wavelength alpha1 of 1.5406 Å and wavelength alpha2 of 1.5444 Å.
2.7 Any of Hydrochloride Salts 2-2.6, wherein said salt crystal exhibits an X-ray powder diffraction pattern comprising at least five peaks having d-spacing values selected from the group consisting of 12.2, 11.8, 7.3, 6.5, 5.7, 5.4, 4.8, 4.4, 4.2 4.1 Å.
2.8 Any of Hydrochloride Salts 2-2.7, wherein said salt crystal exhibits an X-ray powder diffraction pattern comprising peaks having d-spacing values selected from the group consisting of 12.2, 7.3, 6.5, 5.7, 4.8 Å.
2.9 Any of Hydrochloride Salts 2-2.8, wherein said salt crystal exhibits an X-ray powder diffraction pattern comprising at least five peaks having 2-theta angle values or comprising at least five peaks having d-spacing values selected from those set forth in Table 2 as defined herein, wherein the XRPD pattern is measured in a diffractometer using copper anode, at wavelength alpha1 of 1.5406 Å and wavelength alpha2 of 1.5444 Å.
2.10 Any of Hydrochloride Salts 2-2.9, wherein said salt crystal exhibits a Differential Scanning Calorimetry (DSC) pattern comprising an endothermic peak at about 140° C. to 142° C., e.g., at about 141° C., and/or between about 190° C. to 192° C., e.g., at about 191° C.
2.11 Any of Hydrochloride Salts 2-2.10, wherein the Compound A free base and hydrochloride counterion are present in a molar ratio of about 2:1 to about 1:2, e.g., about 1:1, e.g., about 1:0.9.

In a further embodiment, the present disclosure provides for a third hydrochloride salt [Hydrochloride Salt 3] of Compound A.
3.1 Hydrochloride Salt 3, which is in crystalline form.
3.2 Hydrochloride Salt 3 or 3.1, which is in solvate form.
3.3 Any of Hydrochloride Salts 3-3.2, wherein the salt crystal is in the form of a solvate with one or more of acetonitrile, ethyl acetate, acetone, 2-butanone, 2-ethyl-1-butanol, ethyl salicylate, ethyl butyl ketone, acetone, or combinations thereof.
3.4 Any of Hydrochloride Salts 3-3.3, wherein the salt crystal is an acetonitrile solvate.
3.5 Any of Hydrochloride Salts 3-3.4, wherein said salt crystal exhibits an X-ray powder diffraction pattern comprising at least five peaks having 2-theta angle values selected from the group consisting of: 4.9, 6.9, 7.3, 7.4, 12.2, 12.7, 14.6, 20.6, 27.6, and 32.7 degrees, wherein the XRPD pattern is measured in a diffractometer using copper anode, e.g., at wavelength alpha1 of 1.5406 Å and wavelength alpha2 of 1.5444 Å.
3.6 Any of Hydrochloride Salts 3-3.5, wherein said salt crystal exhibits an X-ray powder diffraction pattern comprising at least five peaks having 2-theta angle values selected from the group consisting of: 4.9, 7.3, 7.4, 12.2, and 27.6 degrees, wherein the XRPD pattern is measured in a diffractometer using copper anode, e.g., at wavelength alpha1 of 1.5406 Å and wavelength alpha2 of 1.5444 Å.
3.7 Any of Hydrochloride Salts 3-3.6, wherein said salt crystal exhibits an X-ray powder diffraction pattern comprising at least five peaks having d-spacing values selected from the group consisting of 18.0, 14.4, 12.8, 12.0, 7.3, 6.9, 6.1, 4.3, 3.2, and 2.7 Å.
3.8 Any of Hydrochloride Salts 3-3.7, wherein said salt crystal exhibits an X-ray powder diffraction pattern comprising peaks having d-spacing values selected from the group consisting of 18.0, 12.0, 7.3, 6.9, 3.2 Å.
3.9 Any of Hydrochloride Salts 3-3.8, wherein said salt crystal exhibits an X-ray powder diffraction pattern comprising at least five peaks having 2-theta angle values or comprising at least five peaks having d-spacing values selected from those set forth in Table 3 as defined herein, wherein the XRPD pattern is measured in a diffractometer using copper anode, at wavelength alpha1 of 1.5406 Å and wavelength alpha2 of 1.5444 Å.
3.10 Any of Hydrochloride Salts 3-3.9, wherein said salt crystal exhibits a Differential Scanning Calorimetry (DSC) pattern comprising an endothermic peak between about 155° C. and 157° C., e.g., at about 156° C., and/or between about 275° C. and 277° C., e.g., at about 276° C.
3.11 Any of Hydrochloride Salts 3-3.10, wherein the Compound A free base and hydrochloride counterion are present in a molar ratio of about 2:1 to about 1:2, e.g., about 1:1, e.g., about 1:0.9.

In a further embodiment, the present disclosure provides for a fourth hydrochloride salt [Hydrochloride Salt 4] of Compound A.
4.1 Hydrochloride Salt 4, which is in crystalline form.
4.2 Hydrochloride Salt 4 or 4.1, which is in solvate form.
4.3 Any of Hydrochloride Salts 4-4.2, wherein the salt crystal is in the form of a solvate with one or more of acetonitrile, ethyl acetate, acetone, 2-butanone, 2-ethyl-1-butanol, ethyl salicylate, ethyl butyl ketone, acetone, or combinations thereof.
4.4 Any of Hydrochloride Salts 4-4.3, wherein the salt crystal is a 2-butanone solvate.
4.5 Any of Hydrochloride Salts 4-4.4, wherein said salt crystal exhibits an X-ray powder diffraction pattern comprising at least five peaks having 2-theta angle values selected from the group consisting of: 7.6, 12.0, 12.7, 15.0, 15.1, 17.9, 18.8, 19.3, 23.1, and 24.0 degrees, wherein the XRPD pattern is measured in a diffractometer using copper anode, e.g., at wavelength alpha1 of 1.5406 Å and wavelength alpha2 of 1.5444 Å.
4.6 Any of Hydrochloride Salts 4-4.5, wherein said salt crystal exhibits an X-ray powder diffraction pattern comprising at least five peaks having 2-theta angle values selected from the group consisting of: 7.6, 12.0, 12.7, 18.8, and 23.1 degrees, wherein the XRPD pattern is measured in a diffractometer using copper anode, e.g., at wavelength alpha1 of 1.5406 Å and wavelength alpha2 of 1.5444 Å.
4.7 Any of Hydrochloride Salts 4-4.6, wherein said salt crystal exhibits an X-ray powder diffraction pattern comprising at least five peaks having d-spacing values selected from the group consisting of 11.7, 7.4, 7.0, 5.9, 5.8, 4.9, 4.7, 4.6, 3.8, and 3.7 Å.
4.8 Any of Hydrochloride Salts 4-4.7, wherein said salt crystal exhibits an X-ray powder diffraction pattern comprising peaks having d-spacing values selected from the group consisting of 11.7, 7.4, 7.0, 4.7, and 3.8 Å.
4.9 Any of Hydrochloride Salts 4-4.8, wherein said salt crystal exhibits an X-ray powder diffraction pattern comprising at least five peaks having 2-theta angle values or comprising at least five peaks having d-spacing values selected from those set forth in Table 4 as defined herein, wherein the XRPD pattern is measured in a diffractometer using copper anode, at wavelength alpha1 of 1.5406 Å and wavelength alpha2 of 1.5444 Å.
4.10 Any of Hydrochloride Salts 4-4.9, wherein said salt crystal exhibits a Differential Scanning Calorimetry (DSC) pattern comprising an endothermic peak between about 194° C. and 196° C., e.g., at about 195° C., and/or between about 209° C. and 211° C., e.g., at about 210° C.
4.11 Any of Hydrochloride Salts 4-4.10, wherein the Compound A free base and hydrochloride counterion are present in a molar ratio of about 2:1 to about 1:2, e.g., about 1:1, e.g., about 1:0.9.

In a further embodiment, the present disclosure provides for a fifth hydrochloride salt [Hydrochloride Salt 5] of Compound A.
5.1 Hydrochloride Salt 5, which is in crystalline form.
5.2 Hydrochloride Salt 5 or 5.1, which is in solvate form.
5.3 Any of Hydrochloride Salts 5-5.2, wherein the salt crystal is in the form of a solvate with one or more of acetonitrile, ethyl acetate, acetone, 2-butanone, 2-ethyl-1-butanol, ethyl salicylate, ethyl butyl ketone, acetone, or combinations thereof.
5.4 Any of Hydrochloride Salts 5-5.3, wherein the salt crystal is a 2-ethyl-1-butanol solvate.
5.5 Any of Hydrochloride Salts 5-5.4, wherein said salt crystal exhibits an X-ray powder diffraction pattern comprising at least five peaks having 2-theta angle values selected from the group consisting of: 6.7, 7.7, 8.8, 9.1, 11.4, 16.4, 17.0, 18.4, 21.9, and 24.1 degrees, wherein the XRPD pattern is measured in a diffractometer using copper anode, e.g., at wavelength alpha1 of 1.5406 Å and wavelength alpha2 of 1.5444 Å.
5.6 Any of Hydrochloride Salts 5-5.5, wherein said salt crystal exhibits an X-ray powder diffraction pattern comprising at least five peaks having 2-theta angle values selected from the group consisting of: 6.7, 7.7, 8.8, 9.1, and 16.4 degrees, wherein the XRPD pattern is measured in a diffractometer using copper anode, e.g., at wavelength alpha1 of 1.5406 Å and wavelength alpha2 of 1.5444 Å.
5.7 Any of Hydrochloride Salts 5-5.6, wherein said salt crystal exhibits an X-ray powder diffraction pattern comprising at least five peaks having d-spacing values selected from the group consisting of 13.2, 11.5, 10.0, 9.8, 7.8, 5.4, 5.2, 4.8, 4.1, and 3.7 Å.
5.8 Any of Hydrochloride Salts 5-5.7, wherein said salt crystal exhibits an X-ray powder diffraction pattern comprising peaks having d-spacing values selected from the group consisting of 13.2, 11.5, 10.0, 9.8, and 5.4 Å.
5.9 Any of Hydrochloride Salts 5-5.8, wherein said salt crystal exhibits an X-ray powder diffraction pattern comprising at least five peaks having 2-theta angle values or comprising at least five peaks having d-spacing values selected from those set forth in Table 52 as defined herein, wherein the XRPD pattern is measured in a diffractometer using copper anode, at wavelength alpha1 of 1.5406 Å and wavelength alpha2 of 1.5444 Å.
5.10 Any of Hydrochloride Salts 5-5.9, wherein said salt crystal exhibits a Differential Scanning Calorimetry (DSC) pattern comprising an endothermic peak between about 158° C. and 161° C., e.g., at about 159° C.
5.11 Any of Hydrochloride Salts 5-5.10, wherein the Compound A free base and hydrochloride counterion are present in a molar ratio of about 2:1 to about 1:2, e.g., about 1:1.

In a further embodiment, the present disclosure provides for a sixth hydrochloride salt [Hydrochloride Salt 6] of Compound A.
6.1 Hydrochloride Salt 6, which is in crystalline form.
6.2 Hydrochloride Salt 6 or 6.1, which is in solvate form.
6.3 Any of Hydrochloride Salts 6-6.2, wherein the salt crystal is in the form of a solvate with one or more of acetonitrile, ethyl acetate, acetone, 2-butanone, 2-ethyl-1-butanol, ethyl salicylate, ethyl butyl ketone, acetone, or combinations thereof.
6.4 Any of Hydrochloride Salts 6-6.3, wherein the salt crystal is an ethyl butyl ketone solvate.
6.5 Any of Hydrochloride Salts 6-6.4, wherein said salt crystal exhibits an X-ray powder diffraction pattern comprising at least five peaks having 2-theta angle values selected from the group consisting of: 5.0, 7.1, 7.5, 7.8, 8.5, 12.4, 13.0, 18.7, 18.8, and 20.8 degrees, wherein the XRPD pattern is measured in a diffractometer using copper anode, e.g., at wavelength alpha1 of 1.5406 Å and wavelength alpha2 of 1.5444 Å.
6.6 Any of Hydrochloride Salts 6-6.5, wherein said salt crystal exhibits an X-ray powder diffraction pattern comprising at least five peaks having 2-theta angle values selected from the group consisting of: 5.0, 7.1, 7.8, 12.4, and 18.7 degrees, wherein the XRPD pattern is measured in a diffractometer using copper anode, e.g., at wavelength alpha1 of 1.5406 Å and wavelength alpha2 of 1.5444 Å.
6.7 Any of Hydrochloride Salts 6-6.6, wherein said salt crystal exhibits an X-ray powder diffraction pattern comprising at least five peaks having d-spacing values selected from the group consisting of 17.8, 12.5, 11.7, 11.3, 10.4, 7.1, 6.8, 6.0, 4.7, and 4.3 Å.
6.8 Any of Hydrochloride Salts 6-6.7, wherein said salt crystal exhibits an X-ray powder diffraction pattern comprising peaks having d-spacing values selected from the group consisting of 17.8, 12.5, 11.3, 7.1, and 4.7 Å.
6.9 Any of Hydrochloride Salts 6-6.8, wherein said salt crystal exhibits an X-ray powder diffraction pattern comprising at least five peaks having 2-theta angle values or comprising at least five peaks having d-spacing values selected from those set forth in Table 53 as defined herein, wherein the XRPD pattern is measured in a diffractometer using copper anode, at wavelength alpha1 of 1.5406 Å and wavelength alpha2 of 1.5444 Å.
6.10 Any of Hydrochloride Salts 6-6.9, wherein said salt crystal exhibits a Differential Scanning Calorimetry (DSC) pattern comprising an endothermic peak between about 129° C. and 133° C., e.g., at about 131° C.
6.11 Any of Hydrochloride Salts 6-6.10, wherein the Compound A free base and hydrochloride counterion are present in a molar ratio of about 2:1 to about 1:2, e.g., about 1:1.

In a further embodiment, the present disclosure provides for a seventh hydrochloride salt [Hydrochloride Salt 7] of Compound A.
7.1 Hydrochloride Salt 7, which is in crystalline form.
7.2 Hydrochloride Salt 7 or 7.1, which is in solvate form.
7.3 Any of Hydrochloride Salts 7-7.2, wherein the salt crystal is in the form of a solvate with one or more of acetonitrile, ethyl acetate, acetone, 2-butanone, 2-ethyl-1-butanol, ethyl salicylate, ethyl butyl ketone, acetone, or combinations thereof.
7.4 Any of Hydrochloride Salts 7-7.3, wherein the salt crystal is an anisole solvate.
7.5 Any of Hydrochloride Salts 7-7.4, wherein said salt crystal exhibits an X-ray powder diffraction pattern comprising at least five peaks having 2-theta angle values selected from the group consisting of: 5.6, 8.7, 6.1, 9.2, 9.8, 10.7, 10.9, 18.9, 21.8, and 22.0 degrees, wherein the XRPD pattern is measured in a diffractometer using copper anode, e.g., at wavelength alpha1 of 1.5406 Å and wavelength alpha2 of 1.5444 Å.
7.6 Any of Hydrochloride Salts 7-7.5, wherein said salt crystal exhibits an X-ray powder diffraction pattern comprising at least five peaks having 2-theta angle values selected from the group consisting of: 5.6, 8.7, 9.8, 18.9, and 22.0 degrees, wherein the XRPD pattern is measured in a diffractometer using copper anode, e.g., at wavelength alpha1 of 1.5406 Å and wavelength alpha2 of 1.5444 Å.
7.7 Any of Hydrochloride Salts 7-7.6, wherein said salt crystal exhibits an X-ray powder diffraction pattern comprising at least five peaks having d-spacing values selected from the group consisting of 15.7, 14.4, 14.6, 10.2, 9.6, 9.0, 8.1, 4.7, 4.0, and 4.1 Å.
7.8 Any of Hydrochloride Salts 7-7.7, wherein said salt crystal exhibits an X-ray powder diffraction pattern comprising peaks having d-spacing values selected from the group consisting of 15.7, 10.2, 9.0, 4.7, and 4.0Å.
7.9 Any of Hydrochloride Salts 7-7.8, wherein said salt crystal exhibits an X-ray powder diffraction pattern comprising at least five peaks having 2-theta angle values or comprising at least five peaks having d-spacing values selected from those set forth in Table 54 as defined herein, wherein the XRPD pattern is measured in a diffractometer using copper anode, at wavelength alpha1 of 1.5406 Å and wavelength alpha2 of 1.5444 Å.
7.10 Any of Hydrochloride Salts 7-7.9, wherein said salt crystal exhibits a Differential Scanning Calorimetry (DSC) pattern comprising an endothermic peak between about 144° C. and 147° C., e.g., at about 145° C.
7.11 Any of Hydrochloride Salts 7-7.10, wherein the Compound A free base and hydrochloride counterion are present in a molar ratio of about 2:1 to about 1:2, e.g., about 1:1.

In a further embodiment, the present disclosure provides for an eighth hydrochloride salt [Hydrochloride Salt 8] of Compound A.
8.1 Hydrochloride Salt 8, which is in crystalline form.
8.2 Hydrochloride Salt 8 or 8.1, which is in solvate form.
8.3 Any of Hydrochloride Salts 8-8.2, wherein the salt crystal is in the form of a solvate with one or more of acetonitrile, ethyl acetate, acetone, 2-butanone, 2-ethyl-1-butanol, ethyl salicylate, ethyl butyl ketone, acetone, or combinations thereof.
8.4 Any of Hydrochloride Salts 8-8.3, wherein the salt crystal is an ethyl salicylate solvate.
8.5 Any of Hydrochloride Salts 8-8.4, wherein said salt crystal exhibits an X-ray powder diffraction pattern comprising at least five peaks having 2-theta angle values selected from the group consisting of: 5.7, 11.4, 11.6, 12.5, 18.9, 19.2, 20.2, 20.4, 20.6, and 22.1 degrees, wherein the XRPD pattern is measured in a diffractometer using copper anode, e.g., at wavelength alpha1 of 1.5406 Å and wavelength alpha2 of 1.5444 Å.
8.6 Any of Hydrochloride Salts 8-8.5, wherein said salt crystal exhibits an X-ray powder diffraction pattern comprising at least five peaks having 2-theta angle values selected from the group consisting of: 5.7, 11.4, 11.6, 20.6, and 22.1 degrees, wherein the XRPD pattern is measured in a diffractometer using copper anode, e.g., at wavelength alpha1 of 1.5406 Å and wavelength alpha2 of 1.5444 Å.
8.7 Any of Hydrochloride Salts 8-8.6, wherein said salt crystal exhibits an X-ray powder diffraction pattern comprising at least five peaks having d-spacing values selected from the group consisting of 15.4, 7.8, 7.6, 7.1, 5.2, 4.7, 4.6, 4.4, 4.3, and 4.0 Å.
8.8 Any of Hydrochloride Salts 8-8.7, wherein said salt crystal exhibits an X-ray powder diffraction pattern comprising peaks having d-spacing values selected from the group consisting of 15.4, 7.8, 7.6, 4.3, and 4.0 Å.
8.9 Any of Hydrochloride Salts 8-8.8, wherein said salt crystal exhibits an X-ray powder diffraction pattern comprising at least five peaks having 2-theta angle values or comprising at least five peaks having d-spacing values selected from those set forth in Table 55 as defined herein, wherein the XRPD pattern is measured in a diffractometer using copper anode, at wavelength alpha1 of 1.5406 Å and wavelength alpha2 of 1.5444 Å.
8.10 Any of Hydrochloride Salts 8-8.9, wherein said salt crystal exhibits a Differential Scanning Calorimetry (DSC) pattern comprising an endothermic peak between about 196° C. and 200° C., e.g., at about 198° C.
8.11 Any of Hydrochloride Salts 8-8.10, wherein the Compound A free base and hydrochloride counterion are present in a molar ratio of about 2:1 to about 1:2, e.g., about 1:1.

In a further embodiment, the present disclosure provides for a malate salt [Malate Salt 1] of Compound A.
1.1 Malate Salt 1, which is in crystalline form.
1.2 Malate Salt 1 or 1.1, which is in solvate form.
1.3 Any of Malate Salts 1-1.2, wherein the salt crystal is an ethyl acetate solvate.
1.4 Any of Malate Salts 1-1.3, wherein said salt crystal exhibits an X-ray powder diffraction pattern comprising at least five peaks having 2-theta angle values selected from the group consisting of: 5.9, 7.2, 12.0, 16.0, 17.7, 17.8, 20.9, 21.2, 21.7, and 21.8 degrees, wherein the XRPD pattern is measured in a diffractometer using copper anode, e.g., at wavelength alpha1 of 1.5406 Å and wavelength alpha2 of 1.5444 Å.
1.5 Any of Malate Salts 1-1.4, wherein said salt crystal exhibits an X-ray powder diffraction pattern comprising at least five peaks having 2-theta angle values selected from the group consisting of: 5.9, 16.0, 17.8, 21.7, and 21.8 degrees, wherein the XRPD pattern is measured in a diffractometer using copper anode, e.g., at wavelength alpha1 of 1.5406 Å and wavelength alpha2 of 1.5444 Å.
1.6 Any of Malate Salts 1-1.5, wherein said salt crystal exhibits an X-ray powder diffraction pattern comprising at least five peaks having d-spacing values selected from the group consisting of 15.0, 12.3, 7.4, 5.5, 5.0, 4.5, 4.3, 4.2, 4.1, and 3.1 Å.
1.7 Any of Malate Salts 1-1.6, wherein said salt crystal exhibits an X-ray powder diffraction pattern comprising peaks having d-spacing values selected from the group consisting of 15.0, 5.5, 5.0, 4.2, and 4.1 Å.
1.8 Any of Malate Salts 1-1.7, wherein said salt crystal exhibits an X-ray powder diffraction pattern comprising at least five peaks having 2-theta angle values or comprising at least five peaks having d-spacing values selected from those set forth in Table 5 as defined herein, wherein the XRPD pattern is measured in a diffractometer using copper anode, at wavelength alpha1 of 1.5406 Å and wavelength alpha2 of 1.5444 Å.
1.9 Any of Malate Salts 1-1.8, wherein said salt crystal exhibits a Differential Scanning Calorimetry (DSC) pattern comprising an endothermic peak between about 94° C. and 96° C., e.g., at about 95° C.
1.10 Any of Malate Salts 1-1.9, wherein the Compound A free base and malic acid counterion are present in a molar ratio of about 2:1 to about 1:2, e.g., about 1:1, e.g., about 1:0.7.

In a further embodiment, the present disclosure provides for a fumarate salt [Fumarate Salt 1] of Compound A.
1.1 Fumarate Salt 1, which is in crystalline form.
1.2 Fumarate Salt 1 or 1.1, which is in solvate form.
1.3 Any of Fumarate Salts 1-1.2, wherein the salt crystal is an ethyl acetate solvate.
1.4 Any of Fumarate Salts 1-1.3, wherein said salt crystal exhibits an X-ray powder diffraction pattern comprising at least five peaks having 2-theta angle values selected from the group consisting of: 5.9, 7.4, 11.8, 12.6, 13.8, 17.2, 18.9, 20.6, 21.7, and 21.5 degrees, wherein the XRPD pattern is measured in a diffractometer using copper anode, e.g., at wavelength alpha1 of 1.5406 Å and wavelength alpha2 of 1.5444 Å.
1.5 Any of Fumarate Salts 1-1.4, wherein said salt crystal exhibits an X-ray powder diffraction pattern comprising at least five peaks having 2-theta angle values selected from the group consisting of: 5.9, 7.4, 13.8, 17.2, and 21.7 degrees, wherein the XRPD pattern is measured in a diffractometer using copper anode, e.g., at wavelength alpha1 of 1.5406 Å and wavelength alpha2 of 1.5444 Å.
1.6 Any of Fumarate Salts 1-1.5, wherein said salt crystal exhibits an X-ray powder diffraction pattern comprising at least five peaks having d-spacing values selected from the group consisting of 14.9, 12.0, 7.5, 7.0, 6.4, 5.6, 4.7, 4.3, and 4.1 Å.
1.7 Any of Fumarate Salts 1-1.6, wherein said salt crystal exhibits an X-ray powder diffraction pattern comprising peaks having d-spacing values selected from the group consisting of 14.9, 12.0, 6.4, 5.6, and 4.1 Å.
1.8 Any of Fumarate Salts 1-1.7, wherein said salt crystal exhibits an X-ray powder diffraction pattern comprising at least five peaks having 2-theta angle values or comprising at least five peaks having d-spacing values selected from those set forth in Table 6 as defined herein, wherein the XRPD pattern is measured in a diffractometer using copper anode, at wavelength alpha1 of 1.5406 Å and wavelength alpha2 of 1.5444 Å.
1.9 Any of Fumarate Salts 1-1.8, wherein said salt crystal exhibits a Differential Scanning Calorimetry (DSC) pattern comprising an endothermic peak between about 110° C. and 112° C., e.g., at about 111° C., and/or between about 141° C. and 143° C., e.g., at about 142° C.
1.10 Any of Fumarate Salts 1-1.9, wherein the Compound A free base and fumaric acid counterion are present in a molar ratio of about 2:1 to about 1:2, e.g., about 1:1, e.g., about 1:0.5.

In a further embodiment, the present disclosure provides for a first sulfate salt [Sulfate Salt 1] of Compound A.
1.1 Sulfate Salt 1, which is in crystalline form.
1.2 Sulfate Salt 1 or 1.1, which is in solvate form.
1.3 Any of Sulfate Salts 1-1.2, wherein the salt crystal is an ethyl acetate solvate.
1.4 Any of Sulfate Salts 1-1.3, wherein said salt crystal exhibits an X-ray powder diffraction pattern comprising at least five peaks having 2-theta angle values selected from the group consisting of: 4.9, 5.9, 6.5, 8.0, 8.5, 12.3, 12.5, 18.0, 20.8, and 22.7 degrees, wherein the XRPD pattern is measured in a diffractometer using copper anode, e.g., at wavelength alpha1 of 1.5406 Å and wavelength alpha2 of 1.5444 Å.
1.5 Any of Sulfate Salts 1-1.4, wherein said salt crystal exhibits an X-ray powder diffraction pattern comprising at least five peaks having 2-theta angle values selected from the group consisting of: 4.9, 5.9, 6.5, 8.0, and 22.7 degrees, wherein the XRPD pattern is measured in a diffractometer using copper anode, e.g., at wavelength alpha1 of 1.5406 Å and wavelength alpha2 of 1.5444 Å.
1.6 Any of Sulfate Salts 1-1.5, wherein said salt crystal exhibits an X-ray powder diffraction pattern comprising at least five peaks having d-spacing values selected from the group consisting of 18.1, 15.1, 13.6, 11.1, 10.5, 7.2, 7.1, 4.9, 4.3, and 3.9 Å.
1.7 Any of Sulfate Salts 1-1.6, wherein said salt crystal exhibits an X-ray powder diffraction pattern comprising peaks having d-spacing values selected from the group consisting of 18.1, 15.1, 13.6, 11.1, and 3.9 Å.
1.8 Any of Sulfate Salts 1-1.7, wherein said salt crystal exhibits an X-ray powder diffraction pattern comprising at least five peaks having 2-theta angle values or comprising at least five peaks having d-spacing values selected from those set forth in Table 7 as defined herein, wherein the XRPD pattern is measured in a diffractometer using copper anode, at wavelength alpha1 of 1.5406 Å and wavelength alpha2 of 1.5444 Å.
1.9 Any of Sulfate Salts 1-1.8, wherein said salt crystal exhibits a Differential Scanning Calorimetry (DSC) pattern comprising an endothermic peak between about 132° C. and 134° C., e.g., at about 133° C., and/or between about 227° C. and 229° C., e.g., at about 228° C.
1.10 Any of Sulfate Salts 1-1.9, wherein the Compound A free base and sulfuric acid counterion are present in a molar ratio of about 2:1 to about 1:2, e.g., about 1:1, e.g., about 1:0.4.

In a further embodiment, the present disclosure provides for a second sulfate salt [Sulfate Salt 2] of Compound A.
2.1 Sulfate Salt 2, which is in crystalline form.
2.2 Sulfate Salt 2 or 2.1, which is in solvate form.
2.3 Any of Sulfate Salts 2-2.2, wherein the salt crystal is an ethyl acetate solvate.
2.4 Any of Sulfate Salts 2-2.3, wherein said salt crystal exhibits an X-ray powder diffraction pattern comprising at least five peaks having 2-theta angle values selected from the group consisting of: 4.9, 5.9, 8.4, 9.6, 10.7, 14.8, 17.9, 19.6, 20.8, and 22.8 degrees, wherein the XRPD pattern is measured in a diffractometer using copper anode, e.g., at wavelength alpha1 of 1.5406 Å and wavelength alpha2 of 1.5444 Å.
2.5 Any of Sulfate Salts 2-2.4, wherein said salt crystal exhibits an X-ray powder diffraction pattern comprising at least five peaks having 2-theta angle values selected from the group consisting of: 4.9, 5.9, 8.4, 10.7, and 17.9 degrees, wherein the XRPD pattern is measured in a diffractometer using copper anode, e.g., at wavelength alpha1 of 1.5406 Å and wavelength alpha2 of 1.5444 Å.
2.6 Any of Sulfate Salts 2-2.5, wherein said salt crystal exhibits an X-ray powder diffraction pattern comprising at least five peaks having d-spacing values selected from the group consisting of 18.1, 15.0, 10.6, 9.2, 8.3, 6.0, 5.0, 4.5, 4.3, and 3.9 Å.
2.7 Any of Sulfate Salts 2-2.6, wherein said salt crystal exhibits an X-ray powder diffraction pattern comprising peaks having d-spacing values selected from the group consisting of 18.1, 15.0, 10.6, 8.3, 5.0 Å.
2.8 Any of Sulfate Salts 2-2.7, wherein said salt crystal exhibits an X-ray powder diffraction pattern comprising at least five peaks having 2-theta angle values or comprising at least five peaks having d-spacing values selected from those set forth in Table 8 as defined herein, wherein the XRPD pattern is measured in a diffractometer using copper anode, at wavelength alpha1 of 1.5406 Å and wavelength alpha2 of 1.5444 Å.
2.9 Any of Sulfate Salts 2-2.8, wherein said salt crystal exhibits a Differential Scanning Calorimetry (DSC) pattern comprising an endothermic peak between about 69° C. and 71° C., e.g., at about 70° C., and/or between about 114° C. and 116° C., e.g., at about 115° C.
2.10 Any of Sulfate Salts 2-2.9, wherein the Compound A free base and sulfuric acid counterion are present in a molar ratio of about 2:1 to about 1:2, e.g., about 1:1, e.g., about 1:0.5.

In a further embodiment, the present disclosure provides for an esylate salt [Esylate Salt 1] of Compound A.
1.1 Esylate Salt 1, which is in crystalline form.
1.2 Esylate Salt 1 or 1.1, which is in solvate form.
1.3 Any of Esylate Salts 1-1.2, wherein the salt crystal is an acetone solvate.
1.4 Any of Esylate Salts 1-1.3, wherein said salt crystal exhibits an X-ray powder diffraction pattern comprising at least five peaks having 2-theta angle values selected from the group consisting of: 6.2, 11.7, 17.0, 18.7, 19.1, 19.3, 20.3, 20.5, 22.8, and 23.4 degrees, wherein the XRPD pattern is measured in a diffractometer using copper anode, e.g., at wavelength alpha1 of 1.5406 Å and wavelength alpha2 of 1.5444 Å.
1.5 Any of Esylate Salts 1-1.4, wherein said salt crystal exhibits an X-ray powder diffraction pattern comprising at least five peaks having 2-theta angle values selected from the group consisting of: 6.2, 19.3, 20.3, 20.5, and 22.8 degrees, wherein the XRPD pattern is measured in a diffractometer using copper anode, e.g., at wavelength alpha1 of 1.5406 Å and wavelength alpha2 of 1.5444 Å.
1.6 Any of Esylate Salts 1-1.5, wherein said salt crystal exhibits an X-ray powder diffraction pattern comprising at least five peaks having d-spacing values selected from the group consisting of 14.3, 7.6, 5.2, 4.7, 4.6, 4.4, 4.3, 3.9, 3.8, and 3.7 Å.
1.7 Any of Esylate Salts 1-1.6, wherein said salt crystal exhibits an X-ray powder diffraction pattern comprising peaks having d-spacing values selected from the group consisting of 14.3, 4.6, 4.4, 4.3, and 3.9 Å.
1.8 Any of Esylate Salts 1-1.7, wherein said salt crystal exhibits an X-ray powder diffraction pattern comprising at least five peaks having 2-theta angle values or comprising at least five peaks having d-spacing values selected from those set forth in Table 9 as defined herein, wherein the XRPD pattern is measured in a diffractometer using copper anode, at wavelength alpha1 of 1.5406 Å and wavelength alpha2 of 1.5444 Å.
1.9 Any of Esylate Salts 1-1.8, wherein said salt crystal exhibits a Differential Scanning Calorimetry (DSC) pattern comprising an endothermic peak between about 304° C. and 306° C., e.g., at about 305° C.
1.10 Any of Esylate Salts 1-1.9, wherein the Compound A free base and ethane sulfonic acid counterion are present in a molar ratio of about 2:1 to about 1:2, e.g., about 1:1.

In a further embodiment, the present disclosure provides for a galactarate salt [Galactarate Salt 1] of Compound A.
1.1 Galactarate Salt 1, which is in crystalline form.
1.2 Galactarate Salt 1 or 1.1, which is in solvate form.
1.3 Any of Galactarate Salts 1-1.2, wherein the salt crystal is a methanol solvate.
1.4 Any of Galactarate Salts 1-1.3, wherein said salt crystal exhibits an X-ray powder diffraction pattern comprising at least five peaks having 2-theta angle values selected from the group consisting of: 18.2, 19.7, 21.5, 26.8, 30.7, 34.5, 36.7, 36.8, 37.6, and 37.7 degrees, wherein the XRPD pattern is measured in a diffractometer using copper anode, e.g., at wavelength alpha1 of 1.5406 Å and wavelength alpha2 of 1.5444 Å.
1.5 Any of Galactarate Salts 1-1.4, wherein said salt crystal exhibits an X-ray powder diffraction pattern comprising at least five peaks having 2-theta angle values selected from the group consisting of: 19.7, 30.7, 34.5, 36.7, and 37.6 degrees, wherein the XRPD pattern is measured in a diffractometer using copper anode, e.g., at wavelength alpha1 of 1.5406 Å and wavelength alpha2 of 1.5444 Å.
1.6 Any of Galactarate Salts 1-1.5, wherein said salt crystal exhibits an X-ray powder diffraction pattern comprising at least five peaks having d-spacing values selected from the group consisting of 6.8, 4.9, 4.5, 4.1, 3.4, 3.3, 2.9, 2.6, 2.4, and 2.2 Å.
1.7 Any of Galactarate Salts 1-1.6, wherein said salt crystal exhibits an X-ray powder diffraction pattern comprising peaks having d-spacing values selected from the group consisting of 4.5, 2.9, 2.6, 2.4, and 3.3 Å.
1.8 Any of Galactarate Salts 1-1.7, wherein said salt crystal exhibits an X-ray powder diffraction pattern comprising at least five peaks having 2-theta angle values or comprising at least five peaks having d-spacing values selected from those set forth in Table 10 as defined herein, wherein the XRPD pattern is measured in a diffractometer using copper anode, at wavelength alpha1 of 1.5406 Å and wavelength alpha2 of 1.5444 Å.
1.9 Any of Galactarate Salts 1-1.8, wherein said salt crystal exhibits a Differential Scanning Calorimetry (DSC) pattern comprising an endothermic peak between about 204° C. and 206° C., e.g., at about 205° C.
1.10 Any of Galactarate Salts 1-1.9, wherein the Compound A free base and galactaric acid counterion are present in a molar ratio of about 2:1 to about 1:2, e.g., about 1:1, e.g., about 1:0.9.

In a further embodiment, the present disclosure provides for a first adipate salt [Adipate Salt 1] of Compound A.
1.1 Adipate Salt 1, which is in crystalline form.
1.2 Adipate Salt 1 or 1.1, which is in anhydrous form.
1.3 Any of Adipate Salts 1-1.1, wherein the salt crystal is an ethyl acetate solvate.
1.4 Any of Adipate Salts 1-1.3, wherein said salt crystal exhibits an X-ray powder diffraction pattern comprising at least five peaks having 2-theta angle values selected from the group consisting of: 5.0, 7.2, 10.0, 15.4, 16.3, 16.6, 17.8, 20.5, 22.6, and 23.9 degrees, wherein the XRPD pattern is measured in a diffractometer using copper anode, e.g., at wavelength alpha1 of 1.5406 Å and wavelength alpha2 of 1.5444 Å.
1.5 Any of Adipate Salts 1-1.4, wherein said salt crystal exhibits an X-ray powder diffraction pattern comprising at least five peaks having 2-theta angle values selected from the group consisting of: 7.2, 10.0, 16.3, 17.8, and 23.9 degrees, wherein the XRPD pattern is measured in a diffractometer using copper anode, e.g., at wavelength alpha1 of 1.5406 Å and wavelength alpha2 of 1.5444 Å.
1.6 Any of Adipate Salts 1-1.5, wherein said salt crystal exhibits an X-ray powder diffraction pattern comprising at least five peaks having d-spacing values selected from the group consisting of 17.8, 12.2, 8.8, 5.7, 5.4, 5.3, 5.0, 4.3, 3.9, and 3.7 Å.
1.7 Any of Adipate Salts 1-1.6, wherein said salt crystal exhibits an X-ray powder diffraction pattern comprising peaks having d-spacing values selected from the group consisting of 12.2, 8.8, 5.4, 5.0, and 3.7 Å.
1.8 Any of Adipate Salts 1-1.7, wherein said salt crystal exhibits an X-ray powder diffraction pattern comprising at least five peaks having 2-theta angle values or comprising at least five peaks having d-spacing values selected from those set forth in Table 11 as defined herein, wherein the XRPD pattern is measured in a diffractometer using copper anode, at wavelength alpha1 of 1.5406 Å and wavelength alpha2 of 1.5444 Å.
1.9 Any of Adipate Salts 1-1.8, wherein said salt crystal exhibits a Differential Scanning Calorimetry (DSC) pattern comprising an endothermic peak between about 119° C. and 121° C., e.g., at about 120° C., and/or between about 159° C. and 161° C., e.g., at about 160° C.
1.10 Any of Adipate Salts 1-1.9, wherein the Compound A free base and adipic acid counterion are present in a molar ratio of about 2:1 to about 1:2, e.g., about 1:1.

In a further embodiment, the present disclosure provides for a second adipate salt [Adipate Salt 2] of Compound A.
2.1 Adipate Salt 2, which is in crystalline form.
2.2 Adipate Salt 2 or 2.1, which is in anhydrous form.
2.3 Any of Adipate Salts 2-2.1, wherein the salt crystal is an acetonitrile solvate.
2.4 Any of Adipate Salts 2-2.3, wherein said salt crystal exhibits an X-ray powder diffraction pattern comprising at least five peaks having 2-theta angle values selected from the group consisting of: 4.9, 6.7, 8.9, 10.7, 15.7, 16.9, 17.8, 18.0, 21.3, and 22.3 degrees, wherein the XRPD pattern is measured in a diffractometer using copper anode, e.g., at wavelength alpha1 of 1.5406 Å and wavelength alpha2 of 1.5444 Å.
2.5 Any of Adipate Salts 2-2.4, wherein said salt crystal exhibits an X-ray powder diffraction pattern comprising at least five peaks having 2-theta angle values selected from the group consisting of: 4.9, 6.7, 8.9, 10.7, and 18.0 degrees, wherein the XRPD pattern is measured in a diffractometer using copper anode, e.g., at wavelength alpha1 of 1.5406 Å and wavelength alpha2 of 1.5444 Å.
2.6 Any of Adipate Salts 2-2.5, wherein said salt crystal exhibits an X-ray powder diffraction pattern comprising at least five peaks having d-spacing values selected from the group consisting of 18.1, 13.2, 9.9, 8.3, 5.6, 5.3, 5.0, 4.9, 4.2, and 4.0 Å.
2.7 Any of Adipate Salts 2-2.6, wherein said salt crystal exhibits an X-ray powder diffraction pattern comprising peaks having d-spacing values selected from the group consisting of 18.1, 13.2, 9.9, 8.3, and 4.9 Å.
2.8 Any of Adipate Salts 2-2.7, wherein said salt crystal exhibits an X-ray powder diffraction pattern comprising at least five peaks having 2-theta angle values or comprising at least five peaks having d-spacing values selected from those set forth in Table 12 as defined herein, wherein the XRPD pattern is measured in a diffractometer using copper anode, at wavelength alpha1 of 1.5406 Å and wavelength alpha2 of 1.5444 Å.
2.9 Any of Adipate Salts 2-2.8, wherein said salt crystal exhibits a Differential Scanning Calorimetry (DSC) pattern comprising an endothermic peak between about 159° C. and 161° C., e.g., at about 160° C.
2.10 Any of Adipate Salts 2-2.9, wherein the Compound A free base and adipic acid counterion are present in a molar ratio of about 4:1 to about 1:2, e.g., about 2:1, e.g., about 1:1.

In a further embodiment, the present disclosure provides for a third adipate salt [Adipate Salt 3] of Compound A.
3.1 Adipate Salt 3, which is in crystalline form.
3.2 Adipate Salt 3 or 3.1, which is in anhydrous form.
3.3 Any of Adipate Salts 3-3.1, wherein the salt crystal is an acetone solvate.
3.4 Any of Adipate Salts 3-3.3, wherein said salt crystal exhibits an X-ray powder diffraction pattern comprising at least five peaks having 2-theta angle values selected from the group consisting of: 6.5, 8.1, 10.5, 11.1, 12.2, 12.9, 18.2, 21.5, 23.9, and 24.4 degrees, wherein the XRPD pattern is measured in a diffractometer using copper anode, e.g., at wavelength alpha1 of 1.5406 Å and wavelength alpha2 of 1.5444 Å.
3.5 Any of Adipate Salts 3-3.4, wherein said salt crystal exhibits an X-ray powder diffraction pattern comprising at least five peaks having 2-theta angle values selected from the group consisting of: 6.5, 10.5, 12.2, 18.2, and 24.4 degrees, wherein the XRPD pattern is measured in a diffractometer using copper anode, e.g., at wavelength alpha1 of 1.5406 Å and wavelength alpha2 of 1.5444 Å.
3.6 Any of Adipate Salts 3-3.5, wherein said salt crystal exhibits an X-ray powder diffraction pattern comprising at least five peaks having d-spacing values selected from the group consisting of 13.6, 10.9, 8.4, 8.0, 7.2, 6.8, 4.9, 4.1, 3.7, and 3.6 Å.
3.7 Any of Adipate Salts 3-3.6, wherein said salt crystal exhibits an X-ray powder diffraction pattern comprising peaks having d-spacing values selected from the group consisting of 13.6, 8.4, 7.2, 4.9, and 3.6 Å.
3.8 Any of Adipate Salts 3-3.7, wherein said salt crystal exhibits an X-ray powder diffraction pattern comprising at least five peaks having 2-theta angle values or comprising at least five peaks having d-spacing values selected from those set forth in Table 12 Å as defined herein, wherein the XRPD pattern is measured in a diffractometer using copper anode, at wavelength alpha1 of 1.5406 Å and wavelength alpha2 of 1.5444 Å.
3.9 Any of Adipate Salts 3-3.8, wherein said salt crystal exhibits a Differential Scanning Calorimetry (DSC) pattern comprising an endothermic peak between about 109° C. and 112° C., e.g., at about 109° C.
3.10 Any of Adipate Salts 3-3.9, wherein the Compound A free base and adipic acid counterion are present in a molar ratio of about 2:1 to about 1:2, e.g., about 1:1.

In a further embodiment, the present disclosure provides for a lactate salt [Lactate Salt 1] of Compound A.
1.1 Lactate Salt 1, which is in crystalline form.
1.2 Lactate Salt 1 or 1.1, which is in anhydrous form.
1.3 Any of Lactate Salts 1-1.1, wherein the salt crystal is an ethyl acetate solvate or toluene solvate.
1.4 Any of Lactate Salts 1-1.3, wherein said salt crystal exhibits an X-ray powder diffraction pattern comprising at least five peaks having 2-theta angle values selected from the group consisting of: 6.4, 6.5, 9.0, 11.9, 12.3, 17.1, 19.4, 20.5, 23.3, and 23.2 degrees, wherein the XRPD pattern is measured in a diffractometer using copper anode, e.g., at wavelength alpha1 of 1.5406 Å and wavelength alpha2 of 1.5444 Å.
1.5 Any of Lactate Salts 1-1.4, wherein said salt crystal exhibits an X-ray powder diffraction pattern comprising at least five peaks having 2-theta angle values selected from the group consisting of: 6.4, 6.5, 11.9, 12.3, and 20.5 degrees, wherein the XRPD pattern is measured in a diffractometer using copper anode, e.g., at wavelength alpha1 of 1.5406 Å and wavelength alpha2 of 1.5444 Å.
1.6 Any of Lactate Salts 1-1.5, wherein said salt crystal exhibits an X-ray powder diffraction pattern comprising at least five peaks having d-spacing values selected from the group consisting of 14.0, 13.8, 13.7, 9.8, 7.5, 7.2, 5.2, 4.6, 4.3, and 3.8 Å.
1.7 Any of Lactate Salts 1-1.6, wherein said salt crystal exhibits an X-ray powder diffraction pattern comprising peaks having d-spacing values selected from the group consisting of 13.8, 13.7, 7.5, 7.2, and 4.3 Å.
1.8 Any of Lactate Salts 1-1.7, wherein said salt crystal exhibits an X-ray powder diffraction pattern comprising at least five peaks having 2-theta angle values or comprising at least five peaks having d-spacing values selected from those set forth in Table 13 as defined herein, wherein the XRPD pattern is measured in a diffractometer using copper anode, at wavelength alpha1 of 1.5406 Å and wavelength alpha2 of 1.5444 Å.
1.9 Any of Lactate Salts 1-1.8, wherein said salt crystal exhibits a Differential Scanning Calorimetry (DSC) pattern comprising an endothermic peak between about 187° C. and 190° C., e.g., at about 187° C. or 188° C.
1.10 Any of Lactate Salts 1-1.9, wherein the Compound A free base and lactic acid counterion are present in a molar ratio of about 2:1 to about 1:2, e.g., about 1:1.

In a further embodiment, the present disclosure provides for a first oxalate salt [Oxalate Salt 1] of Compound A.
1.1 Oxalate Salt 1, which is in crystalline form.
1.2 Oxalate Salt 1 or 1.1, which is in anhydrous form.
1.3 Any of Oxalate Salts 1-1.1, wherein the salt crystal is a 3-heptanone solvate.
1.4 Any of Oxalate Salts 1-1.3, wherein said salt crystal exhibits an X-ray powder diffraction pattern comprising at least five peaks having 2-theta angle values selected from the group consisting of: 7.1, 8.5, 12.2, 12.3, 16.3, 19.2, 20.7, 22.9, 24.1, and 25.4 degrees, wherein the XRPD pattern is measured in a diffractometer using copper anode, e.g., at wavelength alpha1 of 1.5406 Å and wavelength alpha2 of 1.5444 Å.
1.5 Any of Oxalate Salts 1-1.4, wherein said salt crystal exhibits an X-ray powder diffraction pattern comprising at least five peaks having 2-theta angle values selected from the group consisting of: 8.5, 12.2, 12.3, 16.3, and 20.7 degrees, wherein the XRPD pattern is measured in a diffractometer using copper anode, e.g., at wavelength alpha1 of 1.5406 Å and wavelength alpha2 of 1.5444 Å.
1.6 Any of Oxalate Salts 1-1.5, wherein said salt crystal exhibits an X-ray powder diffraction pattern comprising at least five peaks having d-spacing values selected from the group consisting of 12.4, 10.4, 7.2, 5.4, 4.8, 4.6, 4.3, 3.9, 3.7, and 3.5 Å.
1.7 Any of Oxalate Salts 1-1.6, wherein said salt crystal exhibits an X-ray powder diffraction pattern comprising peaks having d-spacing values selected from the group consisting of 10.4, 7.2, 5.4, 4.6, and 4.3 Å.
1.8 Any of Oxalate Salts 1-1.7, wherein said salt crystal exhibits an X-ray powder diffraction pattern comprising at least five peaks having 2-theta angle values or comprising at least five peaks having d-spacing values selected from those set forth in Table 14 as defined herein, wherein the XRPD pattern is measured in a diffractometer using copper anode, at wavelength alpha1 of 1.5406 Å and wavelength alpha2 of 1.5444 Å.
1.9 Any of Oxalate Salts 1-1.8, wherein said salt crystal exhibits a Differential Scanning Calorimetry (DSC) pattern comprising an endothermic peak between about 218° C. and 220° C., e.g., at about 219° C.
1.10 Any of Oxalate Salts 1-1.9, wherein the Compound A free base and oxalic acid counterion are present in a molar ratio of about 2:1 to about 1:2, e.g., about 1:1.

In a further embodiment, the present disclosure provides for a second oxalate salt [Oxalate Salt 2] of Compound A.
2.1 Oxalate Salt 2, which is in crystalline form.
2.2 Oxalate Salt 2 or 2.1, which is in anhydrous form.
2.3 Any of Oxalate Salts 2-2.1, wherein the salt crystal is an acetonitrile solvate.
2.4 Any of Oxalate Salts 2-2.3, wherein said salt crystal exhibits an X-ray powder diffraction pattern comprising at least five peaks having 2-theta angle values selected from the group consisting of: 6.5, 6.7, 7.2, 16.3, 16.7, 17.0, 19.5, 20.0, 20.6, and 20.8 degrees, wherein the XRPD pattern is measured in a diffractometer using copper anode, e.g., at wavelength alpha1 of 1.5406 Å and wavelength alpha2 of 1.5444 Å.
2.5 Any of Oxalate Salts 2-2.4, wherein said salt crystal exhibits an X-ray powder diffraction pattern comprising at least five peaks having 2-theta angle values selected from the group consisting of: 6.7, 7.2, 16.7, 17.0, and 20.0 degrees, wherein the XRPD pattern is measured in a diffractometer using copper anode, e.g., at wavelength alpha1 of 1.5406 Å and wavelength alpha2 of 1.5444 Å.
2.6 Any of Oxalate Salts 2-2.5, wherein said salt crystal exhibits an X-ray powder diffraction pattern comprising at least five peaks having d-spacing values selected from the group consisting of 13.7, 13.2, 12.2, 5.4, 5.3, 5.2, 5.1, 4.6, 4.4, and 4.3 Å.
2.7 Any of Oxalate Salts 2-2.6, wherein said salt crystal exhibits an X-ray powder diffraction pattern comprising peaks having d-spacing values selected from the group consisting of 13.2, 12.2, 5.3, 5.2, and 4.4 Å.
2.8 Any of Oxalate Salts 2-2.7, wherein said salt crystal exhibits an X-ray powder diffraction pattern comprising at least five peaks having 2-theta angle values or comprising at least five peaks having d-spacing values selected from those set forth in Table 15 as defined herein, wherein the XRPD pattern is measured in a diffractometer using copper anode, at wavelength alpha1 of 1.5406 Å and wavelength alpha2 of 1.5444 Å.
2.9 Any of Oxalate Salts 2-2.8, wherein said salt crystal exhibits a Differential Scanning Calorimetry (DSC) pattern comprising an endothermic peak between about 165° C. and 167° C., e.g., at about 166° C., between about 205° C. and 207° C., e.g., at about 207° C., and/or between about 214° C. and 216° C., e.g., at about 215° C.
2.10 Any of Oxalate Salts 2-2.9, wherein the Compound A free base and oxalic acid counterion are present in a molar ratio of about 2:1 to about 1:2, e.g., about 1:1.

In a further embodiment, the present disclosure provides for a third oxalate salt [Oxalate Salt 3] of Compound A.
3.1 Oxalate Salt 3, which is in crystalline form.
3.2 Oxalate Salt 3 or 3.1, which is in solvate form.
3.3 Any of Oxalate Salts 3-3.1, wherein the salt crystal is a 3-heptanone solvate, a 2-butanone solvate or an ethyl acetate solvate.
3.4 Any of Oxalate Salts 3-3.3, wherein said salt crystal exhibits an X-ray powder diffraction pattern comprising at least five peaks having 2-theta angle values selected from the group consisting of: 5.3, 6.0, 11.9, 16.6, 17.7, 18.3, 19.6, 20.5, 20.7, and 21.2 degrees, wherein the XRPD pattern is measured in a diffractometer using copper anode, e.g., at wavelength alpha1 of 1.5406 Å and wavelength alpha2 of 1.5444 Å.
3.5 Any of Oxalate Salts 3-3.4, wherein said salt crystal exhibits an X-ray powder diffraction pattern comprising at least five peaks having 2-theta angle values selected from the group consisting of: 5.3, 6.0, 11.9, 20.7, and 21.2 degrees, wherein the XRPD pattern is measured in a diffractometer using copper anode, e.g., at wavelength alpha1 of 1.5406 Å and wavelength alpha2 of 1.5444 Å.
3.6 Any of Oxalate Salts 3-3.5, wherein said salt crystal exhibits an X-ray powder diffraction pattern comprising at least five peaks having d-spacing values selected from the group consisting of 16.6, 14.7, 7.4, 5.3, 5.0, 4.8, 4.5, 4.3, 4.2, and 3.7 Å.
3.7 Any of Oxalate Salts 3-3.6, wherein said salt crystal exhibits an X-ray powder diffraction pattern comprising peaks having d-spacing values selected from the group consisting of 16.6, 14.7, 7.4, 4.3, and 4.2 Å.
3.8 Any of Oxalate Salts 3-3.7, wherein said salt crystal exhibits an X-ray powder diffraction pattern comprising at least five peaks having 2-theta angle values or comprising at least five peaks having d-spacing values selected from those set forth in Table 16 as defined herein, wherein the XRPD pattern is measured in a diffractometer using copper anode, at wavelength alpha1 of 1.5406 Å and wavelength alpha2 of 1.5444 Å.
3.9 Any of Oxalate Salts 3-3.8, wherein said salt crystal exhibits a Differential Scanning Calorimetry (DSC) pattern comprising an endothermic peak between about 214° C. and 220° C., e.g., at about 214° C., 218° C. or 219° C.
3.10 Any of Oxalate Salts 3-3.9, wherein the Compound A free base and oxalic acid counterion are present in a molar ratio of about 2:1 to about 1:2, e.g., about 1:1.

In a further embodiment, the present disclosure provides for a fourth oxalate salt [Oxalate Salt 4] of Compound A.
4.1 Oxalate Salt 4, which is in crystalline form.
4.2 Oxalate Salt 4 or 4.1, which is in solvate form.
4.3 Any of Oxalate Salts 4-4.1, wherein the salt crystal is an ethyl salicylate solvate.
4.4 Any of Oxalate Salts 4-4.3, wherein said salt crystal exhibits an X-ray powder diffraction pattern comprising at least five peaks having 2-theta angle values selected from the group consisting of: 5.8, 11.6, 12.1, 18.1, 18.5, 20.4, 21.4, 21.9, 27.1, and 23.2 degrees, wherein the XRPD pattern is measured in a diffractometer using copper anode, e.g., at wavelength alpha1 of 1.5406 Å and wavelength alpha2 of 1.5444 Å.
4.5 Any of Oxalate Salts 4-4.4, wherein said salt crystal exhibits an X-ray powder diffraction pattern comprising at least five peaks having 2-theta angle values selected from the group consisting of: 5.8, 11.6, 18.1, 20.4, and 21.9 degrees, wherein the XRPD pattern is measured in a diffractometer using copper anode, e.g., at wavelength alpha1 of 1.5406 Å and wavelength alpha2 of 1.5444 Å.
4.6 Any of Oxalate Salts 4-4.5, wherein said salt crystal exhibits an X-ray powder diffraction pattern comprising at least five peaks having d-spacing values selected from the group consisting of 15.3, 7.6, 7.3, 4.9, 4.8, 4.3, 4.1, 3.8, 3.3, and 3.1 Å.
4.7 Any of Oxalate Salts 4-4.6, wherein said salt crystal exhibits an X-ray powder diffraction pattern comprising peaks having d-spacing values selected from the group consisting of 15.3, 7.6, 4.9, 4.3, and 4.1 Å.
4.8 Any of Oxalate Salts 4-4.7, wherein said salt crystal exhibits an X-ray powder diffraction pattern comprising at least five peaks having 2-theta angle values or comprising at least five peaks having d-spacing values selected from those set forth in Table 57 as defined herein, wherein the XRPD pattern is measured in a diffractometer using copper anode, at wavelength alpha1 of 1.5406 Å and wavelength alpha2 of 1.5444 Å.
4.9 Any of Oxalate Salts 4-4.8, wherein said salt crystal exhibits a Differential Scanning Calorimetry (DSC) pattern comprising an endothermic peak between about 125° C. and 128° C., e.g., at about 126° C., and/or between about 138° C. and 148° C., e.g., at about 139° C.
4.10 Any of Oxalate Salts 4-4.9, wherein the Compound A free base and oxalic acid counterion are present in a molar ratio of about 2:1 to about 1:2, e.g., about 1:1.

In a further embodiment, the present disclosure provides for a palmitate salt [Palmitate Salt 1] of Compound A.
1.1 Palmitate Salt 1, which is in crystalline form.
1.2 Palmitate Salt 1 or 1.1, which is in anhydrous form.
1.3 Any of Palmitate Salts 1-1.1, wherein the salt crystal is an ethyl acetate, 2-butanone, acetonitrile or 3-heptanone solvate.
1.4 Any of Palmitate Salts 1-1.3, wherein said salt crystal exhibits an X-ray powder diffraction pattern comprising at least five peaks having 2-theta angle values selected from the group consisting of: 4.3, 5.5, 6.5, 7.3, 8.5, 9.5, 10.9, 19.2, 21.6, and 22.8 degrees, wherein the XRPD pattern is measured in a diffractometer using copper anode, e.g., at wavelength alpha1 of 1.5406 Å and wavelength alpha2 of 1.5444 Å.
1.5 Any of Palmitate Salts 1-1.4, wherein said salt crystal exhibits an X-ray powder diffraction pattern comprising at least five peaks having 2-theta angle values selected from the group consisting of: 4.3, 5.5, 8.5, 9.5, and 22.8 degrees, wherein the XRPD pattern is measured in a diffractometer using copper anode, e.g., at wavelength alpha1 of 1.5406 Å and wavelength alpha2 of 1.5444 Å.
1.6 Any of Palmitate Salts 1-1.5, wherein said salt crystal exhibits an X-ray powder diffraction pattern comprising at least five peaks having d-spacing values selected from the group consisting of 20.5, 16.1, 13.5, 12.0, 10.4, 9.3, 8.1, 4.6, 4.1, and 3.9 Å.
1.7 Any of Palmitate Salts 1-1.6, wherein said salt crystal exhibits an X-ray powder diffraction pattern comprising peaks having d-spacing values selected from the group consisting of 20.5, 16.1, 10.4, 9.3, and 3.9 Å.
1.8 Any of Palmitate Salts 1-1.7, wherein said salt crystal exhibits an X-ray powder diffraction pattern comprising at least five peaks having 2-theta angle values or comprising at least five peaks having d-spacing values selected from those set forth in Table 17 as defined herein, wherein the XRPD pattern is measured in a diffractometer using copper anode, at wavelength alpha1 of 1.5406 Å and wavelength alpha2 of 1.5444 Å.
1.9 Any of Palmitate Salts 1-1.8, wherein said salt crystal exhibits a Differential Scanning Calorimetry (DSC) pattern comprising an endothermic peak between about 59° C. and 66° C., e.g., at about 59° C., 62° C. or 63° C.
1.10 Any of Palmitate Salts 1-1.9, wherein the Compound A free base and palmitic acid counterion are present in a molar ratio of about 2:1 to about 1:2, e.g., about 1:1.

In a further embodiment, the present disclosure provides for a 2-oxo-glutarate salt [2-Oxo-glutarate Salt 1] of Compound A.
1.1 2-Oxo-glutarate Salt 1, which is in crystalline form.
1.2 2-Oxo-glutarate Salt 1 or 1.1, which is in solvate form.
1.3 Any of 2-Oxo-glutarate Salts 1-1.1, wherein the salt crystal is an ethyl acetate solvate.
1.4 Any of 2-Oxo-glutarate Salts 1-1.3, wherein said salt crystal exhibits an X-ray powder diffraction pattern comprising at least five peaks having 2-theta angle values selected from the group consisting of: 5.1, 8.5, 10.1, 11.0, 11.8, 14.5, 15.7, 17.5, 19.8, and 20.2 degrees, wherein the XRPD pattern is measured in a diffractometer using copper anode, e.g., at wavelength alpha1 of 1.5406 Å and wavelength alpha2 of 1.5444 Å.
1.5 Any of 2-Oxo-glutarate Salts 1-1.4, wherein said salt crystal exhibits an X-ray powder diffraction pattern comprising at least five peaks having 2-theta angle values selected from the group consisting of: 5.1, 8.5, 11.0, 17.5, and 20.2 degrees, wherein the XRPD pattern is measured in a diffractometer using copper anode, e.g., at wavelength alpha1 of 1.5406 Å and wavelength alpha2 of 1.5444 Å.
1.6 Any of 2-Oxo-glutarate Salts 1-1.5, wherein said salt crystal exhibits an X-ray powder diffraction pattern comprising at least five peaks having d-spacing values selected from the group consisting of 17.4, 10.4, 8.7, 8.0, 7.5, 6.1, 5.6, 5.1, 4.5, and 4.4 Å.
1.7 Any of 2-Oxo-glutarate Salts 1-1.6, wherein said salt crystal exhibits an X-ray powder diffraction pattern comprising peaks having d-spacing values selected from the group consisting of 17.4, 10.4, 8.0, 5.1, and 4.4 Å.
1.8 Any of 2-Oxo-glutarate Salts 1-1.7, wherein said salt crystal exhibits an X-ray powder diffraction pattern comprising at least five peaks having 2-theta angle values or comprising at least five peaks having d-spacing values selected from those set forth in Table 18 as defined herein, wherein the XRPD pattern is measured in a diffractometer using copper anode, at wavelength alpha1 of 1.5406 Å and wavelength alpha2 of 1.5444 Å.
1.9 Any of 2-Oxo-glutarate Salts 1-1.8, wherein said salt crystal exhibits a Differential Scanning Calorimetry (DSC) pattern comprising an endothermic peak between about 124° C. and 126° C., e.g., at about 125° C., and/or between about 157° C. and 159° C., e.g., at about 158° C.
1.10 Any of 2-Oxo-glutarate Salts 1-1.9, wherein the Compound A free base and 2-oxo-glutaric acid counterion are present in a molar ratio of about 2:1 to about 1:2, e.g., about 1:1, e.g., 1:1.1.

In a further embodiment, the present disclosure provides for a first xinafoate salt [Xinafoate Salt 1] of Compound A.
1.1 Xinafoate Salt 1, which is in crystalline form.
1.2 Xinafoate Salt 1 or 1.1, which is in solvate form.
1.3 Any of Xinafoate Salts 1-1.1, wherein the salt crystal is an acetonitrile solvate.
1.4 Any of Xinafoate Salts 1-1.3, wherein said salt crystal exhibits an X-ray powder diffraction pattern comprising at least five peaks having 2-theta angle values selected from the group consisting of: 5.4, 6.5, 6.7, 12.2, 13.6, 14.4, 14.8, 18.2, 18.8, and 22.9 degrees, wherein the XRPD pattern is measured in a diffractometer using copper anode, e.g., at wavelength alpha1 of 1.5406 Å and wavelength alpha2 of 1.5444 Å.
1.5 Any of Xinafoate Salts 1-1.4, wherein said salt crystal exhibits an X-ray powder diffraction pattern comprising at least five peaks having 2-theta angle values selected from the group consisting of: 5.4, 6.5, 6.7, 12.2, and 14.8 degrees, wherein the XRPD pattern is measured in a diffractometer using copper anode, e.g., at wavelength alpha1 of 1.5406 Å and wavelength alpha2 of 1.5444 Å.
1.6 Any of Xinafoate Salts 1-1.5, wherein said salt crystal exhibits an X-ray powder diffraction pattern comprising at least five peaks having d-spacing values selected from the group consisting of 16.3, 13.5, 13.1, 7.3, 6.5, 6.1, 6.0, 4.9, 4.7, and 3.9 Å.
1.7 Any of Xinafoate Salts 1-1.6, wherein said salt crystal exhibits an X-ray powder diffraction pattern comprising peaks having d-spacing values selected from the group consisting of 16.3, 13.5, 13.1, 7.3, and 6.0 Å.
1.8 Any of Xinafoate Salts 1-1.7, wherein said salt crystal exhibits an X-ray powder diffraction pattern comprising at least five peaks having 2-theta angle values or comprising at least five peaks having d-spacing values selected from those set forth in Table 19 as defined herein, wherein the XRPD pattern is measured in a diffractometer using copper anode, at wavelength alpha1 of 1.5406 Å and wavelength alpha2 of 1.5444 Å.
1.9 Any of Xinafoate Salts 1-1.8, wherein said salt crystal exhibits a Differential Scanning Calorimetry (DSC) pattern comprising an endothermic peak between about 130° C. and 132° C., e.g., at about 131° C., and between about 143° C. and 146° C., e.g., at about 145° C., and/or between about 171° C. and 174° C., e.g., at about 172° C.
1.10 Any of Xinafoate Salts 1-1.9, wherein the Compound A free base and 1-hydroxy-2-napthoic acid counterion are present in a molar ratio of about 2:1 to about 1:2, e.g., about 1:1.

In a further embodiment, the present disclosure provides for a second xinafoate salt [Xinafoate Salt 2] of Compound A.
2.1 Xinafoate Salt 2, which is in crystalline form.
2.2 Xinafoate Salt 2 or 2.1, which is in solvate form.
2.3 Any of Xinafoate Salts 2-2.1, wherein the salt crystal is a toluene solvate.
2.4 Any of Xinafoate Salts 2-2.3, wherein said salt crystal exhibits an X-ray powder diffraction pattern comprising at least five peaks having 2-theta angle values selected from the group consisting of: 4.2, 5.3, 5.4, 6.1, 6.5, and 12.4 degrees, wherein the XRPD pattern is measured in a diffractometer using copper anode, e.g., at wavelength alpha1 of 1.5406 Å and wavelength alpha2 of 1.5444 Å.
2.5 Any of Xinafoate Salts 2-2.4, wherein said salt crystal exhibits an X-ray powder diffraction pattern comprising at least five peaks having d-spacing values selected from the group consisting of 20.9, 16.8, 16.5, 14.6, 14.5, 13.6, and 7.1 Å.
2.6 Any of Xinafoate Salts 2-2.5, wherein said salt crystal exhibits an X-ray powder diffraction pattern comprising at least five peaks having 2-theta angle values or comprising at least five peaks having d-spacing values selected from those set forth in Table 20 as defined herein, wherein the XRPD pattern is measured in a diffractometer using copper anode, at wavelength alpha1 of 1.5406 Å and wavelength alpha2 of 1.5444 Å.
2.7 Any of Xinafoate Salts 2-2.6, wherein said salt crystal exhibits a Differential Scanning Calorimetry (DSC) pattern comprising an endothermic peak between about 117° C. and 119° C., e.g., at about 118° C., between about 163° C. and 166° C., e.g., at about 164° C., and/or between about 174° C. and 177° C., e.g., at about 175° C.
2.8 Any of Xinafoate Salts 2-2.7, wherein the Compound A free base and 1-hydroxy-2-napthoic acid counterion are present in a molar ratio of about 2:1 to about 1:2, e.g., about 1:1.

In a further embodiment, the present disclosure provides for a third xinafoate salt [Xinafoate Salt 3] of Compound A.
3.1 Xinafoate Salt 3, which is in crystalline form.
3.2 Xinafoate Salt 3 or 3.1, which is in solvate form.
3.3 Any of Xinafoate Salts 3-3.1, wherein the salt crystal is an ethyl acetate solvate.
3.4 Any of Xinafoate Salts 3-3.3, wherein said salt crystal exhibits an X-ray powder diffraction pattern comprising at least five peaks having 2-theta angle values selected from the group consisting of: 5.4, 5.9, 6.7, 10.7, 10.8, 13.7, 13.9, 17.0, 21.1, and 21.3 degrees, wherein the XRPD pattern is measured in a diffractometer using copper anode, e.g., at wavelength alpha1 of 1.5406 Å and wavelength alpha2 of 1.5444 Å.
3.5 Any of Xinafoate Salts 3-3.4, wherein said salt crystal exhibits an X-ray powder diffraction pattern comprising at least five peaks having 2-theta angle values selected from the group consisting of: 6.7, 10.8, 13.7, 13.9, and 21.1 degrees, wherein the XRPD pattern is measured in a diffractometer using copper anode, e.g., at wavelength alpha1 of 1.5406 Å and wavelength alpha2 of 1.5444 Å.
3.6 Any of Xinafoate Salts 3-3.5, wherein said salt crystal exhibits an X-ray powder diffraction pattern comprising at least five peaks having d-spacing values selected from the group consisting of 16.3, 15.0, 13.2, 8.2, 6.5, 6.4, 5.2, 4.3, 4.2, and 4.0 Å.
3.7 Any of Xinafoate Salts 3-3.6, wherein said salt crystal exhibits an X-ray powder diffraction pattern comprising peaks having d-spacing values selected from the group consisting of 13.2, 8.2, 6.5, 6.4, and 4.2 Å.
3.8 Any of Xinafoate Salts 3-3.7, wherein said salt crystal exhibits an X-ray powder diffraction pattern comprising at least five peaks having 2-theta angle values or comprising at least five peaks having d-spacing values selected from those set forth in Table 21 as defined herein, wherein the XRPD pattern is measured in a diffractometer using copper anode, at wavelength alpha1 of 1.5406 Å and wavelength alpha2 of 1.5444 Å.
3.9 Any of Xinafoate Salts 3-3.8, wherein said salt crystal exhibits a Differential Scanning Calorimetry (DSC) pattern comprising an endothermic peak between about 131° C. and 133° C., e.g., at about 132° C., and/or between about 170° C. and 173° C., e.g., at about 172° C.
3.10 Any of Xinafoate Salts 3-3.9, wherein the Compound A free base and 1-hydroxy-2-napthoic acid counterion are present in a molar ratio of about 2:1 to about 1:2, e.g., about 1:1.

In a further embodiment, the present disclosure provides for a first tosylate salt [Tosylate Salt 1] of Compound A.
1.1 Tosylate Salt 1, which is in crystalline form.
1.2 Tosylate Salt 1 or 1.1, which is in anhydrous form.
1.3 Any of Tosylate Salts 1-1.1, wherein the salt crystal is a 3-heptanone solvate.
1.4 Any of Tosylate Salts 1-1.3, wherein said salt crystal exhibits an X-ray powder diffraction pattern comprising at least five peaks having 2-theta angle values selected from the group consisting of: 5.1, 5.6, 8.5, 10.5, 15.5, 17.1, 20.1, 20.4, 23.2, and 23.3 degrees, wherein the XRPD pattern is measured in a diffractometer using copper anode, e.g., at wavelength alpha1 of 1.5406 Å and wavelength alpha2 of 1.5444 Å.
1.5 Any of Tosylate Salts 1-1.4, wherein said salt crystal exhibits an X-ray powder diffraction pattern comprising at least five peaks having 2-theta angle values selected from the group consisting of: 5.6, 8.5, 10.5, 15.5, and 20.4 degrees, wherein the XRPD pattern is measured in a diffractometer using copper anode, e.g., at wavelength alpha1 of 1.5406 Å and wavelength alpha2 of 1.5444 Å.
1.6 Any of Tosylate Salts 1-1.5, wherein said salt crystal exhibits an X-ray powder diffraction pattern comprising at least five peaks having d-spacing values selected from the group consisting of 17.2, 15.7, 10.3, 8.4, 5.7, 5.2, 4.4, 4.3, 4.0, and 3.8 Å.
1.7 Any of Tosylate Salts 1-1.6, wherein said salt crystal exhibits an X-ray powder diffraction pattern comprising peaks having d-spacing values selected from the group consisting of 15.7, 10.3, 8.4, 5.7, and 4.3 Å.
1.8 Any of Tosylate Salts 1-1.7, wherein said salt crystal exhibits an X-ray powder diffraction pattern comprising at least five peaks having 2-theta angle values or comprising at least five peaks having d-spacing values selected from those set forth in Table 22 as defined herein, wherein the XRPD pattern is measured in a diffractometer using copper anode, at wavelength alpha1 of 1.5406 Å and wavelength alpha2 of 1.5444 Å.
1.9 Any of Tosylate Salts 1-1.8, wherein said salt crystal exhibits a Differential Scanning Calorimetry (DSC) pattern comprising an endothermic peak between about 216° C. and 218° C., e.g., at about 217° C.
1.10 Any of Tosylate Salts 1-1.9, wherein the Compound A free base and p-toluene sulfonic acid counterion are present in a molar ratio of about 2:1 to about 1:2, e.g., about 1:1.

In a further embodiment, the present disclosure provides for a first tartrate salt [Tartrate Salt 1] of Compound A.
1.1 Tartrate Salt 1, which is in crystalline form.
1.2 Tartrate Salt 1 or 1.1, which is in anhydrous form.
1.3 Any of Tartrate Salts 1-1.1, wherein the salt crystal is in solvate form, e.g., wherein the salt crystal is an acetone solvate.
1.4 Any of Tartrate Salts 1-1.3, wherein said salt crystal exhibits an X-ray powder diffraction pattern comprising at least five peaks having 2-theta angle values selected from the group consisting of: 3.7, 6.0, 6.9, 10.4, 11.6, 15.0, 17.5, 20.3, 20.8, and 21.7 degrees, wherein the XRPD pattern is measured in a diffractometer using copper anode, e.g., at wavelength alpha1 of 1.5406 Å and wavelength alpha2 of 1.5444 Å.
1.5 Any of Tartrate Salts 1-1.4, wherein said salt crystal exhibits an X-ray powder diffraction pattern comprising at least five peaks having 2-theta angle values selected from the group consisting of: 6.0, 6.9, 11.6, 20.3, and 21.7 degrees, wherein the XRPD pattern is measured in a diffractometer using copper anode, e.g., at wavelength alpha1 of 1.5406 Å and wavelength alpha2 of 1.5444 Å.
1.6 Any of Tartrate Salts 1-1.5, wherein said salt crystal exhibits an X-ray powder diffraction pattern comprising at least five peaks having d-spacing values selected from the group consisting of 23.9, 14.8, 12.8, 8.5, 7.6, 5.9, 5.1, 4.4, 4.3, and 4.1 Å.
1.7 Any of Tartrate Salts 1-1.6, wherein said salt crystal exhibits an X-ray powder diffraction pattern comprising peaks having d-spacing values selected from the group consisting of 14.8, 12.8, 7.6, 4.4, and 4.1 Å.
1.8 Any of Tartrate Salts 1-1.7, wherein said salt crystal exhibits an X-ray powder diffraction pattern comprising at least five peaks having 2-theta angle values or comprising at least five peaks having d-spacing values selected from those set forth in Table 24 as defined herein, wherein the XRPD pattern is measured in a diffractometer using copper anode, at wavelength alpha1 of 1.5406 Å and wavelength alpha2 of 1.5444 Å.
1.9 Any of Tartrate Salts 1-1.8, wherein said salt crystal exhibits a Differential Scanning Calorimetry (DSC) pattern comprising an endothermic peak between about 134° C. and 136° C., e.g., at about 135° C.
1.10 Any of Tartrate Salts 1-1.9, wherein the Compound A free base and tartaric acid counterion are present in a molar ratio of about 2:1 to about 1:2, e.g., about 1:1.

In a further embodiment, the present disclosure provides for a first tartrate salt [Tartrate Salt 2] of Compound A.
2.1 Tartrate Salt 2, which is in crystalline form.
2.2 Tartrate Salt 2 or 2.1, which is in solvate form.
2.3 Any of Tartrate Salts 2-2.1, wherein the salt crystal is an ethyl tert butyl ether solvate.
2.4 Any of Tartrate Salts 2-2.3, wherein said salt crystal exhibits an X-ray powder diffraction pattern comprising at least five peaks having 2-theta angle values selected from the group consisting of: 5.9, 6.3, 8.0, 10.2, 11.1, 12.2, 12.6, 17.0, 17.4, and 21.5 degrees, wherein the XRPD pattern is measured in a diffractometer using copper anode, e.g., at wavelength alpha1 of 1.5406 Å and wavelength alpha2 of 1.5444 Å.
2.5 Any of Tartrate Salts 2-2.4, wherein said salt crystal exhibits an X-ray powder diffraction pattern comprising at least five peaks having 2-theta angle values selected from the group consisting of: 5.9, 6.3, 12.2, 12.6, 17.0 degrees, wherein the XRPD pattern is measured in a diffractometer using copper anode, e.g., at wavelength alpha1 of 1.5406 Å and wavelength alpha2 of 1.5444 Å.
2.6 Any of Tartrate Salts 2-2.5, wherein said salt crystal exhibits an X-ray powder diffraction pattern comprising at least five peaks having d-spacing values selected from the group consisting of 14.9, 14.1, 11.0, 8.6, 7.9, 7.2, 7.0, 5.2, 5.1, and 4.1 Å.
2.7 Any of Tartrate Salts 2-2.6, wherein said salt crystal exhibits an X-ray powder diffraction pattern comprising peaks having d-spacing values selected from the group consisting of 14.1, 14.9, 7.2, 7.0, and 5.2 Å.
2.8 Any of Tartrate Salts 2-2.7, wherein said salt crystal exhibits an X-ray powder diffraction pattern comprising at least five peaks having 2-theta angle values or comprising at least five peaks having d-spacing values selected from those set forth in Table 56 as defined herein, wherein the XRPD pattern is measured in a diffractometer using copper anode, at wavelength alpha1 of 1.5406 Å and wavelength alpha2 of 1.5444 Å.
2.9 Any of Tartrate Salts 2-2.8, wherein said salt crystal exhibits a Differential Scanning Calorimetry (DSC) pattern comprising an endothermic peak between about 103° C. and 106° C., e.g., at about 104° C., between about 120° C. and 123° C., e.g., at about 121° C., and/or between about 134° C. and 137° C., e.g., at about 136° C.
2.10 Any of Tartrate Salts 2-2.9, wherein the Compound A free base and tartaric acid counterion are present in a molar ratio of about 2:1 to about 1:2, e.g., about 1:1.

In a further embodiment, the present disclosure provides for a first succinate salt [Succinate Salt 1] of Compound A.
1.1 Succinate Salt 1, which is in crystalline form.
1.2 Succinate Salt 1 or 1.1, which is in solvate form.
1.3 Any of Succinate Salts 1-1.1, wherein the salt crystal is an acetone solvate.
1.4 Any of Succinate Salts 1-1.3, wherein said salt crystal exhibits an X-ray powder diffraction pattern comprising at least five peaks having 2-theta angle values selected from the group consisting of: 6.9, 7.1, 9.4, 9.8, 16.0, 16.4, 17.1, 19.3, 22.8, and 25.8 degrees, wherein the XRPD pattern is measured in a diffractometer using copper anode, e.g., at wavelength alpha1 of 1.5406 Å and wavelength alpha2 of 1.5444 Å.
1.5 Any of Succinate Salts 1-1.4, wherein said salt crystal exhibits an X-ray powder diffraction pattern comprising at least five peaks having 2-theta angle values selected from the group consisting of: 6.9, 7.1, 9.8, 16.4, and 19.3 degrees, wherein the XRPD pattern is measured in a diffractometer using copper anode, e.g., at wavelength alpha1 of 1.5406 Å and wavelength alpha2 of 1.5444 Å.
1.6 Any of Succinate Salts 1-1.5, wherein said salt crystal exhibits an X-ray powder diffraction pattern comprising at least five peaks having d-spacing values selected from the group consisting of 12.8, 12.5, 9.4, 9.0, 5.5, 5.4, 5.2, 4.6, 3.9, and 3.5 Å.
1.7 Any of Succinate Salts 1-1.6, wherein said salt crystal exhibits an X-ray powder diffraction pattern comprising peaks having d-spacing values selected from the group consisting of 12.8, 12.5, 9.0, 5.4, and 4.6 Å.
1.8 Any of Succinate Salts 1-1.7, wherein said salt crystal exhibits an X-ray powder diffraction pattern comprising at least five peaks having 2-theta angle values or comprising at least five peaks having d-spacing values selected from those set forth in Table 25 as defined herein, wherein the XRPD pattern is measured in a diffractometer using copper anode, at wavelength alpha1 of 1.5406 Å and wavelength alpha2 of 1.5444 Å.
1.9 Any of Succinate Salts 1-1.8, wherein said salt crystal exhibits a Differential Scanning Calorimetry (DSC) pattern comprising an endothermic peak between about 153° C. and 155° C., e.g., at about 154° C., between about 172° C. and 175° C., e.g., at about 173° C., and/or between about 178° C. and 181° C., e.g., at about 180° C.
1.10 Any of Succinate Salts 1-1.9, wherein the Compound A free base and succinic acid counterion are present in a molar ratio of about 2:1 to about 1:2, e.g., about 1:0.6.

In a further embodiment, the present disclosure provides for a second succinate salt [Succinate Salt 2] of Compound A.
2.1 Succinate Salt 2, which is in crystalline form.
2.2 Succinate Salt 2 or 2.1, which is in anhydrous form.
2.3 Any of Succinate Salts 2-2.1, wherein the salt crystal is an ethyl acetate solvate.
2.4 Any of Succinate Salts 2-2.3, wherein said salt crystal exhibits an X-ray powder diffraction pattern comprising at least five peaks having 2-theta angle values selected from the group consisting of: 6.9, 9.4, 22.4, 16.3, 19.3, 4.6, 25.7, 21.4, 13.6, and 23.9 degrees, wherein the XRPD pattern is measured in a diffractometer using copper anode, e.g., at wavelength alpha1 of 1.5406 Å and wavelength alpha2 of 1.5444 Å.
2.5 Any of Succinate Salts 2-2.4, wherein said salt crystal exhibits an X-ray powder diffraction pattern comprising at least five peaks having 2-theta angle values selected from the group consisting of: 6.9, 9.4, 22.4, 16.3, and 19.3 degrees, wherein the XRPD pattern is measured in a diffractometer using copper anode, e.g., at wavelength alpha1 of 1.5406 Å and wavelength alpha2 of 1.5444 Å.
2.6 Any of Succinate Salts 2-2.5, wherein said salt crystal exhibits an X-ray powder diffraction pattern comprising at least five peaks having d-spacing values selected from the group consisting of 19.3, 12.8, 9.4, 6.5, 5.4, 4.6, 4.1, 4.0, 3.7, and 3.5 Å.
2.7 Any of Succinate Salts 2-2.6, wherein said salt crystal exhibits an X-ray powder diffraction pattern comprising peaks having d-spacing values selected from the group consisting of 12.8, 9.4, 5.4, 4.6, and 4.0 Å.
2.8 Any of Succinate Salts 2-2.7, wherein said salt crystal exhibits an X-ray powder diffraction pattern comprising at least five peaks having 2-theta angle values or comprising at least five peaks having d-spacing values selected from those set forth in Table 26 as defined herein, wherein the XRPD pattern is measured in a diffractometer using copper anode, at wavelength alpha1 of 1.5406 Å and wavelength alpha2 of 1.5444 Å.
2.9 Any of Succinate Salts 2-2.8, wherein said salt crystal exhibits a Differential Scanning Calorimetry (DSC) pattern comprising an endothermic peak between about 150° C. and 152° C., e.g., at about 151° C., between about 163° C. and 165° C., e.g., at about 164° C., between about 172° C. and 175° C., e.g., at about 174° C., and/or between about 178° C. and 181° C., e.g., at about 179° C.
2.10 Any of Succinate Salts 2-2.9, wherein the Compound A free base and succinic acid counterion are present in a molar ratio of about 2:1 to about 1:2, e.g., about 1:1, e.g., about 1:0.8.

In a further embodiment, the present disclosure provides for a first mesylate salt [Mesylate Salt 1] of Compound A.
1.1 Mesylate Salt 1, which is in crystalline form.
1.2 Mesylate Salt 1 or 1.1, which is in anhydrous form.
1.3 Any of Mesylate Salts 1-1.1, wherein the salt crystal is an acetone solvate.
1.4 Any of Mesylate Salts 1-1.3, wherein said salt crystal exhibits an X-ray powder diffraction pattern comprising at least five peaks having 2-theta angle values selected from the group consisting of: 6.2, 6.4, 11.7, 12.3, 17.1, 18.8, 20.2, 21.2, 29.7, and 29.8 degrees, wherein the XRPD pattern is measured in a diffractometer using copper anode, e.g., at wavelength alpha1 of 1.5406 Å and wavelength alpha2 of 1.5444 Å.
1.5 Any of Mesylate Salts 1-1.4, wherein said salt crystal exhibits an X-ray powder diffraction pattern comprising at least five peaks having 2-theta angle values selected from the group consisting of: 6.4, 18.8, 20.2, 21.2, and 29.7 degrees, wherein the XRPD pattern is measured in a diffractometer using copper anode, e.g., at wavelength alpha1 of 1.5406 Å and wavelength alpha2 of 1.5444 Å.
1.6 Any of Mesylate Salts 1-1.5, wherein said salt crystal exhibits an X-ray powder diffraction pattern comprising at least five peaks having d-spacing values selected from the group consisting of 14.2, 13.8, 7.6, 7.2, 5.2, 4.9 4.7, 4.4, 4.2, and 3.0 Å.
1.7 Any of Mesylate Salts 1-1.6, wherein said salt crystal exhibits an X-ray powder diffraction pattern comprising peaks having d-spacing values selected from the group consisting of 13.8, 4.7, 4.4, 4.2, and 3.0 Å.
1.8 Any of Mesylate Salts 1-1.7, wherein said salt crystal exhibits an X-ray powder diffraction pattern comprising at least five peaks having 2-theta angle values or comprising at least five peaks having d-spacing values selected from those set forth in Table 27 as defined herein, wherein the XRPD pattern is measured in a diffractometer using copper anode, at wavelength alpha1 of 1.5406 Å and wavelength alpha2 of 1.5444 Å.
1.9 Any of Mesylate Salts 1-1.8, wherein said salt crystal exhibits a Differential Scanning Calorimetry (DSC) pattern comprising an endothermic peak between about 310° C. and 312° C., e.g., at about 311° C.
1.10 Any of Mesylate Salts 1-1.9, wherein the Compound A free base and methanesulfonic acid counterion are present in a molar ratio of about 2:1 to about 1:2, e.g., about 1:1.

In a further embodiment, the present disclosure provides for a napadisylate salt [Napadisylate Salt 1] of Compound A.
1.1 Napadisylate Salt 1, which is in crystalline form.
1.2 Napadisylate Salt 1 or 1.1, which is in solvate form.
1.3 Any of Napadisylate Salts 1-1.1, wherein the salt crystal is an acetonitrile solvate.
1.4 Any of Napadisylate Salts 1-1.3, wherein said salt crystal exhibits an X-ray powder diffraction pattern comprising at least five peaks having 2-theta angle values selected from the group consisting of: 3.1, 12.6, 15.8, 16.1, 16.7, 18.6, 25.3, and 30.5 degrees, wherein the XRPD pattern is measured in a diffractometer using copper anode, e.g., at wavelength alpha1 of 1.5406 Å and wavelength alpha2 of 1.5444 Å.
1.5 Any of Napadisylate Salts 1-1.4, wherein said salt crystal exhibits an X-ray powder diffraction pattern comprising at least five peaks having 2-theta angle values selected from the group consisting of: 3.1, 12.6, 15.8, 16.7, and 25.3 degrees, wherein the XRPD pattern is measured in a diffractometer using copper anode, e.g., at wavelength alpha1 of 1.5406 Å and wavelength alpha2 of 1.5444 Å.
1.6 Any of Napadisylate Salts 1-1.5, wherein said salt crystal exhibits an X-ray powder diffraction pattern comprising at least five peaks having d-spacing values selected from the group consisting of 28.3, 7.0, 5.6, 5.5, 5.3, 4.8, 3.5, and 2.9 Å.
1.7 Any of Napadisylate Salts 1-1.6, wherein said salt crystal exhibits an X-ray powder diffraction pattern comprising peaks having d-spacing values selected from the group consisting of 28.3, 7.0, 5.6, 5.3, and 3.5 Å.
1.8 Any of Napadisylate Salts 1-1.7, wherein said salt crystal exhibits an X-ray powder diffraction pattern comprising at least five peaks having 2-theta angle values or comprising at least five peaks having d-spacing values selected from those set forth in Table 28 as defined herein, wherein the XRPD pattern is measured in a diffractometer using copper anode, at wavelength alpha1 of 1.5406 Å and wavelength alpha2 of 1.5444 Å.
1.9 Any of Napadisylate Salts 1-1.8, wherein said salt crystal exhibits a Differential Scanning Calorimetry (DSC) pattern comprising an endothermic peak between about 103° C. and 107° C., e.g., at about 105° C.
1.10 Any of Napadisylate Salts 1-1.9, wherein the Compound A free base and naphthalene disulfonic acid counterion are present in a molar ratio of about 2:1 to about 1:2, e.g., about 1:1, e.g., about 1:1.2.

In a further embodiment, the present disclosure provides for an edisylate salt [Edisylate Salt 1] of Compound A.
1.1 Edisylate Salt 1, which is in crystalline form.
1.2 Edisylate Salt 1 or 1.1, which is in anhydrous form.
1.3 Any of Edisylate Salts 1-1.1, wherein the salt crystal is a 2-butanone solvate.
1.4 Any of Edisylate Salts 1-1.3, wherein said salt crystal exhibits an X-ray powder diffraction pattern comprising at least five peaks having 2-theta angle values selected from the group consisting of: 4.5, 4.7, 11.7, 12.2, 12.8, 17.3, 18.4, 18.7, 21.3, and 25.6 degrees, wherein the XRPD pattern is measured in a diffractometer using copper anode, e.g., at wavelength alpha1 of 1.5406 Å and wavelength alpha2 of 1.5444 Å.
1.5 Any of Edisylate Salts 1-1.4, wherein said salt crystal exhibits an X-ray powder diffraction pattern comprising at least five peaks having 2-theta angle values selected from the group consisting of: 4.7, 12.8, 18.7, 21.3, and 25.6 degrees, wherein the XRPD pattern is measured in a diffractometer using copper anode, e.g., at wavelength alpha1 of 1.5406 Å and wavelength alpha2 of 1.5444 Å.
1.6 Any of Edisylate Salts 1-1.5, wherein said salt crystal exhibits an X-ray powder diffraction pattern comprising at least five peaks having d-spacing values selected from the group consisting of 19.7, 18.7, 7.6, 7.2, 6.9, 5.1, 4.8, 4.7, 4.2, and 3.5 Å.
1.7 Any of Edisylate Salts 1-1.6, wherein said salt crystal exhibits an X-ray powder diffraction pattern comprising peaks having d-spacing values selected from the group consisting of 18.7, 6.9, 4.7, 4.2, and 3.5 Å.
1.8 Any of Edisylate Salts 1-1.7, wherein said salt crystal exhibits an X-ray powder diffraction pattern comprising at least five peaks having 2-theta angle values or comprising at least five peaks having d-spacing values selected from those set forth in Table 29 as defined herein, wherein the XRPD pattern is measured in a diffractometer using copper anode, at wavelength alpha1 of 1.5406 Å and wavelength alpha2 of 1.5444 Å.
1.9 Any of Edisylate Salts 1-1.8, wherein said salt crystal exhibits a Differential Scanning Calorimetry (DSC) pattern comprising an endothermic peak between about 295° C. and 298° C., e.g., at about 296° C.
1.10 Any of Edisylate Salts 1-1.9, wherein the Compound A free base and ethane disulfonic acid counterion are present in a molar ratio of about 2:1 to about 1:2, e.g., about 1:1.

In a further embodiment, the present disclosure provides for a propionate salt [Propionate Salt 1] of Compound A.
1.1 Propionate Salt 1, which is in crystalline form.
1.2 Propionate Salt 1 or 1.1, which is in solvate form.
1.3 Any of Propionate Salts 1-1.1, wherein the salt crystal is a methanol solvate.
1.4 Any of Propionate Salts 1-1.3, wherein said salt crystal exhibits an X-ray powder diffraction pattern comprising at least five peaks having 2-theta angle values selected from the group consisting of: 5.7, 8.8, 9.5, 16.1, 17.0, 17.5, 18.3, 19.0, 22.7, and 32.6 degrees, wherein the XRPD pattern is measured in a diffractometer using copper anode, e.g., at wavelength alpha1 of 1.5406 Å and wavelength alpha2 of 1.5444 Å.
1.5 Any of Propionate Salts 1-1.4, wherein said salt crystal exhibits an X-ray powder diffraction pattern comprising at least five peaks having 2-theta angle values selected from the group consisting of: 16.1, 17.0, 18.3, 22.7, and 32.6 degrees, wherein the XRPD pattern is measured in a diffractometer using copper anode, e.g., at wavelength alpha1 of 1.5406 Å and wavelength alpha2 of 1.5444 Å.
1.6 Any of Propionate Salts 1-1.5, wherein said salt crystal exhibits an X-ray powder diffraction pattern comprising at least five peaks having d-spacing values selected from the group consisting of 15.4, 10.1, 9.3, 5.5, 5.2, 5.1, 4.8, 4.7, 3.9, and 2.7 Å.
1.7 Any of Propionate Salts 1-1.6, wherein said salt crystal exhibits an X-ray powder diffraction pattern comprising peaks having d-spacing values selected from the group consisting of 5.5, 5.2, 4.8, 3.9, and 2.7 Å.
1.8 Any of Propionate Salts 1-1.7, wherein said salt crystal exhibits an X-ray powder diffraction pattern comprising at least five peaks having 2-theta angle values or comprising at least five peaks having d-spacing values selected from those set forth in Table 30 as defined herein, wherein the XRPD pattern is measured in a diffractometer using copper anode, at wavelength alpha1 of 1.5406 Å and wavelength alpha2 of 1.5444 Å.
1.9 Any of Propionate Salts 1-1.8, wherein said salt crystal exhibits a Differential Scanning Calorimetry (DSC) pattern comprising an endothermic peak between about 109° C. and 112° C., e.g., at about 111° C., and/or between about 135° C. and 137° C., e.g., at about 136° C.
1.10 Any of Propionate Salts 1-1.9, wherein the Compound A free base and propionic acid counterion are present in a molar ratio of about 2:1 to about 1:2, e.g., about 1:1, e.g., about 1:0.7.

In a further embodiment, the present disclosure provides for a caprylate salt [Caprylate Salt 1] of Compound A.
1.1 Caprylate Salt 1, which is in crystalline form.
1.2 Caprylate Salt 1 or 1.1, which is in anhydrous form.
1.3 Any of Caprylate Salts 1-1.1, wherein the salt crystal is a methanol solvate.
1.4 Any of Caprylate Salts 1-1.3, wherein said salt crystal exhibits an X-ray powder diffraction pattern comprising at least five peaks having 2-theta angle values selected from the group consisting of: 2.9, 3.0, 4.5, 4.6, 4.7, 4.8, 4.9, 6.0, 22.0, and 22.8 degrees, wherein the XRPD pattern is measured in a diffractometer using copper anode, e.g., at wavelength alpha1 of 1.5406 Å and wavelength alpha2 of 1.5444 Å.
1.5 Any of Caprylate Salts 1-1.4, wherein said salt crystal exhibits an X-ray powder diffraction pattern comprising at least five peaks having 2-theta angle values selected from the group consisting of: 4.6, 4.7, 4.8, 4.9, and 22.8 degrees, wherein the XRPD pattern is measured in a diffractometer using copper anode, e.g., at wavelength alpha1 of 1.5406 Å and wavelength alpha2 of 1.5444 Å.
1.6 Any of Caprylate Salts 1-1.5, wherein said salt crystal exhibits an X-ray powder diffraction pattern comprising at least five peaks having d-spacing values selected from the group consisting of 30.1, 29.2, 19.7, 19.3, 18.7, 18.2, 18.0, 14.8, 4.0, and 3.9 Å.
1.7 Any of Caprylate Salts 1-1.6, wherein said salt crystal exhibits an X-ray powder diffraction pattern comprising peaks having d-spacing values selected from the group consisting of 19.3, 18.7, 18.2, 18.0, and 3.9 Å.
1.8 Any of Caprylate Salts 1-1.7, wherein said salt crystal exhibits an X-ray powder diffraction pattern comprising at least five peaks having 2-theta angle values or comprising at least five peaks having d-spacing values selected from those set forth in Table 31 as defined herein, wherein the XRPD pattern is measured in a diffractometer using copper anode, at wavelength alpha1 of 1.5406 Å and wavelength alpha2 of 1.5444 Å.
1.9 Any of Caprylate Salts 1-1.8, wherein said salt crystal exhibits a Differential Scanning Calorimetry (DSC) pattern comprising an endothermic peak between about 102° C. and 105° C., e.g., at about 104° C.
1.10 Any of Caprylate Salts 1-1.9, wherein the Compound A free base and caprylic acid counterion are present in a molar ratio of about 2:1 to about 1:2, e.g., about 1:1, e.g., about 1:1.4.

In a further embodiment, the present disclosure provides for a besylate salt [Besylate Salt 1] of Compound A.
1.1 Besylate Salt 1, which is in crystalline form.
1.2 Besylate Salt 1 or 1.1, which is in anhydrous form.
1.3 Any of Besylate Salts 1-1.1, wherein the salt crystal is a 3-heptanone solvate.
1.4 Any of Besylate Salts 1-1.3, wherein said salt crystal exhibits an X-ray powder diffraction pattern comprising at least five peaks having 2-theta angle values selected from the group consisting of: 7.7, 7.8, 11.7, 13.9, 15.9, 21.4, 21.9, 22.1, 23.4, and 26.5 degrees, wherein the XRPD pattern is measured in a diffractometer using copper anode, e.g., at wavelength alpha1 of 1.5406 Å and wavelength alpha2 of 1.5444 Å.
1.5 Any of Besylate Salts 1-1.4, wherein said salt crystal exhibits an X-ray powder diffraction pattern comprising at least five peaks having 2-theta angle values selected from the group consisting of: 7.7, 7.8, 11.7, 21.9, and 23.4 degrees, wherein the XRPD pattern is measured in a diffractometer using copper anode, e.g., at wavelength alpha1 of 1.5406 Å and wavelength alpha2 of 1.5444 Å.
1.6 Any of Besylate Salts 1-1.5, wherein said salt crystal exhibits an X-ray powder diffraction pattern comprising at least five peaks having d-spacing values selected from the group consisting of 11.5, 11.4, 7.6, 6.4, 6.3, 5.6, 4.2, 4.1, 3.8, and 3.4 Å.
1.7 Any of Besylate Salts 1-1.6, wherein said salt crystal exhibits an X-ray powder diffraction pattern comprising peaks having d-spacing values selected from the group consisting of 11.5, 11.4, 7.6, 4.1, and 3.8 Å.
1.8 Any of Besylate Salts 1-1.7, wherein said salt crystal exhibits an X-ray powder diffraction pattern comprising at least five peaks having 2-theta angle values or comprising at least five peaks having d-spacing values selected from those set forth in Table 32 as defined herein, wherein the XRPD pattern is measured in a diffractometer using copper anode, at wavelength alpha1 of 1.5406 Å and wavelength alpha2 of 1.5444 Å.
1.9 Any of Besylate Salts 1-1.8, wherein said salt crystal exhibits a Differential Scanning Calorimetry (DSC) pattern comprising an endothermic peak between about 237° C. and 240° C., e.g., at about 238° C.
1.10 Any of Besylate Salts 1-1.9, wherein the Compound A free base and benzenesulfonic acid counterion are present in a molar ratio of about 2:1 to about 1:2, e.g., about 1:1.

In a further embodiment, the present disclosure provides for a benzoate salt [Benzoate Salt 1] of Compound A.
1.1 Benzoate Salt 1, which is in crystalline form.
1.2 Benzoate Salt 1 or 1.1, which is in anhydrous form.
1.3 Any of Benzoate Salts 1-1.1, wherein the salt crystal is a 3-heptanone solvate.
1.4 Any of Benzoate Salts 1-1.3, wherein said salt crystal exhibits an X-ray powder diffraction pattern comprising at least five peaks having 2-theta angle values selected from the group consisting of: 5.3, 5.5, 5.8, 6.3, 6.4, 11.6, 12.3, 13.1, 19.0, and 19.1 degrees, wherein the XRPD pattern is measured in a diffractometer using copper anode, e.g., at wavelength alpha1 of 1.5406 Å and wavelength alpha2 of 1.5444 Å.
1.5 Any of Benzoate Salts 1-1.4, wherein said salt crystal exhibits an X-ray powder diffraction pattern comprising at least five peaks having 2-theta angle values selected from the group consisting of: 5.8, 6.3, 6.4, 11.6, and 13.1 degrees, wherein the XRPD pattern is measured in a diffractometer using copper anode, e.g., at wavelength alpha1 of 1.5406 Å and wavelength alpha2 of 1.5444 Å.
1.6 Any of Benzoate Salts 1-1.5, wherein said salt crystal exhibits an X-ray powder diffraction pattern comprising at least five peaks having d-spacing values selected from the group consisting of 16.5, 16.2, 15.2, 14.0, 13.9, 7.7, 7.2, 6.8, 4.6, and 4.7 Å.
1.7 Any of Benzoate Salts 1-1.6, wherein said salt crystal exhibits an X-ray powder diffraction pattern comprising peaks having d-spacing values selected from the group consisting of 15.2, 14.0, 13.9, 7.7, and 6.8 Å.
1.8 Any of Benzoate Salts 1-1.7, wherein said salt crystal exhibits an X-ray powder diffraction pattern comprising at least five peaks having 2-theta angle values or comprising at least five peaks having d-spacing values selected from those set forth in Table 33 as defined herein, wherein the XRPD pattern is measured in a diffractometer using copper anode, at wavelength alpha1 of 1.5406 Å and wavelength alpha2 of 1.5444 Å.
1.9 Any of Benzoate Salts 1-1.8, wherein said salt crystal exhibits a Differential Scanning Calorimetry (DSC) pattern comprising an endothermic peak between about 59° C. and 62° C., e.g., at about 60° C., between about 81° C. and 84° C., e.g., at about 83° C., and/or between about 115° C. and 118° C., e.g., at about 116° C.
1.10 Any of Benzoate Salts 1-1.9, wherein the Compound A free base and benzoic acid counterion are present in a molar ratio of about 2:1 to about 1:2, e.g., about 1:1, e.g., about 1:1.1.

In a further embodiment, the present disclosure provides for a nicotinate salt [Nicotinate Salt 1] of Compound A.
1.1 Nicotinate Salt 1, which is in crystalline form.
1.2 Nicotinate Salt 1 or 1.1, which is in anhydrous form.
1.3 Any of Nicotinate Salts 1-1.1, wherein the salt crystal is an acetonitrile solvate.
1.4 Any of Nicotinate Salts 1-1.3, wherein said salt crystal exhibits an X-ray powder diffraction pattern comprising at least five peaks having 2-theta angle values selected from the group consisting of: 5.2, 8.7, 10.8, 12.0, 12.7, 17.9, 20.0, 20.5, 20.8, and 21.6 degrees, wherein the XRPD pattern is measured in a diffractometer using copper anode, e.g., at wavelength alpha1 of 1.5406 Å and wavelength alpha2 of 1.5444 Å.
1.5 Any of Nicotinate Salts 1-1.4, wherein said salt crystal exhibits an X-ray powder diffraction pattern comprising at least five peaks having 2-theta angle values selected from the group consisting of: 5.2, 10.8, 12.7, 20.0, and 20.8 degrees, wherein the XRPD pattern is measured in a diffractometer using copper anode, e.g., at wavelength alpha1 of 1.5406 Å and wavelength alpha2 of 1.5444 Å.
1.6 Any of Nicotinate Salts 1-1.5, wherein said salt crystal exhibits an X-ray powder diffraction pattern comprising at least five peaks having d-spacing values selected from the group consisting of 17.0, 10.1, 8.2, 7.4, 6.9, 5.0, 4.4, 4.3, 4.1, and 3.8 Å.
1.7 Any of Nicotinate Salts 1-1.6, wherein said salt crystal exhibits an X-ray powder diffraction pattern comprising peaks having d-spacing values selected from the group consisting of 17.0, 8.2, 6.9, 4.4, and 4.3 Å.
1.8 Any of Nicotinate Salts 1-1.7, wherein said salt crystal exhibits an X-ray powder diffraction pattern comprising at least five peaks having 2-theta angle values or comprising at least five peaks having d-spacing values selected from those set forth in Table 34 as defined herein, wherein the XRPD pattern is measured in a diffractometer using copper anode, at wavelength alpha1 of 1.5406 Å and wavelength alpha2 of 1.5444 Å.
1.9 Any of Nicotinate Salts 1-1.8, wherein said salt crystal exhibits a Differential Scanning Calorimetry (DSC) pattern comprising an endothermic peak between about 135° C. and 138° C., e.g., at about 137° C.
1.10 Any of Nicotinate Salts 1-1.9, wherein the Compound A free base and nicotinic acid counterion are present in a molar ratio of about 2:1 to about 1:2, e.g., about 1:1.

In a further embodiment, the present disclosure provides for an isonicotinate salt [Isonicotinate Salt 1] of Compound A.
1.1 Isonicotinate Salt 1, which is in crystalline form.
1.2 Isonicotinate Salt 1 or 1.1, which is in solvate form.
1.3 Any of Isonicotinate Salts 1-1.1, wherein the salt crystal is a toluene solvate.
1.4 Any of Isonicotinate Salts 1-1.3, wherein said salt crystal exhibits an X-ray powder diffraction pattern comprising at least five peaks having 2-theta angle values selected from the group consisting of: 5.5, 7.3, 11.7, 12.8, 16.7, 17.1, 17.3, 17.9, 20.4, and 28.0 degrees, wherein the XRPD pattern is measured in a diffractometer using copper anode, e.g., at wavelength alpha1 of 1.5406 Å and wavelength alpha2 of 1.5444 Å.
1.5 Any of Isonicotinate Salts 1-1.4, wherein said salt crystal exhibits an X-ray powder diffraction pattern comprising at least five peaks having 2-theta angle values selected from the group consisting of: 5.5, 7.3, 16.7, 17.1, and 17.3 degrees, wherein the XRPD pattern is measured in a diffractometer using copper anode, e.g., at wavelength alpha1 of 1.5406 Å and wavelength alpha2 of 1.5444 Å.
1.6 Any of Isonicotinate Salts 1-1.5, wherein said salt crystal exhibits an X-ray powder diffraction pattern comprising at least five peaks having d-spacing values selected from the group consisting of 16.1, 15.9, 12.1, 7.5, 6.9, 5.3, 5.2, 5.1, 5.0, and 3.2 Å.
1.7 Any of Isonicotinate Salts 1-1.6, wherein said salt crystal exhibits an X-ray powder diffraction pattern comprising peaks having d-spacing values selected from the group consisting of 16.1, 15.9, 5.3, 5.2, and 5.1 Å.
1.8 Any of Isonicotinate Salts 1-1.7, wherein said salt crystal exhibits an X-ray powder diffraction pattern comprising at least five peaks having 2-theta angle values or comprising at least five peaks having d-spacing values selected from those set forth in Table 35 as defined herein, wherein the XRPD pattern is measured in a diffractometer using copper anode, at wavelength alpha1 of 1.5406 Å and wavelength alpha2 of 1.5444 Å.
1.9 Any of Isonicotinate Salts 1-1.8, wherein said salt crystal exhibits a Differential Scanning Calorimetry (DSC) pattern comprising an endothermic peak between about 111° C. and 114° C., e.g., at about 113° C., and/or between about 128° C. and 130° C., e.g., at about 129° C.
1.10 Any of Isonicotinate Salts 1-1.9, wherein the Compound A free base and isonicotinic acid counterion are present in a molar ratio of about 2:1 to about 1:2, e.g., about 1:1, e.g., about 1:0.7.

In a further embodiment, the present disclosure provides for an orotate salt [Orotate Salt 1] of Compound A.
1.1 Orotate Salt 1, which is in crystalline form.
1.2 Orotate Salt 1 or 1.1, which is in solvate form.
1.3 Any of Orotate Salts 1-1.1, wherein the salt crystal is a 2-butanone solvate.
1.4 Any of Orotate Salts 1-1.3, wherein said salt crystal exhibits an X-ray powder diffraction pattern comprising at least five peaks having 2-theta angle values selected from the group consisting of: 5.8, 7.2, 10.4, 11.8, 12.6, 13.5, 16.9, 21.9, 22.5, and 28.9 degrees, wherein the XRPD pattern is measured in a diffractometer using copper anode, e.g., at wavelength alpha1 of 1.5406 Å and wavelength alpha2 of 1.5444 Å.
1.5 Any of Orotate Salts 1-1.4, wherein said salt crystal exhibits an X-ray powder diffraction pattern comprising at least five peaks having 2-theta angle values selected from the group consisting of: 5.8, 7.2, 16.9, 21.9, and 28.9 degrees, wherein the XRPD pattern is measured in a diffractometer using copper anode, e.g., at wavelength alpha1 of 1.5406 Å and wavelength alpha2 of 1.5444 Å.
1.6 Any of Orotate Salts 1-1.5, wherein said salt crystal exhibits an X-ray powder diffraction pattern comprising at least five peaks having d-spacing values selected from the group consisting of 15.3, 12.3, 8.5, 7.5, 7.0, 6.6, 5.2, 4.1, 4.0, and 3.1 Å.
1.7 Any of Orotate Salts 1-1.6, wherein said salt crystal exhibits an X-ray powder diffraction pattern comprising peaks having d-spacing values selected from the group consisting of 15.3, 12.3, 5.2, 4.1, and 3.1 Å.
1.8 Any of Orotate Salts 1-1.7, wherein said salt crystal exhibits an X-ray powder diffraction pattern comprising at least five peaks having 2-theta angle values or comprising at least five peaks having d-spacing values selected from those set forth in Table 36 as defined herein, wherein the XRPD pattern is measured in a diffractometer using copper anode, at wavelength alpha1 of 1.5406 Å and wavelength alpha2 of 1.5444 Å.
1.9 Any of Orotate Salts 1-1.8, wherein said salt crystal exhibits a Differential Scanning Calorimetry (DSC) pattern comprising an endothermic peak between about 137° C. and 140° C., e.g., at about 138° C.
1.10 Any of Orotate Salts 1-1.9, wherein the Compound A free base and orotic acid counterion are present in a molar ratio of about 2:1 to about 1:2, e.g., about 1:1, e.g., about 1:2.

In a further embodiment, the present disclosure provides for a first camsylate salt [Camsylate Salt 1] of Compound A.
1.1 Camsylate Salt 1, which is in crystalline form.
1.2 Camsylate Salt 1 or 1.1, which is in anhydrous form.
1.3 Any of Camsylate Salts 1-1.1, wherein the salt crystal is a 3-heptanone solvate.
1.4 Any of Camsylate Salts 1-1.3, wherein said salt crystal exhibits an X-ray powder diffraction pattern comprising at least five peaks having 2-theta angle values selected from the group consisting of: 5.3, 8.3, 10.0, 12.6, 15.2, 17.8, 18.0, 19.3, 19.4, and 24.5 degrees, wherein the XRPD pattern is measured in a diffractometer using copper anode, e.g., at wavelength alpha1 of 1.5406 Å and wavelength alpha2 of 1.5444 Å.
1.5 Any of Camsylate Salts 1-1.4, wherein said salt crystal exhibits an X-ray powder diffraction pattern comprising at least five peaks having 2-theta angle values selected from the group consisting of: 5.3, 10.0, 15.2, 18.0, and 19.4 degrees, wherein the XRPD pattern is measured in a diffractometer using copper anode, e.g., at wavelength alpha1 of 1.5406 Å and wavelength alpha2 of 1.5444 Å.
1.6 Any of Camsylate Salts 1-1.5, wherein said salt crystal exhibits an X-ray powder diffraction pattern comprising at least five peaks having d-spacing values selected from the group consisting of 16.6, 10.7, 8.9, 7.0, 5.8, 5.6, 5.0, 4.9, 4.6, and 3.6 Å.
1.7 Any of Camsylate Salts 1-1.6, wherein said salt crystal exhibits an X-ray powder diffraction pattern comprising peaks having d-spacing values selected from the group consisting of 16.6, 8.9, 5.8, 4.9, and 4.6 Å.
1.8 Any of Camsylate Salts 1-1.7, wherein said salt crystal exhibits an X-ray powder diffraction pattern comprising at least five peaks having 2-theta angle values or comprising at least five peaks having d-spacing values selected from those set forth in Table 37 as defined herein, wherein the XRPD pattern is measured in a diffractometer using copper anode, at wavelength alpha1 of 1.5406 Å and wavelength alpha2 of 1.5444 Å.
1.9 Any of Camsylate Salts 1-1.8, wherein said salt crystal exhibits a Differential Scanning Calorimetry (DSC) pattern comprising an endothermic peak between about 227° C. and 230° C., e.g., at about 228° C., and/or between about 253° C. and 256° C., e.g., at about 254° C.
1.10 Any of Camsylate Salts 1-1.9, wherein the Compound A free base and camphorsulfonic acid counterion are present in a molar ratio of about 2:1 to about 1:2, e.g., about 1:1.

In a further embodiment, the present disclosure provides for a second camsylate salt [Camsylate Salt 2] of Compound A.
2.1 Camsylate Salt 2, which is in crystalline form.
2.2 Camsylate Salt 2 or 2.1, which is in solvate form.
2.3 Any of Camsylate Salts 2-2.1, wherein the salt crystal is a toluene solvate.
2.4 Any of Camsylate Salts 2-2.3, wherein said salt crystal exhibits an X-ray powder diffraction pattern comprising at least five peaks having 2-theta angle values selected from the group consisting of: 4.7, 5.4, 9.1, 9.4, 12.6, 16.0, 16.5, 17.6, 18.3, and 20.2 degrees, wherein the XRPD pattern is measured in a diffractometer using copper anode, e.g., at wavelength alpha1 of 1.5406 Å and wavelength alpha2 of 1.5444 Å.
2.5 Any of Camsylate Salts 2-2.4, wherein said salt crystal exhibits an X-ray powder diffraction pattern comprising at least five peaks having 2-theta angle values selected from the group consisting of: 4.7, 5.4, 9.1, 17.6, and 18.3 degrees, wherein the XRPD pattern is measured in a diffractometer using copper anode, e.g., at wavelength alpha1 of 1.5406 Å and wavelength alpha2 of 1.5444 Å.
2.6 Any of Camsylate Salts 2-2.5, wherein said salt crystal exhibits an X-ray powder diffraction pattern comprising at least five peaks having d-spacing values selected from the group consisting of 18.7, 16.5, 9.8, 9.4, 7.0, 5.5, 5.4, 5.0, 4.8, and 4.4 Å.
2.7 Any of Camsylate Salts 2-2.6, wherein said salt crystal exhibits an X-ray powder diffraction pattern comprising peaks having d-spacing values selected from the group consisting of 18.7, 16.5, 9.8, 5.0, and 4.8 Å.
2.8 Any of Camsylate Salts 2-2.7, wherein said salt crystal exhibits an X-ray powder diffraction pattern comprising at least five peaks having 2-theta angle values or comprising at least five peaks having d-spacing values selected from those set forth in Table 38 as defined herein, wherein the XRPD pattern is measured in a diffractometer using copper anode, at wavelength alpha1 of 1.5406 Å and wavelength alpha2 of 1.5444 Å.
2.9 Any of Camsylate Salts 2-2.8, wherein the Compound A free base and camphorsulfonic acid counterion are present in a molar ratio of about 2:1 to about 1:2, e.g., about 1:1.

In a further embodiment, the present disclosure provides for a first salicylate salt [Salicylate Salt 1] of Compound A.
1.1 Salicylate Salt 1, which is in crystalline form.
1.2 Salicylate Salt 1 or 1.1, which is in anhydrous form.
1.3 Any of Salicylate Salts 1-1.1, wherein the salt crystal is an acetonitrile solvate.
1.4 Any of Salicylate Salts 1-1.3, wherein said salt crystal exhibits an X-ray powder diffraction pattern comprising at least five peaks having 2-theta angle values selected from the group consisting of: 7.0, 11.0, 11.5, 13.3, 13.8, 17.2, 18.9, 19.8, 20.3, and 21.0 degrees, wherein the XRPD pattern is measured in a diffractometer using copper anode, e.g., at wavelength alpha1 of 1.5406 Å and wavelength alpha2 of 1.5444 Å.
1.5 Any of Salicylate Salts 1-1.4, wherein said salt crystal exhibits an X-ray powder diffraction pattern comprising at least five peaks having 2-theta angle values selected from the group consisting of: 7.0, 11.0, 13.3, 13.8, and 17.2 degrees, wherein the XRPD pattern is measured in a diffractometer using copper anode, e.g., at wavelength alpha1 of 1.5406 Å and wavelength alpha2 of 1.5444 Å.
1.6 Any of Salicylate Salts 1-1.5, wherein said salt crystal exhibits an X-ray powder diffraction pattern comprising at least five peaks having d-spacing values selected from the group consisting of 12.7, 8.1, 7.7, 6.7, 6.4, 5.1, 4.7, 4.5, 4.4, and 4.2 Å.
1.7 Any of Salicylate Salts 1-1.6, wherein said salt crystal exhibits an X-ray powder diffraction pattern comprising peaks having d-spacing values selected from the group consisting of 12.7, 8.1, 6.7, 6.4, and 5.1 Å.
1.8 Any of Salicylate Salts 1-1.7, wherein said salt crystal exhibits an X-ray powder diffraction pattern comprising at least five peaks having 2-theta angle values or comprising at least five peaks having d-spacing values selected from those set forth in Table 39 as defined herein, wherein the XRPD pattern is measured in a diffractometer using copper anode, at wavelength alpha1 of 1.5406 Å and wavelength alpha2 of 1.5444 Å.
1.9 Any of Salicylate Salts 1-1.8, wherein said salt crystal exhibits a Differential Scanning Calorimetry (DSC) pattern comprising an endothermic peak between about 146° C. and 150° C., e.g., at about 147° C., between about 153° C. and 156° C., e.g., at about 155° C., between about 196° C. and 199° C., e.g., at about 197° C., and/or between about 244° C. and 247° C., e.g., at about 245° C.
1.10 Any of Salicylate Salts 1-1.9, wherein the Compound A free base and salicylic acid counterion are present in a molar ratio of about 2:1 to about 1:2, e.g., about 1:1.

In a further embodiment, the present disclosure provides for a second salicylate salt [Salicylate Salt 2] of Compound A.
2.1 Salicylate Salt 2, which is in crystalline form.
2.2 Salicylate Salt 2 or 2.1, which is in solvate form.
2.3 Any of Salicylate Salts 2-2.1, wherein the salt crystal is a toluene solvate.
2.4 Any of Salicylate Salts 2-2.3, wherein said salt crystal exhibits an X-ray powder diffraction pattern comprising at least five peaks having 2-theta angle values selected from the group consisting of: 6.9, 11.3, 12.4, 14.3, 16.7, 19.7, 21.3, 22.0, 24.5, and 25.0 degrees, wherein the XRPD pattern is measured in a diffractometer using copper anode, e.g., at wavelength alpha1 of 1.5406 Å and wavelength alpha2 of 1.5444 Å.
2.5 Any of Salicylate Salts 2-2.4, wherein said salt crystal exhibits an X-ray powder diffraction pattern comprising at least five peaks having 2-theta angle values selected from the group consisting of: 6.9, 12.4, 14.3, 16.7, and 19.7 degrees, wherein the XRPD pattern is measured in a diffractometer using copper anode, e.g., at wavelength alpha1 of 1.5406 Å and wavelength alpha2 of 1.5444 Å.
2.6 Any of Salicylate Salts 2-2.5, wherein said salt crystal exhibits an X-ray powder diffraction pattern comprising at least five peaks having d-spacing values selected from the group consisting of 14.1, 12.7, 7.8, 7.1, 6.2, 5.3, 4.5, 4.2, 4.0, and 3.6 Å.
2.7 Any of Salicylate Salts 2-2.6, wherein said salt crystal exhibits an X-ray powder diffraction pattern comprising peaks having d-spacing values selected from the group consisting of 12.7, 7.1, 6.2, 5.3, and 4.5 Å.
2.8 Any of Salicylate Salts 2-2.7, wherein said salt crystal exhibits an X-ray powder diffraction pattern comprising at least five peaks having 2-theta angle values or comprising at least five peaks having d-spacing values selected from those set forth in Table 40 as defined herein, wherein the XRPD pattern is measured in a diffractometer using copper anode, at wavelength alpha1 of 1.5406 Å and wavelength alpha2 of 1.5444 Å.
2.9 Any of Salicylate Salts 2-2.8, wherein said salt crystal exhibits a Differential Scanning Calorimetry (DSC) pattern comprising an endothermic peak between about 127° C. and 130° C., e.g., at about 128° C., between about 143° C. and 146° C., e.g., at about 144° C., between about 180° C. and 183° C., e.g., at about 181° C., between about 196° C. and 199° C., e.g., at about 197° C., and between about 244° C. and 247° C., e.g., at about 247° C.
2.10 Any of Salicylate Salts 2-2.9, wherein the Compound A free base and salicylic acid counterion are present in a molar ratio of about 2:1 to about 1:2, e.g., about 1:1.

In a further embodiment, the present disclosure provides for an aminosalicylate salt [Aminosalicylate Salt 1] of Compound A.
1.1 Aminosalicylate Salt 1, which is in crystalline form.
1.2 Aminosalicylate Salt 1 or 1.1, which is in anhydrous form.
1.3 Any of Aminosalicylate Salts 1-1.1, wherein the salt crystal is an acetonitrile solvate.
1.4 Any of Aminosalicylate Salts 1-1.3, wherein said salt crystal exhibits an X-ray powder diffraction pattern comprising at least five peaks having 2-theta angle values selected from the group consisting of: 6.8, 10.7, 13.5, 13.9, 17.0, 20.4, 20.7, 20.8, 21.3, and 21.5 degrees, wherein the XRPD pattern is measured in a diffractometer using copper anode, e.g., at wavelength alpha1 of 1.5406 Å and wavelength alpha2 of 1.5444 Å.
1.5 Any of Aminosalicylate Salts 1-1.4, wherein said salt crystal exhibits an X-ray powder diffraction pattern comprising at least five peaks having 2-theta angle values selected from the group consisting of: 6.8, 10.7, 13.9, 17.0, and 20.7 degrees, wherein the XRPD pattern is measured in a diffractometer using copper anode, e.g., at wavelength alpha1 of 1.5406 Å and wavelength alpha2 of 1.5444 Å.
1.6 Any of Aminosalicylate Salts 1-1.5, wherein said salt crystal exhibits an X-ray powder diffraction pattern comprising at least five peaks having d-spacing values selected from the group consisting of 13.0, 8.3, 7.0, 6.5, 6.4, 5.3, 5.2, 4.3, 4.2, and 4.1 Å.
1.7 Any of Aminosalicylate Salts 1-1.6, wherein said salt crystal exhibits an X-ray powder diffraction pattern comprising peaks having d-spacing values selected from the group consisting of 13.0, 8.3, 6.4, 5.2, and 4.3 Å.
1.8 Any of Aminosalicylate Salts 1-1.7, wherein said salt crystal exhibits an X-ray powder diffraction pattern comprising at least five peaks having 2-theta angle values or comprising at least five peaks having d-spacing values selected from those set forth in Table 42 as defined herein, wherein the XRPD pattern is measured in a diffractometer using copper anode, at wavelength alpha1 of 1.5406 Å and wavelength alpha2 of 1.5444 Å.
1.9 Any of Aminosalicylate Salts 1-1.8, wherein said salt crystal exhibits a Differential Scanning Calorimetry (DSC) pattern comprising an endothermic peak between about 130° C. and 133° C., e.g., at about 132° C., and/or between about 161° C. and 164° C., e.g., at about 162° C.
1.10 Any of Aminosalicylate Salts 1-1.9, wherein the Compound A free base and amino salicylic acid counterion are present in a molar ratio of about 2:1 to about 1:2, e.g., about 1:1.

In a further embodiment, the present disclosure provides for a first mandelate salt [Mandelate Salt 1] of Compound A.
1.1 Mandelate Salt 1, which is in crystalline form.
1.2 Mandelate Salt 1 or 1.1, which is in solvate form.
1.3 Any of Mandelate Salts 1-1.1, wherein the salt crystal is a toluene solvate.
1.4 Any of Mandelate Salts 1-1.3, wherein said salt crystal exhibits an X-ray powder diffraction pattern comprising at least five peaks having 2-theta angle values selected from the group consisting of: 6.5, 7.7, 8.5, 10.0, 11.4, 11.9, 19.8, 20.0, 20.1, and 20.8 degrees, wherein the XRPD pattern is measured in a diffractometer using copper anode, e.g., at wavelength alpha1 of 1.5406 Å and wavelength alpha2 of 1.5444 Å.
1.5 Any of Mandelate Salts 1-1.4, wherein said salt crystal exhibits an X-ray powder diffraction pattern comprising at least five peaks having 2-theta angle values selected from the group consisting of: 6.5, 8.5, 11.4, 20.0, and 20.1 degrees, wherein the XRPD pattern is measured in a diffractometer using copper anode, e.g., at wavelength alpha1 of 1.5406 Å and wavelength alpha2 of 1.5444 Å.
1.6 Any of Mandelate Salts 1-1.5, wherein said salt crystal exhibits an X-ray powder diffraction pattern comprising at least five peaks having d-spacing values selected from the group consisting of 13.6, 11.5, 10.3, 8.9, 7.8, 7.4, 4.5, 4.4, 4.3, and 4.0 Å.
1.7 Any of Mandelate Salts 1-1.6, wherein said salt crystal exhibits an X-ray powder diffraction pattern comprising peaks having d-spacing values selected from the group consisting of 13.6, 10.3, 8.9 7.8, and 4.4 Å.
1.8 Any of Mandelate Salts 1-1.7, wherein said salt crystal exhibits an X-ray powder diffraction pattern comprising at least five peaks having 2-theta angle values or comprising at least five peaks having d-spacing values selected from those set forth in Table 43 as defined herein, wherein the XRPD pattern is measured in a diffractometer using copper anode, at wavelength alpha1 of 1.5406 Å and wavelength alpha2 of 1.5444 Å.
1.9 Any of Mandelate Salts 1-1.8, wherein said salt crystal exhibits a Differential Scanning Calorimetry (DSC) pattern comprising an endothermic peak between about 119° C. and 128° C., e.g., at about 120° C. or 126° C.
1.10 Any of Mandelate Salts 1-1.9, wherein the Compound A free base and mandelic acid counterion are present in a molar ratio of about 2:1 to about 1:2, e.g., about 1:1.

In a further embodiment, the present disclosure provides for a first mandelate salt [Mandelate Salt 2] of Compound A.
2.1 Mandelate Salt 2, which is in crystalline form.
2.2 Mandelate Salt 2 or 2.1, which is in solvate form.
2.3 Any of Mandelate Salts 2-2.1, wherein the salt crystal is a 3-heptanone solvate.
2.4 Any of Mandelate Salts 2-2.3, wherein said salt crystal exhibits an X-ray powder diffraction pattern comprising at least five peaks having 2-theta angle values selected from the group consisting of: 6.0, 8.1, 8.5, 8.6, 8.7, 12.6, 17.3, 20.7, 22.2, and 22.1 degrees, wherein the XRPD pattern is measured in a diffractometer using copper anode, e.g., at wavelength alpha1 of 1.5406 Å and wavelength alpha2 of 1.5444 Å.
2.5 Any of Mandelate Salts 2-2.4, wherein said salt crystal exhibits an X-ray powder diffraction pattern comprising at least five peaks having 2-theta angle values selected from the group consisting of: 8.1, 8.6, 8.7, 12.6, and 22.2 degrees, wherein the XRPD pattern is measured in a diffractometer using copper anode, e.g., at wavelength alpha1 of 1.5406 Å and wavelength alpha2 of 1.5444 Å.
2.6 Any of Mandelate Salts 2-2.5, wherein said salt crystal exhibits an X-ray powder diffraction pattern comprising at least five peaks having d-spacing values selected from the group consisting of 14.6, 11.0, 10.4, 10.3, 10.2, 7.0, 5.1, 4.3, 4.0, and 3.5 Å.
2.7 Any of Mandelate Salts 2-2.6, wherein said salt crystal exhibits an X-ray powder diffraction pattern comprising peaks having d-spacing values selected from the group consisting of 11.0, 10.3, 10.2, 7.0, and 4.0 Å.
2.8 Any of Mandelate Salts 2-2.7, wherein said salt crystal exhibits an X-ray powder diffraction pattern comprising at least five peaks having 2-theta angle values or comprising at least five peaks having d-spacing values selected from those set forth in Table 44 as defined herein, wherein the XRPD pattern is measured in a diffractometer using copper anode, at wavelength alpha1 of 1.5406 Å and wavelength alpha2 of 1.5444 Å.
2.9 Any of Mandelate Salts 2-2.8, wherein said salt crystal exhibits a Differential Scanning Calorimetry (DSC) pattern comprising an endothermic peak between about 102° C. and 105° C., e.g., at about 103° C.
2.10 Any of Mandelate Salts 2-2.9, wherein the Compound A free base and mandelic acid counterion are present in a molar ratio of about 2:1 to about 1:2, e.g., about 1:1.

In a further embodiment, the present disclosure provides for a first acetamido benzoate salt [Acetamido Benzoate Salt 1] of Compound A.
1.1 Acetamido Benzoate Salt 1, which is in crystalline form.
1.2 Acetamido Benzoate Salt 1 or 1.1, which is in anhydrous form.
1.3 Any of Acetamido Benzoate Salts 1-1.1, wherein the salt crystal is an ethyl acetate solvate.
1.4 Any of Acetamido Benzoate Salts 1-1.3, wherein said salt crystal exhibits an X-ray powder diffraction pattern comprising at least five peaks having 2-theta angle values selected from the group consisting of: 6.0, 8.3, 11.8, 12.9, 14.2, 15.2, 18.7, 19.6, 21.4, and 23.4 degrees, wherein the XRPD pattern is measured in a diffractometer using copper anode, e.g., at wavelength alpha1 of 1.5406 Å and wavelength alpha2 of 1.5444 Å.
1.5 Any of Acetamido Benzoate Salts 1-1.4, wherein said salt crystal exhibits an X-ray powder diffraction pattern comprising at least five peaks having 2-theta angle values selected from the group consisting of: 6.0, 8.3, 14.2, 15.2, and 18.7 degrees, wherein the XRPD pattern is measured in a diffractometer using copper anode, e.g., at wavelength alpha1 of 1.5406 Å and wavelength alpha2 of 1.5444 Å.
1.6 Any of Acetamido Benzoate Salts 1-1.5, wherein said salt crystal exhibits an X-ray powder diffraction pattern comprising at least five peaks having d-spacing values selected from the group consisting of 14.7, 10.6, 7.5, 6.8, 6.2, 5.8, 4.7, 4.5, 4.2, and 3.8 Å.
1.7 Any of Acetamido Benzoate Salts 1-1.6, wherein said salt crystal exhibits an X-ray powder diffraction pattern comprising peaks having d-spacing values selected from the group consisting of 14.7, 10.6, 6.2, 5.8, and 4.7 Å.
1.8 Any of Acetamido Benzoate Salts 1-1.7, wherein said salt crystal exhibits an X-ray powder diffraction pattern comprising at least five peaks having 2-theta angle values or comprising at least five peaks having d-spacing values selected from those set forth in Table 45 as defined herein, wherein the XRPD pattern is measured in a diffractometer using copper anode, at wavelength alpha1 of 1.5406 Å and wavelength alpha2 of 1.5444 Å.
1.9 Any of Acetamido Benzoate Salts 1-1.8, wherein said salt crystal exhibits a Differential Scanning Calorimetry (DSC) pattern comprising an endothermic peak between about 168° C. and 171° C., e.g., at about 170° C.
1.10 Any of Acetamido Benzoate Salts 1-1.9, wherein the Compound A free base and 4-Acetamido-benzoate acid counterion are present in a molar ratio of about 2:1 to about 1:2, e.g., about 1:1, e.g., about 1:1.3.

In a further embodiment, the present disclosure provides for a first acetamido benzoate salt [Acetamido Benzoate Salt 2] of Compound A.
1.1 Acetamido Benzoate Salt 2, which is in crystalline form.
1.2 Acetamido Benzoate Salt 2 or 2.1, which is in solvate form.
1.3 Any of Acetamido Benzoate Salts 2-2.1, wherein the salt crystal is a 3-heptanone solvate.
1.4 Any of Acetamido Benzoate Salts 2-2.3, wherein said salt crystal exhibits an X-ray powder diffraction pattern comprising at least five peaks having 2-theta angle values selected from the group consisting of: 4.9, 6.8, 10.7, 11.6, 13.8, 14.3, 15.8, 16.4, 19.2, and 21.5 degrees, wherein the XRPD pattern is measured in a diffractometer using copper anode, e.g., at wavelength alpha1 of 1.5406 Å and wavelength alpha2 of 1.5444 Å.
1.5 Any of Acetamido Benzoate Salts 2-2.4, wherein said salt crystal exhibits an X-ray powder diffraction pattern comprising at least five peaks having 2-theta angle values selected from the group consisting of: 4.9, 6.8, 14.3, 15.8, and 21.5 degrees, wherein the XRPD pattern is measured in a diffractometer using copper anode, e.g., at wavelength alpha1 of 1.5406 Å and wavelength alpha2 of 1.5444 Å.
1.6 Any of Acetamido Benzoate Salts 2-2.5, wherein said salt crystal exhibits an X-ray powder diffraction pattern comprising at least five peaks having d-spacing values selected from the group consisting of 18.1, 13.1, 8.3, 7.7, 6.4, 6.2, 5.6, 5.4, 4.6, and 4.1 Å.
1.7 Any of Acetamido Benzoate Salts 2-2.6, wherein said salt crystal exhibits an X-ray powder diffraction pattern comprising peaks having d-spacing values selected from the group consisting of 18.1, 13.1, 6.2, 5.6, and 4.1 Å.
1.8 Any of Acetamido Benzoate Salts 2-2.7, wherein said salt crystal exhibits an X-ray powder diffraction pattern comprising at least five peaks having 2-theta angle values or comprising at least five peaks having d-spacing values selected from those set forth in Table 46 as defined herein, wherein the XRPD pattern is measured in a diffractometer using copper anode, at wavelength alpha1 of 1.5406 Å and wavelength alpha2 of 1.5444 Å.
1.9 Any of Acetamido Benzoate Salts 2-2.8, wherein said salt crystal exhibits a Differential Scanning Calorimetry (DSC) pattern comprising an endothermic peak between about 127° C. and 130° C., e.g., at about 129° C., and/or between about 170° C. and 173° C., e.g., at about 172° C.
1.10 Any of Acetamido Benzoate Salts 2-2.9, wherein the Compound A free base and 4-Acetamido-benzoate acid counterion are present in a molar ratio of about 2:1 to about 1:2, e.g., about 1:1.

In a further embodiment, the present disclosure provides for a trifluoroacetate salt [Trifluoroacetate Salt 1] of Compound A.
1.1 Trifluoroacetate Salt 1, which is in crystalline form.
1.2 Trifluoroacetate Salt 1 or 1.1, which is in anhydrous form.
1.3 Any of Trifluoroacetate Salts 1-1.1, wherein the salt crystal is an acetonitrile solvate.
1.4 Any of Trifluoroacetate Salts 1-1.3, wherein said salt crystal exhibits an X-ray powder diffraction pattern comprising at least five peaks having 2-theta angle values selected from the group consisting of: 6.5, 6.6, 8.7, 8.9, 17.0, 18.8, 19.5, 20.4, 23.4, and 24.6 degrees, wherein the XRPD pattern is measured in a diffractometer using copper anode, e.g., at wavelength alpha1 of 1.5406 Å and wavelength alpha2 of 1.5444 Å.
1.5 Any of Trifluoroacetate Salts 1-1.4, wherein said salt crystal exhibits an X-ray powder diffraction pattern comprising at least five peaks having 2-theta angle values selected from the group consisting of: 6.5, 6.6, 8.9, 17.0, and 23.4 degrees, wherein the XRPD pattern is measured in a diffractometer using copper anode, e.g., at wavelength alpha1 of 1.5406 Å and wavelength alpha2 of 1.5444 Å.
1.6 Any of Trifluoroacetate Salts 1-1.5, wherein said salt crystal exhibits an X-ray powder diffraction pattern comprising at least five peaks having d-spacing values selected from the group consisting of 13.6, 13.3, 10.2, 10.0, 5.2, 4.7, 4.6, 4.3, 3.8, and 3.6 Å.
1.7 Any of Trifluoroacetate Salts 1-1.6, wherein said salt crystal exhibits an X-ray powder diffraction pattern comprising peaks having d-spacing values selected from the group consisting of 13.6, 13.3, 10.0, 5.2, and 3.8 Å.
1.8 Any of Trifluoroacetate Salts 1-1.7, wherein said salt crystal exhibits an X-ray powder diffraction pattern comprising at least five peaks having 2-theta angle values or comprising at least five peaks having d-spacing values selected from those set forth in Table 47 as defined herein, wherein the XRPD pattern is measured in a diffractometer using copper anode, at wavelength alpha1 of 1.5406 Å and wavelength alpha2 of 1.5444 Å.
1.9 Any of Trifluoroacetate Salts 1-1.8, wherein said salt crystal exhibits a Differential Scanning Calorimetry (DSC) pattern comprising an endothermic peak between about 253° C. and 257° C., e.g., at about 255° C.
1.10 Any of Trifluoroacetate Salts 1-1.9, wherein the Compound A free base and trifluoroacetic acid counterion are present in a molar ratio of about 2:1 to about 1:2, e.g., about 1:1, e.g., about 1:0.9.

In a further embodiment, the present disclosure provides for a dichloroacetate salt [Dichloroacetate Salt 1] of Compound A.
1.1 Dichloroacetate Salt 1, which is in crystalline form.
1.2 Dichloroacetate Salt 1 or 1.1, which is in anhydrous form.
1.3 Any of Dichloroacetate Salts 1-1.1, wherein the salt crystal is an acetonitrile solvate.
1.4 Any of Dichloroacetate Salts 1-1.3, wherein said salt crystal exhibits an X-ray powder diffraction pattern comprising at least five peaks having 2-theta angle values selected from the group consisting of: 6.5, 8.8, 11.9, 12.3, 17.2, 17.7, 18.9, 19.3, 23.6, and 28.2 degrees, wherein the XRPD pattern is measured in a diffractometer using copper anode, e.g., at wavelength alpha1 of 1.5406 Å and wavelength alpha2 of 1.5444 Å.
1.5 Any of Dichloroacetate Salts 1-1.4, wherein said salt crystal exhibits an X-ray powder diffraction pattern comprising at least five peaks having 2-theta angle values selected from the group consisting of: 6.5, 11.9, 17.2, 17.7, and 18.9 degrees, wherein the XRPD pattern is measured in a diffractometer using copper anode, e.g., at wavelength alpha1 of 1.5406 Å and wavelength alpha2 of 1.5444 Å.
1.6 Any of Dichloroacetate Salts 1-1.5, wherein said salt crystal exhibits an X-ray powder diffraction pattern comprising at least five peaks having d-spacing values selected from the group consisting of 13.7, 10.1, 7.4, 7.2, 5.2, 5.0, 4.7, 4.6, 3.8, and 3.2 Å.
1.7 Any of Dichloroacetate Salts 1-1.6, wherein said salt crystal exhibits an X-ray powder diffraction pattern comprising peaks having d-spacing values selected from the group consisting of 13.7, 7.4, 5.2, 5.0, and 4.7 Å.
1.8 Any of Dichloroacetate Salts 1-1.7, wherein said salt crystal exhibits an X-ray powder diffraction pattern comprising at least five peaks having 2-theta angle values or comprising at least five peaks having d-spacing values selected from those set forth in Table 48 as defined herein, wherein the XRPD pattern is measured in a diffractometer using copper anode, at wavelength alpha1 of 1.5406 Å and wavelength alpha2 of 1.5444 Å.
1.9 Any of Dichloroacetate Salts 1-1.8, wherein said salt crystal exhibits a Differential Scanning Calorimetry (DSC) pattern comprising an endothermic peak between about 225° C. and 228° C., e.g., at about 227° C., and/or between about 229° C. and 232° C., e.g., at about 230° C.
1.10 Any of Dichloroacetate Salts 1-1.9, wherein the Compound A free base and dichloroacetic acid counterion are present in a molar ratio of about 2:1 to about 1:2, e.g., about 1:1, e.g., about 1:0.8.

In a further embodiment, the present disclosure provides for a caproate salt [Caproate Salt 1] of Compound A.
1.1 Caproate Salt 1, which is in crystalline form.
1.2 Caproate Salt 1 or 1.1, which is in anhydrous form.
1.3 Any of Caproate Salts 1-1.1, wherein the salt crystal is a 2-butanone solvate.
1.4 Any of Caproate Salts 1-1.3, wherein said salt crystal exhibits an X-ray powder diffraction pattern comprising at least five peaks having 2-theta angle values selected from the group consisting of: 5.2, 7.5, 7.8, 10.2, 11.3, 12.2, 12.6, 19.6, 23.0, and 23.4 degrees, wherein the XRPD pattern is measured in a diffractometer using copper anode, e.g., at wavelength alpha1 of 1.5406 Å and wavelength alpha2 of 1.5444 Å.
1.5 Any of Caproate Salts 1-1.4, wherein said salt crystal exhibits an X-ray powder diffraction pattern comprising at least five peaks having 2-theta angle values selected from the group consisting of: 5.2, 7.8, 10.2, 11.3, and 23.0 degrees, wherein the XRPD pattern is measured in a diffractometer using copper anode, e.g., at wavelength alpha1 of 1.5406 Å and wavelength alpha2 of 1.5444 Å.
1.6 Any of Caproate Salts 1-1.5, wherein said salt crystal exhibits an X-ray powder diffraction pattern comprising at least five peaks having d-spacing values selected from the group consisting of 17.1, 11.8, 11.3, 8.6, 7.8, 7.2, 7.0, 4.5, 3.9, and 3.8 Å.
1.7 Any of Caproate Salts 1-1.6, wherein said salt crystal exhibits an X-ray powder diffraction pattern comprising peaks having d-spacing values selected from the group consisting of 17.1, 11.3, 8.6, 7.8, and 3.9 Å.
1.8 Any of Caproate Salts 1-1.7, wherein said salt crystal exhibits an X-ray powder diffraction pattern comprising at least five peaks having 2-theta angle values or comprising at least five peaks having d-spacing values selected from those set forth in Table 49 as defined herein, wherein the XRPD pattern is measured in a diffractometer using copper anode, at wavelength alpha1 of 1.5406 Å and wavelength alpha2 of 1.5444 Å.
1.9 Any of Caproate Salts 1-1.8, wherein said salt crystal exhibits a Differential Scanning Calorimetry (DSC) pattern comprising an endothermic peak between about 89° C. and 92° C., e.g., at about 90° C., and/or between about 104° C. and 107° C., e.g., at about 105° C.
1.10 Any of Caproate Salts 1-1.9, wherein the Compound A free base and caproic acid counterion are present in a molar ratio of about 2:1 to about 1:2, e.g., about 1:1, e.g., about 1:0.9.

In a further embodiment, the present disclosure provides for a laurate salt [Laurate Salt 1] of Compound A.
1.1 Laurate Salt 1, which is in crystalline form.
1.2 Laurate Salt 1 or 1.1, which is in anhydrous form.
1.3 Any of Laurate Salts 1-1.1, wherein the salt crystal is a 2-propanol solvate.
1.4 Any of Laurate Salts 1-1.3, wherein said salt crystal exhibits an X-ray powder diffraction pattern comprising at least five peaks having 2-theta angle values selected from the group consisting of: 4.3, 4.6, 5.5, 5.9, 9.2, 10.9, 11.0, 22.1, 22.0, and 22.7 degrees, wherein the XRPD pattern is measured in a diffractometer using copper anode, e.g., at wavelength alpha1 of 1.5406 Å and wavelength alpha2 of 1.5444 Å.
1.5 Any of Laurate Salts 1-1.4, wherein said salt crystal exhibits an X-ray powder diffraction pattern comprising at least five peaks having 2-theta angle values selected from the group consisting of: 4.3, 4.6, 5.5, 5.9, and 22.7 degrees, wherein the XRPD pattern is measured in a diffractometer using copper anode, e.g., at wavelength alpha1 of 1.5406 Å and wavelength alpha2 of 1.5444 Å.
1.6 Any of Laurate Salts 1-1.5, wherein said salt crystal exhibits an X-ray powder diffraction pattern comprising at least five peaks having d-spacing values selected from the group consisting of 20.4, 19.1, 16.1, 14.9, 10.4 9.6, 8.1, 8.0, 4.0, and 3.9 Å.
1.7 Any of Laurate Salts 1-1.6, wherein said salt crystal exhibits an X-ray powder diffraction pattern comprising peaks having d-spacing values selected from the group consisting of 20.4, 19.1, 16.1, 14.9, and 3.9 Å.
1.8 Any of Laurate Salts 1-1.7, wherein said salt crystal exhibits an X-ray powder diffraction pattern comprising at least five peaks having 2-theta angle values or comprising at least five peaks having d-spacing values selected from those set forth in Table 50 as defined herein, wherein the XRPD pattern is measured in a diffractometer using copper anode, at wavelength alpha1 of 1.5406 Å and wavelength alpha2 of 1.5444 Å.
1.9 Any of Laurate Salts 1-1.8, wherein said salt crystal exhibits a Differential Scanning Calorimetry (DSC) pattern comprising an endothermic peak between about 81° C. and 84° C., e.g., at about 83° C.
1.10 Any of Laurate Salts 1-1.9, wherein the Compound A free base and lauric acid counterion are present in a molar ratio of about 2:1 to about 1:2, e.g., about 1:1, e.g., about 1:1.4.

Collectively, the salt crystals named above (i.e., Hydrochloride Salts 1-8, et seq.; Malate Salt 1, et seq.; Fumarate Salt 1, et seq.; Sulfate Salt 1, et seq.; Esylate Salt 1, et seq.; Galactarate Salt 1, et seq.; Adipate Salts 1-3, et seq.; Lactate Salt 1, et seq.; Oxalate Salts 1-4, et seq.; Palmitate Salt 1, et seq.; 2-Oxo-glutarate Salt 1, et seq.; Xinafoate Salts 1-3, et seq.; tosylate Salts 1-2, et seq.; Tartrate Salts 1-2, et seq.; Succinate Salts 1-2, et seq.; Mesylate Salt 1, et seq.; Napadisylate Salt 1, et seq.; Edisylate Salt 1, et seq.; Propionate Salt 1, et seq.; Caprylate Salt 1, et seq.; Besylate Salt 1, et seq.; Benzoate Salt 1, et seq.; Nicotinate Salt 1, et seq.; Isonicotinate Salt 1, et seq.; Orotate Salt 1, et seq.; Camsylate Salts 1-2, et seq.; Salicylate Salts 1-2, et seq.; Aminosalicylate Salt 1, et seq.; Mandelate Salts 1-2, et seq.; Acetamido Benzoate Salts 1-2, et seq.; Trifluoroacetate Salt 1, et seq.; Dichloroacetate Salt 1, et seq.; Caproate Salt 1, et seq.; and Laurate Salt 1, el seq.) are referred to as the Salt Crystals of the Disclosure.

In some embodiments, the Salt Crystals of the Disclosure are in a single crystal form and are free or substantially free of any other form, e.g., less than 10 wt. %, preferably less than about 5 wt. %, more preferably less than about 2 wt. %, still preferably less than about 1 wt. %, still preferably less than about 0.1%, most preferably less than about 0.01 wt. % of amorphous form.

In some embodiments, the Salt Crystals of the Disclosure are in a single crystal form and are free or substantially free of any other form, e.g., less than 10 wt. %, preferably less than about 5 wt. %, more preferably less than about 2 wt. %, still preferably less than about 1 wt. %, still preferably less than about 0.1%, most preferably less than about 0.01 wt. % of other crystal forms.

In some embodiments, the Salt Crystals of the Disclosure are in a single crystal form and are free or substantially free of any other form, e.g., less than 10 wt. %, preferably less than about 5 wt. %, more preferably less than about 2 wt. %, still preferably less than about 1 wt. %, still preferably less than about 0.1%, most preferably less than about 0.01 wt. % of amorphous and other crystal forms.

### Methods of Making and Use of the Salt Crystals of the Invention

The present disclosure further provides a method [Method 1] for the production of stable acid addition salts of (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one (“Compound A”), e.g., crystallinic acid addition salts with particular acids comprising the steps of reacting Compound A in free base form with an acid in a solvent and isolating the salt obtained. In particular embodiments, the present disclosure provides the following:
1.1 Method 1, further comprising the step of forming a slurry of Compound A with the acid in the solvent at a temperature between about 30° C. to 70° C.
1.2 Method 1.1, wherein the slurry is formed at a temperature of 30° C. to 70° C. for a period of at least one hour (e.g., 1-5 hours).
1.3 Method 1.1 or 1.2, wherein the slurry is formed at a temperature of 30° C. to 70° C. for a period of at least 1-3 hours.
1.4 Any of Methods 1.1-1.3, wherein the slurry is formed at a temperature of about 40° C. to 60° C., e.g., about 45° C. to 65° C., e.g., about 50° C.
1.5 Any of Methods 1.1-1.4, wherein the slurry is formed at a temperature of about 50° C.
1.6 Any of the preceding Methods, wherein the acid is hydrochloric acid, malic acid, fumaric acid, sulfuric acid, ethane sulfonic acid, galactaric acid, adipic acid, lactic acid, oxalic acid, palmitic acid, 2-oxo-glutaric acid, xinafoic acid, toluene sulfonic acid, tartaric acid, succinic acid, methane sulfonic acid, naphthalene disulfonic acid, ethane disulfonic acid, propionic naphthalene disulfonic acid, caprylic naphthalene disulfonic acid, benzenesulfonic acid, benzoic acid, nicotinic acid, isonicotinic acid, orotic acid, camsylic acid, salicylic acid, aminosalicylic acid, mandelic acid, acetamido-benzoic acid, trifluoroacetic acid, dichloroacetic acid, caproic acid, or lauric acid.
1.7 Any of the preceding Methods, wherein the acid is hydrochloric acid, malic acid, tartaric acid, or oxalic acid.
1.8 Any of the preceding Methods, wherein the acid is hydrochloric acid.
1.9 Any of the preceding Methods, wherein the acid is malic acid.
1.10 Any of the preceding Methods, wherein the acid is tartaric acid.
1.11 Any of the preceding Methods, wherein the acid is oxalic acid.
1.12 Any of the preceding Methods, wherein the acid and Compound A in free base form are combined in a molar ratio of 1:1.
1.13 Any of the preceding Methods, wherein the solvent is water, acetonitrile, ethyl acetate, acetone, 2-butanone, 2-ethyl-1-butanol, ethyl salicylate, ethyl butyl ketone, acetone, 3-heptanone, toluene, methanol, ethanol, propanol (e.g., isopropanol, 2-propanol), butanol (e.g., 2-ethyl-1-butanol), dimethyl sulfoxide (DMSO), anisole, ethyl butyl ketone, or combinations thereof
1.14 Any of the preceding Methods, wherein the solvent is acetonitrile, ethyl acetate, 2-butanone, acetone, 3-heptanone, 2-butanone, or combinations thereof.
1.15 Any of the preceding Methods, further comprising the step of cooling the solution to a temperature of about −10° C. to about 20° C.
1.16 Method 1.15, wherein the solution is cooled to a temperature of about 0° C. to about 10° C.
1.17 Method 1.15 or 1.16, wherein the solution is cooled to a temperature of about 5° C.
1.18 Any of Methods 1.15-1.17, wherein the solution is cooled for a period of at least about 5 hours.
1.19 Any of Methods 1.15-1.18, wherein the solution is cooled for a period of about 5 hours to about 24 hours.
1.20 Any of Methods 1.15-1.18, wherein the solution is cooled for a period of about 8 hours.
1.21 Any of the preceding Methods, comprising the step of drying the solution by evaporation.
1.22 Method 1.21, wherein the solution is placed under vacuum to evaporate the solvent.
1.23 Any of the preceding Methods, wherein the salt is crystalline.
1.24 Any of the preceding Methods, wherein the obtained salt is crystalline, and are dissolved in a second solvent and are subjected to one or more cooling cycles.
1.25 Method 1.24, wherein the second solvent is water, acetonitrile, ethyl acetate, acetone, 2-butanone, 2-ethyl-1-butanol, ethyl salicylate, ethyl butyl ketone, acetone, 3-heptanone, toluene, methanol, ethanol, propanol (e.g., isopropanol, 2-propanol), butanol (e.g., 2-ethyl-1-butanol), dimethyl sulfoxide (DMSO), anisole, ethyl butyl ketone, or combinations thereof
1.26 Method 1.24 or 1.25, wherein the cooling cycle comprises heating then cooling the solution for at least 2 cycles (e.g., at least 3 cycles, at least 4 cycles, at least 5 cycles).
1.27 Any of Methods 1.24-1.26, wherein the cooling cycle comprises heating then cooling the solution for 4 cycles.
1.28 Any of Methods 1.24-1.27, wherein the cooling cycle comprises:
   a. heating the solution to about 50° C.;
   b. cooling the solution to about 0° C.;
   c. heating the solution to about 40° C.;
   d. cooling the solution to about 0° C.;
   e. heating the solution to about 30° C.;
   f. cooling the solution to about 0° C.;
   g. heating the solution to about 20° C.; and
   h. cooling the solution to about 0° C.
1.29 Any of the preceding Methods, wherein the salt is crystalline.

The present disclosure further provides for Salt Crystals of the Disclosure which are obtained or obtainable by any of Methods 1, et seq. or any of Examples 1-4.

A method [Method 2] for the prophylaxis or treatment of a patient, e.g., a human, suffering from a disorder selected from the following disorders:
A. Neurodegenerative diseases, including Parkinson's disease, restless leg, tremors, dyskinesias, Huntington's disease, Alzheimer's disease, and drug-induced movement disorders;
B. Mental disorders, including depression, attention deficit disorder, attention deficit hyperactivity disorder, bipolar illness, anxiety, sleep disorders, e.g., narcolepsy, cognitive impairment, e.g., cognitive impairment of schizophrenia, dementia, Tourette's syndrome, autism, fragile X syndrome, psychostimulant withdrawal, and drug addiction;
C. Circulatory and cardiovascular disorders, including cerebrovascular disease, stroke, congestive heart disease, hypertension, pulmonary hypertension, e.g., pulmonary arterial hypertension, and sexual dysfunction, including cardiovascular diseases and related disorders as described in International Application No. PCT/US2014/16741, the contents of which are incorporated herein by reference;
D. Respiratory and inflammatory disorders, including asthma, chronic obstructive pulmonary disease, and allergic rhinitis, as well as autoimmune and inflammatory diseases;
E. Diseases that may be alleviated by the enhancement of progesterone-signaling such as female sexual dysfunction;
F. A disease or disorder such as psychosis, glaucoma, or elevated intraocular pressure;
G. Traumatic brain injury;
H. Cancers or tumors, e.g., brain tumors, a glioma (e.g., ependymoma, astrocytoma, oligodendrogliomas, brain stem glioma, optic nerve glioma, or mixed gliomas, e.g., oligoastrocytomas), an astrocytoma (e.g., glioblastoma multiforme), osteosarcoma, melanoma, leukemia, neuroblastoma or leukemia;
I. Renal disorders, e.g., kidney fibrosis, chronic kidney disease, renal failure, glomerulosclerosis and nephritis;
J. Any disease or condition characterized by low levels of cAMP and/or cGMP (or inhibition of cAMP and/or cGMP signaling pathways) in cells expressing PDE1; and/or
K. Any disease or condition characterized by reduced dopamine D1 receptor signaling activity,
   comprising administering to a patient in need thereof a therapeutically effective amount of (a) the compound (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one (Compound A) in acid addition salt form according to any of the Salt Crystals of the Disclosure.
2.1 Å pharmaceutical composition comprising any of the Salt Crystals of the Disclosure for use as a medicament, e.g., for use in the manufacture of a medicament for the treatment or prophylaxis of a disease as described in Method 2.
2.2 Use of any of the Salt Crystals of the Disclosure in the treatment or prophylaxis of a disease as described in Method 2.
2.3 Method 2, wherein the co-crystal is administered to a patient in an amount equivalent to 1-300 mg of Compound 1 per day.
2.4 Any of the preceding methods, wherein the co-crystal is administered to a patient in an amount equivalent to 15-180 mg per day, e.g., 30-90 mg per day, e.g., 30-60 mg of Compound 1 per day.

### EXAMPLES

Studies are carried out to identify crystal forms for salts of Compound 1, which is (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one, having the following structure:

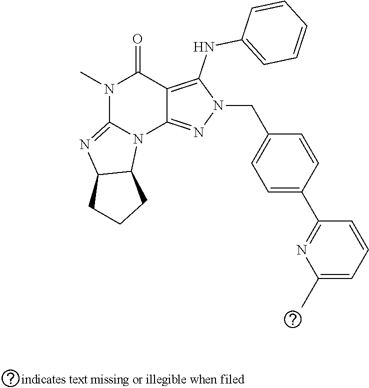 [SMILES: Cc1cccc(-c2ccc(Cn3nc4c(c3Nc3ccccc3)C(=O)N(C)C3=N[C@@H]5CCC[C@@H]5N34)cc2)n1]

Counter ions are dissolved in either methanol or water to obtain 2M solutions. The counter ions that do not dissolve are added as solid. 20 mg of Compound 1 Free Base is dispensed in wells of a master plate, followed by the counter ions such that the mixtures have a molar ratio of 1:1. Solvents (800 μl) as identified below are subsequently added to the wells. The master plate is then stored on a thermoshaker and is shaken at 50° C. for 2 hours.

The clear liquids in the master plate wells are divided and placed over a cooling plate, an evaporation plate or an HPLC plate. The cooling plate is cooled to 5° C. and the sample is stored at this temperature for about 16 hours. The evaporation plate is stored in a vacuum oven to obtain complete evaporation of the solvents. The remaining liquids are absorbed by filter paper and the solids were dried in vacuum. All samples on each of the master plate, cooling plate and evaporation plate are analyzed using High Throughput (HT)-XRPD.

XRPD data analyses show various new crystalline patterns. Some counter ions show multiple patterns, which is influenced by the solvent and may indicate polymorphism or solvate formation. After defining all XRPD patterns, the crystalline patterns are analyzed by thermogravimetric analysis and differential scanning calorimetry.

XRPD is measured using a Bruker AXS D2 PHASER in Bragg-Brentano configuration with a scan range of 5-45° 2-theta. TGA measurements are performed using a Mettler Toledo TGA/DSC-3+ machine. DSC measurements are performed using a Mettler Toledo DSC-3+ machine. The sample is heated from 20° C. to 350° C. in an aluminium (pierced) cup, and a heating rate of 10° C./min is applied.

### Hydrochloride Salt

Aqueous hydrochloric acid is combined with 25 mg of Compound 1 in ethyl acetate, and is subsequently slurried at 50° C. for 2 hours. The resultant salt is a solvate and is obtained as a white to off-white powder. The XRPD pattern of Hydrochloride Salt 1 has peaks as set forth in Table 1 below.

TABLE 1

| | | | | Rel. |
|---|---|---|---|---|
| Index | Angle | d-Value | Intensity | Intensity |
| 1 | 4.94 | 17.87539 | 4.73E+03 | 100.00% |
| 2 | 7.276 | 12.14047 | 1.03E+03 | 21.80% |
| 3 | 9.488 | 9.31424 | 360 | 7.60% |
| 4 | 9.678 | 9.13149 | 378 | 8.00% |
| 5 | 12.266 | 7.20992 | 1.14E+03 | 24.00% |
| 6 | 13.19 | 6.70714 | 309 | 6.50% |
| 7 | 14.384 | 6.153 | 362 | 7.70% |
| 8 | 14.63 | 6.0498 | 478 | 10.10% |
| 9 | 15.657 | 5.6554 | 186 | 3.90% |
| 10 | 16.783 | 5.27826 | 140 | 3.00% |
| 11 | 18.5 | 4.7921 | 217 | 4.60% |
| 12 | 19.006 | 4.66577 | 501 | 10.60% |
| 13 | 19.563 | 4.53415 | 695 | 14.70% |
| 14 | 21.372 | 4.15421 | 330 | 7.00% |
| 15 | 21.708 | 4.09063 | 322 | 6.80% |
| 16 | 23.118 | 3.84431 | 225 | 4.80% |
| 17 | 24.57 | 3.62031 | 79.4 | 1.70% |

The above crystal exhibits a thermal melting event between about 169° C. and 172° C., e.g., at about 170° C., according to differential scanning calorimetry. Hydrochloride Salt 1 is in solvate form, and the free base and counter ion are present in the crystal in a ratio of 1:0.9.

A second hydrochloride salt is obtained when hydrochloric acid in cyclopentyl methyl ether is combined with 25 mg of Compound 1 in acetonitrile, and is cooled to 5° C. over an 8 hour period. The resultant salt is a solvate and is obtained as an off-white powder following evaporation. The XRPD pattern of Hydrochloride Salt 2 has peaks as set forth in Table 2 below.

TABLE 2

| Index | Angle | d-Value | Intensity | Rel.Intensity |
|---|---|---|---|---|
| 1 | 7.253 | 12.17766 | 3.90E+03 | 74.30% |
| 2 | 7.506 | 11.76906 | 656 | 12.50% |
| 3 | 7.89 | 11.19678 | 441 | 8.40% |
| 4 | 9.117 | 9.69196 | 536 | 10.20% |
| 5 | 9.199 | 9.60638 | 647 | 12.30% |
| 6 | 9.266 | 9.53703 | 466 | 8.90% |
| 7 | 10.378 | 8.51707 | 134 | 2.60% |
| 8 | 11.097 | 7.96687 | 653 | 12.50% |
| 9 | 11.123 | 7.9481 | 642 | 12.20% |
| 10 | 11.62 | 7.60952 | 369 | 7.00% |
| 11 | 12.068 | 7.3282 | 1.58E+03 | 30.20% |
| 12 | 13.578 | 6.51633 | 1.76E+03 | 33.50% |
| 13 | 14.434 | 6.13176 | 212 | 4.00% |
| 14 | 15.621 | 5.6681 | 5.24E+03 | 100.00% |
| 15 | 16.02 | 5.52806 | 628 | 12.00% |
| 16 | 16.427 | 5.39207 | 805 | 15.40% |
| 17 | 18.157 | 4.88193 | 421 | 8.00% |
| 18 | 18.485 | 4.796 | 705 | 13.50% |
| 19 | 18.538 | 4.78239 | 944 | 18.00% |
| 20 | 19.174 | 4.62517 | 430 | 8.20% |
| 21 | 19.99 | 4.43828 | 897 | 17.10% |
| 22 | 20.007 | 4.43455 | 862 | 16.40% |
| 23 | 20.119 | 4.41008 | 612 | 11.70% |
| 24 | 20.737 | 4.27993 | 354 | 6.80% |
| 25 | 21.346 | 4.15925 | 694 | 13.20% |
| 26 | 21.441 | 4.1409 | 930 | 17.70% |
| 27 | 21.533 | 4.12359 | 670 | 12.80% |
| 28 | 22.303 | 3.98293 | 241 | 4.60% |
| 29 | 22.858 | 3.88732 | 202 | 3.90% |
| 30 | 22.958 | 3.87074 | 289 | 5.50% |
| 31 | 23.671 | 3.75563 | 331 | 6.30% |
| 32 | 25.54 | 3.48492 | 229 | 4.40% |
| 33 | 26.031 | 3.42024 | 202 | 3.80% |
| 34 | 28.665 | 3.11168 | 119 | 2.30% |
| 35 | 29.936 | 2.9824 | 207 | 3.90% |
| 36 | 29.976 | 2.9785 | 216 | 4.10% |
| 37 | 37.377 | 2.40401 | 48.6 | 0.90% |

The above crystal exhibits thermal events between about 140° C. and 142° C., e.g., at about 141° C., and between about 190° C. and 192° C., e.g., at about 191° C., according to differential scanning calorimetry. Hydrochloride Salt 2 is in solvate form, and the free base and counter ion are present in the crystal in a ratio of 1:0.9.

A third hydrochloride salt is obtained when aqueous hydrochloric acid is combined with 25 mg of Compound 1 in acetonitrile, and is subsequently cooled to 5° C. over an 8 hour period. The resultant salt is a solvate and is obtained as an off-white powder following evaporation. The XRPD pattern of Hydrochloride Salt 3 has peaks as set forth in Table 3 below.

TABLE 3

| Index | Angle | d-Value | Intensity | Rel.Intensity |
|---|---|---|---|---|
| 1 | 4.901 | 18.01604 | 4.18E+03 | 24.70% |
| 2 | 6.035 | 14.63338 | 287 | 1.70% |
| 3 | 6.117 | 14.43727 | 528 | 3.10% |
| 4 | 6.901 | 12.79908 | 666 | 3.90% |
| 5 | 7.33 | 12.05051 | 1.36E+03 | 8.10% |
| 6 | 7.384 | 11.96268 | 1.42E+03 | 8.40% |
| 7 | 9.592 | 9.21355 | 343 | 2.00% |
| 8 | 9.733 | 9.0797 | 523 | 3.10% |
| 9 | 9.991 | 8.84648 | 205 | 1.20% |
| 10 | 12.195 | 7.25176 | 1.69E+04 | 100.00% |
| 11 | 12.731 | 6.94786 | 1.31E+03 | 7.70% |
| 12 | 13.751 | 6.43449 | 237 | 1.40% |
| 13 | 14.609 | 6.05857 | 580 | 3.40% |
| 14 | 14.744 | 6.0034 | 444 | 2.60% |
| 15 | 15.271 | 5.79751 | 223 | 1.30% |
| 16 | 18.504 | 4.79101 | 447 | 2.60% |
| 17 | 18.566 | 4.77523 | 415 | 2.50% |
| 18 | 19.013 | 4.66403 | 258 | 1.50% |
| 19 | 19.516 | 4.54487 | 187 | 1.10% |
| 20 | 19.797 | 4.48105 | 196 | 1.20% |
| 21 | 20.646 | 4.29864 | 813 | 4.80% |
| 22 | 22.05 | 4.02791 | 207 | 1.20% |
| 23 | 23.234 | 3.82535 | 183 | 1.10% |
| 24 | 24.535 | 3.6254 | 403 | 2.40% |
| 25 | 25.04 | 3.5534 | 388 | 2.30% |
| 26 | 25.258 | 3.52319 | 413 | 2.40% |
| 27 | 25.53 | 3.48624 | 391 | 2.30% |
| 28 | 25.938 | 3.43238 | 167 | 1.00% |
| 29 | 27.623 | 3.22672 | 3.35E+03 | 19.80% |
| 30 | 32.698 | 2.73651 | 533 | 3.20% |
| 31 | 32.873 | 2.72234 | 277 | 1.60% |
| 32 | 34.684 | 2.58427 | 375 | 2.20% |
| 33 | 38.674 | 2.3263 | 137 | 0.80% |
| 34 | 39.508 | 2.2791 | 88.4 | 0.50% |

The above crystal exhibits thermal events between about 155° C. and 157° C., e.g., at about 156° C., and between about 275° C. and 277° C., e.g., at about 276° C., according to differential scanning calorimetry. Hydrochloride Salt 3 is in solvate form, and the free base and counter ion are present in the crystal in a ratio of 1:0.9.

A fourth hydrochloride salt is obtained when hydrochloric acid in isopropyl alcohol is combined with 25 mg of Compound 1 in 2-butanone, and is cooled to 5° C. over an 8 hour period. The resultant salt is a solvate and is obtained as a crystalline slurry. The XRPD pattern of Hydrochloride Salt 4 has peaks as set forth in Table 4 below.

TABLE 4

| | | | | Rel. |
|---|---|---|---|---|
| Index | Angle | d-Value | Intensity | Intensity |
| 1 | 7.353 | 12.0132 | 533 | 10.00% |
| 2 | 7.553 | 11.69507 | 2.67E+03 | 50.30% |
| 3 | 8.016 | 11.02138 | 414 | 7.80% |
| 4 | 8.28 | 10.66967 | 166 | 3.10% |
| 5 | 8.441 | 10.46677 | 720 | 13.60% |
| 6 | 9.368 | 9.43251 | 180 | 3.40% |
| 7 | 9.548 | 9.25523 | 254 | 4.80% |
| 8 | 9.701 | 9.11017 | 746 | 14.00% |
| 9 | 11.199 | 7.89456 | 657 | 12.40% |
| 10 | 11.506 | 7.68478 | 301 | 5.70% |
| 11 | 11.569 | 7.64297 | 414 | 7.80% |
| 12 | 12.004 | 7.36698 | 3.46E+03 | 65.10% |
| 13 | 12.275 | 7.20468 | 277 | 5.20% |
| 14 | 12.376 | 7.14599 | 712 | 13.40% |
| 15 | 12.572 | 7.03499 | 651 | 12.30% |
| 16 | 12.711 | 6.95857 | 5.31E+03 | 100.00% |
| 17 | 13.464 | 6.57126 | 361 | 6.80% |
| 18 | 13.489 | 6.55918 | 375 | 7.10% |
| 19 | 15.028 | 5.89061 | 1.34E+03 | 25.20% |
| 20 | 15.146 | 5.84508 | 1.18E+03 | 22.30% |
| 21 | 15.387 | 5.75391 | 569 | 10.70% |
| 22 | 15.671 | 5.65028 | 327 | 6.20% |
| 23 | 15.943 | 5.55466 | 237 | 4.50% |
| 24 | 16.304 | 5.43219 | 191 | 3.60% |
| 25 | 16.814 | 5.26867 | 207 | 3.90% |
| 26 | 17.542 | 5.05165 | 252 | 4.80% |
| 27 | 17.923 | 4.94506 | 934 | 17.60% |
| 28 | 18.142 | 4.88595 | 239 | 4.50% |
| 29 | 18.235 | 4.86131 | 219 | 4.10% |
| 30 | 18.524 | 4.786 | 141 | 2.60% |
| 31 | 18.756 | 4.72723 | 1.38E+03 | 26.00% |
| 32 | 19.098 | 4.64348 | 773 | 14.60% |
| 33 | 19.307 | 4.5936 | 1.18E+03 | 22.20% |
| 34 | 19.791 | 4.48227 | 377 | 7.10% |
| 35 | 19.916 | 4.45448 | 491 | 9.20% |
| 36 | 20.504 | 4.32817 | 564 | 10.60% |
| 37 | 21.291 | 4.16982 | 367 | 6.90% |
| 38 | 21.705 | 4.09122 | 288 | 5.40% |
| 39 | 22.064 | 4.0255 | 355 | 6.70% |
| 40 | 22.372 | 3.97079 | 226 | 4.30% |
| 41 | 22.416 | 3.96298 | 185 | 3.50% |
| 42 | 23.099 | 3.8474 | 1.58E+03 | 29.70% |
| 43 | 23.611 | 3.76508 | 507 | 9.60% |
| 44 | 23.991 | 3.70635 | 1.16E+03 | 21.80% |
| 45 | 24.443 | 3.6388 | 771 | 14.50% |
| 46 | 24.637 | 3.61057 | 602 | 11.30% |
| 47 | 24.715 | 3.59929 | 655 | 12.30% |
| 48 | 24.913 | 3.57121 | 263 | 5.00% |
| 49 | 25.048 | 3.55218 | 224 | 4.20% |
| 50 | 25.207 | 3.53023 | 129 | 2.40% |
| 51 | 25.46 | 3.49568 | 114 | 2.20% |
| 52 | 25.811 | 3.44889 | 530 | 10.00% |
| 53 | 26.801 | 3.3237 | 404 | 7.60% |
| 54 | 27.096 | 3.28823 | 452 | 8.50% |
| 55 | 27.155 | 3.28127 | 407 | 7.70% |
| 56 | 27.244 | 3.27068 | 190 | 3.60% |
| 57 | 27.737 | 3.21371 | 169 | 3.20% |
| 58 | 28.08 | 3.17519 | 202 | 3.80% |
| 59 | 28.45 | 3.13476 | 237 | 4.50% |
| 60 | 28.912 | 3.08572 | 196 | 3.70% |
| 61 | 29.022 | 3.07429 | 428 | 8.10% |
| 62 | 29.142 | 3.06182 | 456 | 8.60% |
| 63 | 30.519 | 2.92678 | 109 | 2.10% |
| 64 | 30.907 | 2.89089 | 257 | 4.80% |
| 65 | 31.093 | 2.87404 | 158 | 3.00% |
| 66 | 31.54 | 2.83432 | 107 | 2.00% |
| 67 | 32.701 | 2.73629 | 67.6 | 1.30% |
| 68 | 33.21 | 2.6955 | 118 | 2.20% |
| 69 | 33.326 | 2.68636 | 178 | 3.30% |
| 70 | 33.733 | 2.6549 | 100 | 1.90% |
| 71 | 33.767 | 2.65228 | 95.1 | 1.80% |
| 72 | 33.949 | 2.63852 | 180 | 3.40% |
| 73 | 34.01 | 2.63389 | 215 | 4.00% |
| 74 | 34.316 | 2.61111 | 66.4 | 1.30% |
| 75 | 35.056 | 2.55769 | 107 | 2.00% |
| 76 | 35.318 | 2.5393 | 73.1 | 1.40% |
| 77 | 36.036 | 2.49031 | 52.9 | 1.00% |
| 78 | 36.293 | 2.47331 | 54.6 | 1.00% |
| 79 | 36.59 | 2.45388 | 69 | 1.30% |
| 80 | 37.488 | 2.39717 | 69.3 | 1.30% |
| 81 | 39.819 | 2.262 | 74.5 | 1.40% |
| 82 | 40.026 | 2.2508 | 109 | 2.00% |
| 83 | 40.138 | 2.2448 | 105 | 2.00% |
| 84 | 40.42 | 2.22978 | 35.3 | 0.70% |
| 85 | 44.099 | 2.05191 | 99.6 | 1.90% |
| 86 | 44.113 | 2.05129 | 96.2 | 1.80% |

The above crystal exhibits thermal events between about 194° C. and 196° C., e.g., at about 195° C., and between about 209° C. and 211° C., e.g., at about 210° C., according to differential scanning calorimetry. Hydrochloride Salt 4 is in solvate form, and the free base and counter ion are present in the crystal in a ratio of 1:0.9.

### Malate Salt

An L-malate salt is obtained when L-malic acid is combined with 25 mg of Compound 1 in ethyl acetate, and is subsequently slurried at 50° C. for 2 hours. The resulting salt is a solvate and is obtained as an off-white powder. The XRPD pattern of L-Malate Salt 1 has peaks as set forth in Table 5 below.

TABLE 5

| | | | | Rel. |
|---|---|---|---|---|
| Index | Angle | d-Value | Intensity | Intensity |
| 1 | 5.9 | 14.96729 | 1.90E+03 | 83.30% |
| 2 | 6.518 | 13.54924 | 334 | 14.60% |
| 3 | 7.171 | 12.31797 | 953 | 41.70% |
| 4 | 10.404 | 8.49623 | 90.2 | 3.90% |
| 5 | 11.969 | 7.38838 | 1.26E+03 | 55.20% |
| 6 | 13.071 | 6.76791 | 408 | 17.90% |
| 7 | 13.372 | 6.61615 | 306 | 13.40% |
| 8 | 13.602 | 6.50454 | 339 | 14.90% |
| 9 | 14.124 | 6.2655 | 219 | 9.60% |
| 10 | 14.586 | 6.06802 | 225 | 9.90% |
| 11 | 15.618 | 5.6692 | 370 | 16.20% |
| 12 | 15.762 | 5.61794 | 491 | 21.50% |
| 13 | 16.026 | 5.52575 | 1.76E+03 | 77.00% |
| 14 | 16.541 | 5.35498 | 202 | 8.80% |
| 15 | 17.65 | 5.02088 | 1.20E+03 | 52.70% |
| 16 | 17.797 | 4.97989 | 2.28E+03 | 100.00% |
| 17 | 18.531 | 4.78407 | 220 | 9.70% |
| 18 | 18.793 | 4.71797 | 415 | 18.20% |
| 19 | 19.552 | 4.5366 | 625 | 27.40% |
| 20 | 20.511 | 4.32656 | 685 | 30.00% |
| 21 | 20.388 | 4.35234 | 452 | 19.80% |
| 22 | 20.865 | 4.254 | 810 | 35.50% |
| 23 | 21.229 | 4.18188 | 1.27E+03 | 55.60% |
| 24 | 21.664 | 4.09879 | 1.81E+03 | 79.20% |
| 25 | 21.842 | 4.06582 | 2.07E+03 | 90.40% |
| 26 | 23.096 | 3.84784 | 432 | 18.90% |
| 27 | 23.56 | 3.77316 | 195 | 8.50% |
| 28 | 24.22 | 3.67172 | 155 | 6.80% |
| 29 | 24.867 | 3.57767 | 140 | 6.10% |
| 30 | 25.837 | 3.44559 | 459 | 20.10% |
| 31 | 25.927 | 3.43372 | 352 | 15.40% |
| 32 | 26.293 | 3.38675 | 115 | 5.00% |
| 33 | 27.045 | 3.29435 | 188 | 8.30% |
| 34 | 26.935 | 3.3075 | 223 | 9.80% |
| 35 | 26.921 | 3.30926 | 218 | 9.50% |
| 36 | 27.341 | 3.2593 | 239 | 10.50% |
| 37 | 27.621 | 3.22687 | 167 | 7.30% |
| 38 | 28.563 | 3.12255 | 565 | 24.80% |
| 39 | 29.562 | 3.0193 | 276 | 12.10% |
| 40 | 31.208 | 2.86366 | 71.4 | 3.10% |
| 41 | 31.388 | 2.84767 | 152 | 6.70% |
| 42 | 32.909 | 2.7195 | 91.6 | 4.00% |
| 43 | 34.466 | 2.60013 | 68.9 | 3.00% |
| 44 | 36.713 | 2.44597 | 103 | 4.50% |

The above crystal exhibits a thermal melting event between about 94° C. and 96° C., e.g., at about 95° C., according to differential scanning calorimetry. L-Malate Salt 1 is in solvate form, and the free base and counter ion are present in the crystal in a ratio of 1:0.7.

### Fumarate Salt

A fumarate salt is obtained when fumaric acid is combined with 25 mg of Compound 1 in ethyl acetate, and is subsequently slurried at 50° C. for 2 hours. The resulting salt is a solvate and is obtained as a white powder. The XRPD pattern of Fumarate Salt 1 has peaks as set forth in Table 6 below.

TABLE 6

| | | | | Rel. |
|---|---|---|---|---|
| Index | Angle | d-Value | Intensity | Intensity |
| 1 | 5.931 | 14.89041 | 8.46E+03 | 100.00% |
| 2 | 7.367 | 11.99026 | 2.59E+03 | 30.60% |
| 3 | 10.306 | 8.57608 | 210 | 2.50% |
| 4 | 10.694 | 8.26579 | 459 | 5.40% |
| 5 | 11.825 | 7.47781 | 1.46E+03 | 17.20% |
| 6 | 12.299 | 7.19078 | 501 | 5.90% |
| 7 | 12.62 | 7.00871 | 1.92E+03 | 22.70% |
| 00 | 13.47 | 6.56816 | 631 | 7.50% |
| 9 | 13.764 | 6.42835 | 2.01E+03 | 23.70% |
| 10 | 14.959 | 5.91764 | 199 | 2.40% |
| 11 | 15.084 | 5.86876 | 510 | 6.00% |
| 12 | 15.584 | 5.68162 | 659 | 7.80% |
| 13 | 16.191 | 5.46987 | 650 | 7.70% |
| 14 | 16.943 | 5.22898 | 589 | 7.00% |
| 15 | 17.176 | 5.15832 | 2.70E+03 | 31.90% |
| 16 | 17.863 | 4.96159 | 423 | 5.00% |
| 17 | 17.921 | 4.94571 | 467 | 5.50% |
| 18 | 18.919 | 4.68686 | 1.08E+03 | 12.80% |
| 19 | 19.266 | 4.60319 | 223 | 2.60% |
| 20 | 20.446 | 4.34025 | 650 | 7.70% |
| 21 | 20.572 | 4.31393 | 1.09E+03 | 12.90% |
| 22 | 21.524 | 4.12526 | 1.26E+03 | 15.00% |
| 23 | 21.496 | 4.13043 | 1.35E+03 | 15.90% |
| 24 | 21.695 | 4.09316 | 3.63E+03 | 42.90% |
| 25 | 22.454 | 3.9564 | 876 | 10.40% |
| 26 | 22.862 | 3.88679 | 224 | 2.60% |
| 27 | 23.38 | 3.80177 | 282 | 3.30% |
| 28 | 23.423 | 3.79492 | 283 | 3.30% |
| 29 | 23.618 | 3.76401 | 365 | 4.30% |
| 30 | 24.641 | 3.60995 | 555 | 6.60% |
| 31 | 24.825 | 3.58364 | 550 | 6.50% |
| 32 | 25.357 | 3.50963 | 457 | 5.40% |
| 33 | 25.707 | 3.46261 | 419 | 5.00% |
| 34 | 27.465 | 3.2449 | 512 | 6.10% |
| 35 | 27.401 | 3.25229 | 413 | 4.90% |
| 36 | 28.169 | 3.16537 | 88.3 | 1.00% |
| 37 | 28.935 | 3.08327 | 179 | 2.10% |
| 38 | 29.11 | 3.06516 | 399 | 4.70% |
| 39 | 29.129 | 3.06323 | 360 | 4.30% |
| 40 | 29.608 | 3.01476 | 308 | 3.60% |
| 41 | 30.244 | 2.95272 | 108 | 1.30% |
| 42 | 31.192 | 2.86514 | 169 | 2.00% |
| 43 | 31.31 | 2.85463 | 308 | 3.60% |
| 44 | 31 335 | 2.85243 | 293 | 3.50% |
| 45 | 37.851 | 2.375 | 71.4 | 0.80% |
| 46 | 42.454 | 2.1275 | 46.1 | 0.50% |

The above crystal exhibits thermal events between about 110° C. and 112° C., e.g., at about 111° C., and between about 141° C. and 143° C., e.g., at about 142° C., and between about 164° C. and 166° C., e.g., at about 165° C., according to differential scanning calorimetry. Fumarate Salt 1 is in solvate form, and the free base and counter ion are present in the crystal in a ratio of 1:0.5.

### Sulfate Salt

A sulfate salt is obtained when sulfuric acid is combined with 25 mg of Compound 1 in ethyl acetate, and is subsequently cooled to 5° C. over an 8 hour period. The resulting salt is a solvate and is obtained as an off-white powder after evaporation. The XRPD pattern of Sulfate Salt 1 has peaks as set forth in Table 7 below.

TABLE 7

| | | | | Rel. |
|---|---|---|---|---|
| Index | Angle | d-Value | Intensity | Intensity |
| 1 | 4.881 | 18.09126 | 400 | 64.20% |
| 2 | 5.852 | 15.09011 | 623 | 100.00% |
| 3 | 6.477 | 13.63466 | 536 | 86.00% |
| 4 | 7.966 | 11.08919 | 284 | 45.70% |
| 5 | 8.455 | 10.44947 | 230 | 36.90% |
| 6 | 9.648 | 9.15998 | 126 | 20.30% |
| 7 | 10.528 | 8.39597 | 158 | 25.40% |
| 8 | 11.133 | 7.94088 | 173 | 27.70% |
| 9 | 12.287 | 7.19764 | 228 | 36.50% |
| 10 | 12.463 | 7.09643 | 207 | 33.20% |
| 11 | 14.692 | 6.02467 | 104 | 16.70% |
| 12 | 17.982 | 4.92907 | 216 | 34.60% |
| 13 | 19.42 | 4.56715 | 185 | 29.70% |
| 14 | 20.836 | 4.2598 | 243 | 39.10% |
| 15 | 21.215 | 4.18453 | 169 | 27.20% |
| 16 | 22.053 | 4.02752 | 73.9 | 11.90% |
| 17 | 22.716 | 3.9114 | 260 | 41.70% |
| 18 | 23.851 | 3.72781 | 56.2 | 9.00% |
| 19 | 24.433 | 3.64024 | 115 | 18.50% |
| 20 | 25.147 | 3.53852 | 96.1 | 15.40% |
| 21 | 28.77 | 3.10062 | 65.7 | 10.50% |

The above crystal exhibits thermal events between about 132° C. and 134° C., e.g., at about 133° C., and between about 227° C. and 229° C., e.g., at about 228° C., according to differential scanning calorimetry. Fumarate Salt 1 is in solvate form, and the free base and counter ion are present in the crystal in a ratio of 1:0.4.

A second sulfate salt is obtained when sulfuric acid is combined with 25 mg of Compound 1 in ethyl acetate, and is subsequently slurried at 50° C. for 2 hours. The resultant salt is a solvate and is obtained as an off-white powder after evaporation. The XRPD pattern of Sulfate Salt 2 has peaks as set forth in Table 8 below.

TABLE 8

| | | | | Rel. |
|---|---|---|---|---|
| Index | Angle | d-Value | Intensity | Intensity |
| 1 | 4.885 | 18.07685 | 849 | 42.70% |
| 2 | 5.889 | 14.99628 | 1.99E+03 | 100.00% |
| 3 | 8.366 | 10.56019 | 907 | 45.60% |
| 4 | 9.097 | 9.71389 | 112 | 5.60% |
| 5 | 9.576 | 9.22831 | 807 | 40.50% |
| 6 | 10.662 | 8.29096 | 1.16E+03 | 58.20% |
| 7 | 11.417 | 7.74424 | 345 | 17.30% |
| 8 | 12.153 | 7.27709 | 325 | 16.30% |
| 9 | 12.263 | 7.21201 | 355 | 17.80% |
| 10 | 12.543 | 7.05173 | 289 | 14.50% |
| 11 | 14.349 | 6.16764 | 120 | 6.00% |
| 12 | 14.546 | 6.08464 | 229 | 11.50% |
| 13 | 14.763 | 5.99587 | 559 | 28.10% |
| 14 | 15.383 | 5.75531 | 325 | 16.30% |
| 15 | 15.812 | 5.60024 | 156 | 7.80% |
| 16 | 17.877 | 4.95759 | 928 | 46.60% |
| 17 | 18.223 | 4.8643 | 416 | 20.90% |
| 18 | 18.753 | 4.72802 | 427 | 21.40% |
| 19 | 19.592 | 4.52745 | 487 | 24.50% |
| 20 | 19.936 | 4.45001 | 443 | 22.30% |
| 21 | 20.244 | 4.38309 | 156 | 7.80% |
| 22 | 20.797 | 4.26783 | 719 | 36.10% |
| 23 | 21.392 | 4.15035 | 402 | 20.20% |
| 24 | 21.987 | 4.03943 | 252 | 12.60% |
| 25 | 22.24 | 3.99402 | 166 | 8.40% |
| 26 | 22.582 | 3.9343 | 484 | 24.30% |
| 27 | 22.756 | 3.90466 | 650 | 32.70% |
| 28 | 23.521 | 3.77937 | 269 | 13.50% |
| 29 | 24.094 | 3.69075 | 107 | 5.40% |
| 30 | 24.93 | 3.56873 | 200 | 10.00% |
| 31 | 25.146 | 3.53868 | 412 | 20.70% |
| 32 | 25.439 | 3.49848 | 272 | 13.70% |
| 33 | 25.99 | 3.42561 | 251 | 12.60% |
| 34 | 26.466 | 3.36502 | 267 | 13.40% |
| 35 | 26.523 | 3.358 | 234 | 11.70% |
| 36 | 26.889 | 3.31302 | 140 | 7.10% |
| 37 | 27.438 | 3.24799 | 68.2 | 3.40% |
| 38 | 27.876 | 3.19794 | 66.9 | 3.40% |
| 39 | 28.794 | 3.09804 | 140 | 7.10% |
| 40 | 29.13 | 3.06305 | 138 | 7.00% |
| 41 | 29.698 | 3.00578 | 130 | 6.60% |
| 42 | 29.868 | 2.98902 | 120 | 6.00% |
| 43 | 31.584 | 2.83049 | 75.6 | 3.80% |

The above crystal exhibits thermal events between about 69° C. and 71° C., e.g., at about 70° C., and between about 114° C. and 116° C., e.g., at about 115° C., according to differential scanning calorimetry. Sulfate Salt 2 is in solvate form, and the free base and counter ion are present in the crystal in a ratio of 1:0.5.

### Esylate Salt

An esylate salt is obtained when ethane sulfonic acid is combined with 25 mg of Compound 1 in acetone, and is subsequently slurried at 50° C. for 2 hours. The resulting salt is obtained as a white powder. The XRPD pattern of Esylate Salt 1 has peaks as set forth in Table 9 below.

TABLE 9

| | | | | Rel. |
|---|---|---|---|---|
| Index | Angle | d-Value | Intensity | Intensity |
| 1 | 6.196 | 14.25239 | 1.87E+03 | 97.00% |
| 2 | 6.985 | 12.64457 | 272 | 14.10% |
| 3 | 8.771 | 10.07414 | 783 | 40.70% |
| 4 | 9.062 | 9.75116 | 138 | 7.20% |
| 5 | 9.24 | 9.56382 | 223 | 11.60% |
| 6 | 9.656 | 9.15205 | 200 | 10.40% |
| 7 | 11.7 | 7.55728 | 999 | 52.00% |
| 8 | 11.974 | 7.38531 | 216 | 11.20% |
| 9 | 11.978 | 7.38257 | 184 | 9.60% |
| 10 | 12.134 | 7.28816 | 375 | 19.50% |
| 11 | 12.252 | 7.21806 | 551 | 28.60% |
| 12 | 13.605 | 6.50351 | 170 | 8.80% |
| 13 | 14.46 | 6.12071 | 342 | 17.80% |
| 14 | 14.591 | 6.06605 | 341 | 17.70% |
| 15 | 16.081 | 5.50711 | 266 | 13.80% |
| 16 | 16.301 | 5.4332 | 244 | 12.70% |
| 17 | 16.765 | 5.28407 | 148 | 7.70% |
| 18 | 17.009 | 5.20867 | 973 | 50.60% |
| 19 | 17.094 | 5.18287 | 399 | 20.70% |
| 20 | 17.337 | 5.11095 | 602 | 31.30% |
| 21 | 17.717 | 5.00204 | 211 | 11.00% |
| 22 | 18.281 | 4.84913 | 369 | 19.20% |
| 23 | 18.4 | 4.8179 | 642 | 33.40% |
| 24 | 18.739 | 4.73153 | 978 | 50.90% |
| 25 | 19.149 | 4.63112 | 826 | 42.90% |
| 26 | 19.163 | 4.62771 | 796 | 41.40% |
| 27 | 19.262 | 4.60434 | 1.92E+03 | 100.00% |
| 28 | 19.849 | 4.46934 | 216 | 11.20% |
| 29 | 20.296 | 4.37197 | 1.23E+03 | 64.00% |
| 30 | 20.463 | 4.33673 | 1.50E+03 | 78.00% |
| 31 | 20.717 | 4.28401 | 174 | 9.10% |
| 32 | 21.19 | 4.18953 | 674 | 35.00% |
| 33 | 21.386 | 4.15161 | 162 | 8.40% |
| 34 | 21.663 | 4.09914 | 338 | 17.60% |
| 35 | 22.117 | 4.01592 | 283 | 14.70% |
| 36 | 22.469 | 3.95374 | 119 | 6.20% |
| 37 | 22.767 | 3.90265 | 1.06E+03 | 55.20% |
| 38 | 23.445 | 3.79136 | 825 | 42.90% |
| 39 | 23.741 | 3.7447 | 808 | 42.00% |
| 40 | 23.951 | 3.71247 | 293 | 15.20% |
| 41 | 24.958 | 3.56485 | 115 | 6.00% |
| 42 | 25.539 | 3.4851 | 55.1 | 2.90% |
| 43 | 26.048 | 3.41811 | 702 | 36.50% |
| 44 | 26.619 | 3.34602 | 115 | 6.00% |
| 45 | 26.738 | 3.33149 | 159 | 8.20% |
| 46 | 27.793 | 3.20729 | 87.1 | 4.50% |
| 47 | 27.921 | 3.1929 | 243 | 12.60% |
| 48 | 28.162 | 3.16614 | 421 | 21.90% |
| 49 | 28.225 | 3.1592 | 292 | 15.20% |
| 50 | 28.742 | 3.10353 | 361 | 18.80% |
| 51 | 28.811 | 3.09624 | 272 | 14.20% |
| 52 | 29.002 | 3.0763 | 198 | 10.30% |
| 53 | 29.052 | 3.07117 | 151 | 7.80% |
| 54 | 29.793 | 2.99645 | 218 | 11.30% |
| 55 | 29.808 | 2.99491 | 194 | 10.10% |
| 56 | 33.232 | 2.6938 | 206 | 10.70% |
| 57 | 34.183 | 2.62099 | 103 | 5.40% |
| 58 | 36.781 | 2.44158 | 88.4 | 4.60% |
| 59 | 37.969 | 2.36787 | 84 | 4.40% |

The above crystal exhibits a thermal event between about 304° C. and 306° C., e.g., at about 305° C., according to differential scanning calorimetry. Esylate Salt 1 is in solvate form, and the free base and counter ion are present in the crystal in a ratio of 1:1.

### Galactarate Salt

A galactarate salt is obtained when galactaric acid is combined with 25 mg of Compound 1 in methanol and water (9:1), and is subsequently slurried at 50° C. for 2 hours. The resulting salt is obtained as an off-white powder. The XRPD pattern of Galactarate Salt 1 has peaks as set forth in Table 10 below.

TABLE 10

| | | d- | | Rel. |
|---|---|---|---|---|
| Index | Angle | Value | Intensity | Intensity |
| 1 | 3.231 | 27.3233 | 187 | 2.30% |
| 2 | 13.002 | 6.8033 | 1.03E+03 | 12.80% |
| 3 | 15.302 | 5.7856 | 512 | 6.40% |
| 4 | 18.156 | 4.88225 | 1.80E+03 | 22.30% |
| 5 | 19.656 | 4.51292 | 8.04E+03 | 100.00% |
| 6 | 20.514 | 4.3259 | 576 | 7.20% |
| 7 | 21.514 | 4.12706 | 1.41E+03 | 17.60% |
| 8 | 22.983 | 3.86661 | 833 | 10.40% |
| 9 | 23.151 | 3.83891 | 256 | 3.20% |
| 10 | 24.558 | 3.62198 | 729 | 9.10% |
| 11 | 26.082 | 3.41373 | 975 | 12.10% |
| 12 | 26.827 | 3.32055 | 1.90E+03 | 23.60% |
| 13 | 29.721 | 3.00353 | 296 | 3.70% |
| 14 | 30.738 | 2.90639 | 6.18E+03 | 76.80% |
| 15 | 33.061 | 2.7073 | 619 | 7.70% |
| 16 | 33.144 | 2.70076 | 353 | 4.40% |
| 17 | 34.463 | 2.60034 | 5.60E+03 | 69.70% |
| 18 | 34.513 | 2.59669 | 3.50E+03 | 43.50% |
| 19 | 34.847 | 2.57256 | 753 | 9.40% |
| 20 | 36.66 | 2.44935 | 2.10E+03 | 26.10% |
| 21 | 36.75 | 2.44355 | 1.22E+03 | 15.20% |
| 22 | 36.964 | 2.42994 | 277 | 3.40% |
| 23 | 37.208 | 2.41454 | 158 | 2.00% |
| 24 | 37.29 | 2.40945 | 245 | 3.00% |
| 25 | 37.595 | 2.39057 | 2.74E+03 | 34.00% |
| 26 | 37.676 | 2.3856 | 1.64E+03 | 20.40% |
| 27 | 39.797 | 2.26324 | 453 | 5.60% |
| 28 | 40.282 | 2.23711 | 291 | 3.60% |
| 29 | 40.887 | 2.20539 | 994 | 12.40% |
| 30 | 43.767 | 2.06669 | 121 | 1.50% |
| 31 | 44.011 | 2.05578 | 570 | 7.10% |
| 32 | 44.125 | 2.05077 | 369 | 4.60% |

The above crystal exhibits a thermal event between about 204° C. and 206° C., e.g., at about 205° C., according to differential scanning calorimetry. Galactarate Salt 1 is in solvate form, and the free base and counter ion are present in the crystal in a ratio of 1:0.9.

### Adipate Salt

An adipate salt is obtained when adipic acid is combined with 25 mg of Compound 1 in ethyl acetate, and is subsequently slurried at 50° C. for 2 hours. The resulting salt is obtained as a white powder after evaporation. The XRPD pattern of Adipate Salt 1 has peaks as set forth in Table 11 below.

TABLE 11

| | | | | Rel. |
|---|---|---|---|---|
| Index | Angle | d-Value | Intensity | Intensity |
| 1 | 4.955 | 17.81898 | 1.37E+03 | 61.40% |
| 2 | 6.068 | 14.5547 | 612 | 27.40% |
| 3 | 6.911 | 12.77989 | 451 | 20.20% |
| 4 | 7.189 | 12.28586 | 2.23E+03 | 100.00% |
| 5 | 8.708 | 10.14651 | 229 | 10.30% |
| 6 | 9.883 | 8.94231 | 635 | 28.50% |
| 7 | 9.996 | 8.8416 | 1.59E+03 | 71.10% |
| 8 | 13.965 | 6.3364 | 695 | 31.20% |
| 9 | 14.714 | 6.01544 | 621 | 27.80% |
| 10 | 15.002 | 5.90053 | 308 | 13.80% |
| 11 | 15.4 | 5.74908 | 976 | 43.70% |
| 12 | 16.253 | 5.44913 | 1.49E+03 | 66.70% |
| 13 | 16.612 | 5.33237 | 1.01E+03 | 45.20% |
| 14 | 17.775 | 4.98602 | 1.82E+03 | 81.40% |
| 15 | 18.505 | 4.79073 | 184 | 8.30% |
| 16 | 18.621 | 4.76125 | 360 | 16.10% |
| 17 | 19.929 | 4.45163 | 237 | 10.60% |
| 18 | 20.451 | 4.33916 | 1.00E+03 | 45.00% |
| 19 | 20.88 | 4.25094 | 435 | 19.50% |
| 20 | 21.45 | 4.13921 | 126 | 5.70% |
| 21 | 21.524 | 4.12525 | 67.5 | 3.00% |
| 22 | 21.935 | 4.04888 | 302 | 13.60% |
| 23 | 22.144 | 4.01101 | 772 | 34.60% |
| 24 | 22.597 | 3.93164 | 868 | 38.90% |
| 25 | 23.164 | 3.83676 | 319 | 14.30% |
| 26 | 23.606 | 3.76583 | 717 | 32.20% |
| 27 | 23.891 | 3.72155 | 1.71E+03 | 76.50% |
| 28 | 24.564 | 3.62109 | 114 | 5.10% |
| 29 | 24.823 | 3.58392 | 458 | 20.50% |
| 30 | 25.111 | 3.54342 | 295 | 13.20% |
| 31 | 25.669 | 3.46767 | 387 | 17.30% |
| 32 | 25.707 | 3.46262 | 452 | 20.30% |
| 33 | 25.754 | 3.45651 | 418 | 18.70% |
| 34 | 25.701 | 3.46344 | 461 | 20.70% |
| 35 | 27.469 | 3.24438 | 161 | 7.20% |
| 36 | 27.853 | 3.20055 | 292 | 13.10% |
| 37 | 27.872 | 3.19847 | 308 | 13.80% |
| 38 | 28.2 | 3.16192 | 98.9 | 4.40% |
| 39 | 28.949 | 3.08185 | 129 | 5.80% |
| 40 | 29.961 | 2.97996 | 140 | 6.30% |
| 41 | 30.795 | 2.90118 | 177 | 7.90% |
| 42 | 33.449 | 2.67679 | 158 | 7.10% |
| 43 | 33.457 | 2.6762 | 130 | 5.80% |
| 44 | 33.658 | 2.66063 | 111 | 5.00% |
| 45 | 33.969 | 2.63697 | 62.3 | 2.80% |
| 46 | 35.018 | 2.56038 | 138 | 6.20% |
| 47 | 36.534 | 2.45751 | 132 | 5.90% |
| 48 | 38.316 | 2.34722 | 385 | 17.30% |
| 49 | 38.39 | 2.34288 | 222 | 10.00% |
| 50 | 41.517 | 2.17334 | 71.5 | 3.20% |

The above crystal exhibits thermal events between about 119° C. and 121° C., e.g., at about 120° C., and between about 159° C. and 161° C., e.g., at about 160° C., according to differential scanning calorimetry. Adipate Salt 1 is in anhydrous form, and the free base and counter ion are present in the crystal in a ratio of 1:1.

A second adipate salt is obtained when adipic acid is combined with 25 mg of Compound 1 in acetonitrile, and is subsequently slurried at 50° C. for 2 hours. The resulting salt is obtained as an off-white powder. The XRPD pattern of Adipate Salt 2 has peaks as set forth in Table 12 below.

TABLE 12

| | | | | Rel. |
|---|---|---|---|---|
| Index | Angle | d-Value | Intensity | Intensity |
| 1 | 4.887 | 18.06727 | 580 | 63.50% |
| 2 | 6.692 | 13.19851 | 593 | 64.90% |
| 3 | 8.92 | 9.90534 | 524 | 57.40% |
| 4 | 9.686 | 9.124 | 165 | 18.00% |
| 5 | 10.703 | 8.25927 | 395 | 43.20% |
| 6 | 12.688 | 6.97118 | 177 | 19.30% |
| 7 | 15.743 | 5.62474 | 343 | 37.50% |
| 8 | 15.795 | 5.60605 | 306 | 33.50% |
| 9 | 16.859 | 5.25458 | 315 | 34.50% |
| 10 | 17.847 | 4.96599 | 346 | 37.80% |
| 11 | 18.028 | 4.91663 | 913 | 100.00% |
| 12 | 20.169 | 4.39921 | 311 | 34.10% |
| 13 | 20.324 | 4.36597 | 120 | 13.20% |
| 14 | 21.252 | 4.17733 | 268 | 29.40% |
| 15 | 21.296 | 4.16891 | 328 | 35.90% |
| 16 | 22.343 | 3.97575 | 353 | 38.70% |
| 17 | 26.336 | 3.38132 | 163 | 17.90% |

The above crystal exhibits thermal events between about 159° C. and 161° C., e.g., at about 160° C., according to differential scanning calorimetry. Adipate Salt 2 is in anhydrous form, and the free base and counter ion are present in the crystal in a ratio of 2:1.

A third adipate salt is obtained when adipic acid is combined with 25 mg of Compound 1 in acetone, and is subsequently slurried at 50° C. for 2 hours. The resulting salt is obtained as an off-white powder. The XRPD pattern of Adipate Salt 2 has peaks as set forth in Table 12 Å below.

TABLE 12A

| Index | Angle | d-Value | Intensity | Rel.Intensity |
|---|---|---|---|---|
| 1 | 6.23 | 14.17652 | 889 | 14.30% |
| 2 | 6.3 | 14.018 | 1.10E+03 | 17.60% |
| 3 | 6.488 | 13.6129 | 5.25E+03 | 84.60% |
| 4 | 7.262 | 12.16303 | 594 | 9.60% |
| 5 | 7.94 | 11.12578 | 824 | 13.30% |
| 6 | 8.077 | 10.93777 | 1.99E+03 | 32.10% |
| 7 | 8.608 | 10.26424 | 211 | 3.40% |
| 8 | 8.725 | 10.1267 | 642 | 10.30% |
| 9 | 10.461 | 8.44984 | 4.84E+03 | 77.90% |
| 10 | 10.479 | 8.43528 | 6.21E+03 | 100.00% |
| 11 | 11.116 | 7.95343 | 1.90E+03 | 30.60% |
| 12 | 12.212 | 7.24202 | 4.83E+03 | 77.90% |
| 13 | 12.673 | 6.9794 | 344 | 5.50% |
| 14 | 12.892 | 6.86159 | 1.49E+03 | 24.00% |
| 15 | 12.936 | 6.83811 | 1.79E+03 | 28.80% |
| 16 | 13.883 | 6.37372 | 73.3 | 1.20% |
| 17 | 14.09 | 6.28043 | 208 | 3.40% |
| 18 | 14.233 | 6.21775 | 660 | 10.60% |
| 19 | 14.592 | 6.0657 | 200 | 3.20% |
| 20 | 15.142 | 5.8465 | 104 | 1.70% |
| 21 | 15.966 | 5.54642 | 657 | 10.60% |
| 22 | 16.112 | 5.49647 | 134 | 2.20% |
| 23 | 16.745 | 5.29032 | 828 | 13.30% |
| 24 | 17.41 | 5.0896 | 792 | 12.80% |
| 25 | 17.715 | 5.00257 | 1.48E+03 | 23.90% |
| 26 | 18.074 | 4.90404 | 996 | 16.00% |
| 27 | 18.233 | 4.86182 | 3.72E+03 | 59.80% |
| 28 | 18.417 | 4.81353 | 1.27E+03 | 20.50% |
| 29 | 18.754 | 4.72788 | 339 | 5.50% |
| 30 | 18.945 | 4.68068 | 1.44E+03 | 23.10% |
| 31 | 19.356 | 4.58212 | 556 | 9.00% |
| 32 | 19.71 | 4.50062 | 985 | 15.90% |
| 33 | 19.942 | 4.44882 | 1.54E+03 | 24.80% |
| 34 | 20.211 | 4.39018 | 307 | 4.90% |
| 35 | 20.494 | 4.33026 | 1.02E+03 | 16.40% |
| 36 | 20.557 | 4.31693 | 1.05E+03 | 16.90% |
| 37 | 20.789 | 4.26937 | 1.68E+03 | 27.00% |
| 38 | 21.382 | 4.15225 | 843 | 13.60% |
| 39 | 21.511 | 4.12769 | 1.90E+03 | 30.50% |
| 40 | 21.667 | 4.09837 | 1.45E+03 | 23.30% |
| 41 | 22.077 | 4.02317 | 114 | 1.80% |
| 42 | 22.273 | 3.98821 | 1.11E+03 | 17.90% |
| 43 | 22.672 | 3.9189 | 1.10E+03 | 17.70% |
| 44 | 23.037 | 3.85759 | 455 | 7.30% |
| 45 | 23.321 | 3.81122 | 256 | 4.10% |
| 46 | 23.369 | 3.80359 | 239 | 3.90% |
| 47 | 23.399 | 3.79872 | 162 | 2.60% |
| 48 | 23.854 | 3.72721 | 1.58E+03 | 25.40% |
| 49 | 23.879 | 3.72339 | 1.76E+03 | 28.30% |
| 50 | 24.438 | 3.63946 | 2.76E+03 | 44.40% |
| 51 | 24.58 | 3.61878 | 1.40E+03 | 22.50% |
| 52 | 24.935 | 3.56816 | 232 | 3.70% |
| 53 | 25.854 | 3.44331 | 679 | 10.90% |
| 54 | 26.834 | 3.31978 | 513 | 8.30% |
| 55 | 26.877 | 3.31449 | 417 | 6.70% |
| 56 | 27.132 | 3.28399 | 333 | 5.40% |
| 57 | 27.768 | 3.21015 | 772 | 12.40% |
| 58 | 28.639 | 3.11445 | 176 | 2.80% |
| 59 | 28.957 | 3.08098 | 286 | 4.60% |
| 60 | 29.544 | 3.02112 | 566 | 9.10% |
| 61 | 29.886 | 2.9873 | 589 | 9.50% |
| 62 | 29.916 | 2.98436 | 596 | 9.60% |
| 63 | 30.178 | 2.95902 | 479 | 7.70% |
| 64 | 30.424 | 2.93569 | 256 | 4.10% |
| 65 | 30.456 | 2.93272 | 249 | 4.00% |
| 66 | 30.893 | 2.89218 | 275 | 4.40% |
| 67 | 31.388 | 2.84766 | 145 | 2.30% |
| 68 | 31.444 | 2.84272 | 79.4 | 1.30% |
| 69 | 32.057 | 2.7898 | 138 | 2.20% |
| 70 | 32.081 | 2.7877 | 141 | 2.30% |
| 71 | 33.014 | 2.7111 | 71.1 | 1.10% |
| 72 | 33.45 | 2.67675 | 95 | 1.50% |
| 73 | 34.123 | 2.62542 | 171 | 2.80% |
| 74 | 34.176 | 2.62151 | 107 | 1.70% |
| 75 | 34.767 | 2.5783 | 167 | 2.70% |
| 76 | 35.645 | 2.51678 | 233 | 3.80% |
| 77 | 36.047 | 2.48962 | 114 | 1.80% |
| 78 | 36.188 | 2.48023 | 117 | 1.90% |
| 79 | 36.949 | 2.43087 | 71.4 | 1.20% |
| 80 | 39.125 | 2.30054 | 107 | 1.70% |
| 81 | 40.597 | 2.22047 | 90.6 | 1.50% |
| 82 | 44.167 | 2.04892 | 96.1 | 1.50% |

The above crystal exhibits thermal events between about 109° C. and 112° C., e.g., at about 109° C., according to differential scanning calorimetry. Adipate Salt 3 is in anhydrous form, and the free base and counter ion are present in the crystal in a ratio of 1:1.

### Lactate Salt

A lactate salt is obtained when lactic acid is combined with 25 mg of Compound 1 in ethyl acetate or toluene, and is subsequently slurried at 50° C. for 2 hours. The resulting salt is obtained as a white powder. The XRPD pattern of Lactate Salt 1 has peaks as set forth in Table 13 below.

TABLE 13

| | | | | Rel. |
|---|---|---|---|---|
| Index | Angle | d-Value | Intensity | Intensity |
| 1 | 3.078 | 28.68012 | 120 | 3.30% |
| 2 | 6.288 | 14.04515 | 1.41E+03 | 38.90% |
| 3 | 6.397 | 13.80531 | 2.49E+03 | 68.60% |
| 4 | 6.46 | 13.67145 | 3.60E+03 | 99.40% |
| 5 | 7.981 | 11.0688 | 130 | 3.60% |
| 6 | 8.731 | 10.12029 | 1.40E+03 | 38.70% |
| 7 | 9.05 | 9.76373 | 1.58E+03 | 43.70% |
| 8 | 9.717 | 9.09481 | 1.20E+03 | 33.00% |
| 9 | 9.873 | 8.95125 | 493 | 13.60% |
| 10 | 11.849 | 7.46289 | 2.80E+03 | 77.40% |
| 11 | 12.264 | 7.21139 | 3.62E+03 | 100.00% |
| 12 | 12.453 | 7.10199 | 761 | 21.00% |
| 13 | 13.975 | 6.33187 | 572 | 15.80% |
| 14 | 14.271 | 6.20106 | 339 | 9.30% |
| 15 | 16.455 | 5.38296 | 1.30E+03 | 35.80% |
| 16 | 16.939 | 5.22994 | 198 | 5.50% |
| 17 | 17.124 | 5.17387 | 1.90E+03 | 52.50% |
| 18 | 17.27 | 5.13062 | 446 | 12.30% |
| 19 | 17.616 | 5.0306 | 956 | 26.40% |
| 20 | 17.793 | 4.98082 | 655 | 18.10% |
| 21 | 17.971 | 4.93207 | 205 | 5.70% |
| 22 | 18.693 | 4.7431 | 165 | 4.50% |
| 23 | 18.844 | 4.70538 | 1.14E+03 | 31.40% |
| 24 | 19.403 | 4.57113 | 1.92E+03 | 53.00% |
| 25 | 19.708 | 4.50106 | 1.30E+03 | 35.90% |
| 26 | 20.468 | 4.33553 | 2.86E+03 | 78.80% |
| 27 | 20.678 | 4.29213 | 1.16E+03 | 31.90% |
| 28 | 21.142 | 4.19884 | 659 | 18.20% |
| 29 | 21.595 | 4.11178 | 1.16E+03 | 31.90% |
| 30 | 21.815 | 4.07089 | 500 | 13.80% |
| 31 | 22.381 | 3.96919 | 512 | 14.10% |
| 32 | 22.607 | 3.92998 | 573 | 15.80% |
| 33 | 23.151 | 3.83883 | 1.66E+03 | 45.70% |
| 34 | 23.329 | 3.80991 | 1.86E+03 | 51.30% |
| 35 | 24.433 | 3.64023 | 836 | 23.10% |
| 36 | 24.761 | 3.59279 | 388 | 10.70% |
| 37 | 25.374 | 3.50728 | 552 | 15.20% |
| 38 | 25.894 | 3.43807 | 148 | 4.10% |
| 39 | 27.191 | 3.27694 | 93.4 | 2.60% |
| 40 | 27.814 | 3.20497 | 540 | 14.90% |
| 41 | 28.046 | 3.17896 | 838 | 23.10% |
| 42 | 28.114 | 3.17148 | 569 | 15.70% |
| 43 | 28.606 | 3.11799 | 137 | 3.80% |
| 44 | 29.215 | 3.05442 | 246 | 6.80% |
| 45 | 29.26 | 3.04974 | 271 | 7.50% |
| 46 | 29.369 | 3.03875 | 455 | 12.50% |
| 47 | 29.555 | 3.02 | 276 | 7.60% |
| 48 | 30.945 | 2.88747 | 468 | 12.90% |
| 49 | 32.376 | 2.763 | 125 | 3.50% |
| 50 | 32.678 | 2.7382 | 220 | 6.10% |
| 51 | 33.182 | 2.69773 | 262 | 7.20% |
| 52 | 33.42 | 2.67908 | 171 | 4.70% |
| 53 | 34.665 | 2.5856 | 159 | 4.40% |
| 54 | 34.728 | 2.58108 | 199 | 5.50% |
| 55 | 36.646 | 2.45025 | 81.8 | 2.30% |
| 56 | 38.61 | 2.33001 | 122 | 3.40% |
| 57 | 38.68 | 2.32595 | 97.6 | 2.70% |
| 58 | 41.423 | 2.17807 | 71.1 | 2.00% |

The above crystal exhibits thermal events between about 187° C. and 190° C., e.g., at about 187° C. or 188° C., according to differential scanning calorimetry. Lactate Salt 1 is in anhydrous form, and the free base and counter ion are present in the crystal in a ratio of 1:1.

### Oxalate Salt

An oxalate salt is obtained when oxalic acid is combined with 25 mg of Compound 1 in 3-heptanone, and is subsequently cooled to 5° C. over an 8 hour period. The resulting salt is obtained as an off-white powder. The XRPD pattern of Oxalate Salt 1 has peaks as set forth in Table 14 below.

TABLE 14

| | | d- | | Rel. |
|---|---|---|---|---|
| Index | Angle | Value | Intensity | Intensity |
| 1 | 7.132 | 12.384 | 2.73E+03 | 45.80% |
| 2 | 8.504 | 10.39 | 4.64E+03 | 77.70% |
| 3 | 8.87 | 9.9615 | 2.11E+03 | 35.30% |
| 4 | 9.644 | 9.1636 | 462 | 7.70% |
| 5 | 9.885 | 8.9407 | 411 | 6.90% |
| 6 | 12.24 | 7.2251 | 5.97E+03 | 100.00% |
| 7 | 12.309 | 7.1852 | 4.39E+03 | 73.50% |
| 8 | 12.659 | 6.9871 | 266 | 4.50% |
| 9 | 13.757 | 6.4318 | 600 | 10.00% |
| 10 | 14.124 | 6.2656 | 463 | 7.80% |
| 11 | 16.325 | 5.4253 | 3.38E+03 | 56.60% |
| 12 | 16.71 | 5.3012 | 2.04E+03 | 34.20% |
| 13 | 16.919 | 5.2362 | 1.65E+03 | 27.60% |
| 14 | 17.325 | 5.1143 | 1.81E+03 | 30.30% |
| 15 | 17.651 | 5.0206 | 915 | 15.30% |
| 16 | 18.268 | 4.8525 | 522 | 8.70% |
| 17 | 18.584 | 4.7708 | 2.11E+03 | 35.40% |
| 18 | 19.246 | 4.608 | 2.94E+03 | 49.20% |
| 19 | 19.755 | 4.4905 | 1.94E+03 | 32.50% |
| 20 | 20.327 | 4.3654 | 360 | 6.00% |
| 21 | 20.662 | 4.2953 | 3.14E+03 | 52.70% |
| 22 | 20.947 | 4.2376 | 449 | 7.50% |
| 23 | 21.366 | 4.1553 | 654 | 11.00% |
| 24 | 21.725 | 4.0876 | 767 | 12.90% |
| 25 | 22.057 | 4.0268 | 191 | 3.20% |
| 26 | 22.435 | 3.9597 | 1.04E+03 | 17.50% |
| 27 | 22.939 | 3.8738 | 2.42E+03 | 40.50% |
| 28 | 23.488 | 3.7845 | 762 | 12.80% |
| 29 | 23.582 | 3.7696 | 1.25E+03 | 21.00% |
| 30 | 23.776 | 3.7393 | 2.14E+03 | 35.90% |
| 31 | 24.109 | 3.6884 | 2.16E+03 | 36.20% |
| 32 | 24.824 | 3.5838 | 675 | 11.30% |
| 33 | 25.399 | 3.504 | 2.16E+03 | 36.20% |
| 34 | 25.802 | 3.4502 | 1.04E+03 | 17.40% |
| 35 | 26.677 | 3.3389 | 136 | 2.30% |
| 36 | 27.366 | 3.2564 | 363 | 6.10% |
| 37 | 27.71 | 3.2167 | 508 | 8.50% |
| 38 | 29.06 | 3.0703 | 470 | 7.90% |
| 39 | 29.634 | 3.0122 | 1.09E+03 | 18.20% |
| 40 | 30.072 | 2.9693 | 192 | 3.20% |
| 41 | 30.539 | 2.9249 | 717 | 12.00% |
| 42 | 31.166 | 2.8675 | 841 | 14.10% |
| 43 | 31.977 | 2.7966 | 80.7 | 1.40% |
| 44 | 33.733 | 2.6549 | 311 | 5.20% |
| 45 | 34.219 | 2.6183 | 422 | 7.10% |
| 46 | 34.722 | 2.5815 | 155 | 2.60% |
| 47 | 35.303 | 2.5403 | 209 | 3.50% |
| 48 | 35.85 | 2.5028 | 248 | 4.20% |
| 49 | 36.936 | 2.4317 | 149 | 2.50% |
| 50 | 39.038 | 2.3055 | 82.9 | 1.40% |
| 51 | 39.644 | 2.2716 | 104 | 1.70% |
| 52 | 41.142 | 2.1923 | 139 | 2.30% |

The above crystal exhibits a thermal event between about 218° C. and 220° C., e.g., at about 219° C., according to differential scanning calorimetry. Oxalate Salt 1 is in anhydrous form.

A second oxalate salt is obtained when oxalic acid is combined with 25 mg of Compound 1 in acetonitrile, and is subsequently cooled to 5° C. over an 8 hour period. The resulting salt is obtained as in the form of white needles. The XRPD pattern of Oxalate Salt 2 has peaks as set forth in Table 15 below.

TABLE 15

| | | d- | | Rel. |
|---|---|---|---|---|
| Index | Angle | Value | Intensity | Intensity |
| 1 | 6.451 | 13.691 | 819 | 15.80% |
| 2 | 6.711 | 13.161 | 1.36E+03 | 26.20% |
| 3 | 7.213 | 12.246 | 5.19E+03 | 100.00% |
| 4 | 10.12 | 8.7339 | 136 | 2.60% |
| 5 | 14.37 | 6.1581 | 259 | 5.00% |
| 6 | 16.29 | 5.4368 | 755 | 14.60% |
| 7 | 16.51 | 5.3641 | 295 | 5.70% |
| 8 | 16.71 | 5.3003 | 1.02E+03 | 19.60% |
| 9 | 16.95 | 5.2274 | 2.30E+03 | 44.40% |
| 10 | 17.24 | 5.1382 | 390 | 7.50% |
| 11 | 17.26 | 5.134 | 361 | 7.00% |
| 12 | 17.4 | 5.092 | 452 | 8.70% |
| 13 | 17.78 | 4.9844 | 228 | 4.40% |
| 14 | 18.03 | 4.9161 | 444 | 8.60% |
| 15 | 18.23 | 4.8635 | 228 | 4.40% |
| 16 | 19.46 | 4.5571 | 844 | 16.30% |
| 17 | 19.66 | 4.5124 | 441 | 8.50% |
| 18 | 19.86 | 4.4666 | 410 | 7.90% |
| 19 | 20.03 | 4.429 | 958 | 18.50% |
| 20 | 20.46 | 4.337 | 390 | 7.50% |
| 21 | 20.63 | 4.3026 | 667 | 12.90% |
| 22 | 20.85 | 4.2575 | 557 | 10.70% |
| 23 | 21.1 | 4.2076 | 250 | 4.80% |
| 24 | 21.56 | 4.1181 | 187 | 3.60% |
| 25 | 22.16 | 4.0084 | 102 | 2.00% |
| 26 | 25.83 | 3.4467 | 194 | 3.70% |
| 27 | 26.68 | 3.3384 | 311 | 6.00% |
| 28 | 26.89 | 3.3125 | 186 | 3.60% |
| 29 | 27.2 | 3.2762 | 438 | 8.40% |
| 30 | 32.79 | 2.7291 | 404 | 7.80% |
| 31 | 33.78 | 2.6515 | 183 | 3.50% |
| 32 | 34.14 | 2.624 | 330 | 6.40% |
| 33 | 34.2 | 2.6195 | 432 | 8.30% |
| 34 | 34.58 | 2.592 | 159 | 3.10% |
| 35 | 35.28 | 2.5419 | 57.5 | 1.10% |
| 36 | 36.62 | 2.4521 | 93.2 | 1.80% |

The above crystal exhibits thermal events between about 165° C. and 167° C., e.g., at about 166° C., between about 205° C. and 207° C., e.g., at about 207° C., and between about 214° C. and 216° C., e.g., at about 215° C., according to differential scanning calorimetry. Oxalate Salt 2 is in anhydrous form.

A third oxalate salt is obtained when oxalic acid is combined with 25 mg of Compound 1 in 3-heptanone, 2-butanone or ethyl acetate. The mixture is then either slurried at 50° C. for 2 hours, cooled to 5° C. over an 8 hour period, or subjected to evaporation under vacuum. The resulting salt is obtained as in the form of white needles. The XRPD pattern of Oxalate Salt 3 has peaks as set forth in Table 16 below.

TABLE 16

| Index | Angle | d-Value | Intensity | Rel.Intensity |
|---|---|---|---|---|
| 1 | 6.077 | 14.53212 | 1.88E+03 | 3.30% |
| 2 | 5.334 | 16.55597 | 4.17E+04 | 72.30% |
| 3 | 5.989 | 14.74539 | 5.77E+04 | 100.00% |
| 4 | 7.106 | 12.42925 | 486 | 0.80% |
| 5 | 7.388 | 11.95675 | 298 | 0.50% |
| 6 | 8.507 | 10.38561 | 802 | 1.40% |
| 7 | 8.917 | 9.90908 | 280 | 0.50% |
| 8 | 9.227 | 9.57676 | 1.67E+03 | 2.90% |
| 9 | 9.907 | 8.92067 | 343 | 0.60% |
| 10 | 10.571 | 8.3624 | 586 | 1.00% |
| 11 | 11.89 | 7.43723 | 7.21E+03 | 12.50% |
| 12 | 12.269 | 7.20802 | 1.37E+03 | 2.40% |
| 13 | 12.538 | 7.0541 | 2.88E+03 | 5.00% |
| 14 | 13.447 | 6.5796 | 522 | 0.90% |
| 15 | 13.747 | 6.43625 | 131 | 0.20% |
| 16 | 14.39 | 6.15035 | 139 | 0.20% |
| 17 | 15.858 | 5.58417 | 2.42E+03 | 4.20% |
| 18 | 16.339 | 5.42077 | 446 | 0.80% |
| 19 | 16.61 | 5.33299 | 4.73E+03 | 8.20% |
| 20 | 16.854 | 5.25641 | 528 | 0.90% |
| 21 | 17.398 | 5.09298 | 2.47E+03 | 4.30% |
| 22 | 17.717 | 5.00216 | 5.94E+03 | 10.30% |
| 23 | 18.287 | 4.84757 | 4.45E+03 | 7.70% |
| 24 | 18.63 | 4.75895 | 691 | 1.20% |
| 25 | 18.975 | 4.67316 | 857 | 1.50% |
| 26 | 19.288 | 4.59812 | 330 | 0.60% |
| 27 | 19.649 | 4.51434 | 4.64E+03 | 8.00% |
| 28 | 20.185 | 4.39576 | 550 | 1.00% |
| 29 | 20.534 | 4.32184 | 4.29E+03 | 7.40% |
| 30 | 20.721 | 4.2832 | 6.78E+03 | 11.80% |
| 31 | 21.231 | 4.18156 | 6.46E+03 | 11.20% |
| 32 | 21.627 | 4.10587 | 747 | 1.30% |
| 33 | 22.135 | 4.01278 | 1.73E+03 | 3.00% |
| 34 | 22.392 | 3.9672 | 1.98E+03 | 3.40% |
| 35 | 22.643 | 3.92385 | 1.04E+03 | 1.80% |
| 36 | 22.916 | 3.87771 | 306 | 0.50% |
| 37 | 23.423 | 3.79486 | 353 | 0.60% |
| 38 | 23.738 | 3.74519 | 3.10E+03 | 5.40% |
| 39 | 24.22 | 3.67181 | 2.84E+03 | 4.90% |
| 40 | 24.97 | 3.56313 | 550 | 1.00% |
| 41 | 25.238 | 3.5259 | 1.79E+03 | 3.10% |
| 42 | 25.804 | 3.44993 | 82.9 | 0.10% |
| 43 | 26.534 | 3.35656 | 3.13E+03 | 5.40% |
| 44 | 27.003 | 3.29939 | 365 | 0.60% |
| 45 | 27.219 | 3.27366 | 788 | 1.40% |
| 46 | 27.67 | 3.2213 | 586 | 1.00% |
| 47 | 27.878 | 3.19772 | 216 | 0.40% |
| 48 | 28.479 | 3.13163 | 326 | 0.60% |
| 49 | 28.977 | 3.07892 | 188 | 0.30% |
| 50 | 29.586 | 3.01691 | 1.40E+03 | 2.40% |
| 51 | 30.359 | 2.94187 | 484 | 0.80% |
| 52 | 30.902 | 2.89138 | 703 | 1.20% |
| 53 | 31.351 | 2.851 | 1.25E+03 | 2.20% |
| 54 | 32.077 | 2.78804 | 829 | 1.40% |
| 55 | 32.618 | 2.74308 | 225 | 0.40% |
| 56 | 33.102 | 2.70406 | 216 | 0.40% |
| 57 | 33.458 | 2.67607 | 186 | 0.30% |
| 58 | 33.937 | 2.63939 | 130 | 0.20% |
| 59 | 34.289 | 2.61314 | 446 | 0.80% |
| 60 | 34.573 | 2.59229 | 171 | 0.30% |
| 61 | 35.011 | 2.56087 | 183 | 0.30% |
| 62 | 35.738 | 2.51041 | 315 | 0.50% |
| 63 | 36.478 | 2.46119 | 325 | 0.60% |
| 64 | 36.856 | 2.43681 | 250 | 0.40% |
| 65 | 37.461 | 2.39881 | 134 | 0.20% |
| 66 | 38.401 | 2.34223 | 207 | 0.40% |
| 67 | 39.392 | 2.28555 | 273 | 0.50% |
| 68 | 40.187 | 2.24217 | 145 | 0.30% |
| 69 | 41.788 | 2.1599 | 123 | 0.20% |
| 70 | 42.071 | 2.14599 | 112 | 0.20% |
| 71 | 43.141 | 2.09521 | 366 | 0.60% |
| 72 | 43.35 | 2.08562 | 297 | 0.50% |
| 73 | 44.028 | 2.05506 | 70.5 | 0.10% |

The above crystal exhibits a thermal event between about 214° C. and 220° C., e.g., at about 214° C., 218° C. or 219° C., according to differential scanning calorimetry. Oxalate Salt 3 is in solvate form.

### Palmitate Salt

A palmitate salt is obtained when palmitic acid is combined with 25 mg of Compound 1 in ethyl acetate, 2-butanone, acetonitrile or 3-heptanone. The mixture is then either slurried at 50° C. for 2 hours, cooled to 5° C. over an 8 hour period, or subjected to evaporation under vacuum. The resulting salt is obtained as a white to off-white powder. The XRPD pattern of Palmitate Salt 1 has peaks as set forth in Table 17 below.

TABLE 17

| | | | | Rel. |
|---|---|---|---|---|
| Index | Angle | d-Value | Intensity | Intensity |
| 1 | 2.077 | 42.49554 | 114 | 1.50% |
| 2 | 2.162 | 40.82731 | 161 | 2.00% |
| 3 | 2.354 | 37.5071 | 227 | 2.90% |
| 4 | 2.562 | 34.45097 | 948 | 12.00% |
| 5 | 3.082 | 28.64018 | 132 | 1.70% |
| 6 | 4.304 | 20.51272 | 7.87E+03 | 100.00% |
| 7 | 5.487 | 16.09433 | 6.83E+03 | 86.80% |
| 8 | 6.525 | 13.53615 | 2.15E+03 | 27.40% |
| 9 | 7.347 | 12.0222 | 1.97E+03 | 25.10% |
| 10 | 8.253 | 10.70432 | 181 | 2.30% |
| 11 | 8.524 | 10.36448 | 3.11E+03 | 39.50% |
| 12 | 9.531 | 9.27225 | 2.24E+03 | 28.50% |
| 13 | 10.669 | 8.28576 | 886 | 11.30% |
| 14 | 10.918 | 8.0972 | 1.75E+03 | 22.30% |
| 15 | 12.182 | 7.25941 | 1.13E+03 | 14.30% |
| 16 | 12.76 | 6.93185 | 477 | 6.10% |
| 17 | 14.467 | 6.11768 | 1.07E+03 | 13.60% |
| 18 | 14.967 | 5.91442 | 1.39E+03 | 17.60% |
| 19 | 14.944 | 5.92351 | 1.33E+03 | 17.00% |
| 20 | 15.411 | 5.74501 | 782 | 9.90% |
| 21 | 16.099 | 5.50092 | 518 | 6.60% |
| 22 | 16.366 | 5.41183 | 438 | 5.60% |
| 23 | 17.011 | 5.20806 | 784 | 10.00% |
| 24 | 17.205 | 5.14966 | 872 | 11.10% |
| 25 | 17.547 | 5.05019 | 440 | 5.60% |
| 26 | 19.044 | 4.65648 | 857 | 10.90% |
| 27 | 19.184 | 4.62268 | 1.50E+03 | 19.10% |
| 28 | 19.788 | 4.48299 | 1.08E+03 | 13.70% |
| 29 | 20.829 | 4.26126 | 799 | 10.10% |
| 30 | 20.839 | 4.25928 | 766 | 9.70% |
| 31 | 21.633 | 4.10473 | 1.47E+03 | 18.60% |
| 32 | 21.686 | 4.09478 | 1.18E+03 | 15.00% |
| 33 | 21.922 | 4.05122 | 1.24E+03 | 15.80% |
| 34 | 22.755 | 3.90482 | 3.96E+03 | 50.40% |
| 35 | 23.462 | 3.78871 | 667 | 8.50% |
| 36 | 23.52 | 3.77948 | 619 | 7.90% |
| 37 | 24.173 | 3.6788 | 345 | 4.40% |
| 38 | 25.045 | 3.55268 | 389 | 4.90% |
| 39 | 25.063 | 3.55017 | 433 | 5.50% |
| 40 | 25.082 | 3.54754 | 421 | 5.30% |
| 41 | 25.617 | 3.47456 | 252 | 3.20% |
| 42 | 26.833 | 3.31983 | 75.5 | 1.00% |

The above crystal exhibits thermal events between about 59° C. and 66° C., e.g., at about 59° C., 62° C. or 63° C., according to differential scanning calorimetry. Palmitate Salt 1 is in anhydrous form, and the free base and counter ion are present in the crystal in a ratio of about 1:1.

### 2-Oxo-Glutarate Salt

A palmitate salt is obtained when palmitic acid is combined with 25 mg of Compound 1 in ethyl acetate. The mixture is then subjected to evaporation under vacuum. The resulting salt is obtained as an off-white powder. The XRPD pattern of 2-Oxo-glutarate Salt has peaks as set forth in Table 18 below.

TABLE 18

| | | | | Rel. |
|---|---|---|---|---|
| Index | Angle | d-Value | Intensity | Intensity |
| 1 | 5.086 | 17.36104 | 2.39E+03 | 100.00% |
| 2 | 8.517 | 10.37362 | 801 | 33.50% |
| 3 | 10.106 | 8.74579 | 207 | 8.70% |
| 4 | 11.047 | 8.00256 | 662 | 27.70% |
| 5 | 11.826 | 7.47744 | 543 | 22.70% |
| 6 | 14.497 | 6.10527 | 443 | 18.60% |
| 7 | 15.685 | 5.64536 | 238 | 10.00% |
| 8 | 17.453 | 5.07724 | 1.57E+03 | 65.80% |
| 9 | 19.793 | 4.48184 | 438 | 18.40% |
| 10 | 20.186 | 4.39542 | 684 | 28.70% |
| 11 | 21.207 | 4.18622 | 200 | 8.40% |
| 12 | 22.271 | 3.98858 | 147 | 6.20% |
| 13 | 23.182 | 3.83377 | 86.9 | 3.60% |
| 14 | 23.787 | 3.73758 | 183 | 7.70% |
| 15 | 24.986 | 3.56088 | 126 | 5.30% |
| 16 | 25.98 | 3.42691 | 114 | 4.80% |
| 17 | 28.779 | 3.09965 | 123 | 5.20% |
| 18 | 30.712 | 2.90879 | 96.6 | 4.00% |
| 19 | 33.248 | 2.69251 | 51.2 | 2.10% |

The above crystal exhibits thermal events between about 124° C. and 126° C., e.g., at about 125° C., and between about 157° C. and 159° C., e.g., at about 158° C., according to differential scanning calorimetry. 2-Oxo-glutarate Salt is in solvate form, and the free base and counter ion are present in the crystal in a ratio of about 1:1.1.

### Xinafoate Salt

A xinafoate salt is obtained when 1-hydroxy-2-napthoic acid is combined with 25 mg of Compound 1 in acetonitrile, and is subsequently slurried at 50° C. for 2 hours. The resulting salt is obtained as a brown powder. The XRPD pattern of Xinafoate Salt 1 has peaks as set forth in Table 19 below.

TABLE 19

| | | | | Rel. |
|---|---|---|---|---|
| Index | Angle | d-Value | Intensity | Intensity |
| 1 | 5.086 | 17.36104 | 2.39E+03 | 100.00% |
| 2 | 8.517 | 10.37362 | 801 | 33.50% |
| 3 | 10.106 | 8.74579 | 207 | 8.70% |
| 4 | 11.047 | 8.00256 | 662 | 27.70% |
| 5 | 11.826 | 7.47744 | 543 | 22.70% |
| 6 | 14.497 | 6.10527 | 443 | 18.60% |
| 7 | 15.685 | 5.64536 | 238 | 10.00% |
| 8 | 17.453 | 5.07724 | 1.57E+03 | 65.80% |
| 9 | 19.793 | 4.48184 | 438 | 18.40% |
| 10 | 20.186 | 4.39542 | 684 | 28.70% |
| 11 | 21.207 | 4.18622 | 200 | 8.40% |
| 12 | 22.271 | 3.98858 | 147 | 6.20% |
| 13 | 23.182 | 3.83377 | 86.9 | 3.60% |
| 14 | 23.787 | 3.73758 | 183 | 7.70% |
| 15 | 24.986 | 3.56088 | 126 | 5.30% |
| 16 | 25.98 | 3.42691 | 114 | 4.80% |
| 17 | 28.779 | 3.09965 | 123 | 5.20% |
| 18 | 30.712 | 2.90879 | 96.6 | 4.00% |
| 19 | 33.248 | 2.69251 | 51.2 | 2.10% |

The above crystal exhibits thermal events between about 130° C. and 132° C., e.g., at about 131° C., and between about 143° C. and 146° C., e.g., at about 145° C., and between about 171° C. and 174° C., e.g., at about 172° C., according to differential scanning calorimetry. Xinafoate Salt 1 is in solvate form, and the free base and counter ion are present in the crystal in a ratio of about 1:1.

A second xinafoate salt is obtained when 1-hydroxy-2-napthoic acid is combined with 25 mg of Compound 1 in toluene, and is subsequently slurried at 50° C. for 2 hours. The resulting salt is obtained as a white powder. The XRPD pattern of Xinafoate Salt 2 has peaks as set forth in Table 20 below.

TABLE 20

| | | | | Rel. |
|---|---|---|---|---|
| Index | Angle | d-Value | Intensity | Intensity |
| 1 | 4.231 | 20.86738 | 331 | 22.20% |
| 2 | 5.258 | 16.7938 | 353 | 23.70% |
| 3 | 5.354 | 16.4933 | 409 | 27.40% |
| 4 | 6.083 | 14.517 | 1.45E+03 | 97.50% |
| 5 | 6.063 | 14.56524 | 1.49E+03 | 100.00% |
| 6 | 6.476 | 13.63804 | 611 | 41.00% |
| 7 | 12.4 | 7.13252 | 294 | 19.70% |
| 8 | 12.441 | 7.109 | 286 | 19.20% |

The above crystal exhibits thermal events between about 117° C. and 119° C., e.g., at about 118° C., between about 163° C. and 166° C., e.g., at about 164° C., and between about 174° C. and 177° C., e.g., at about 175° C., according to differential scanning calorimetry. Xinafoate Salt 2 is in solvate form, and the free base and counter ion are present in the crystal in a ratio of about 1:1.

A third xinafoate salt is obtained when 1-hydroxy-2-napthoic acid is combined with 25 mg of Compound 1 in ethyl acetate, and is subsequently cooled to 5° C. over an 8 hour period. The resulting salt is obtained as a brown powder. The XRPD pattern of Xinafoate Salt 3 has peaks as set forth in Table 21 below.

TABLE 21

| | | | | Rel. |
|---|---|---|---|---|
| Index | Angle | d-Value | Intensity | Intensity |
| 1 | 2.594 | 34.03096 | 106 | 7.90% |
| 2 | 4.674 | 18.88963 | 302 | 22.70% |
| 3 | 5.401 | 16.3486 | 460 | 34.50% |
| 4 | 5.882 | 15.01232 | 548 | 41.10% |
| 5 | 6.684 | 13.21405 | 1.06E+03 | 79.50% |
| 6 | 9.087 | 9.72358 | 105 | 7.90% |
| 7 | 10.157 | 8.70182 | 173 | 13.00% |
| 8 | 10.748 | 8.22489 | 559 | 42.00% |
| 9 | 10.754 | 8.22029 | 581 | 43.60% |
| 10 | 10.805 | 8.18169 | 613 | 46.00% |
| 11 | 11.101 | 7.96397 | 119 | 8.90% |
| 12 | 11.936 | 7.40894 | 321 | 24.10% |
| 13 | 13.127 | 6.73907 | 87.4 | 6.60% |
| 14 | 13.259 | 6.6721 | 206 | 15.50% |
| 15 | 13.263 | 6.67023 | 193 | 14.50% |
| 16 | 13.677 | 6.46944 | 1.33E+03 | 100.00% |
| 17 | 13.688 | 6.46385 | 1.28E+03 | 95.70% |
| 18 | 13.901 | 6.36538 | 758 | 56.90% |
| 19 | 15.057 | 5.87939 | 144 | 10.80% |
| 20 | 16.009 | 5.53166 | 157 | 11.80% |
| 21 | 16.961 | 5.22333 | 529 | 39.70% |
| 22 | 16.991 | 5.21414 | 601 | 45.10% |
| 23 | 18.637 | 4.7571 | 302 | 22.70% |
| 24 | 18.821 | 4.71102 | 117 | 8.80% |
| 25 | 19.479 | 4.55335 | 243 | 18.20% |
| 26 | 20.636 | 4.3007 | 454 | 34.10% |
| 27 | 21.061 | 4.21481 | 613 | 46.00% |
| 28 | 21.322 | 4.16376 | 560 | 42.00% |
| 29 | 21.817 | 4.0705 | 209 | 15.70% |
| 30 | 22.416 | 3.96311 | 413 | 31.00% |
| 31 | 23.608 | 3.7656 | 97.9 | 7.30% |
| 32 | 24.041 | 3.69867 | 379 | 28.50% |
| 33 | 24.365 | 3.65022 | 146 | 10.90% |
| 34 | 25.232 | 3.52676 | 66.5 | 5.00% |
| 35 | 31.224 | 2.86231 | 41.6 | 3.10% |
| 36 | 31.642 | 2.82539 | 75.5 | 5.70% |
| 37 | 33.32 | 2.68685 | 39.1 | 2.90% |
| 38 | 33.451 | 2.67662 | 53 | 4.00% |
| 39 | 37.771 | 2.37986 | 38.7 | 2.90% |
| 40 | 42.161 | 2.14161 | 38.1 | 2.90% |

The above crystal exhibits thermal events between about 131° C. and 133° C., e.g., at about 132° C., and between about 170° C. and 173° C., e.g., at about 172° C., according to differential scanning calorimetry. Xinafoate Salt 3 is in solvate form, and the free base and counter ion are present in the crystal in a ratio of about 1:1.

### Tosylate Salt

A tosylate salt is obtained when p-toluene sulfonic acid is combined with 25 mg of Compound 1 in 3-heptanone, and is subsequently slurried at 50° C. for 2 hours. The resulting salt is obtained as a white powder. The XRPD pattern of Tosylate Salt 1 has peaks as set forth in Table 22 below.

TABLE 22

| | | | | Rel. |
|---|---|---|---|---|
| Index | Angle | d-Value | Intensity | Intensity |
| 1 | 5.129 | 17.21435 | 2.26E+03 | 18.50% |
| 2 | 5.628 | 15.68921 | 1.22E+04 | 100.00% |
| 3 | 8.549 | 10.33474 | 4.89E+03 | 40.00% |
| 4 | 9.02 | 9.79619 | 181 | 1.50% |
| 5 | 10.085 | 8.76385 | 1.20E+03 | 9.80% |
| 6 | 10.463 | 8.44834 | 3.74E+03 | 30.50% |
| 7 | 10.678 | 8.27829 | 164 | 1.30% |
| 8 | 11.133 | 7.94133 | 280 | 2.30% |
| 9 | 11.818 | 7.4822 | 1.74E+03 | 14.20% |
| 10 | 12.069 | 7.32743 | 344 | 2.80% |
| 11 | 12.28 | 7.20179 | 1.21E+03 | 9.90% |
| 12 | 13.09 | 6.75797 | 181 | 1.50% |
| 13 | 13.506 | 6.55065 | 1.16E+03 | 9.50% |
| 14 | 13.887 | 6.37189 | 364 | 3.00% |
| 15 | 14.424 | 6.13575 | 1.28E+03 | 10.50% |
| 16 | 14.676 | 6.03107 | 612 | 5.00% |
| 17 | 15.089 | 5.86704 | 1.36E+03 | 11.10% |
| 18 | 15.526 | 5.70273 | 3.98E+03 | 32.50% |
| 19 | 15.803 | 5.60335 | 232 | 1.90% |
| 20 | 16.859 | 5.25481 | 979 | 8.00% |
| 21 | 17.086 | 5.1855 | 2.92E+03 | 23.90% |
| 22 | 17.266 | 5.13161 | 450 | 3.70% |
| 23 | 17.604 | 5.03395 | 679 | 5.60% |
| 24 | 17.802 | 4.97842 | 1.49E+03 | 12.20% |
| 25 | 18.009 | 4.92155 | 1.26E+03 | 10.30% |
| 26 | 18.24 | 4.85995 | 1.63E+03 | 13.30% |
| 27 | 18.37 | 4.82587 | 1.10E+03 | 9.00% |
| 28 | 18.749 | 4.72908 | 895 | 7.30% |
| 29 | 19.127 | 4.6364 | 1.92E+03 | 15.70% |
| 30 | 19.433 | 4.564 | 1.92E+03 | 15.70% |
| 31 | 19.829 | 4.47375 | 1.61E+03 | 13.10% |
| 32 | 20.099 | 4.41441 | 2.66E+03 | 21.70% |
| 33 | 20.427 | 4.34415 | 5.67E+03 | 46.40% |
| 34 | 20.592 | 4.3097 | 1.06E+03 | 8.60% |
| 35 | 20.93 | 4.24097 | 129 | 1.10% |
| 36 | 21.26 | 4.17582 | 1.20E+03 | 9.80% |
| 37 | 21.373 | 4.15399 | 992 | 8.10% |
| 38 | 21.865 | 4.06168 | 1.47E+03 | 12.00% |
| 39 | 22.351 | 3.97436 | 2.07E+03 | 17.00% |
| 40 | 22.632 | 3.92569 | 175 | 1.40% |
| 41 | 23.217 | 3.8281 | 3.17E+03 | 25.90% |
| 42 | 23.34 | 3.80822 | 2.60E+03 | 21.30% |
| 43 | 23.625 | 3.76288 | 755 | 6.20% |
| 44 | 23.829 | 3.73111 | 704 | 5.80% |
| 45 | 24.363 | 3.65051 | 58.8 | 0.50% |
| 46 | 24.63 | 3.61155 | 118 | 1.00% |
| 47 | 25.017 | 3.55664 | 376 | 3.10% |
| 48 | 25.399 | 3.50392 | 130 | 1.10% |
| 49 | 25.63 | 3.47295 | 127 | 1.00% |
| 50 | 25.99 | 3.42564 | 1.17E+03 | 9.60% |
| 51 | 26.56 | 3.35335 | 173 | 1.40% |
| 52 | 27.162 | 3.28041 | 982 | 8.00% |
| 53 | 27.495 | 3.24146 | 413 | 3.40% |
| 54 | 27.76 | 3.21104 | 784 | 6.40% |
| 55 | 28.242 | 3.15735 | 320 | 2.60% |
| 56 | 28.43 | 3.13695 | 146 | 1.20% |
| 57 | 28.826 | 3.09473 | 88.4 | 0.70% |
| 58 | 29.052 | 3.07112 | 371 | 3.00% |
| 59 | 29.341 | 3.0415 | 286 | 2.30% |
| 60 | 29.508 | 3.02474 | 747 | 6.10% |
| 61 | 29.886 | 2.98727 | 703 | 5.80% |
| 62 | 30.393 | 2.93865 | 413 | 3.40% |
| 63 | 30.504 | 2.92822 | 342 | 2.80% |
| 64 | 31.262 | 2.85887 | 128 | 1.00% |
| 65 | 31.674 | 2.82267 | 256 | 2.10% |
| 66 | 32.47 | 2.75524 | 270 | 2.20% |
| 67 | 32.601 | 2.74449 | 247 | 2.00% |
| 68 | 33.01 | 2.7114 | 243 | 2.00% |
| 69 | 33.295 | 2.68884 | 157 | 1.30% |
| 70 | 33.806 | 2.64935 | 215 | 1.80% |
| 71 | 34.717 | 2.58187 | 90.2 | 0.70% |
| 72 | 35.163 | 2.55013 | 131 | 1.10% |
| 73 | 35.904 | 2.4992 | 88.8 | 0.70% |
| 74 | 36.297 | 2.47304 | 132 | 1.10% |
| 75 | 36.946 | 2.43105 | 200 | 1.60% |
| 76 | 37.469 | 2.39833 | 156 | 1.30% |
| 77 | 38.065 | 2.36211 | 66.2 | 0.50% |
| 78 | 40.251 | 2.23874 | 89.9 | 0.70% |
| 79 | 40.681 | 2.21607 | 105 | 0.90% |
| 80 | 41.517 | 2.17334 | 178 | 1.50% |
| 81 | 43.273 | 2.08914 | 82.2 | 0.70% |

The above crystal exhibits a thermal event between about 216° C. and 218° C., e.g., at about 217° C., according to differential scanning calorimetry. Tosylate Salt 1 is in anhydrous form, and the free base and counter ion are present in the crystal in a ratio of about 1:1.

A second tosylate salt is obtained when p-toluene sulfonic acid is combined with 25 mg of Compound 1 in ethyl acetate, and is subsequently slurried at 50° C. for 2 hours. The resulting salt is obtained as a white powder. The XRPD pattern of Tosylate Salt 2 has peaks as set forth in Table 23 below.

TABLE 23

| Index | Angle | d-Value | Intensity | Rel.Intensity |
|---|---|---|---|---|
| 1 | 3.988 | 22.13894 | 315 | 10.60% |
| 2 | 4.51 | 19.57871 | 554 | 18.60% |
| 3 | 4.756 | 18.56321 | 2.98E+03 | 100.00% |
| 4 | 5.155 | 17.12745 | 720 | 24.10% |
| 5 | 5.16 | 17.11206 | 700 | 23.50% |
| 6 | 5.595 | 15.7823 | 874 | 29.30% |
| 7 | 8.527 | 10.36123 | 305 | 10.20% |
| 8 | 8.572 | 10.3075 | 359 | 12.00% |
| 9 | 8.708 | 10.14591 | 530 | 17.80% |
| 10 | 9.386 | 9.41484 | 399 | 13.40% |
| 11 | 10.216 | 8.65162 | 317 | 10.60% |
| 12 | 10.262 | 8.61288 | 313 | 10.50% |
| 13 | 10.577 | 8.35759 | 169 | 5.70% |
| 14 | 11.131 | 7.94256 | 161 | 5.40% |
| 15 | 11.337 | 7.79858 | 106 | 3.50% |
| 16 | 11.915 | 7.42156 | 470 | 15.70% |
| 17 | 11.981 | 7.38065 | 782 | 26.20% |
| 18 | 13.58 | 6.51527 | 937 | 31.40% |
| 19 | 14.592 | 6.0656 | 670 | 22.50% |
| 20 | 15.551 | 5.69345 | 158 | 5.30% |
| 21 | 16.197 | 5.46807 | 142 | 4.80% |
| 22 | 17.437 | 5.08194 | 543 | 18.20% |
| 23 | 17.627 | 5.02752 | 544 | 18.20% |
| 24 | 18.421 | 4.81241 | 460 | 15.40% |
| 25 | 20.117 | 4.41037 | 503 | 16.90% |
| 26 | 20.608 | 4.30636 | 472 | 15.80% |
| 27 | 20.816 | 4.26394 | 722 | 24.20% |
| 28 | 21.411 | 4.14679 | 469 | 15.70% |
| 29 | 22.025 | 4.03251 | 436 | 14.60% |
| 30 | 22.059 | 4.02642 | 446 | 14.90% |
| 31 | 22.212 | 3.999 | 639 | 21.40% |
| 32 | 22.769 | 3.90241 | 124 | 4.20% |
| 33 | 23.753 | 3.74284 | 950 | 31.80% |
| 34 | 23.98 | 3.70799 | 373 | 12.50% |
| 35 | 25.218 | 3.52868 | 81.5 | 2.70% |
| 36 | 25.975 | 3.42756 | 383 | 12.90% |
| 37 | 28.757 | 3.10201 | 91.6 | 3.10% |
| 38 | 29.566 | 3.01893 | 144 | 4.80% |
| 39 | 32.134 | 2.78325 | 72.6 | 2.40% |

The above crystal exhibits thermal events between about 87° C. and 90° C., e.g., at about 89° C., between about 109° C. and 112° C., e.g., at about 111° C., and between about 217° C. and 220° C., e.g., at about 219° C., according to differential scanning calorimetry. Tosylate Salt 2 is in solvate form, and the free base and counter ion are present in the crystal in a ratio of about 1:1.

### Tartrate Salt

A tartrate salt is obtained when tartaric acid is combined with 25 mg of Compound 1 in acetone, and is subsequently slurried at 50° C. for 2 hours. The resulting salt is obtained as a white powder. The XRPD pattern of Tartrate Salt 1 has peaks as set forth in Table 24 below.

TABLE 24

| | | | | Rel. |
|---|---|---|---|---|
| Index | Angle | d-Value | Intensity | Intensity |
| 1 | 3.699 | 23.86707 | 221 | 49.20% |
| 2 | 5.975 | 14.77997 | 449 | 100.00% |
| 3 | 6.882 | 12.8342 | 313 | 69.80% |
| 4 | 10.405 | 8.49508 | 155 | 34.60% |
| 5 | 11.563 | 7.64691 | 329 | 73.20% |
| 6 | 13.212 | 6.69582 | 110 | 24.40% |
| 7 | 15.004 | 5.89995 | 234 | 52.00% |
| 8 | 15.854 | 5.58534 | 133 | 29.50% |
| 9 | 17.515 | 5.0594 | 174 | 38.70% |
| 10 | 20.309 | 4.36926 | 353 | 78.60% |
| 11 | 20.763 | 4.27459 | 216 | 48.00% |
| 12 | 21.65 | 4.10144 | 303 | 67.40% |
| 13 | 22.799 | 3.89733 | 93.5 | 20.80% |
| 14 | 24.067 | 3.69483 | 72.1 | 16.00% |
| 15 | 25.999 | 3.42444 | 70 | 15.60% |
| 16 | 27.941 | 3.19062 | 76.7 | 17.10% |

The above crystal exhibits a thermal event between about 134° C. and 136° C., e.g., at about 135° C., according to differential scanning calorimetry. The free base and counter ion of Tartrate Salt 1 are present in the crystal in a ratio of about 1:1.

### Succinate Salt

A succinate salt is obtained when succinic acid is combined with 25 mg of Compound 1 in acetone, and is subsequently slurried at 50° C. for 2 hours. The resulting salt is obtained as a white powder. The XRPD pattern of Succinate Salt 1 has peaks as set forth in Table 25 below.

TABLE 25

| | | | | Rel. |
|---|---|---|---|---|
| Index | Angle | d-Value | Intensity | Intensity |
| 1 | 3.955 | 22.32042 | 186 | 6.60% |
| 2 | 4.697 | 18.79975 | 826 | 29.20% |
| 3 | 6.908 | 12.78538 | 1.93E+03 | 68.20% |
| 4 | 7.068 | 12.49701 | 1.11E+03 | 39.20% |
| 5 | 9.42 | 9.38117 | 907 | 32.10% |
| 6 | 9.774 | 9.04201 | 1.79E+03 | 63.30% |
| 7 | 13.119 | 6.74322 | 196 | 6.90% |
| 8 | 13.156 | 6.724 | 114 | 4.00% |
| 9 | 13.589 | 6.51098 | 709 | 25.10% |
| 10 | 13.972 | 6.33327 | 358 | 12.70% |
| 11 | 15.805 | 5.60274 | 206 | 7.30% |
| 12 | 16.041 | 5.52069 | 865 | 30.60% |
| 13 | 16.355 | 5.41547 | 2.82E+03 | 100.00% |
| 14 | 17.069 | 5.19065 | 939 | 33.20% |
| 15 | 17.89 | 4.95415 | 708 | 25.10% |
| 16 | 18.51 | 4.78963 | 606 | 21.50% |
| 17 | 18.901 | 4.69145 | 510 | 18.10% |
| 18 | 19.26 | 4.60483 | 2.78E+03 | 98.60% |
| 19 | 20.383 | 4.35352 | 273 | 9.70% |
| 20 | 20.633 | 4.30129 | 282 | 10.00% |
| 21 | 21.111 | 4.20498 | 667 | 23.60% |
| 22 | 21.789 | 4.07555 | 65.5 | 2.30% |
| 23 | 22.223 | 3.99697 | 861 | 30.50% |
| 24 | 22.759 | 3.9041 | 1.05E+03 | 37.30% |
| 25 | 23.229 | 3.82608 | 594 | 21.00% |
| 26 | 23.633 | 3.7617 | 164 | 5.80% |
| 27 | 23.705 | 3.75032 | 187 | 6.60% |
| 28 | 23.915 | 3.71796 | 647 | 22.90% |
| 29 | 25.642 | 3.47135 | 741 | 26.20% |
| 30 | 25.772 | 3.45412 | 1.01E+03 | 35.70% |
| 31 | 26.288 | 3.38743 | 143 | 5.10% |
| 32 | 26.867 | 3.3157 | 102 | 3.60% |
| 33 | 26.904 | 3.3113 | 117 | 4.20% |
| 34 | 27.291 | 3.26512 | 218 | 7.70% |
| 35 | 27.87 | 3.1986 | 238 | 8.40% |
| 36 | 27.928 | 3.19214 | 222 | 7.80% |
| 37 | 28.318 | 3.14909 | 496 | 17.60% |
| 38 | 29.269 | 3.04889 | 493 | 17.40% |
| 39 | 29.499 | 3.02563 | 348 | 12.30% |
| 40 | 30.67 | 2.91275 | 200 | 7.10% |
| 41 | 30.702 | 2.90974 | 185 | 6.50% |
| 42 | 31.098 | 2.87362 | 104 | 3.70% |
| 43 | 31.252 | 2.85974 | 163 | 5.80% |
| 44 | 33.122 | 2.70244 | 173 | 6.10% |
| 45 | 33.208 | 2.69564 | 137 | 4.80% |
| 46 | 34.197 | 2.61996 | 112 | 4.00% |
| 47 | 34.437 | 2.60223 | 225 | 8.00% |
| 48 | 34.449 | 2.60132 | 249 | 8.80% |
| 49 | 34.488 | 2.59851 | 232 | 8.20% |
| 50 | 37.937 | 2.3698 | 85.8 | 3.00% |

The above crystal exhibits thermal events between about 153° C. and 155° C., e.g., at about 154° C., between about 172° C. and 175° C., e.g., at about 173° C., and between about 178° C. and 181° C., e.g., at about 180° C., according to differential scanning calorimetry. Succinate Salt 1 is in solvate form, and the free base and counter ion are present in the crystal in a ratio of about 1:0.7.

A second succinate salt is obtained when succinic acid is combined with 25 mg of Compound 1 in ethyl acetate, and is subsequently slurried at 50° C. for 2 hours. The resulting salt is obtained as a white powder. The XRPD pattern of Succinate Salt 2 has peaks as set forth in Table 26 below.

TABLE 26

| | | | | Rel. |
|---|---|---|---|---|
| Index | Angle | d-Value | Intensity | Intensity |
| 1 | 4.564 | 19.34444 | 452 | 37.50% |
| 2 | 6.881 | 12.83505 | 1.20E+03 | 100.00% |
| 3 | 9.417 | 9.38435 | 737 | 61.20% |
| 4 | 13.052 | 6.77737 | 162 | 13.50% |
| 5 | 13.578 | 6.51641 | 275 | 22.80% |
| 6 | 15.874 | 5.57854 | 240 | 19.90% |
| 7 | 16.304 | 5.43215 | 597 | 49.50% |
| 8 | 17.172 | 5.15975 | 125 | 10.40% |
| 9 | 18.051 | 4.91037 | 201 | 16.70% |
| 10 | 18.73 | 4.73388 | 148 | 12.30% |
| 11 | 19.262 | 4.60415 | 572 | 47.50% |
| 12 | 20.625 | 4.3029 | 240 | 20.00% |
| 13 | 20.892 | 4.24852 | 199 | 16.50% |
| 14 | 21.185 | 4.19044 | 131 | 10.90% |
| 15 | 21.435 | 4.14208 | 306 | 25.40% |
| 16 | 22.357 | 3.97329 | 645 | 53.60% |
| 17 | 22.836 | 3.89114 | 208 | 17.30% |
| 18 | 23.876 | 3.7239 | 261 | 21.70% |
| 19 | 24.522 | 3.62724 | 76.5 | 6.30% |
| 20 | 25.742 | 3.45804 | 413 | 34.30% |
| 21 | 26.255 | 3.39158 | 91.5 | 7.60% |
| 22 | 29.119 | 3.06424 | 116 | 9.60% |
| 23 | 29.159 | 3.06012 | 105 | 8.80% |
| 24 | 29.15 | 3.06098 | 91.1 | 7.60% |

The above crystal exhibits thermal events between about 150° C. and 152° C., e.g., at about 151° C., between about 163° C. and 165° C., e.g., at about 164° C., between about 172° C. and 175° C., e.g., at about 174° C., and between about 178° C. and 181° C., e.g., at about 179° C., according to differential scanning calorimetry. Succinate Salt 2 is in anhydrous form, and he free base and counter ion are present in the crystal in a ratio of about 1:0.8.

### Mesylate Salt

A mesylate salt is obtained when methanesulfonic acid is combined with 25 mg of Compound 1 in acetone, and is subsequently slurried at 50° C. for 2 hours. The resulting salt is obtained as a white powder. The XRPD pattern of Mesylate Salt 1 has peaks as set forth in Table 27 below.

TABLE 27

| Index | Angle | d-Value | Intensity | Rel.Intensity |
|---|---|---|---|---|
| 1 | 6.201 | 14.24194 | 658 | 27.50% |
| 2 | 6.386 | 13.82916 | 702 | 29.30% |
| 3 | 8.666 | 10.19504 | 294 | 12.30% |
| 4 | 9.28 | 9.52224 | 214 | 8.90% |
| 5 | 9.681 | 9.12882 | 151 | 6.30% |
| 6 | 10.106 | 8.74587 | 147 | 6.20% |
| 7 | 11.687 | 7.56582 | 566 | 23.60% |
| 8 | 12.339 | 7.16766 | 576 | 24.10% |
| 9 | 12.526 | 7.06075 | 83 | 3.50% |
| 10 | 13.923 | 6.35568 | 71 | 3.00% |
| 11 | 14.06 | 6.29405 | 65.2 | 2.70% |
| 12 | 14.178 | 6.2418 | 337 | 14.10% |
| 13 | 16.48 | 5.3746 | 278 | 11.60% |
| 14 | 16.753 | 5.28776 | 151 | 6.30% |
| 15 | 16.925 | 5.23445 | 118 | 4.90% |
| 16 | 17.077 | 5.18808 | 421 | 17.60% |
| 17 | 17.11 | 5.17826 | 604 | 25.20% |
| 18 | 18.029 | 4.91631 | 453 | 18.90% |
| 19 | 18.282 | 4.84872 | 164 | 6.90% |
| 20 | 18.824 | 4.71038 | 2.39E+03 | 100.00% |
| 21 | 19.406 | 4.57037 | 130 | 5.40% |
| 22 | 19.796 | 4.48129 | 224 | 9.40% |
| 23 | 20.225 | 4.38706 | 1.18E+03 | 49.40% |
| 24 | 20.413 | 4.34705 | 58.2 | 2.40% |
| 25 | 20.752 | 4.2769 | 388 | 16.20% |
| 26 | 20.797 | 4.26767 | 267 | 11.20% |
| 27 | 21.215 | 4.18456 | 932 | 38.90% |
| 28 | 21.436 | 4.14202 | 178 | 7.50% |
| 29 | 22.242 | 3.99361 | 118 | 4.90% |
| 30 | 22.479 | 3.95204 | 172 | 7.20% |
| 31 | 23.077 | 3.85098 | 306 | 12.80% |
| 32 | 23.711 | 3.74946 | 128 | 5.30% |
| 33 | 23.763 | 3.74133 | 127 | 5.30% |
| 34 | 24.522 | 3.62723 | 91.2 | 3.80% |
| 35 | 24.923 | 3.56982 | 401 | 16.80% |
| 36 | 24.974 | 3.56257 | 244 | 10.20% |
| 37 | 25.786 | 3.45229 | 270 | 11.30% |
| 38 | 25.841 | 3.445 | 234 | 9.80% |
| 39 | 27.869 | 3.19871 | 85.4 | 3.60% |
| 40 | 27.932 | 3.19166 | 65 | 2.70% |
| 41 | 28.583 | 3.12041 | 345 | 14.40% |
| 42 | 28.659 | 3.1123 | 212 | 8.80% |
| 43 | 29.708 | 3.00483 | 1.14E+03 | 47.40% |
| 44 | 29.778 | 2.99784 | 693 | 29.00% |
| 45 | 31.662 | 2.8237 | 39.9 | 1.70% |
| 46 | 33.047 | 2.70846 | 198 | 8.30% |
| 47 | 33.136 | 2.70136 | 130 | 5.40% |
| 48 | 37.221 | 2.41375 | 38.4 | 1.60% |
| 49 | 39.493 | 2.27993 | 56.2 | 2.30% |
| 50 | 39.915 | 2.25681 | 380 | 15.90% |
| 51 | 40.012 | 2.25157 | 222 | 9.30% |
| 52 | 41.234 | 2.18763 | 45.2 | 1.90% |
| 53 | 41.461 | 2.17618 | 90.3 | 3.80% |
| 54 | 41.579 | 2.17025 | 42.7 | 1.80% |
| 55 | 44.882 | 2.01793 | 52.6 | 2.20% |

The above crystal exhibits a thermal event between about 310° C. and 312° C., e.g., at about 311° C., according to differential scanning calorimetry. Mesylate Salt 1 is in anhydrous form, and the free base and counter ion are present in the crystal in a ratio of about 1:1.

### Napadisylate Salt

A mesylate salt is obtained when naphthalene disulfonic acid is combined with 25 mg of Compound 1 in acetonitrile, and is subsequently slurried at 50° C. for 2 hours. The resulting salt is obtained as a brown sticky solid. The XRPD pattern of Napadisylate Salt 1 has peaks as set forth in Table 28 below.

TABLE 28

| | | | | Rel. |
|---|---|---|---|---|
| Index | Angle | d-Value | Intensity | Intensity |
| 1 | 3.116 | 28.33576 | 240 | 45.30% |
| 2 | 12.571 | 7.03587 | 200 | 37.90% |
| 3 | 15.821 | 5.59722 | 315 | 59.50% |
| 4 | 16.087 | 5.50514 | 140 | 26.40% |
| 5 | 16.726 | 5.29608 | 312 | 59.00% |
| 6 | 18.627 | 4.75977 | 92.8 | 17.50% |
| 7 | 25.255 | 3.52362 | 529 | 100.00% |
| 8 | 25.306 | 3.51664 | 351 | 66.30% |
| 9 | 30.509 | 2.92766 | 81.8 | 15.50% |

The above crystal exhibits a thermal event between about 103° C. and 107° C., e.g., at about 105° C., according to differential scanning calorimetry. Napadisylate Salt 1 is in solvate form, and the free base and counter ion are present in the crystal in a ratio of about 1:1.2.

### Edisylate Salt

An edisylate salt is obtained when ethane disulfonic acid is combined with 25 mg of Compound 1 in 2-butanone, and is subsequently slurried at 50° C. for 2 hours. The resulting salt is obtained as an off-white powder. The XRPD pattern of Edisylate Salt 1 has peaks as set forth in Table 29 below.

TABLE 29

| | | | | Rel. |
|---|---|---|---|---|
| Index | Angle | d-Value | Intensity | Intensity |
| 1 | 4.326 | 20.40769 | 463 | 17.00% |
| 2 | 4.488 | 19.67467 | 772 | 28.30% |
| 3 | 4.726 | 18.68469 | 2.73E+03 | 100.00% |
| 4 | 9.762 | 9.05276 | 159 | 5.80% |
| 5 | 10.924 | 8.09252 | 314 | 11.50% |
| 6 | 11.654 | 7.58714 | 778 | 28.60% |
| 7 | 12.225 | 7.23393 | 905 | 33.20% |
| 8 | 12.79 | 6.91597 | 1.36E+03 | 49.80% |
| 9 | 13.338 | 6.63305 | 708 | 26.00% |
| 10 | 13.67 | 6.47245 | 427 | 15.70% |
| 11 | 14.501 | 6.10342 | 90 | 3.30% |
| 12 | 15.352 | 5.76701 | 323 | 11.80% |
| 13 | 15.496 | 5.71356 | 223 | 8.20% |
| 14 | 17.265 | 5.13198 | 725 | 26.60% |
| 15 | 17.547 | 5.0503 | 151 | 5.50% |
| 16 | 17.694 | 5.00871 | 180 | 6.60% |
| 17 | 18.037 | 4.91396 | 377 | 13.80% |
| 18 | 18.367 | 4.82645 | 859 | 31.50% |
| 19 | 18.74 | 4.73126 | 1.76E+03 | 64.80% |
| 20 | 19.508 | 4.54676 | 367 | 13.50% |
| 21 | 19.821 | 4.47568 | 232 | 8.50% |
| 22 | 20.178 | 4.39719 | 395 | 14.50% |
| 23 | 20.528 | 4.323 | 168 | 6.20% |
| 24 | 20.878 | 4.25128 | 286 | 10.50% |
| 25 | 21.313 | 4.16555 | 1.94E+03 | 71.20% |
| 26 | 21.461 | 4.13712 | 626 | 23.00% |
| 27 | 22.036 | 4.03059 | 376 | 13.80% |
| 28 | 22.587 | 3.93336 | 343 | 12.60% |
| 29 | 22.821 | 3.89362 | 574 | 21.10% |
| 30 | 23.362 | 3.80463 | 271 | 9.90% |
| 31 | 23.502 | 3.78229 | 384 | 14.10% |
| 32 | 23.794 | 3.73662 | 158 | 5.80% |
| 33 | 23.842 | 3.72909 | 124 | 4.50% |
| 34 | 24.066 | 3.69493 | 198 | 7.30% |
| 35 | 24.491 | 3.63172 | 294 | 10.80% |
| 36 | 24.53 | 3.62608 | 351 | 12.90% |
| 37 | 25.213 | 3.5294 | 90.5 | 3.30% |
| 38 | 25.614 | 3.47506 | 1.36E+03 | 49.90% |
| 39 | 26.329 | 3.38226 | 167 | 6.10% |
| 40 | 27.003 | 3.29933 | 93.7 | 3.40% |
| 41 | 28.201 | 3.16186 | 124 | 4.60% |
| 42 | 28.318 | 3.1491 | 149 | 5.50% |
| 43 | 28.787 | 3.09876 | 99 | 3.60% |
| 44 | 28.974 | 3.0792 | 391 | 14.30% |
| 45 | 29.438 | 3.03175 | 137 | 5.00% |
| 46 | 29.889 | 2.98706 | 124 | 4.50% |
| 47 | 30.472 | 2.93121 | 76.9 | 2.80% |
| 48 | 30.866 | 2.89462 | 58.7 | 2.20% |
| 49 | 32.329 | 2.76695 | 120 | 4.40% |
| 50 | 32.387 | 2.76212 | 94.6 | 3.50% |
| 51 | 32.361 | 2.76429 | 110 | 4.00% |
| 52 | 33.182 | 2.69775 | 58.9 | 2.20% |
| 53 | 34.845 | 2.57269 | 190 | 7.00% |
| 54 | 38.902 | 2.31321 | 99.9 | 3.70% |
| 55 | 40.541 | 2.2234 | 57.5 | 2.10% |
| 56 | 43.266 | 2.08948 | 84.5 | 3.10% |

The above crystal exhibits a thermal event between about 295° C. and 298° C., e.g., at about 296° C. or 297° C., according to differential scanning calorimetry. Edisylate Salt 1 is in anhydrous form, and the free base and counter ion are present in the crystal in a ratio of about 1:1.

### Propionate Salt

A propionate salt is obtained when propionic acid is combined with 25 mg of Compound 1 in methanol and water (9:1), and is subsequently slurried at 50° C. for 2 hours. The resulting salt is obtained as a brown powder obtained following evaporation. The XRPD pattern of Propionate Salt 1 has peaks as set forth in Table 30 below.

TABLE 30

| | | | | Rel. |
|---|---|---|---|---|
| Index | Angle | d-Value | Intensity | Intensity |
| 1 | 2.489 | 35.47115 | 69.1 | 1.30% |
| 2 | 5.735 | 15.3977 | 879 | 16.50% |
| 3 | 7.31 | 12.08297 | 575 | 10.80% |
| 4 | 8.514 | 10.37745 | 773 | 14.50% |
| 5 | 8.781 | 10.06242 | 983 | 18.50% |
| 6 | 9.499 | 9.30297 | 918 | 17.30% |
| 7 | 11.397 | 7.75788 | 260 | 4.90% |
| 8 | 11.762 | 7.51811 | 225 | 4.20% |
| 9 | 13.636 | 6.48845 | 443 | 8.30% |
| 10 | 15.014 | 5.89598 | 308 | 5.80% |
| 11 | 15.854 | 5.58559 | 794 | 14.90% |
| 12 | 16.115 | 5.49563 | 1.59E+03 | 30.00% |
| 13 | 17.025 | 5.20369 | 5.32E+03 | 100.00% |
| 14 | 17.469 | 5.07243 | 904 | 17.00% |
| 15 | 18.346 | 4.83198 | 2.08E+03 | 39.20% |
| 16 | 19.032 | 4.65943 | 946 | 17.80% |
| 17 | 19.687 | 4.50572 | 114 | 2.10% |
| 18 | 20.47 | 4.33528 | 312 | 5.90% |
| 19 | 20.709 | 4.28571 | 817 | 15.40% |
| 20 | 21.441 | 4.14101 | 405 | 7.60% |
| 21 | 22.466 | 3.9543 | 238 | 4.50% |
| 22 | 22.81 | 3.89551 | 794 | 14.90% |
| 23 | 22.865 | 3.88617 | 1.02E+03 | 19.10% |
| 24 | 22.848 | 3.88906 | 859 | 16.10% |
| 25 | 23.287 | 3.81676 | 142 | 2.70% |
| 26 | 23.56 | 3.77311 | 228 | 4.30% |
| 27 | 24.907 | 3.57211 | 290 | 5.40% |
| 28 | 25.734 | 3.45909 | 361 | 6.80% |
| 29 | 25.749 | 3.45714 | 311 | 5.90% |
| 30 | 27.064 | 3.29208 | 132 | 2.50% |
| 31 | 27.5 | 3.24087 | 127 | 2.40% |
| 32 | 27.746 | 3.2127 | 70.3 | 1.30% |
| 33 | 27.81 | 3.2054 | 49.6 | 0.90% |
| 34 | 29.661 | 3.00944 | 204 | 3.80% |
| 35 | 29.858 | 2.99005 | 225 | 4.20% |
| 36 | 29.868 | 2.98905 | 207 | 3.90% |
| 37 | 30.565 | 2.92246 | 85.8 | 1.60% |
| 38 | 32.583 | 2.74589 | 1.01E+03 | 19.00% |
| 39 | 34.089 | 2.62799 | 139 | 2.60% |
| 40 | 37.287 | 2.40958 | 63.5 | 1.20% |

The above crystal exhibits a thermal event between about 109° C. and 112° C., e.g., at about 111° C., and between about 135° C. and 137° C., e.g., at about 136° C. according to differential scanning calorimetry. Propionate Salt 1 is in solvate form, and the free base and counter ion are present in the crystal in a ratio of about 1:0.7.

### Caprylate Salt

A caprylate salt is obtained when caprylic acid is combined with 25 mg of Compound 1 in methanol and water (9:1), and is subsequently slurried at 50° C. for 2 hours. The resulting salt is obtained initially as a clear liquid, which presents as a hard solid following evaporation. The XRPD pattern of Caprylate Salt 1 has peaks as set forth in Table 31 below.

TABLE 31

| | | | | Rel. |
|---|---|---|---|---|
| Index | Angle | d-Value | Intensity | Intensity |
| 1 | 2.934 | 30.08777 | 1.12E+03 | 22.80% |
| 2 | 3.026 | 29.17317 | 1.37E+03 | 28.00% |
| 3 | 3.894 | 22.67281 | 519 | 10.60% |
| 4 | 4.477 | 19.72177 | 1.63E+03 | 33.20% |
| 5 | 4.578 | 19.28461 | 2.30E+03 | 46.80% |
| 6 | 4.712 | 18.73702 | 3.19E+03 | 65.10% |
| 7 | 4.838 | 18.24884 | 4.28E+03 | 87.10% |
| 8 | 4.895 | 18.03891 | 4.91E+03 | 100.00% |
| 9 | 5.962 | 14.81147 | 1.40E+03 | 28.50% |
| 10 | 8.013 | 11.02466 | 191 | 3.90% |
| 11 | 8.975 | 9.84476 | 473 | 9.60% |
| 12 | 9.726 | 9.08701 | 276 | 5.60% |
| 13 | 9.755 | 9.05916 | 280 | 5.70% |
| 14 | 11.026 | 8.0183 | 978 | 19.90% |
| 15 | 11.351 | 7.78933 | 170 | 3.50% |
| 16 | 11.533 | 7.66678 | 190 | 3.90% |
| 17 | 12.224 | 7.23477 | 426 | 8.70% |
| 18 | 12.255 | 7.21666 | 387 | 7.90% |
| 19 | 15.693 | 5.64229 | 1.03E+03 | 20.90% |
| 20 | 16.047 | 5.51866 | 371 | 7.60% |
| 21 | 16.544 | 5.35409 | 827 | 16.90% |
| 22 | 16.674 | 5.31269 | 1.01E+03 | 20.50% |
| 23 | 17.182 | 5.15663 | 299 | 6.10% |
| 24 | 17.696 | 5.00811 | 472 | 9.60% |
| 25 | 17.699 | 5.00705 | 489 | 10.00% |
| 26 | 18.632 | 4.75859 | 253 | 5.20% |
| 27 | 19.317 | 4.59129 | 639 | 13.00% |
| 28 | 19.343 | 4.5852 | 603 | 12.30% |
| 29 | 19.362 | 4.58074 | 672 | 13.70% |
| 30 | 20.125 | 4.40868 | 634 | 12.90% |
| 31 | 20.165 | 4.40002 | 683 | 13.90% |
| 32 | 20.21 | 4.39039 | 723 | 14.70% |
| 33 | 20.196 | 4.39347 | 710 | 14.50% |
| 34 | 21.563 | 4.11783 | 679 | 13.80% |
| 35 | 21.563 | 4.11782 | 679 | 13.80% |
| 36 | 21.608 | 4.10939 | 631 | 12.90% |
| 37 | 22.043 | 4.02929 | 1.27E+03 | 25.90% |
| 38 | 22.796 | 3.89775 | 1.63E+03 | 33.30% |
| 39 | 25.106 | 3.5442 | 456 | 9.30% |
| 40 | 25.414 | 3.50198 | 235 | 4.80% |
| 41 | 26.24 | 3.39348 | 241 | 4.90% |
| 42 | 27.806 | 3.20587 | 129 | 2.60% |
| 43 | 29.497 | 3.0258 | 83.9 | 1.70% |
| 44 | 32.456 | 2.75642 | 75.7 | 1.50% |

The above crystal exhibits a thermal event between about 102° C. and 105° C., e.g., at about 104° C., according to differential scanning calorimetry. Caprylate Salt 1 is in anhydrous form, and the free base and counter ion are present in the crystal in a ratio of about 1:1.4.

### Besylate Salt

A besylate salt is obtained when benzenesulfonic acid is combined with 25 mg of Compound 1 in 3-heptanone, and is subsequently slurried at 50° C. for 2 hours. The resulting salt is obtained as a brown powder. The XRPD pattern of Besylate Salt 1 has peaks as set forth in Table 32 below.

TABLE 32

| | | | | Rel. |
|---|---|---|---|---|
| Index | Angle | d-Value | Intensity | Intensity |
| 1 | 6.039 | 14.62421 | 197 | 2.40% |
| 2 | 6.666 | 13.24833 | 429 | 5.20% |
| 3 | 6.837 | 12.9187 | 1.36E+03 | 16.60% |
| 4 | 7.22 | 12.23374 | 202 | 2.50% |
| 5 | 7.579 | 11.65508 | 1.39E+03 | 17.00% |
| 6 | 7.665 | 11.52415 | 4.52E+03 | 55.00% |
| 7 | 7.753 | 11.39437 | 8.21E+03 | 100.00% |
| 8 | 8.672 | 10.18868 | 859 | 10.50% |
| 9 | 9.293 | 9.50908 | 103 | 1.30% |
| 10 | 9.613 | 9.19269 | 270 | 3.30% |
| 11 | 9.745 | 9.06865 | 880 | 10.70% |
| 12 | 10.668 | 8.28605 | 120 | 1.50% |
| 13 | 11.712 | 7.55005 | 7.32E+03 | 89.20% |
| 14 | 11.712 | 7.55005 | 7.32E+03 | 89.20% |
| 15 | 13.226 | 6.68884 | 1.41E+03 | 17.20% |
| 16 | 13.508 | 6.55 | 1.01E+03 | 12.30% |
| 17 | 13.944 | 6.34616 | 2.16E+03 | 26.30% |
| 18 | 13.933 | 6.35092 | 2.03E+03 | 24.70% |
| 19 | 14.923 | 5.93178 | 167 | 2.00% |
| 20 | 15.441 | 5.73386 | 224 | 2.70% |
| 21 | 15.857 | 5.5843 | 1.81E+03 | 22.10% |
| 22 | 15.971 | 5.54483 | 1.49E+03 | 18.10% |
| 23 | 16.967 | 5.22155 | 323 | 3.90% |
| 24 | 17.44 | 5.08081 | 200 | 2.40% |
| 25 | 17.525 | 5.05651 | 565 | 6.90% |
| 26 | 18.058 | 4.90846 | 879 | 10.70% |
| 27 | 18.486 | 4.79578 | 1.48E+03 | 18.10% |
| 28 | 18.621 | 4.76134 | 1.32E+03 | 16.10% |
| 29 | 18.832 | 4.70841 | 990 | 12.10% |
| 30 | 19.012 | 4.66421 | 1.48E+03 | 18.10% |
| 31 | 19.497 | 4.54921 | 1.54E+03 | 18.80% |
| 32 | 19.648 | 4.51456 | 551 | 6.70% |
| 33 | 19.844 | 4.47056 | 276 | 3.40% |
| 34 | 20.406 | 4.34859 | 826 | 10.10% |
| 35 | 20.848 | 4.25744 | 328 | 4.00% |
| 36 | 21.36 | 4.15652 | 2.24E+03 | 27.30% |
| 37 | 21.891 | 4.05681 | 2.33E+03 | 28.40% |
| 38 | 22.095 | 4.01979 | 2.19E+03 | 26.70% |
| 39 | 22.418 | 3.96269 | 128 | 1.60% |
| 40 | 22.587 | 3.93338 | 127 | 1.50% |
| 41 | 23.038 | 3.85739 | 902 | 11.00% |
| 42 | 23.082 | 3.85013 | 990 | 12.10% |
| 43 | 23.207 | 3.82963 | 1.23E+03 | 15.00% |
| 44 | 23.353 | 3.80612 | 2.62E+03 | 31.90% |
| 45 | 24.578 | 3.6191 | 1.20E+03 | 14.70% |
| 46 | 24.637 | 3.61063 | 607 | 7.40% |
| 47 | 25.218 | 3.52871 | 858 | 10.50% |
| 48 | 25.26 | 3.52285 | 651 | 7.90% |
| 49 | 25.655 | 3.46951 | 247 | 3.00% |
| 50 | 25.691 | 3.46472 | 265 | 3.20% |
| 51 | 25.995 | 3.42493 | 464 | 5.70% |
| 52 | 25.907 | 3.43639 | 269 | 3.30% |
| 53 | 26.48 | 3.36327 | 1.77E+03 | 21.50% |
| 54 | 27.094 | 3.28841 | 951 | 11.60% |
| 55 | 27.375 | 3.25538 | 195 | 2.40% |
| 56 | 28.267 | 3.15457 | 335 | 4.10% |
| 57 | 28.294 | 3.15165 | 373 | 4.50% |
| 58 | 28.827 | 3.09457 | 55 | 0.70% |
| 59 | 29.348 | 3.04084 | 1.01E+03 | 12.30% |
| 60 | 29.384 | 3.03724 | 1.01E+03 | 12.30% |
| 61 | 29.781 | 2.99756 | 213 | 2.60% |
| 62 | 30.287 | 2.94861 | 52.6 | 0.60% |
| 63 | 30.973 | 2.88486 | 329 | 4.00% |
| 64 | 31.209 | 2.86357 | 132 | 1.60% |
| 65 | 31.825 | 2.80958 | 373 | 4.50% |
| 66 | 31.91 | 2.80226 | 235 | 2.90% |
| 67 | 32.183 | 2.7791 | 311 | 3.80% |
| 68 | 32.265 | 2.77227 | 241 | 2.90% |
| 69 | 33.442 | 2.6773 | 248 | 3.00% |
| 70 | 33.471 | 2.67508 | 304 | 3.70% |
| 71 | 33.94 | 2.63916 | 231 | 2.80% |
| 72 | 34.014 | 2.63361 | 168 | 2.00% |
| 73 | 34.386 | 2.60593 | 142 | 1.70% |
| 74 | 34.453 | 2.60104 | 87 | 1.10% |
| 75 | 34.932 | 2.56646 | 97.5 | 1.20% |
| 76 | 35.687 | 2.51389 | 126 | 1.50% |
| 77 | 36.958 | 2.4303 | 59.8 | 0.70% |
| 78 | 37.302 | 2.40868 | 121 | 1.50% |
| 79 | 37.825 | 2.37653 | 238 | 2.90% |
| 80 | 39.082 | 2.30296 | 63.2 | 0.80% |
| 81 | 41.35 | 2.18172 | 178 | 2.20% |
| 82 | 41.43 | 2.17773 | 138 | 1.70% |

The above crystal exhibits a thermal event between about 237° C. and 240° C., e.g., at about 238° C., according to differential scanning calorimetry. Besylate Salt 1 is in anhydrous form, and the free base and counter ion are present in the crystal in a ratio of about 1:1.

### Benzoate Salt

A benzoate salt is obtained when benzoic acid is combined with 25 mg of Compound 1 in 3-heptanone, and is subsequently subjected to evaporation under vacuum. The resulting salt is obtained as a brown/red powder. The XRPD pattern of Benzoate Salt 1 has peaks as set forth in Table 33 below.

TABLE 33

| | | | | Rel. |
|---|---|---|---|---|
| Index | Angle | d-Value | Intensity | Intensity |
| 1 | 2.65 | 33.31246 | 87.3 | 0.30% |
| 2 | 4.836 | 18.25892 | 505 | 1.90% |
| 3 | 5.337 | 16.54672 | 739 | 2.90% |
| 4 | 5.453 | 16.19337 | 886 | 3.40% |
| 5 | 5.817 | 15.1802 | 2.59E+04 | 100.00% |
| 6 | 6.326 | 13.96069 | 1.24E+03 | 4.80% |
| 7 | 6.37 | 13.86512 | 1.38E+03 | 5.30% |
| 8 | 6.658 | 13.26494 | 462 | 1.80% |
| 9 | 9.596 | 9.20913 | 391 | 1.50% |
| 10 | 10.811 | 8.17658 | 113 | 0.40% |
| 11 | 11.55 | 7.65546 | 6.66E+03 | 25.70% |
| 12 | 11.986 | 7.37811 | 746 | 2.90% |
| 13 | 12.318 | 7.17999 | 969 | 3.70% |
| 14 | 12.839 | 6.88979 | 139 | 0.50% |
| 15 | 13.088 | 6.75924 | 1.67E+03 | 6.40% |
| 16 | 14.287 | 6.19419 | 275 | 1.10% |
| 17 | 14.947 | 5.92232 | 226 | 0.90% |
| 18 | 15.314 | 5.78112 | 350 | 1.40% |
| 19 | 16.439 | 5.38794 | 381 | 1.50% |
| 20 | 16.491 | 5.37119 | 455 | 1.80% |
| 21 | 16.889 | 5.24552 | 260 | 1.00% |
| 22 | 17.455 | 5.07656 | 332 | 1.30% |
| 23 | 17.515 | 5.05942 | 418 | 1.60% |
| 24 | 19.034 | 4.65895 | 814 | 3.10% |
| 25 | 19.094 | 4.64448 | 862 | 3.30% |
| 26 | 19.969 | 4.44284 | 482 | 1.90% |
| 27 | 21.54 | 4.12227 | 662 | 2.60% |
| 28 | 22.13 | 4.01353 | 472 | 1.80% |
| 29 | 22.938 | 3.87403 | 301 | 1.20% |
| 30 | 26.956 | 3.30496 | 360 | 1.40% |
| 31 | 28.513 | 3.12796 | 456 | 1.80% |
| 32 | 31.59 | 2.8299 | 63.9 | 0.20% |
| 33 | 42.436 | 2.12838 | 215 | 0.80% |
| 34 | 42.515 | 2.12463 | 300 | 1.20% |
| 35 | 42.59 | 2.12103 | 247 | 1.00% |
| 36 | 43.417 | 2.08253 | 91.8 | 0.40% |

The above crystal exhibits thermal events between about 59° C. and 62° C., e.g., at about 60° C., between about 81° C. and 84° C., e.g., at about 83° C., and between about 115° C. and 118° C., e.g., at about 116° C., according to differential scanning calorimetry. Benzoate Salt 1 is in anhydrous form, and the free base and counter ion are present in the crystal in a ratio of about 1:1.1.

### Nicotinate Salt

A nicotinate salt is obtained when nicotinic acid is combined with 25 mg of Compound 1 in acetonitrile, and is subsequently cooled to 5° C. over an 8 hour period. The resulting salt is obtained as a white powder. The XRPD pattern of Nicotinate Salt 1 has peaks as set forth in Table 34 below.

TABLE 34

| | | | | Rel. |
|---|---|---|---|---|
| Index | Angle | d-Value | Intensity | Intensity |
| 1 | 4.426 | 19.95 | 347 | 3.90% |
| 2 | 5.209 | 16.9506 | 8.99E+03 | 100.00% |
| 3 | 8.326 | 10.61097 | 343 | 3.80% |
| 4 | 8.747 | 10.10133 | 2.25E+03 | 25.00% |
| 5 | 10.768 | 8.20948 | 3.98E+03 | 44.30% |
| 6 | 11.248 | 7.86005 | 1.36E+03 | 15.20% |
| 7 | 11.978 | 7.38307 | 2.91E+03 | 32.40% |
| 8 | 12.766 | 6.92884 | 3.57E+03 | 39.70% |
| 9 | 13.244 | 6.67967 | 1.49E+03 | 16.60% |
| 10 | 14.663 | 6.03617 | 1.40E+03 | 15.60% |
| 11 | 15.481 | 5.71923 | 555 | 6.20% |
| 12 | 15.857 | 5.58444 | 505 | 5.60% |
| 13 | 16.218 | 5.46106 | 1.77E+03 | 19.70% |
| 14 | 16.624 | 5.32848 | 904 | 10.10% |
| 15 | 17.224 | 5.1442 | 1.90E+03 | 21.20% |
| 16 | 17.457 | 5.07609 | 1.17E+03 | 13.00% |
| 17 | 17.904 | 4.95031 | 3.10E+03 | 34.50% |
| 18 | 18.424 | 4.81171 | 1.93E+03 | 21.50% |
| 19 | 19.235 | 4.61075 | 141 | 1.60% |
| 20 | 20.006 | 4.43461 | 3.48E+03 | 38.80% |
| 21 | 20.453 | 4.3387 | 2.56E+03 | 28.50% |
| 22 | 20.754 | 4.27651 | 3.32E+03 | 36.90% |
| 23 | 20.906 | 4.24566 | 1.81E+03 | 20.20% |
| 24 | 21.413 | 4.14628 | 1.12E+03 | 12.40% |
| 25 | 21.592 | 4.1124 | 2.88E+03 | 32.00% |
| 26 | 22.475 | 3.95273 | 1.63E+03 | 18.10% |
| 27 | 22.762 | 3.90352 | 420 | 4.70% |
| 28 | 23.406 | 3.79758 | 2.24E+03 | 24.90% |
| 29 | 24.04 | 3.6989 | 591 | 6.60% |
| 30 | 24.265 | 3.66503 | 318 | 3.50% |
| 31 | 24.87 | 3.57725 | 681 | 7.60% |
| 32 | 25.524 | 3.48712 | 1.08E+03 | 12.00% |
| 33 | 26.041 | 3.41903 | 312 | 3.50% |
| 34 | 26.506 | 3.36003 | 205 | 2.30% |
| 35 | 26.881 | 3.31399 | 333 | 3.70% |
| 36 | 27.483 | 3.24279 | 1.46E+03 | 16.30% |
| 37 | 27.979 | 3.18642 | 748 | 8.30% |
| 38 | 28.681 | 3.11006 | 445 | 5.00% |
| 39 | 29.026 | 3.07387 | 169 | 1.90% |
| 40 | 29.377 | 3.03792 | 550 | 6.10% |
| 41 | 29.557 | 3.01984 | 744 | 8.30% |
| 42 | 30.136 | 2.96311 | 104 | 1.20% |
| 43 | 30.621 | 2.91728 | 235 | 2.60% |
| 44 | 31.514 | 2.83659 | 137 | 1.50% |
| 45 | 31.859 | 2.80668 | 237 | 2.60% |
| 46 | 31.912 | 2.80208 | 158 | 1.80% |
| 47 | 32.208 | 2.77706 | 118 | 1.30% |
| 48 | 33.905 | 2.64179 | 102 | 1.10% |
| 49 | 34.074 | 2.62908 | 170 | 1.90% |
| 50 | 34.888 | 2.56963 | 194 | 2.20% |
| 51 | 36.434 | 2.46403 | 170 | 1.90% |
| 52 | 40.814 | 2.20916 | 124 | 1.40% |
| 53 | 41.614 | 2.16853 | 90.7 | 1.00% |

The above crystal exhibits a thermal event between about 135° C. and 138° C., e.g., at about 137° C., according to differential scanning calorimetry. Nicotinate Salt 1 is in anhydrous form, and the free base and counter ion are present in the crystal in a ratio of about 1:1.

### Isonicotinate Salt

An isonicotinate salt is obtained when isonicotinic acid is combined with 25 mg of Compound 1 in toluene, and is subsequently slurried at 50° C. for 2 hours. The resulting salt is obtained as a light brown powder. The XRPD pattern of Isonicotinate Salt 1 has peaks as set forth in Table 35 below.

TABLE 35

| | | | | Rel. |
|---|---|---|---|---|
| Index | Angle | d-Value | Intensity | Intensity |
| 1 | 2.811 | 31.40434 | 420 | 12.10% |
| 2 | 5.49 | 16.08358 | 3.02E+03 | 87.00% |
| 3 | 5.543 | 15.9312 | 3.47E+03 | 100.00% |
| 4 | 7.318 | 12.07095 | 1.44E+03 | 41.40% |
| 5 | 8.733 | 10.117 | 296 | 8.50% |
| 6 | 10.509 | 8.41091 | 254 | 7.30% |
| 7 | 11.722 | 7.54342 | 1.22E+03 | 35.20% |
| 8 | 12.845 | 6.88617 | 1.14E+03 | 32.80% |
| 9 | 13.528 | 6.54039 | 392 | 11.30% |
| 10 | 13.757 | 6.43159 | 238 | 6.90% |
| 11 | 14.802 | 5.98014 | 563 | 16.20% |
| 12 | 15.396 | 5.75041 | 545 | 15.70% |
| 13 | 15.412 | 5.74474 | 498 | 14.40% |
| 14 | 16.721 | 5.29791 | 1.51E+03 | 43.40% |
| 15 | 17.114 | 5.17706 | 1.72E+03 | 49.40% |
| 16 | 17.29 | 5.12463 | 1.63E+03 | 47.00% |
| 17 | 17.301 | 5.12159 | 1.58E+03 | 45.40% |
| 18 | 17.858 | 4.96281 | 1.05E+03 | 30.20% |
| 19 | 18.808 | 4.71426 | 202 | 5.80% |
| 20 | 19.81 | 4.4781 | 525 | 15.10% |
| 21 | 19.849 | 4.46935 | 723 | 20.80% |
| 22 | 20.166 | 4.39976 | 319 | 9.20% |
| 23 | 20.294 | 4.37227 | 355 | 10.20% |
| 24 | 20.189 | 4.39492 | 324 | 9.30% |
| 25 | 20.834 | 4.26021 | 167 | 4.80% |
| 26 | 20.447 | 4.33991 | 967 | 27.80% |
| 27 | 20.963 | 4.23434 | 519 | 15.00% |
| 28 | 21.821 | 4.06969 | 741 | 21.30% |
| 29 | 22.081 | 4.02245 | 516 | 14.80% |
| 30 | 22.33 | 3.97813 | 338 | 9.70% |
| 31 | 23.061 | 3.85369 | 551 | 15.90% |
| 32 | 25.251 | 3.5242 | 338 | 9.70% |
| 33 | 25.762 | 3.45543 | 164 | 4.70% |
| 34 | 26.285 | 3.38784 | 798 | 23.00% |
| 35 | 26.294 | 3.38664 | 702 | 20.20% |
| 36 | 27.538 | 3.23647 | 117 | 3.40% |
| 37 | 27.635 | 3.22535 | 191 | 5.50% |
| 38 | 28.076 | 3.17559 | 997 | 28.70% |
| 39 | 28.849 | 3.09227 | 105 | 3.00% |
| 40 | 29.922 | 2.98376 | 203 | 5.80% |
| 41 | 37.525 | 2.39484 | 99.8 | 2.90% |
| 42 | 37.533 | 2.3944 | 81.5 | 2.30% |
| 43 | 39.988 | 2.25284 | 328 | 9.50% |
| 44 | 40.079 | 2.24792 | 204 | 5.90% |

The above crystal exhibits thermal events between about 111° C. and 114° C., e.g., at about 113° C., and between about 128° C. and 130° C., e.g., at about 129° C., according to differential scanning calorimetry. Isonicotinate Salt 1 is in solvate form, and the free base and counter ion are present in the crystal in a ratio of about 1:0.7.

### Orotate Salt

An orotate salt is obtained when orotic acid is combined with 25 mg of Compound 1 in 2-butanone, and is subsequently slurried at 50° C. for 2 hours. The resulting salt is obtained as a white powder. The XRPD pattern of Orotate Salt 1 has peaks as set forth in Table 36 below.

TABLE 36

| | | | | Rel. |
|---|---|---|---|---|
| Index | Angle | d-Value | Intensity | Intensity |
| 1 | 3.414 | 25.85846 | 193 | 29.90% |
| 2 | 5.762 | 15.32639 | 647 | 100.00% |
| 3 | 7.18 | 12.30221 | 412 | 63.60% |
| 4 | 10.418 | 8.48467 | 253 | 39.10% |
| 5 | 11.815 | 7.48419 | 195 | 30.10% |
| 6 | 12.604 | 7.01731 | 386 | 59.60% |
| 7 | 13.508 | 6.55002 | 206 | 31.90% |
| 8 | 16.879 | 5.24848 | 516 | 79.70% |
| 9 | 21.033 | 4.22033 | 155 | 24.00% |
| 10 | 21.869 | 4.06086 | 519 | 80.20% |
| 11 | 22.463 | 3.95479 | 201 | 31.00% |
| 12 | 24.9 | 3.57304 | 161 | 24.90% |
| 13 | 28.938 | 3.08293 | 438 | 67.70% |
| 14 | 34.093 | 2.62771 | 62.7 | 9.70% |

The above crystal exhibits a thermal event between about 137° C. and 140° C., e.g., at about 138° C., according to differential scanning calorimetry. Orotate Salt 1 is in solvate form, and the free base and counter ion are present in the crystal in a ratio of about 1:2.

### Camsylate Salt

A camsylate salt is obtained when camphor-10-sulfonic acid is combined with 25 mg of Compound 1 in 3-heptanone, and is subsequently slurried at 50° C. for 2 hours. The resulting salt is obtained as a white powder. The XRPD pattern of Camsylate Salt 1 has peaks as set forth in Table 37 below.

TABLE 37

| | | | | Rel. |
|---|---|---|---|---|
| Index | Angle | d-Value | Intensity | Intensity |
| 1 | 4.553 | 19.39166 | 294 | 3.80% |
| 2 | 4.68 | 18.86681 | 381 | 5.00% |
| 3 | 5.329 | 16.57068 | 7.69E+03 | 100.00% |
| 4 | 8.27 | 10.6826 | 2.03E+03 | 26.40% |
| 5 | 8.988 | 9.83096 | 75.9 | 1.00% |
| 6 | 9.53 | 9.27286 | 216 | 2.80% |
| 7 | 9.682 | 9.1282 | 326 | 4.20% |
| 8 | 9.964 | 8.86965 | 6.81E+03 | 88.50% |
| 9 | 10.576 | 8.35781 | 144 | 1.90% |
| 10 | 10.741 | 8.22983 | 458 | 6.00% |
| 11 | 11.217 | 7.88165 | 299 | 3.90% |
| 12 | 11.202 | 7.8927 | 356 | 4.60% |
| 13 | 11.383 | 7.76709 | 250 | 3.20% |
| 14 | 12.599 | 7.01999 | 2.30E+03 | 29.90% |
| 15 | 13.473 | 6.56672 | 429 | 5.60% |
| 16 | 13.509 | 6.5492 | 558 | 7.30% |
| 17 | 13.63 | 6.49126 | 822 | 10.70% |
| 18 | 13.793 | 6.41523 | 205 | 2.70% |
| 19 | 14.02 | 6.31183 | 308 | 4.00% |
| 20 | 14.131 | 6.26227 | 1.21E+03 | 15.80% |
| 21 | 15.156 | 5.84094 | 4.63E+03 | 60.10% |
| 22 | 15.938 | 5.55625 | 1.80E+03 | 23.50% |
| 23 | 16.479 | 5.37502 | 850 | 11.00% |
| 24 | 16.799 | 5.27333 | 337 | 4.40% |
| 25 | 16.905 | 5.24066 | 897 | 11.70% |
| 26 | 17.416 | 5.08789 | 1.64E+03 | 21.30% |
| 27 | 17.839 | 4.96817 | 1.94E+03 | 25.20% |
| 28 | 17.995 | 4.92537 | 6.50E+03 | 84.50% |
| 29 | 18.255 | 4.85593 | 1.62E+03 | 21.10% |
| 30 | 19.279 | 4.60017 | 3.35E+03 | 43.60% |
| 31 | 19.409 | 4.56964 | 4.90E+03 | 63.70% |
| 32 | 19.562 | 4.53432 | 1.54E+03 | 20.00% |
| 33 | 19.906 | 4.45681 | 200 | 2.60% |
| 34 | 20.205 | 4.39152 | 751 | 9.80% |
| 35 | 20.482 | 4.33276 | 989 | 12.90% |
| 36 | 21.168 | 4.19387 | 650 | 8.50% |
| 37 | 21.496 | 4.13049 | 521 | 6.80% |
| 38 | 21.796 | 4.07442 | 1.39E+03 | 18.10% |
| 39 | 22.289 | 3.98538 | 874 | 11.40% |
| 40 | 22.493 | 3.94966 | 1.57E+03 | 20.40% |
| 41 | 22.767 | 3.90271 | 1.51E+03 | 19.70% |
| 42 | 23.492 | 3.78385 | 167 | 2.20% |
| 43 | 24.062 | 3.69554 | 137 | 1.80% |
| 44 | 23.999 | 3.70512 | 55.5 | 0.70% |
| 45 | 24.532 | 3.62579 | 1.96E+03 | 25.50% |
| 46 | 24.874 | 3.57676 | 279 | 3.60% |
| 47 | 25.277 | 3.52062 | 1.23E+03 | 16.10% |
| 48 | 25.536 | 3.48547 | 255 | 3.30% |
| 49 | 26.044 | 3.41863 | 213 | 2.80% |
| 50 | 26.417 | 3.37117 | 532 | 6.90% |
| 51 | 26.691 | 3.33719 | 152 | 2.00% |
| 52 | 26.926 | 3.30858 | 139 | 1.80% |
| 53 | 27.268 | 3.26785 | 584 | 7.60% |
| 54 | 27.629 | 3.22601 | 365 | 4.70% |
| 55 | 28.068 | 3.17655 | 345 | 4.50% |
| 56 | 28.373 | 3.14304 | 852 | 11.10% |
| 57 | 28.796 | 3.09787 | 111 | 1.40% |
| 58 | 29.035 | 3.07286 | 391 | 5.10% |
| 59 | 29.37 | 3.03863 | 319 | 4.10% |
| 60 | 30.043 | 2.97201 | 134 | 1.70% |
| 61 | 31.394 | 2.84716 | 203 | 2.60% |
| 62 | 31.419 | 2.84498 | 189 | 2.50% |
| 63 | 32.227 | 2.77542 | 191 | 2.50% |
| 64 | 32.441 | 2.75761 | 134 | 1.70% |
| 65 | 33.225 | 2.69433 | 80.3 | 1.00% |
| 66 | 33.463 | 2.67571 | 90.2 | 1.20% |
| 67 | 33.528 | 2.6707 | 164 | 2.10% |
| 68 | 33.824 | 2.64794 | 191 | 2.50% |
| 69 | 33.882 | 2.64358 | 171 | 2.20% |
| 70 | 34.548 | 2.59414 | 143 | 1.90% |
| 71 | 34.851 | 2.57224 | 73.5 | 1.00% |
| 72 | 34.973 | 2.56354 | 131 | 1.70% |
| 73 | 35.001 | 2.56159 | 163 | 2.10% |
| 74 | 35.873 | 2.50129 | 80 | 1.00% |
| 75 | 36.421 | 2.46492 | 137 | 1.80% |
| 76 | 36.466 | 2.46195 | 135 | 1.80% |
| 77 | 36.471 | 2.46162 | 158 | 2.10% |
| 78 | 41.542 | 2.1721 | 81 | 1.10% |
| 79 | 43.983 | 2.05703 | 156 | 2.00% |
| 80 | 44.805 | 2.02121 | 100 | 1.30% |

The above crystal exhibits thermal events between about 227° C. and 230° C., e.g., at about 228° C., and between about 253° C. and 256° C., e.g., at about 254° C., according to differential scanning calorimetry. Camsylate Salt 1 is in anhydrous form, and the free base and counter ion are present in the crystal in a ratio of about 1:1.

A second camsylate salt is obtained when camphor-10-sulfonic acid is combined with 25 mg of Compound 1 in toluene, and is subsequently slurried at 50° C. for 2 hours. The resulting salt is obtained as a white powder. The XRPD pattern of Camsylate Salt 2 has peaks as set forth in Table 38 below.

TABLE 38

| | | | | Rel. |
|---|---|---|---|---|
| Index | Angle | d-Value | Intensity | Intensity |
| 1 | 4.721 | 18.7008 | 2.56E+03 | 97.40% |
| 2 | 5.354 | 16.49163 | 1.70E+03 | 64.80% |
| 3 | 5.743 | 15.37661 | 610 | 23.20% |
| 4 | 7.43 | 11.88803 | 257 | 9.80% |
| 5 | 7.795 | 11.33208 | 735 | 28.00% |
| 6 | 8.632 | 10.23559 | 201 | 7.60% |
| 7 | 9.062 | 9.75058 | 1.88E+03 | 71.30% |
| 8 | 9.431 | 9.36968 | 1.09E+03 | 41.40% |
| 9 | 10.461 | 8.45 | 191 | 7.30% |
| 10 | 11.456 | 7.71786 | 64.3 | 2.40% |
| 11 | 11.255 | 7.85531 | 232 | 8.80% |
| 12 | 11.486 | 7.69769 | 129 | 4.90% |
| 13 | 12.377 | 7.14564 | 318 | 12.10% |
| 14 | 12.623 | 7.00706 | 809 | 30.80% |
| 15 | 13.186 | 6.70892 | 348 | 13.20% |
| 16 | 13.699 | 6.45911 | 537 | 20.40% |
| 17 | 14.247 | 6.21158 | 131 | 5.00% |
| 18 | 15.247 | 5.80635 | 86.2 | 3.30% |
| 19 | 15.569 | 5.68689 | 167 | 6.30% |
| 20 | 16.02 | 5.52811 | 840 | 31.90% |
| 21 | 16.512 | 5.36431 | 1.01E+03 | 38.40% |
| 22 | 17.087 | 5.18517 | 675 | 25.70% |
| 23 | 17.566 | 5.04477 | 2.63E+03 | 100.00% |
| 24 | 18.338 | 4.83416 | 1.55E+03 | 58.80% |
| 25 | 19.274 | 4.6015 | 383 | 14.60% |
| 26 | 19.706 | 4.50145 | 707 | 26.90% |
| 27 | 20.15 | 4.40334 | 1.09E+03 | 41.60% |
| 28 | 22.045 | 4.02897 | 485 | 18.40% |
| 29 | 22.495 | 3.94925 | 362 | 13.80% |
| 30 | 23.342 | 3.80789 | 172 | 6.60% |
| 31 | 24.715 | 3.5993 | 476 | 18.10% |
| 32 | 24.899 | 3.57314 | 525 | 20.00% |
| 33 | 25.282 | 3.51984 | 359 | 13.70% |
| 34 | 26.663 | 3.34065 | 144 | 5.50% |
| 35 | 27.577 | 3.23196 | 190 | 7.20% |
| 36 | 29.667 | 3.0089 | 79.1 | 3.00% |

Camsylate Salt 2 is in solvate form, and the free base and counter ion are present in the crystal in a ratio of about 1:1.

### Salicylate Salt

A salicylate salt is obtained when salicylic acid is combined with 25 mg of Compound 1 in acetonitrile, and is subsequently slurried at 50° C. for 2 hours. The resulting salt is obtained as a white powder. The XRPD pattern of Salicylate Salt 1 has peaks as set forth in Table 39 below.

TABLE 39

| | | | | Rel. |
|---|---|---|---|---|
| Index | Angle | d-Value | Intensity | Intensity |
| 1 | 5.925 | 14.90445 | 344 | 13.80% |
| 2 | 6.961 | 12.68897 | 2.49E+03 | 100.00% |
| 3 | 7.326 | 12.05739 | 456 | 18.30% |
| 4 | 9.567 | 9.23682 | 439 | 17.70% |
| 5 | 10.35 | 8.53971 | 437 | 17.60% |
| 6 | 10.971 | 8.05814 | 1.55E+03 | 62.30% |
| 7 | 11.509 | 7.68272 | 492 | 19.80% |
| 8 | 12.118 | 7.29751 | 145 | 5.80% |
| 9 | 12.344 | 7.16452 | 251 | 10.10% |
| 10 | 12.679 | 6.97586 | 465 | 18.70% |
| 11 | 13.278 | 6.66262 | 1.00E+03 | 40.20% |
| 12 | 13.79 | 6.41646 | 1.17E+03 | 47.10% |
| 13 | 15.1 | 5.86274 | 358 | 14.40% |
| 14 | 15.838 | 5.59112 | 324 | 13.00% |
| 15 | 17.229 | 5.14262 | 1.49E+03 | 59.90% |
| 16 | 18.112 | 4.8939 | 434 | 17.50% |
| 17 | 18.572 | 4.77383 | 330 | 13.30% |
| 18 | 18.932 | 4.68372 | 574 | 23.10% |
| 19 | 19.215 | 4.61544 | 415 | 16.70% |
| 20 | 19.798 | 4.48085 | 475 | 19.10% |
| 21 | 20.346 | 4.36139 | 491 | 19.70% |
| 22 | 20.567 | 4.31502 | 312 | 12.60% |
| 23 | 20.967 | 4.23344 | 739 | 29.70% |
| 24 | 21.341 | 4.1602 | 350 | 14.10% |
| 25 | 21.815 | 4.0708 | 298 | 12.00% |
| 26 | 21.996 | 4.03772 | 234 | 9.40% |
| 27 | 22.83 | 3.89211 | 432 | 17.40% |
| 28 | 22.929 | 3.87547 | 438 | 17.60% |
| 29 | 23.139 | 3.84076 | 291 | 11.70% |
| 30 | 23.476 | 3.78639 | 262 | 10.50% |
| 31 | 24.109 | 3.68845 | 320 | 12.90% |
| 32 | 24.235 | 3.66955 | 360 | 14.50% |
| 33 | 25.847 | 3.4442 | 359 | 14.40% |
| 34 | 27.133 | 3.28381 | 227 | 9.10% |
| 35 | 28.341 | 3.14659 | 118 | 4.70% |
| 36 | 28.812 | 3.09621 | 139 | 5.60% |
| 37 | 29.682 | 3.00741 | 55.6 | 2.20% |
| 38 | 32.436 | 2.75806 | 104 | 4.20% |

The above crystal exhibits thermal events between about 146° C. and 150° C., e.g., at about 147° C., between about 153° C. and 156° C., e.g., at about 155° C., between about 196° C. and 199° C., e.g., at about 197° C., and between about 244° C. and 247° C., e.g., at about 245° C., according to differential scanning calorimetry. Salicylate Salt 1 is in anhydrous form, and the free base and counter ion are present in the crystal in a ratio of about 1:1.

A second salicylate salt is obtained when salicylic acid is combined with 25 mg of Compound 1 in toluene, which is subsequently cooled to 5° C. over an 8 hour period. The resulting salt is obtained as a white powder. The XRPD pattern of Salicylate Salt 2 has peaks as set forth in Table 40 below.

TABLE 40

| | | | | Rel. |
|---|---|---|---|---|
| Index | Angle | d-Value | Intensity | Intensity |
| 1 | 4.37 | 20.20544 | 446 | 14.10% |
| 2 | 6.051 | 14.59552 | 839 | 26.50% |
| 3 | 6.258 | 14.11245 | 872 | 27.50% |
| 4 | 6.949 | 12.71007 | 2.51E+03 | 79.40% |
| 5 | 8.673 | 10.18783 | 103 | 3.20% |
| 6 | 9.838 | 8.98359 | 188 | 5.90% |
| 7 | 10.871 | 8.13168 | 846 | 26.70% |
| 8 | 11.277 | 7.84007 | 904 | 28.60% |
| 9 | 12.449 | 7.10463 | 1.30E+03 | 41.20% |
| 10 | 12.817 | 6.90124 | 273 | 8.60% |
| 11 | 14.304 | 6.18698 | 3.17E+03 | 100.00% |
| 12 | 15.225 | 5.81472 | 745 | 23.50% |
| 13 | 15.885 | 5.57467 | 170 | 5.40% |
| 14 | 16.255 | 5.44843 | 279 | 8.80% |
| 15 | 16.654 | 5.31891 | 2.43E+03 | 76.70% |
| 16 | 17.099 | 5.18136 | 257 | 8.10% |
| 17 | 17.306 | 5.11991 | 342 | 10.80% |
| 18 | 17.95 | 4.93774 | 164 | 5.20% |
| 19 | 18.018 | 4.91925 | 221 | 7.00% |
| 20 | 18.609 | 4.76442 | 395 | 12.50% |
| 21 | 18.706 | 4.7397 | 395 | 12.50% |
| 22 | 19.295 | 4.59638 | 86.4 | 2.70% |
| 23 | 19.69 | 4.50521 | 1.79E+03 | 56.40% |
| 24 | 20.274 | 4.37656 | 265 | 8.40% |
| 25 | 20.574 | 4.31356 | 170 | 5.40% |
| 26 | 20.856 | 4.25581 | 618 | 19.50% |
| 27 | 21.089 | 4.20927 | 553 | 17.50% |
| 28 | 21.31 | 4.16611 | 1.24E+03 | 39.30% |
| 29 | 21.336 | 4.16119 | 1.14E+03 | 35.90% |
| 30 | 21.692 | 4.09371 | 867 | 27.40% |
| 31 | 21.971 | 4.04227 | 1.29E+03 | 40.60% |
| 32 | 22.264 | 3.98973 | 430 | 13.60% |
| 33 | 23.644 | 3.75986 | 530 | 16.80% |
| 34 | 24.1 | 3.68972 | 253 | 8.00% |
| 35 | 24.461 | 3.63618 | 1.26E+03 | 39.70% |
| 36 | 25.04 | 3.55342 | 921 | 29.10% |
| 37 | 25.923 | 3.43427 | 173 | 5.50% |
| 38 | 26.24 | 3.39354 | 195 | 6.10% |
| 39 | 26.756 | 3.32918 | 99.1 | 3.10% |
| 40 | 27.628 | 3.22615 | 360 | 11.40% |
| 41 | 28.128 | 3.16988 | 344 | 10.90% |
| 42 | 28.162 | 3.16609 | 417 | 13.20% |
| 43 | 28.791 | 3.09837 | 242 | 7.60% |

The above crystal exhibits thermal events between about 127° C. and 130° C., e.g., at about 128° C., between about 143° C. and 146° C., e.g., at about 144° C., between about 180° C. and 183° C., e.g., at about 181° C., between about 196° C. and 199° C., e.g., at about 197° C., and between about 244° C. and 247° C., e.g., at about 247° C., according to differential scanning calorimetry. Salicylate Salt 2 is in solvate form, and the free base and counter ion are present in the crystal in a ratio of about 1:1.

### Aminosalicylate Salt

An aminosalicylate salt is obtained when amino salicylic acid is combined with 25 mg of Compound 1 in acetonitrile, and is subsequently slurried at 50° C. for 2 hours. The resulting salt is obtained as an off-white powder. The XRPD pattern of Aminosalicylate Salt 1 has peaks as set forth in Table 42 below.

TABLE 42

| | | | | Rel. |
|---|---|---|---|---|
| Index | Angle | d-Value | Intensity | Intensity |
| 1 | 6.785 | 13.01739 | 4.39E+03 | 100.00% |
| 2 | 8.943 | 9.88022 | 511 | 11.60% |
| 3 | 9.433 | 9.36847 | 168 | 3.80% |
| 4 | 10.619 | 8.32404 | 2.10E+03 | 47.90% |
| 5 | 11.124 | 7.94748 | 789 | 17.90% |
| 6 | 11.743 | 7.52992 | 445 | 10.10% |
| 7 | 12.203 | 7.24735 | 825 | 18.80% |
| 8 | 12.452 | 7.10272 | 876 | 19.90% |
| 9 | 12.695 | 6.96727 | 962 | 21.90% |
| 10 | 13.513 | 6.54724 | 1.42E+03 | 32.30% |
| 11 | 13.85 | 6.38886 | 2.89E+03 | 65.80% |
| 12 | 14.501 | 6.10342 | 178 | 4.10% |
| 13 | 14.852 | 5.9601 | 269 | 6.10% |
| 14 | 15.156 | 5.84094 | 619 | 14.10% |
| 15 | 15.954 | 5.55055 | 185 | 4.20% |
| 16 | 16.748 | 5.28926 | 906 | 20.60% |
| 17 | 16.992 | 5.21378 | 1.55E+03 | 35.30% |
| 18 | 17.844 | 4.96693 | 484 | 11.00% |
| 19 | 19.03 | 4.65993 | 534 | 12.10% |
| 20 | 19.14 | 4.63336 | 631 | 14.40% |
| 21 | 20.412 | 4.34734 | 1.10E+03 | 25.10% |
| 22 | 20.66 | 4.29568 | 1.57E+03 | 35.70% |
| 23 | 20.748 | 4.27765 | 1.53E+03 | 34.90% |
| 24 | 21.283 | 4.17145 | 1.08E+03 | 24.60% |
| 25 | 21.517 | 4.12651 | 1.26E+03 | 28.60% |
| 26 | 21.476 | 4.13424 | 1.29E+03 | 29.30% |
| 27 | 22.159 | 4.00845 | 750 | 17.10% |
| 28 | 22.559 | 3.93826 | 358 | 8.10% |
| 29 | 23.448 | 3.79082 | 332 | 7.50% |
| 30 | 23.982 | 3.70762 | 373 | 8.50% |
| 31 | 24.444 | 3.63858 | 328 | 7.50% |
| 32 | 27.03 | 3.29615 | 388 | 8.80% |
| 33 | 27.085 | 3.28953 | 410 | 9.30% |
| 34 | 27.309 | 3.26311 | 369 | 8.40% |

The above crystal exhibits thermal events between about 130° C. and 133° C., e.g., at about 132° C., and between about 161° C. and 164° C., e.g., at about 162° C., according to differential scanning calorimetry. Aminosalicylate Salt 1 is in anhydrous form, and the free base and counter ion are present in the crystal in a ratio of about 1:1.

### Mandelate Salt

A mandelate salt is obtained when mandelic acid is combined with 25 mg of Compound 1 in toluene, and is subsequently cooled to 5° C. over an 8 hour period. An off-white powder is obtained. Alternatively, the salt is obtained by subjecting the mixture to evaporation under vacuum. Under this method, the material is not dissolved prior to evaporation. After centrifugation, the supernatant is separated using a pipette and placed under vacuum until a dry solid is obtained. The XRPD pattern of Mandelate Salt 1 has peaks as set forth in Table 43 below.

TABLE 43

| | | | | Rel. |
|---|---|---|---|---|
| Index | Angle | d-Value | Intensity | Intensity |
| 1 | 5.297 | 16.66867 | 622 | 9.90% |
| 2 | 5.988 | 14.74762 | 1.18E+03 | 18.70% |
| 3 | 6.473 | 13.64401 | 5.59E+03 | 88.90% |
| 4 | 7.229 | 12.21885 | 1.57E+03 | 25.00% |
| 5 | 7.511 | 11.76049 | 1.28E+03 | 20.40% |
| 6 | 7.678 | 11.50542 | 1.71E+03 | 27.30% |
| 7 | 8.055 | 10.9669 | 864 | 13.70% |
| 8 | 8.545 | 10.33955 | 6.28E+03 | 100.00% |
| 9 | 9.757 | 9.05778 | 758 | 12.10% |
| 10 | 9.973 | 8.86198 | 2.28E+03 | 36.30% |
| 11 | 10.73 | 8.2386 | 609 | 9.70% |
| 12 | 11.179 | 7.90836 | 1.28E+03 | 20.40% |
| 13 | 11.249 | 7.85942 | 1.66E+03 | 26.50% |
| 14 | 11.359 | 7.78336 | 3.13E+03 | 49.80% |
| 15 | 11.928 | 7.41352 | 2.03E+03 | 32.30% |
| 16 | 12.748 | 6.93877 | 189 | 3.00% |
| 17 | 13.183 | 6.71069 | 466 | 7.40% |
| 18 | 13.658 | 6.47818 | 213 | 3.40% |
| 19 | 14.07 | 6.28929 | 398 | 6.30% |
| 20 | 14.124 | 6.26559 | 364 | 5.80% |
| 21 | 14.415 | 6.13978 | 801 | 12.70% |
| 22 | 14.735 | 6.00717 | 220 | 3.50% |
| 23 | 14.908 | 5.93785 | 511 | 8.10% |
| 24 | 15.195 | 5.82622 | 847 | 13.50% |
| 25 | 16.037 | 5.5221 | 620 | 9.90% |
| 26 | 16.369 | 5.41103 | 582 | 9.30% |
| 27 | 16.656 | 5.31835 | 1.37E+03 | 21.90% |
| 28 | 16.826 | 5.26497 | 422 | 6.70% |
| 29 | 17.393 | 5.09459 | 274 | 4.40% |
| 30 | 17.738 | 4.99613 | 576 | 9.20% |
| 31 | 17.978 | 4.92995 | 338 | 5.40% |
| 32 | 18.129 | 4.88937 | 589 | 9.40% |
| 33 | 18.275 | 4.85069 | 746 | 11.90% |
| 34 | 18.476 | 4.79834 | 1.22E+03 | 19.30% |
| 35 | 19.031 | 4.65962 | 388 | 6.20% |
| 36 | 19.45 | 4.5601 | 1.02E+03 | 16.20% |
| 37 | 19.816 | 4.47683 | 2.21E+03 | 35.10% |
| 38 | 20.036 | 4.42815 | 4.54E+03 | 72.30% |
| 39 | 20.135 | 4.40658 | 2.95E+03 | 46.90% |
| 40 | 20.459 | 4.33751 | 539 | 8.60% |
| 41 | 20.848 | 4.2574 | 1.72E+03 | 27.40% |
| 42 | 21.093 | 4.20848 | 352 | 5.60% |
| 43 | 21.628 | 4.10552 | 181 | 2.90% |
| 44 | 21.96 | 4.04427 | 287 | 4.60% |
| 45 | 22.243 | 3.99339 | 1.70E+03 | 27.10% |
| 46 | 22.774 | 3.90157 | 907 | 14.40% |
| 47 | 22.848 | 3.88907 | 1.03E+03 | 16.40% |
| 48 | 23.244 | 3.82365 | 1.31E+03 | 20.80% |
| 49 | 23.755 | 3.7426 | 1.64E+03 | 26.20% |
| 50 | 24.104 | 3.68915 | 1.08E+03 | 17.10% |
| 51 | 25.566 | 3.48148 | 317 | 5.00% |
| 52 | 26.008 | 3.4233 | 146 | 2.30% |
| 53 | 26.396 | 3.37386 | 622 | 9.90% |
| 54 | 26.791 | 3.32492 | 585 | 9.30% |
| 55 | 27.186 | 3.27758 | 344 | 5.50% |
| 56 | 27.472 | 3.24407 | 532 | 8.50% |
| 57 | 27.602 | 3.22904 | 359 | 5.70% |
| 58 | 27.945 | 3.19025 | 238 | 3.80% |
| 59 | 28.435 | 3.13639 | 159 | 2.50% |
| 60 | 29.021 | 3.07436 | 272 | 4.30% |
| 61 | 29.886 | 2.98729 | 257 | 4.10% |
| 62 | 30.238 | 2.95328 | 229 | 3.60% |
| 63 | 31.099 | 2.8735 | 113 | 1.80% |
| 64 | 32.137 | 2.78305 | 233 | 3.70% |
| 65 | 32.999 | 2.71224 | 107 | 1.70% |
| 66 | 33.69 | 2.6582 | 135 | 2.10% |

The above crystal exhibits a thermal event between about 119° C. and 128° C., e.g., at about 120° C. or 126° C., according to differential scanning calorimetry. Mandelate Salt 1 is in solvate form, and the free base and counter ion are present in the crystal in a ratio of about 1:1.

A second mandelate salt is obtained when mandelic acid is combined with 25 mg of Compound 1 in 3-heptanone, and is subsequently subjected to evaporation under vacuum. The resulting salt is obtained as an orange/brown powder. The XRPD pattern of Mandelate Salt 2 has peaks as set forth in Table 44 below.

TABLE 44

| | | d- | | Rel. |
|---|---|---|---|---|
| Index | Angle | Value | Intensity | Intensity |
| 1 | 6.042 | 14.616 | 5.50E+03 | 9.50% |
| 2 | 7.063 | 12.50588 | 346 | 0.60% |
| 3 | 8.055 | 10.96736 | 1.77E+04 | 30.60% |
| 4 | 8.488 | 10.40837 | 5.61E+03 | 9.70% |
| 5 | 8.567 | 10.31256 | 1.85E+04 | 32.00% |
| 6 | 8.663 | 10.19859 | 5.79E+04 | 100.00% |
| 7 | 9.041 | 9.77346 | 237 | 0.40% |
| 8 | 9.333 | 9.46821 | 121 | 0.20% |
| 9 | 10.023 | 8.81802 | 2.43E+03 | 4.20% |
| 10 | 10.609 | 8.33234 | 526 | 0.90% |
| 11 | 10.96 | 8.06638 | 184 | 0.30% |
| 12 | 11.105 | 7.96121 | 533 | 0.90% |
| 13 | 11.267 | 7.84715 | 426 | 0.70% |
| 14 | 12.553 | 7.04566 | 7.00E+03 | 12.10% |
| 15 | 13.12 | 6.74278 | 297 | 0.50% |
| 16 | 13.775 | 6.42334 | 405 | 0.70% |
| 17 | 14.103 | 6.27488 | 174 | 0.30% |
| 18 | 14.458 | 6.12151 | 299 | 0.50% |
| 19 | 15.037 | 5.88724 | 218 | 0.40% |
| 20 | 15.538 | 5.69834 | 177 | 0.30% |
| 21 | 15.652 | 5.65718 | 276 | 0.50% |
| 22 | 16.009 | 5.5318 | 787 | 1.40% |
| 23 | 17.263 | 5.13259 | 3.32E+03 | 5.70% |
| 24 | 17.73 | 4.99834 | 375 | 0.60% |
| 25 | 18.113 | 4.89353 | 309 | 0.50% |
| 26 | 18.3 | 4.84393 | 264 | 0.50% |
| 27 | 18.67 | 4.74884 | 227 | 0.40% |
| 28 | 19.043 | 4.65662 | 125 | 0.20% |
| 29 | 19.687 | 4.50576 | 639 | 1.10% |
| 30 | 20.006 | 4.43461 | 156 | 0.30% |
| 31 | 20.351 | 4.36027 | 331 | 0.60% |
| 32 | 20.698 | 4.28802 | 3.43E+03 | 5.90% |
| 33 | 21.073 | 4.21239 | 144 | 0.20% |
| 34 | 22.087 | 4.0213 | 3.95E+03 | 6.80% |
| 35 | 22.201 | 4.00089 | 9.45E+03 | 16.30% |
| 36 | 22.374 | 3.97038 | 2.22E+03 | 3.80% |
| 37 | 22.656 | 3.92159 | 2.36E+03 | 4.10% |
| 38 | 22.996 | 3.86436 | 147 | 0.30% |
| 39 | 23.471 | 3.78726 | 107 | 0.20% |
| 40 | 23.776 | 3.73931 | 135 | 0.20% |
| 41 | 24.566 | 3.62078 | 110 | 0.20% |
| 42 | 25.171 | 3.53514 | 3.27E+03 | 5.60% |
| 43 | 25.951 | 3.43065 | 734 | 1.30% |
| 44 | 26.333 | 3.38178 | 156 | 0.30% |
| 45 | 26.717 | 3.33402 | 247 | 0.40% |
| 46 | 27.59 | 3.23049 | 548 | 0.90% |
| 47 | 27.848 | 3.20107 | 141 | 0.20% |
| 48 | 28.575 | 3.12127 | 163 | 0.30% |
| 49 | 28.912 | 3.08566 | 162 | 0.30% |
| 50 | 30.287 | 2.94863 | 236 | 0.40% |
| 51 | 32.265 | 2.77229 | 212 | 0.40% |
| 52 | 36.251 | 2.47608 | 434 | 0.70% |
| 53 | 36.503 | 2.45956 | 128 | 0.20% |
| 54 | 39.652 | 2.27115 | 146 | 0.30% |
| 55 | 41.727 | 2.1629 | 1.62E+03 | 2.80% |
| 56 | 41.83 | 2.15779 | 903 | 1.60% |
| 57 | 42.056 | 2.14674 | 217 | 0.40% |
| 58 | 44.656 | 2.02758 | 104 | 0.20% |

The above crystal exhibits a thermal event between about 102° C. and 105° C., e.g., at about 103° C., according to differential scanning calorimetry. Mandelate Salt 2 is in solvate form, and the free base and counter ion are present in the crystal in a ratio of about 1:1.

### 4-Acetamido-Benzoate Salt

A 4-acetamido-benzoate salt is obtained when 4-acetamido-benzoic acid is combined with 25 mg of Compound 1 in ethyl acetate, and is subsequently slurried at 50° C. for 2 hours. The resulting salt is obtained as a white powder. The XRPD pattern of 4-Acetamido-benzoate Salt 1 has peaks as set forth in Table 45 below.

TABLE 45

| | | d- | | Rel. |
|---|---|---|---|---|
| Index | Angle | Value | Intensity | Intensity |
| 1 | 5.987 | 14.74927 | 2.21E+03 | 54.30% |
| 2 | 6.558 | 13.46799 | 1.79E+03 | 43.80% |
| 3 | 8.304 | 10.63884 | 2.34E+03 | 57.40% |
| 4 | 9.393 | 9.40762 | 1.74E+03 | 42.60% |
| 5 | 10.702 | 8.25965 | 1.21E+03 | 29.70% |
| 6 | 11.549 | 7.65633 | 1.01E+03 | 24.90% |
| 7 | 11.848 | 7.46327 | 1.83E+03 | 44.80% |
| 8 | 12.232 | 7.22999 | 1.19E+03 | 29.10% |
| 9 | 12.917 | 6.8482 | 1.82E+03 | 44.60% |
| 10 | 14.214 | 6.22594 | 2.29E+03 | 56.20% |
| 11 | 15.015 | 5.89565 | 1.58E+03 | 38.70% |
| 12 | 15.156 | 5.84096 | 2.77E+03 | 67.90% |
| 13 | 15.845 | 5.58859 | 1.16E+03 | 28.30% |
| 14 | 16.095 | 5.50252 | 193 | 4.70% |
| 15 | 17.966 | 4.93347 | 1.04E+03 | 25.40% |
| 16 | 18.324 | 4.83779 | 929 | 22.80% |
| 17 | 18.73 | 4.7337 | 4.08E+03 | 100.00% |
| 18 | 19.585 | 4.52912 | 2.17E+03 | 53.10% |
| 19 | 21.393 | 4.15023 | 2.17E+03 | 53.20% |
| 20 | 21.59 | 4.11275 | 963 | 23.60% |
| 21 | 22.928 | 3.87569 | 1.15E+03 | 28.10% |
| 22 | 23.114 | 3.84489 | 946 | 23.20% |
| 23 | 23.484 | 3.78524 | 1.74E+03 | 42.70% |
| 24 | 23.388 | 3.8005 | 1.69E+03 | 41.30% |
| 25 | 23.441 | 3.79208 | 1.89E+03 | 46.30% |
| 26 | 24.2 | 3.67481 | 675 | 16.60% |
| 27 | 24.444 | 3.63864 | 846 | 20.70% |
| 28 | 24.618 | 3.61326 | 526 | 12.90% |
| 29 | 25.197 | 3.53155 | 631 | 15.50% |
| 30 | 25.757 | 3.456 | 437 | 10.70% |
| 31 | 26.405 | 3.37274 | 275 | 6.70% |
| 32 | 26.853 | 3.31746 | 553 | 13.60% |
| 33 | 27.355 | 3.25764 | 94.2 | 2.30% |
| 34 | 27.92 | 3.19298 | 165 | 4.00% |
| 35 | 28.775 | 3.10011 | 147 | 3.60% |
| 36 | 29.555 | 3.02 | 551 | 13.50% |
| 37 | 29.628 | 3.01277 | 460 | 11.30% |
| 38 | 30.496 | 2.92888 | 256 | 6.30% |
| 39 | 30.537 | 2.9251 | 313 | 7.70% |
| 40 | 30.563 | 2.92264 | 282 | 6.90% |
| 41 | 32.78 | 2.72991 | 366 | 9.00% |
| 42 | 33.705 | 2.65702 | 279 | 6.80% |
| 43 | 33.764 | 2.65257 | 241 | 5.90% |
| 44 | 34.044 | 2.63139 | 72 | 1.80% |

The above crystal exhibits a thermal event between about 168° C. and 171° C., e.g., at about 170° C., according to differential scanning calorimetry. 4-Acetamido-benzoate Salt 1 is in anhydrous form, and the free base and counter ion are present in the crystal in a ratio of about 1:1.3.

A second 4-acetamido-benzoate salt is obtained when 4-acetamido-benzoic acid is combined with 25 mg of Compound 1 in 3-heptanone, and is subsequently slurried at 50° C. for 2 hours. The resulting salt is obtained as a white powder. The XRPD pattern of 4-Acetamido-benzoate Salt 2 has peaks as set forth in Table 46 below.

TABLE 46

| | | d- | | Rel. |
|---|---|---|---|---|
| Index | Angle | Value | Intensity | Intensity |
| 1 | 4.878 | 18.10059 | 7.13E+03 | 100.00% |
| 2 | 6.766 | 13.05435 | 6.19E+03 | 86.80% |
| 3 | 6.996 | 12.62504 | 274 | 3.80% |
| 4 | 9.967 | 8.86778 | 1.12E+03 | 15.70% |
| 5 | 10.654 | 8.29675 | 1.35E+03 | 19.00% |
| 6 | 10.988 | 8.0458 | 1.00E+03 | 14.00% |
| 7 | 11.554 | 7.65247 | 1.55E+03 | 21.70% |
| 8 | 12.085 | 7.31766 | 205 | 2.90% |
| 9 | 12.362 | 7.15424 | 519 | 7.30% |
| 10 | 13.205 | 6.69919 | 574 | 8.00% |
| 11 | 13.825 | 6.40052 | 1.49E+03 | 20.90% |
| 12 | 14.319 | 6.18054 | 3.82E+03 | 53.60% |
| 13 | 15.416 | 5.74305 | 278 | 3.90% |
| 14 | 15.761 | 5.61812 | 1.89E+03 | 26.50% |
| 15 | 16.061 | 5.51402 | 200 | 2.80% |
| 16 | 16.444 | 5.38647 | 1.66E+03 | 23.30% |
| 17 | 17.671 | 5.01498 | 1.15E+03 | 16.10% |
| 18 | 17.894 | 4.95298 | 951 | 13.30% |
| 19 | 18.658 | 4.75195 | 601 | 8.40% |
| 20 | 19.208 | 4.61701 | 1.43E+03 | 20.00% |
| 21 | 19.248 | 4.60757 | 1.45E+03 | 20.30% |
| 22 | 19.937 | 4.44979 | 1.35E+03 | 18.90% |
| 23 | 20.211 | 4.39011 | 454 | 6.40% |
| 24 | 20.533 | 4.32206 | 561 | 7.90% |
| 25 | 21.071 | 4.21295 | 290 | 4.10% |
| 26 | 21.487 | 4.13221 | 2.38E+03 | 33.30% |
| 27 | 21.767 | 4.07971 | 395 | 5.50% |
| 28 | 22 | 4.03706 | 354 | 5.00% |
| 29 | 22.6 | 3.93117 | 235 | 3.30% |
| 30 | 23.138 | 3.84095 | 1.03E+03 | 14.40% |
| 31 | 23.161 | 3.83723 | 1.04E+03 | 14.60% |
| 32 | 23.457 | 3.78942 | 1.27E+03 | 17.80% |
| 33 | 23.868 | 3.72507 | 886 | 12.40% |
| 34 | 24.209 | 3.67348 | 571 | 8.00% |
| 35 | 24.435 | 3.63999 | 741 | 10.40% |
| 36 | 24.534 | 3.62551 | 746 | 10.50% |
| 37 | 25.093 | 3.54594 | 429 | 6.00% |
| 38 | 25.969 | 3.4283 | 172 | 2.40% |
| 39 | 26.14 | 3.40622 | 322 | 4.50% |
| 40 | 26.457 | 3.36622 | 549 | 7.70% |
| 41 | 26.924 | 3.30885 | 362 | 5.10% |
| 42 | 26.998 | 3.29995 | 476 | 6.70% |
| 43 | 27.245 | 3.27055 | 427 | 6.00% |
| 44 | 28.143 | 3.16827 | 1.02E+03 | 14.30% |
| 45 | 28.77 | 3.10061 | 125 | 1.80% |
| 46 | 29.103 | 3.06583 | 177 | 2.50% |
| 47 | 29.434 | 3.03218 | 76.3 | 1.10% |
| 48 | 29.752 | 3.00048 | 68.1 | 1.00% |
| 49 | 30.069 | 2.96954 | 130 | 1.80% |
| 50 | 31.493 | 2.8384 | 117 | 1.60% |
| 51 | 31.673 | 2.82276 | 212 | 3.00% |
| 52 | 31.955 | 2.79846 | 201 | 2.80% |
| 53 | 32.14 | 2.78277 | 192 | 2.70% |
| 54 | 32.69 | 2.73716 | 140 | 2.00% |
| 55 | 33.258 | 2.69174 | 170 | 2.40% |
| 56 | 33.404 | 2.68032 | 236 | 3.30% |
| 57 | 34.054 | 2.6306 | 106 | 1.50% |
| 58 | 35.369 | 2.53578 | 116 | 1.60% |
| 59 | 35.747 | 2.50981 | 107 | 1.50% |
| 60 | 37.215 | 2.41408 | 90.9 | 1.30% |
| 61 | 38.752 | 2.3218 | 71.4 | 1.00% |
| 62 | 41.012 | 2.19894 | 116 | 1.60% |
| 63 | 42.698 | 2.11595 | 92.4 | 1.30% |
| 64 | 42.753 | 2.11336 | 121 | 1.70% |
| 65 | 43.682 | 2.07052 | 105 | 1.50% |

The above crystal exhibits a thermal event between about 127° C. and 130° C., e.g., at about 129° C., and between about 170° C. and 173° C., e.g., at about 172° C., according to differential scanning calorimetry. 4-Acetamido-benzoate Salt 2 is in solvate form, and the free base and counter ion are present in the crystal in a ratio of about 1:1.

### Trifluoroacetate Salt

A trifluoroacetate salt is obtained when trifluoroacetic acid is combined with 25 mg of Compound 1 in acetonitrile, and is subsequently slurried at 50° C. for 2 hours. The resulting salt is obtained initially as a clear liquid, and subsequent to evaporation, a white powder. The XRPD pattern of Trifluoroacetate Salt 1 has peaks as set forth in Table 47 below.

TABLE 47

| | | d- | | Rel. |
|---|---|---|---|---|
| Index | Angle | Value | Intensity | Intensity |
| 1 | 6.402 | 13.7954 | 1.14E+03 | 18.80% |
| 2 | 6.477 | 13.63492 | 3.21E+03 | 53.20% |
| 3 | 6.641 | 13.29871 | 6.03E+03 | 100.00% |
| 4 | 8.368 | 10.55807 | 181 | 3.00% |
| 5 | 8.505 | 10.38764 | 436 | 7.20% |
| 6 | 8.659 | 10.20399 | 2.27E+03 | 37.70% |
| 7 | 8.878 | 9.95278 | 2.31E+03 | 38.20% |
| 8 | 8.944 | 9.87903 | 1.02E+03 | 16.90% |
| 9 | 9.735 | 9.07862 | 390 | 6.50% |
| 10 | 10.506 | 8.41386 | 163 | 2.70% |
| 11 | 11.92 | 7.41834 | 1.81E+03 | 30.00% |
| 12 | 12.245 | 7.22233 | 1.71E+03 | 28.40% |
| 13 | 12.498 | 7.07693 | 1.22E+03 | 20.20% |
| 14 | 13.203 | 6.70033 | 73.4 | 1.20% |
| 15 | 13.81 | 6.40706 | 404 | 6.70% |
| 16 | 14.021 | 6.31143 | 602 | 10.00% |
| 17 | 14.096 | 6.27799 | 640 | 10.60% |
| 18 | 16.363 | 5.41273 | 220 | 3.60% |
| 19 | 16.465 | 5.37953 | 390 | 6.50% |
| 20 | 16.594 | 5.33812 | 429 | 7.10% |
| 21 | 16.673 | 5.31294 | 687 | 11.40% |
| 22 | 17.035 | 5.20066 | 3.43E+03 | 56.90% |
| 23 | 17.168 | 5.16071 | 1.15E+03 | 19.00% |
| 24 | 17.463 | 5.07425 | 1.24E+03 | 20.60% |
| 25 | 17.535 | 5.05358 | 1.26E+03 | 20.90% |
| 26 | 18.818 | 4.71187 | 2.25E+03 | 37.30% |
| 27 | 19.454 | 4.55922 | 1.84E+03 | 30.50% |
| 28 | 19.605 | 4.52453 | 1.31E+03 | 21.70% |
| 29 | 19.763 | 4.48871 | 658 | 10.90% |
| 30 | 19.822 | 4.4755 | 586 | 9.70% |
| 31 | 20.336 | 4.3634 | 792 | 13.10% |
| 32 | 20.409 | 4.34807 | 1.85E+03 | 30.70% |
| 33 | 20.564 | 4.31553 | 158 | 2.60% |
| 34 | 20.872 | 4.25249 | 1.55E+03 | 25.70% |
| 35 | 21.78 | 4.07727 | 1.69E+03 | 28.00% |
| 36 | 22.204 | 4.00031 | 607 | 10.10% |
| 37 | 22.394 | 3.96689 | 1.21E+03 | 20.00% |
| 38 | 22.454 | 3.95648 | 1.21E+03 | 20.10% |
| 39 | 23.263 | 3.82066 | 1.19E+03 | 19.80% |
| 40 | 23.446 | 3.79125 | 5.77E+03 | 95.60% |
| 41 | 24.552 | 3.62291 | 1.90E+03 | 31.40% |
| 42 | 24.943 | 3.5669 | 101 | 1.70% |
| 43 | 25.176 | 3.53452 | 93.4 | 1.50% |
| 44 | 25.359 | 3.50944 | 745 | 12.40% |
| 45 | 25.919 | 3.43476 | 159 | 2.60% |
| 46 | 26.1 | 3.41146 | 129 | 2.10% |
| 47 | 26.414 | 3.3716 | 221 | 3.70% |
| 48 | 26.843 | 3.31862 | 216 | 3.60% |
| 49 | 27.624 | 3.22655 | 566 | 9.40% |
| 50 | 27.663 | 3.22216 | 563 | 9.30% |
| 51 | 28.212 | 3.1606 | 672 | 11.10% |
| 52 | 28.362 | 3.14429 | 787 | 13.10% |
| 53 | 28.62 | 3.11645 | 204 | 3.40% |
| 54 | 28.971 | 3.07952 | 80.9 | 1.30% |
| 55 | 29.303 | 3.04538 | 403 | 6.70% |
| 56 | 29.582 | 3.0173 | 527 | 8.70% |
| 57 | 29.785 | 2.99725 | 347 | 5.70% |
| 58 | 30.532 | 2.92555 | 86.4 | 1.40% |
| 59 | 30.947 | 2.88727 | 661 | 11.00% |
| 60 | 32.223 | 2.77581 | 296 | 4.90% |
| 61 | 32.665 | 2.7392 | 157 | 2.60% |
| 62 | 33.249 | 2.69245 | 186 | 3.10% |
| 63 | 33.991 | 2.63533 | 285 | 4.70% |
| 64 | 34.519 | 2.59621 | 102 | 1.70% |
| 65 | 34.691 | 2.58372 | 978 | 16.20% |
| 66 | 34.774 | 2.5778 | 590 | 9.80% |
| 67 | 36.397 | 2.46648 | 104 | 1.70% |
| 68 | 36.48 | 2.46103 | 47.8 | 0.80% |
| 69 | 37.86 | 2.37443 | 57.4 | 1.00% |
| 70 | 38.285 | 2.34906 | 56.3 | 0.90% |
| 71 | 38.676 | 2.32623 | 140 | 2.30% |
| 72 | 39.103 | 2.3018 | 175 | 2.90% |
| 73 | 39.162 | 2.29844 | 176 | 2.90% |
| 74 | 39.493 | 2.27997 | 143 | 2.40% |
| 75 | 40.05 | 2.2495 | 73.2 | 1.20% |
| 76 | 40.317 | 2.23523 | 150 | 2.50% |
| 77 | 40.404 | 2.23061 | 92.3 | 1.50% |
| 78 | 41.584 | 2.16999 | 99.4 | 1.60% |
| 79 | 42.167 | 2.14134 | 61.9 | 1.00% |
| 80 | 43.358 | 2.08524 | 295 | 4.90% |
| 81 | 43.479 | 2.0797 | 142 | 2.40% |

The above crystal exhibits a thermal event between about 253° C. and 257° C., e.g., at about 255° C., according to differential scanning calorimetry. Trifluoroacetate Salt 1 is in anhydrous form, and the free base and counter ion are present in the crystal in a ratio of about 1:0.9.

### Dichloroacetate Salt

A dichloroacetate salt is obtained when trifluoroacetic acid is combined with 25 mg of Compound 1 in acetonitrile, and is subsequently slurried at 50° C. for 2 hours. The resulting salt is obtained as a white powder. The XRPD pattern of Dichloroacetate Salt 1 has peaks as set forth in Table 48 below.

TABLE 48

| | | d- | | Rel. |
|---|---|---|---|---|
| Index | Angle | Value | Intensity | Intensity |
| 1 | 6.122 | 14.425 | 320 | 4.10% |
| 2 | 6.218 | 14.203 | 627 | 8.00% |
| 3 | 6.293 | 14.034 | 968 | 12.30% |
| 4 | 6.453 | 13.687 | 3.99E+03 | 50.70% |
| 5 | 8.772 | 10.073 | 2.04E+03 | 25.90% |
| 6 | 8.908 | 9.9187 | 1.01E+03 | 12.90% |
| 7 | 9.682 | 9.128 | 366 | 4.70% |
| 8 | 10.33 | 8.5599 | 248 | 3.20% |
| 9 | 11.92 | 7.4171 | 3.82E+03 | 48.60% |
| 10 | 12.04 | 7.3452 | 925 | 11.80% |
| 11 | 12.25 | 7.2179 | 2.04E+03 | 25.90% |
| 12 | 13.86 | 6.3837 | 403 | 5.10% |
| 13 | 14.13 | 6.2614 | 765 | 9.70% |
| 14 | 14.3 | 6.1882 | 770 | 9.80% |
| 15 | 16.62 | 5.3309 | 636 | 8.10% |
| 16 | 16.93 | 5.2331 | 1.51E+03 | 19.10% |
| 17 | 17.15 | 5.1655 | 2.29E+03 | 29.00% |
| 18 | 17.27 | 5.132 | 1.07E+03 | 13.70% |
| 19 | 17.66 | 5.0176 | 7.87E+03 | 100.00% |
| 20 | 18.85 | 4.7048 | 2.24E+03 | 28.50% |
| 21 | 19.32 | 4.5898 | 1.52E+03 | 19.30% |
| 22 | 19.61 | 4.5231 | 490 | 6.20% |
| 23 | 19.84 | 4.4705 | 1.20E+03 | 15.30% |
| 24 | 20 | 4.4354 | 1.35E+03 | 17.10% |
| 25 | 20.91 | 4.2442 | 1.02E+03 | 12.90% |
| 26 | 21.33 | 4.1615 | 896 | 11.40% |
| 27 | 21.49 | 4.1322 | 1.25E+03 | 15.90% |
| 28 | 22.01 | 4.0348 | 384 | 4.90% |
| 29 | 22.32 | 3.9808 | 969 | 12.30% |
| 30 | 22.53 | 3.9438 | 565 | 7.20% |
| 31 | 22.82 | 3.8943 | 1.06E+03 | 13.40% |
| 32 | 23.06 | 3.8537 | 1.15E+03 | 14.60% |
| 33 | 23.61 | 3.7651 | 1.86E+03 | 23.60% |
| 34 | 23.86 | 3.7258 | 343 | 4.40% |
| 35 | 24.54 | 3.6248 | 414 | 5.30% |
| 36 | 25.39 | 3.5052 | 86.2 | 1.10% |
| 37 | 25.58 | 3.4792 | 349 | 4.40% |
| 38 | 25.72 | 3.4613 | 1.15E+03 | 14.60% |
| 39 | 26.11 | 3.4099 | 442 | 5.60% |
| 40 | 26.75 | 3.3299 | 366 | 4.60% |
| 41 | 26.95 | 3.3064 | 617 | 7.80% |
| 42 | 27.42 | 3.2506 | 174 | 2.20% |
| 43 | 28.2 | 3.1617 | 1.51E+03 | 19.20% |
| 44 | 28.39 | 3.1409 | 834 | 10.60% |
| 45 | 29.1 | 3.0661 | 511 | 6.50% |
| 46 | 29.75 | 3.0007 | 206 | 2.60% |
| 47 | 30.48 | 2.9304 | 138 | 1.80% |
| 48 | 31.7 | 2.8201 | 192 | 2.40% |
| 49 | 31.74 | 2.8173 | 207 | 2.60% |
| 50 | 31.93 | 2.8004 | 999 | 12.70% |
| 51 | 32.02 | 2.7933 | 565 | 7.20% |
| 52 | 32.29 | 2.7702 | 167 | 2.10% |
| 53 | 32.83 | 2.7258 | 187 | 2.40% |
| 54 | 32.89 | 2.7209 | 152 | 1.90% |
| 55 | 33.06 | 2.7074 | 92 | 1.20% |
| 56 | 33.54 | 2.6701 | 143 | 1.80% |
| 57 | 34.12 | 2.626 | 206 | 2.60% |
| 58 | 34.64 | 2.5876 | 111 | 1.40% |
| 59 | 35.66 | 2.5158 | 143 | 1.80% |
| 60 | 35.71 | 2.5124 | 142 | 1.80% |
| 61 | 42.74 | 2.114 | 50.9 | 0.60% |
| 62 | 44.34 | 2.0412 | 83.6 | 1.10% |

The above crystal exhibits thermal events between about 225° C. and 228° C., e.g., at about 227° C., and between about 229° C. and 232° C., e.g., at about 230° C., according to differential scanning calorimetry. Dichloroacetate Salt 1 is in anhydrous form, and the free base and counter ion are present in the crystal in a ratio of about 1:0.8.

### Caproate Salt

A caproate salt is obtained when caproic acid is combined with 25 mg of Compound 1 in 2-butanone, and is subsequently slurried at 50° C. for 2 hours. The resulting salt is obtained initially as a clear liquid, and as an off-white powder after evaporation. The XRPD pattern of Caproate Salt 1 has peaks as set forth in Table 49 below.

TABLE 49

| | | d- | | Rel. |
|---|---|---|---|---|
| Index | Angle | Value | Intensity | Intensity |
| 1 | 5.156 | 17.12512 | 2.15E+04 | 100.00% |
| 2 | 5.704 | 15.48206 | 592 | 2.70% |
| 3 | 7.488 | 11.79715 | 2.32E+03 | 10.80% |
| 4 | 7.798 | 11.32866 | 4.06E+03 | 18.80% |
| 5 | 10.223 | 8.6461 | 3.75E+03 | 17.40% |
| 6 | 11.328 | 7.80459 | 6.03E+03 | 28.00% |
| 7 | 11.436 | 7.73135 | 771 | 3.60% |
| 8 | 11.873 | 7.44781 | 867 | 4.00% |
| 9 | 12.224 | 7.23471 | 2.01E+03 | 9.30% |
| 10 | 12.607 | 7.01554 | 1.78E+03 | 8.20% |
| 11 | 14.944 | 5.92351 | 684 | 3.20% |
| 12 | 15.133 | 5.84982 | 995 | 4.60% |
| 13 | 15.551 | 5.69349 | 1.07E+03 | 5.00% |
| 14 | 16.124 | 5.4927 | 568 | 2.60% |
| 15 | 16.531 | 5.35822 | 1.66E+03 | 7.70% |
| 16 | 16.755 | 5.28719 | 476 | 2.20% |
| 17 | 16.967 | 5.22153 | 299 | 1.40% |
| 18 | 17.6 | 5.0351 | 825 | 3.80% |
| 19 | 17.806 | 4.97734 | 452 | 2.10% |
| 20 | 19.428 | 4.56528 | 701 | 3.30% |
| 21 | 19.638 | 4.51694 | 2.22E+03 | 10.30% |
| 22 | 20.095 | 4.41532 | 177 | 0.80% |
| 23 | 20.305 | 4.37003 | 555 | 2.60% |
| 24 | 20.583 | 4.31167 | 575 | 2.70% |
| 25 | 20.974 | 4.23221 | 394 | 1.80% |
| 26 | 21.423 | 4.14436 | 198 | 0.90% |
| 27 | 22.424 | 3.96157 | 654 | 3.00% |
| 28 | 22.673 | 3.91866 | 1.55E+03 | 7.20% |
| 29 | 23.038 | 3.85746 | 3.18E+03 | 14.70% |
| 30 | 23.416 | 3.79601 | 2.18E+03 | 10.10% |
| 31 | 23.882 | 3.72297 | 1.03E+03 | 4.80% |
| 32 | 24.181 | 3.67765 | 644 | 3.00% |
| 33 | 24.785 | 3.58937 | 234 | 1.10% |
| 34 | 25.618 | 3.47449 | 244 | 1.10% |
| 35 | 26.338 | 3.3811 | 105 | 0.50% |
| 36 | 27.23 | 3.27234 | 334 | 1.60% |
| 37 | 27.445 | 3.24718 | 303 | 1.40% |
| 38 | 27.895 | 3.19588 | 203 | 0.90% |
| 39 | 27.989 | 3.18526 | 210 | 1.00% |
| 40 | 28.455 | 3.13419 | 183 | 0.90% |
| 41 | 28.648 | 3.11351 | 169 | 0.80% |
| 42 | 29.389 | 3.0367 | 185 | 0.90% |
| 43 | 31.567 | 2.832 | 46.9 | 0.20% |
| 44 | 31.999 | 2.79467 | 95.6 | 0.40% |
| 45 | 32.691 | 2.73712 | 104 | 0.50% |

The above crystal exhibits thermal events between about 89° C. and 92° C., e.g., at about 90° C., and between about 104° C. and 107° C., e.g., at about 105° C., according to differential scanning calorimetry. Caproate Salt 1 is in anhydrous form, and the free base and counter ion are present in the crystal in a ratio of about 1:0.9.

### Laurate Salt

A laurate salt is obtained when lauric acid is combined with 25 mg of Compound 1 in 2-propanol, and is subsequently subjected to evaporation under vacuum. The resulting salt is obtained as a white powder. The XRPD pattern of Laurate Salt 1 has peaks as set forth in Table 50 below.

TABLE 50

| | | d- | | Rel. |
|---|---|---|---|---|
| Index | Angle | Value | Intensity | Intensity |
| 1 | 2.574 | 34.29293 | 300 | 6.00% |
| 2 | 4.315 | 20.4628 | 2.54E+03 | 51.10% |
| 3 | 4.62 | 19.1131 | 4.97E+03 | 100.00% |
| 4 | 5.494 | 16.07362 | 1.60E+03 | 32.10% |
| 5 | 5.908 | 14.9483 | 1.70E+03 | 34.10% |
| 6 | 6.517 | 13.55199 | 648 | 13.00% |
| 7 | 7.343 | 12.02957 | 440 | 8.90% |
| 8 | 8.53 | 10.35786 | 862 | 17.30% |
| 9 | 8.66 | 10.20238 | 567 | 11.40% |
| 10 | 9.163 | 9.64341 | 1.13E+03 | 22.70% |
| 11 | 9.535 | 9.26823 | 442 | 8.90% |
| 12 | 10.662 | 8.29083 | 264 | 5.30% |
| 13 | 10.944 | 8.07792 | 930 | 18.70% |
| 14 | 10.998 | 8.03824 | 1.14E+03 | 23.00% |
| 15 | 12.047 | 7.34058 | 707 | 14.20% |
| 16 | 12.787 | 6.91728 | 401 | 8.10% |
| 17 | 12.821 | 6.89942 | 371 | 7.50% |
| 18 | 14.477 | 6.11359 | 181 | 3.60% |
| 19 | 14.973 | 5.91226 | 616 | 12.40% |
| 20 | 15.05 | 5.88197 | 708 | 14.30% |
| 21 | 16.754 | 5.28736 | 460 | 9.30% |
| 22 | 19.136 | 4.63428 | 546 | 11.00% |
| 23 | 19.413 | 4.56881 | 442 | 8.90% |
| 24 | 20.006 | 4.43459 | 525 | 10.60% |
| 25 | 21.588 | 4.11319 | 346 | 7.00% |
| 26 | 22.023 | 4.03278 | 938 | 18.90% |
| 27 | 22.061 | 4.02592 | 948 | 19.10% |
| 28 | 22.657 | 3.92138 | 2.67E+03 | 53.80% |

The above crystal exhibits a thermal event between about 81° C. and 84° C., e.g., at about 83° C., according to differential scanning calorimetry. Laurate Salt 1 is in anhydrous form, and the free base and counter ion are present in the crystal in a ratio of about 1:1.4.

### Example 2—Solubility Study of Obtained Salt Crystals

The aqueous solubilities of the salt crystals generated in Example 1 are determined by shaking the salt crystals in water for 24 hours. Samples are filtered and diluted (in a mixture of acetonitrile/water (1/1)) for LC analysis. Solubilities are calculated with the use of a calibration line. Furthermore, the pH values of the filtrated solutions are determined using pH indication paper. The solubilities of the salts are compared against Compound 1 in free base form, as well as a phosphate salt comparator of Compound 1, as disclosed in WO2013192556 Å2.

TABLE 51

| | Dilution | Injection | Area | Solubility | | |
|---|---|---|---|---|---|---|
| Salt | factor | Vol. (uL) | (mAU*sec) | (mg/mL) | Appearance | pH |
| Free Base | 20 | 5 | 1 | 0 | Slurry | 7-8 |
| Phosphate Salt | 20 | 0.5 | 7951 | 53.66 | Slurry | 5 |
| Control | | | | | | |
| Malate Salt 1 | 20 | 0.1 | 2419 | >81.63 | Clear | 4 |
| Tartrate Salt 1 | 20 | 0.1 | 1561 | >52.68 | Clear | 4-5 |
| Fumarate Salt 1 | 20 | 5 | 6796 | 4.59 | Slurry | 2 |
| Succinate Salt 1 | 20 | 5 | 2185 | 1.47 | Slurry | 4 |
| Succinate Salt 2 | 20 | 5 | 2728 | 1.84 | Slurry | 4 |
| Galactarate Salt 1 | 20 | 0.2 | 499 | 8.41 | Slurry | 5 |
| Adipate Salt 1 | 20 | 5 | 4380 | 2.96 | Slurry | 4 |
| Adipate Salt 2 | 20 | 5 | 3063 | 2.07 | Slurry | 4-5 |
| Adipate Salt 3 | 50 | 0.5 | 2664 | 44.95 | Slurry | 4-5 |
| Esylate Salt 1 | 20 | 5 | 4261 | 2.88 | Slurry | 5 |
| Mesylate Salt 1 | 20 | 5 | 8273 | 5.58 | Slurry | 5 |
| Propionate Salt 1 | 20 | 5 | 3655 | 2.47 | Slurry | 5 |
| Lactate Salt 1 | 50 | 0.5 | 1488 | 25.11 | Slurry | 4-5 |
| Caprylate Salt 1 | 20 | 5 | 75 | 0.05 | Slurry | 5 |
| Sulfate Salt 1 | 50 | 5 | 2407 | 4.06 | Slurry | 1 |
| Laurate Salt 1 | 50 | 5 | 448 | 0.76 | Slurry | 5 |
| Palmitate Salt 1 | 50 | 5 | 118 | 0.2 | Slurry | 5-6 |
| Napadisylate Salt 1 | 20 | 5 | 289 | 0.19 | Slurry | 1 |
| Edisylate Salt 1 | 20 | 2 | 87 | 0.15 | Slurry | 1 |
| Tosylate Salt 1 | 50 | 5 | 72 | 0.12 | Slurry | 1 |
| Tosylate Salt 2 | 50 | 5 | 582 | 0.98 | Slurry | 5 |
| Besylate Salt 1 | 50 | 5 | 41 | 0.07 | Slurry | 1 |
| Oxalate Salt 1 | 50 | 5 | 1986 | 3.35 | Slurry | 4 |
| Oxalate Salt 2 | 20 | 0.1 | 969 | 32.69 | Slurry | 1 |
| Oxalate Salt 3 | 50 | 0.5 | 2939 | 49.59 | Slurry | 1 |
| Xinafoate Salt 1 | 50 | 5 | 10 | 0.02 | Slurry | 7 |
| Xinafoate Salt 2 | 50 | 5 | 5 | 0.01 | Slurry | 7 |
| Xinafoate Salt 3 | 50 | 5 | 18 | 0.03 | Slurry | 7 |
| Benzoate Salt 1 | 50 | 5 | 2653 | 4.48 | Slurry | 5 |
| Nicotinate Salt 1 | 50 | 1 | 4113 | 34.7 | Slurry | 5 |
| Isonicotinate Salt 1 | 50 | 5 | 4519 | 7.63 | Slurry | 5 |
| Orotate Salt 1 | 50 | 5 | 5778 | 9.75 | Slurry | 5 |
| Camsylate Salt 1 | 50 | 5 | 246 | 0.42 | Slurry | 1 |
| Camsylate Salt 2 | 50 | 5 | 105 | 0.18 | Slurry | 1 |
| Salicylate Salt 1 | 50 | 5 | 135 | 0.23 | Slurry | 5 |
| Salicylate Salt 2 | 50 | 5 | 90 | 0.15 | Slurry | 4-5 |
| Aminosalicylate | 50 | 5 | 394 | 0.67 | Slurry | 5 |
| Salt 1 | | | | | | |
| Mandelate Salt 2 | 50 | 5 | 1317 | 2.22 | Slurry | 4 |
| Mandelate Salt 1 | 50 | 5 | 677 | 1.14 | Slurry | 2 |
| 4-acetamido- | 50 | 5 | 156 | 0.26 | Slurry | 4 |
| benzoate Salt 2 | | | | | | |
| 4-acetamido- | 50 | 5 | 420 | 0.71 | Slurry | 5 |
| benzoate Salt 1 | | | | | | |
| HCl Salt 3 | 50 | 0.5 | 6648 | >112.17 | Clear | 3 |
| HCl Salt 1 | 50 | 0.5 | 9332 | >157.46 | Clear | 4 |
| HCl Salt 4 | 50 | 0.5 | 8681 | >146.48 | Clear | 4 |
| HCl Salt 2 | 50 | 0.5 | 9582 | 161.69 | Slurry | 4-5 |
| Phosphate Salt 2 | 50 | 5 | 840 | 1.42 | Slurry | 1 |
| Trifluoroacetate | 100 | 5 | 30 | 0.1 | Slurry | 7 |
| Salt 1 | | | | | | |
| Dichloroacetate | 50 | 5 | 57 | 0.1 | Slurry | 4-5 |
| Salt 1 | | | | | | |
| Caproate Salt 1 | 50 | 5 | 445 | 0.75 | Slurry | 5 |
| Oxo-glutarate | 50 | 0.5 | 1950 | 32.91 | Slurry | 3 |
| Salt 1 | | | | | | |

For the malate, tartrate, oxalate, hydrochloride and the obtained phosphate salts, the aqueous solubility is similar to or higher than the Phosphate Salt Comparator. The hydrochloride salts in particular have good aqueous solubilities (i.e., 112 mg/ml and above).

### Example 3—Scale Up of Hydrochloride Salts

Further hydrochloride salts are generated following the methods as generally described above in Example 1. 100 mg of Hydrochloride Salt 1 is dissolved in 2-ethyl-1-butanol and subjected to temperature cycles using a Technobis Crystal 16 machine. The salt is subjected to consecutive cycles from 50° C. to 0° C., 40° C. to 0° C., 30° C. to 0° C. and 20° C. to 0° C., with a heating rate of 10° C./min and a cooling rate of 0.5° C./min. The obtained materials were analyzed using XRPD and TGA-DSC. The resulting salt is obtained as an off-white powder. The XRPD pattern of Hydrochloride Salt 5 has peaks as set forth in Table 52 below.

TABLE 52

| | | d- | | Rel. |
|---|---|---|---|---|
| Index | Angle | Value | Intensity | Intensity |
| 1 | 4.607 | 19.16342 | 266 | 13.60% |
| 2 | 6.684 | 13.21449 | 1.36E+03 | 69.70% |
| 3 | 7.709 | 11.45952 | 1.46E+03 | 74.80% |
| 4 | 8.847 | 9.98688 | 1.95E+03 | 100.00% |
| 5 | 9.051 | 9.76297 | 1.31E+03 | 67.30% |
| 6 | 9.611 | 9.19525 | 69.2 | 3.60% |
| 7 | 11.355 | 7.78639 | 739 | 37.90% |
| 8 | 11.783 | 7.50458 | 239 | 12.30% |
| 9 | 12.236 | 7.22779 | 284 | 14.60% |
| 10 | 12.912 | 6.8506 | 692 | 35.50% |
| 11 | 13.621 | 6.49592 | 86 | 4.40% |
| 12 | 14.128 | 6.26389 | 182 | 9.30% |
| 13 | 16.355 | 5.41545 | 1.49E+03 | 76.30% |
| 14 | 16.965 | 5.22209 | 893 | 45.80% |
| 15 | 17.66 | 5.01802 | 411 | 21.10% |
| 16 | 18.426 | 4.81129 | 834 | 42.80% |
| 17 | 19.535 | 4.54054 | 374 | 19.20% |
| 18 | 21.297 | 4.1686 | 441 | 22.60% |
| 19 | 21.29 | 4.17004 | 440 | 22.60% |
| 20 | 21.921 | 4.05148 | 720 | 36.90% |
| 21 | 21.958 | 4.04461 | 607 | 31.20% |
| 22 | 23.071 | 3.85202 | 102 | 5.20% |
| 23 | 23.08 | 3.85049 | 124 | 6.40% |
| 24 | 23.624 | 3.76308 | 141 | 7.20% |
| 25 | 24.133 | 3.68489 | 721 | 37.00% |
| 26 | 25.343 | 3.51153 | 111 | 5.70% |
| 27 | 26.663 | 3.34058 | 278 | 14.30% |
| 28 | 26.73 | 3.33237 | 297 | 15.20% |
| 29 | 27.437 | 3.24817 | 342 | 17.50% |
| 30 | 27.489 | 3.24207 | 410 | 21.00% |
| 31 | 28.191 | 3.16295 | 192 | 9.90% |
| 32 | 28.83 | 3.09423 | 213 | 11.00% |
| 33 | 33.777 | 2.65152 | 76.9 | 3.90% |

The above crystal exhibits a thermal event between about 158° C. and 161° C., e.g., at about 159° C., according to differential scanning calorimetry. Hydrochloride Salt 5 is in solvate form. This form is reproducible using an amorphous hydrochloride salt of Compound 1 as well.

A sixth hydrochloride salt is generated following the methods as generally described above in Example 1. 100 mg of Hydrochloride Salt 1 is dissolved in ethyl butyl ketone and subjected to temperature cycles as described above. The obtained materials were analyzed using XRPD and TGA-DSC. The resulting salt is obtained as an off-white powder. The XRPD pattern of Hydrochloride Salt 6 has peaks as set forth in Table 53 below.

TABLE 53

| | | d- | | Rel. |
|---|---|---|---|---|
| Index | Angle | Value | Intensity | Intensity |
| 1 | 3.734 | 23.64643 | 220 | 10.00% |
| 2 | 4.965 | 17.7826 | 2.20E+03 | 100.00% |
| 3 | 7.087 | 12.46325 | 969 | 44.10% |
| 4 | 7.538 | 11.71791 | 427 | 19.40% |
| 5 | 7.799 | 11.32673 | 624 | 28.40% |
| 6 | 8.514 | 10.37703 | 589 | 26.80% |
| 7 | 8.732 | 10.1181 | 364 | 16.60% |
| 8 | 9.338 | 9.46276 | 310 | 14.10% |
| 9 | 12.438 | 7.11093 | 1.23E+03 | 55.90% |
| 10 | 12.852 | 6.88264 | 257 | 11.70% |
| 11 | 13.008 | 6.80029 | 559 | 25.40% |
| 12 | 13.109 | 6.7482 | 296 | 13.50% |
| 13 | 13.964 | 6.33695 | 128 | 5.80% |
| 14 | 14.659 | 6.03806 | 421 | 19.20% |
| 15 | 14.97 | 5.91345 | 362 | 16.40% |
| 16 | 15.348 | 5.76851 | 262 | 11.90% |
| 17 | 16.444 | 5.38636 | 159 | 7.20% |
| 18 | 16.963 | 5.22284 | 194 | 8.80% |
| 19 | 17.539 | 5.0526 | 353 | 16.00% |
| 20 | 18.731 | 4.7335 | 612 | 27.80% |
| 21 | 18.766 | 4.72492 | 589 | 26.80% |
| 22 | 19.216 | 4.6152 | 327 | 14.90% |
| 23 | 19.665 | 4.51073 | 344 | 15.60% |
| 24 | 19.735 | 4.49497 | 315 | 14.30% |
| 25 | 20.114 | 4.41108 | 152 | 6.90% |
| 26 | 20.342 | 4.36211 | 99.4 | 4.50% |
| 27 | 20.775 | 4.27225 | 569 | 25.90% |
| 28 | 21.226 | 4.18242 | 263 | 11.90% |
| 29 | 21.607 | 4.1096 | 261 | 11.90% |
| 30 | 22.173 | 4.00594 | 268 | 12.20% |
| 31 | 22.219 | 3.99771 | 221 | 10.10% |
| 32 | 22.929 | 3.87543 | 403 | 18.30% |
| 33 | 24.107 | 3.68879 | 181 | 8.20% |
| 34 | 24.597 | 3.61638 | 124 | 5.70% |
| 35 | 25.38 | 3.50654 | 177 | 8.10% |
| 36 | 26.431 | 3.36949 | 81.3 | 3.70% |

The above crystal exhibits a thermal event between about 129° C. and 133° C., e.g., at about 131° C., according to differential scanning calorimetry. Hydrochloride Salt 6 is in solvate form. This form is reproducible using an amorphous hydrochloride salt of Compound 1 as well.

A seventh hydrochloride salt is generated following the methods as generally described above in Example 1. 100 mg of Hydrochloride Salt 1 is dissolved in anisole and subjected to temperature cycles as described above. The obtained materials were analyzed using XRPD and TGA-DSC. The resulting salt is obtained as an off-white powder. The XRPD pattern of Hydrochloride Salt 7 has peaks as set forth in Table 54 below.

TABLE 54

| | | d- | | Rel. |
|---|---|---|---|---|
| Index | Angle | Value | Intensity | Intensity |
| 1 | 4.88 | 18.09226 | 463 | 33.00% |
| 2 | 5.265 | 16.77225 | 450 | 32.10% |
| 3 | 5.633 | 15.67682 | 1.16E+03 | 82.80% |
| 4 | 6.058 | 14.57813 | 604 | 43.10% |
| 5 | 6.118 | 14.43386 | 553 | 39.40% |
| 6 | 8.663 | 10.19865 | 1.40E+03 | 100.00% |
| 7 | 9.201 | 9.60372 | 615 | 43.90% |
| 8 | 9.772 | 9.04347 | 933 | 66.50% |
| 9 | 10.709 | 8.25459 | 512 | 36.50% |
| 10 | 10.908 | 8.1048 | 556 | 39.70% |
| 11 | 11.513 | 7.67967 | 79.9 | 5.70% |
| 12 | 11.55 | 7.65525 | 107 | 7.60% |
| 13 | 12.126 | 7.29273 | 252 | 17.90% |
| 14 | 12.375 | 7.14687 | 398 | 28.40% |
| 15 | 12.736 | 6.94529 | 163 | 11.60% |
| 16 | 12.87 | 6.87294 | 331 | 23.60% |
| 17 | 13.169 | 6.71738 | 220 | 15.70% |
| 18 | 14.222 | 6.2227 | 107 | 7.60% |
| 19 | 14.619 | 6.05436 | 138 | 9.80% |
| 20 | 14.911 | 5.93668 | 150 | 10.70% |
| 21 | 15.546 | 5.69547 | 130 | 9.20% |
| 22 | 16.022 | 5.52732 | 249 | 17.70% |
| 23 | 16.557 | 5.34973 | 235 | 16.80% |
| 24 | 17.487 | 5.06726 | 174 | 12.40% |
| 25 | 18.17 | 4.87843 | 257 | 18.30% |
| 26 | 18.892 | 4.69365 | 668 | 47.60% |
| 27 | 19.211 | 4.61643 | 208 | 14.90% |
| 28 | 19.259 | 4.60494 | 250 | 17.90% |
| 29 | 19.387 | 4.57492 | 317 | 22.60% |
| 30 | 19.464 | 4.55699 | 383 | 27.30% |
| 31 | 19.537 | 4.54001 | 296 | 21.10% |
| 32 | 19.93 | 4.4515 | 250 | 17.80% |
| 33 | 19.945 | 4.44816 | 237 | 16.90% |
| 34 | 20.213 | 4.38979 | 217 | 15.50% |
| 35 | 20.842 | 4.25868 | 227 | 16.20% |
| 36 | 21.65 | 4.10153 | 340 | 24.30% |
| 37 | 21.696 | 4.09298 | 406 | 29.00% |
| 38 | 21.832 | 4.06771 | 581 | 41.50% |
| 39 | 22.027 | 4.03206 | 665 | 47.40% |
| 40 | 22.529 | 3.94334 | 239 | 17.10% |
| 41 | 23.888 | 3.72206 | 189 | 13.50% |
| 42 | 23.933 | 3.71517 | 233 | 16.60% |
| 43 | 25.021 | 3.55607 | 459 | 32.70% |
| 44 | 25.702 | 3.46338 | 177 | 12.60% |
| 45 | 27.154 | 3.28137 | 217 | 15.40% |
| 46 | 27.202 | 3.27568 | 216 | 15.40% |
| 47 | 28.225 | 3.15918 | 231 | 16.40% |
| 48 | 28.277 | 3.15352 | 256 | 18.20% |
| 49 | 28.291 | 3.15195 | 244 | 17.40% |
| 50 | 28.319 | 3.14897 | 259 | 18.50% |

The above crystal exhibits a thermal event between about 144° C. and 147° C., e.g., at about 145° C., according to differential scanning calorimetry. Hydrochloride Salt 7 is in solvate form. This form is reproducible using an amorphous hydrochloride salt of Compound 1 as well.

An eighth hydrochloride salt is generated following the methods as generally described above in Example 1. 100 mg of Hydrochloride Salt 1 is dissolved in ethyl salicylate and subjected to temperature cycles as described above. The obtained materials were analyzed using XRPD and TGA-DSC. The resulting salt is obtained as an off-white powder. The XRPD pattern of Hydrochloride Salt 8 has peaks as set forth in Table 55 below.

TABLE 55

| | | d- | | Rel. |
|---|---|---|---|---|
| Index | Angle | Value | Intensity | Intensity |
| 1 | 5.739 | 15.38731 | 3.23E+03 | 100.00% |
| 2 | 7.632 | 11.57373 | 317 | 9.80% |
| 3 | 10.094 | 8.75588 | 268 | 8.30% |
| 4 | 10.485 | 8.43082 | 352 | 10.90% |
| 5 | 11.387 | 7.7646 | 1.07E+03 | 33.00% |
| 6 | 11.584 | 7.6328 | 1.42E+03 | 44.00% |
| 7 | 12.458 | 7.09912 | 945 | 29.20% |
| 8 | 15.096 | 5.86417 | 169 | 5.20% |
| 9 | 16.877 | 5.24915 | 331 | 10.20% |
| 10 | 17.105 | 5.17975 | 461 | 14.30% |
| 11 | 17.391 | 5.09505 | 270 | 8.40% |
| 12 | 17.399 | 5.09281 | 256 | 7.90% |
| 13 | 17.784 | 4.98334 | 452 | 14.00% |
| 14 | 18.931 | 4.68395 | 465 | 14.40% |
| 15 | 19.235 | 4.61073 | 465 | 14.40% |
| 16 | 19.792 | 4.48208 | 331 | 10.20% |
| 17 | 19.889 | 4.46045 | 367 | 11.40% |
| 18 | 20.184 | 4.39606 | 651 | 20.10% |
| 19 | 20.372 | 4.35582 | 759 | 23.50% |
| 20 | 20.565 | 4.31544 | 1.13E+03 | 34.90% |
| 21 | 20.955 | 4.23597 | 187 | 5.80% |
| 22 | 22.134 | 4.0129 | 1.33E+03 | 41.20% |
| 23 | 22.805 | 3.89632 | 353 | 10.90% |
| 24 | 22.887 | 3.88259 | 266 | 8.20% |
| 25 | 23.678 | 3.7546 | 398 | 12.30% |
| 26 | 23.797 | 3.73611 | 328 | 10.20% |
| 27 | 24.864 | 3.57814 | 261 | 8.10% |
| 28 | 25.967 | 3.42855 | 374 | 11.60% |
| 29 | 26.473 | 3.36417 | 393 | 12.10% |
| 30 | 26.694 | 3.33687 | 285 | 8.80% |
| 31 | 28.254 | 3.15606 | 223 | 6.90% |
| 32 | 28.439 | 3.13592 | 200 | 6.20% |
| 33 | 30.587 | 2.92045 | 174 | 5.40% |
| 34 | 30.972 | 2.88496 | 112 | 3.50% |
| 35 | 31.219 | 2.86271 | 255 | 7.90% |
| 36 | 31.342 | 2.85176 | 197 | 6.10% |
| 37 | 34.265 | 2.61487 | 49.9 | 1.50% |
| 38 | 41.338 | 2.18235 | 67.8 | 2.10% |
| 39 | 43.742 | 2.0678 | 88.4 | 2.70% |

The above crystal exhibits a thermal event between about 196° C. and 200° C., e.g., at about 198° C., according to differential scanning calorimetry. Hydrochloride Salt 8 is in solvate form. This form is reproducible using an amorphous hydrochloride salt of Compound 1 as well.

### Example 4—Scale Up of Tartrate Salt

A further tartrate salt is generated following the methods as generally described above in Example 1. 100 mg of Tartrate Salt 1 is dissolved in methyl tert-butyl ether and subjected to temperature cycles using a Technobis Crystal 16 machine. The salt is subjected to consecutive cycles from 50° C. to 0° C., 40° C. to 0° C., 30° C. to 0° C. and 20° C. to 0° C., with a heating rate of 10° C./min and a cooling rate of 0.5° C./min. The obtained materials were analyzed using XRPD and TGA-DSC. The resulting salt is obtained as an off-white solid. The XRPD pattern of Tartrate Salt 2 has peaks as set forth in Table 56 below.

TABLE 56

| | | d- | | Rel. |
|---|---|---|---|---|
| Index | Angle | Value | Intensity | Intensity |
| 1 | 3.11 | 28.38948 | 188 | 5.20% |
| 2 | 3.157 | 27.96149 | 220 | 6.10% |
| 3 | 5.935 | 14.87894 | 2.05E+03 | 57.40% |
| 4 | 6.27 | 14.08582 | 3.58E+03 | 100.00% |
| 5 | 6.987 | 12.64062 | 266 | 7.40% |
| 6 | 8.009 | 11.02972 | 362 | 10.10% |
| 7 | 10.249 | 8.62436 | 291 | 8.10% |
| 8 | 11.131 | 7.94292 | 739 | 20.60% |
| 9 | 12.241 | 7.22472 | 870 | 24.30% |
| 10 | 12.589 | 7.02566 | 818 | 22.80% |
| 11 | 13.549 | 6.52984 | 245 | 6.80% |
| 12 | 13.75 | 6.43526 | 163 | 4.50% |
| 13 | 14.265 | 6.20389 | 107 | 3.00% |
| 14 | 14.952 | 5.92047 | 120 | 3.30% |
| 15 | 15.399 | 5.74943 | 202 | 5.60% |
| 16 | 17.019 | 5.20565 | 806 | 22.50% |
| 17 | 17.377 | 5.09934 | 525 | 14.70% |
| 18 | 17.449 | 5.07841 | 555 | 15.50% |
| 19 | 18.695 | 4.74261 | 165 | 4.60% |
| 20 | 18.76 | 4.7264 | 181 | 5.00% |
| 21 | 19.43 | 4.56481 | 80.9 | 2.30% |
| 22 | 19.965 | 4.44367 | 133 | 3.70% |
| 23 | 20.401 | 4.34961 | 184 | 5.10% |
| 24 | 21.181 | 4.19113 | 280 | 7.80% |
| 25 | 21.456 | 4.13808 | 772 | 21.60% |
| 26 | 22.846 | 3.88932 | 171 | 4.80% |
| 27 | 22.961 | 3.8702 | 123 | 3.40% |
| 28 | 23.573 | 3.77112 | 142 | 4.00% |
| 29 | 24.559 | 3.62181 | 188 | 5.20% |
| 30 | 24.974 | 3.56267 | 118 | 3.30% |
| 31 | 29.081 | 3.06818 | 57.6 | 1.60% |
| 32 | 29.484 | 3.02708 | 102 | 2.80% |
| 33 | 31.51 | 2.83694 | 88.2 | 2.50% |

The above crystal exhibits a thermal event between about 103° C. and 106° C., e.g., at about 104° C., according to differential scanning calorimetry. Tartrate Salt 2 is in solvate form, and the free base and counter ion are present in the crystal in a ratio of about 1:0.6. Tartrate Salt 2 is reproducible at 1 g scale of Tartrate Salt 1, but exhibits thermal events between about 120° C. and 123° C., e.g., at about 121° C., and between about 134° C. and 137° C., e.g., at about 136° C.

### Example 5—Scale Up of Oxalate Salt

A further tartrate salt is generated following the methods as generally described above in Example 1. 100 mg of Oxalate Salt 3 is dissolved in ethyl salicylate and subjected to temperature cycles using a Technobis Crystal 16 machine. The salt is subjected to consecutive cycles from 50° C. to 0° C., 40° C. to 0° C., 30° C. to 0° C. and 20° C. to 0° C., with a heating rate of 10° C./min and a cooling rate of 0.5° C./min. The obtained materials were analyzed using XRPD and TGA-DSC. The resulting salt is obtained as an off-white solid. The XRPD pattern of Oxalate Salt 4 has peaks as set forth in Table 57 below.

TABLE 57

| | | d- | | Rel. |
|---|---|---|---|---|
| Index | Angle | Value | Intensity | Intensity |
| 1 | 3.658 | 24.1349 | 156 | 3.90% |
| 2 | 5.151 | 17.14139 | 588 | 14.70% |
| 3 | 5.772 | 15.29813 | 1.36E+03 | 34.00% |
| 4 | 8.904 | 9.92404 | 446 | 11.20% |
| 5 | 9.643 | 9.16428 | 538 | 13.50% |
| 6 | 11.566 | 7.64479 | 2.74E+03 | 68.60% |
| 7 | 12.093 | 7.3128 | 667 | 16.70% |
| 8 | 15.331 | 5.77498 | 584 | 14.60% |
| 9 | 16.656 | 5.31822 | 445 | 11.10% |
| 10 | 17.295 | 5.12329 | 417 | 10.40% |
| 11 | 18.088 | 4.90048 | 1.27E+03 | 31.70% |
| 12 | 18.497 | 4.79282 | 780 | 19.50% |
| 13 | 18.862 | 4.70109 | 394 | 9.90% |
| 14 | 20.445 | 4.34037 | 3.99E+03 | 100.00% |
| 15 | 21.445 | 4.14017 | 1.20E+03 | 29.90% |
| 16 | 21.922 | 4.05115 | 1.55E+03 | 38.80% |
| 17 | 23.237 | 3.82484 | 814 | 20.40% |
| 18 | 23.781 | 3.73864 | 421 | 10.50% |
| 19 | 24.798 | 3.58755 | 503 | 12.60% |
| 20 | 25.815 | 3.44839 | 287 | 7.20% |
| 21 | 26.652 | 3.34196 | 179 | 4.50% |
| 22 | 27.065 | 3.29193 | 887 | 22.20% |
| 23 | 28.477 | 3.13187 | 202 | 5.10% |
| 24 | 28.917 | 3.08516 | 632 | 15.80% |
| 25 | 30.145 | 2.96224 | 340 | 8.50% |
| 26 | 31.235 | 2.86134 | 399 | 10.00% |
| 27 | 31.842 | 2.80808 | 282 | 7.10% |
| 28 | 32.625 | 2.74248 | 147 | 3.70% |
| 29 | 33.377 | 2.68239 | 297 | 7.40% |
| 30 | 34.352 | 2.60846 | 83.3 | 2.10% |
| 31 | 34.931 | 2.56653 | 159 | 4.00% |
| 32 | 36.272 | 2.47467 | 227 | 5.70% |
| 33 | 37.379 | 2.40387 | 121 | 3.00% |
| 34 | 38.054 | 2.3628 | 64.1 | 1.60% |
| 35 | 39.04 | 2.30535 | 131 | 3.30% |
| 36 | 41.339 | 2.18232 | 73.2 | 1.80% |
| 37 | 41.838 | 2.1574 | 134 | 3.40% |
| 38 | 43.634 | 2.07269 | 40.2 | 1.00% |
| 39 | 44.433 | 2.03725 | 93.7 | 2.30% |

The above crystal exhibits a thermal event between about 125° C. and 128° C., e.g., at about 126° C., and between about 138° C. and 148° C., e.g., at about 139° C., according to differential scanning calorimetry. Oxalate Salt 4 is in solvate form.

1. A salt comprising (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one (Compound A) in an acid addition salt form selected from hydrochloride, malate, fumarate, sulfate, esylate, galactarate, adipate, lactate, oxalate, palmitate, 2-oxo-glutarate, xinafoate, tosylate, tartrate, succinate, mesylate, napadisylate, edisylate, propionate, caprylate, besylate, benzoate, nicotinate, isonicotinate, orotate, camsylate, salicylate, aminosalicylate, mandelate, acetamido-benzoate, trifluoroacetate, dichloroacetate, caproate, or laurate salt form.

2. The salt according to claim 1, wherein the salt is crystalline.

3. The salt according to claim 2, wherein the salt is a hydrochloride salt, optionally wherein the salt is in the form of a solvate with acetonitrile, ethyl acetate, acetone, 2-butanone, 2-ethyl-1-butanol, ethyl salicylate, ethyl butyl ketone, acetone, or combinations thereof.

4. The salt according to claim 3, wherein said salt crystal exhibits an X-ray powder diffraction pattern comprising:
a. at least five peaks having 2-theta angle values selected from the group consisting of: 4.9, 7.3, 9.5, 9.7, 12.3, 14.4, 14.6, 19.0, 19.6, and 21.4 degrees, wherein the XRPD pattern is measured in a diffractometer using copper anode, e.g., at wavelength alpha1 of 1.5406 Å and wavelength alpha2 of 1.5444 Å;
b. at least five peaks having 2-theta angle values selected from the group consisting of: 7.3, 12.1, 13.6, 15.6, 16.4, 18.5, 20.0, 21.3, 21.4, and 21.5 degrees, wherein the XRPD pattern is measured in a diffractometer using copper anode, e.g., at wavelength alpha1 of 1.5406 Å and wavelength alpha2 of 1.5444 Å;
c. at least five peaks having 2-theta angle values selected from the group consisting of: 4.9, 6.9, 7.3, 7.4, 12.2, 12.7, 14.6, 20.6, 27.6, and 32.7 degrees, wherein the XRPD pattern is measured in a diffractometer using copper anode, e.g., at wavelength alpha1 of 1.5406 Å and wavelength alpha2 of 1.5444 Å;
d. at least five peaks having 2-theta angle values selected from the group consisting of: 7.6, 12.0, 12.7, 15.0, 15.1, 17.9, 18.8, 19.3, 23.1, and 24.0 degrees, wherein the XRPD pattern is measured in a diffractometer using copper anode, e.g., at wavelength alpha1 of 1.5406 Å and wavelength alpha2 of 1.5444 Å;
e. at least five peaks having 2-theta angle values selected from the group consisting of: 6.7, 7.7, 8.8, 9.1, 11.4, 16.4, 17.0, 18.4, 21.9, and 24.1 degrees, wherein the XRPD pattern is measured in a diffractometer using copper anode, e.g., at wavelength alpha1 of 1.5406 Å and wavelength alpha2 of 1.5444 Å;
f. at least five peaks having 2-theta angle values selected from the group consisting of: 5.0, 7.1, 7.5, 7.8, 8.5, 12.4, 13.0, 18.7, 18.8, and 20.8 degrees, wherein the XRPD pattern is measured in a diffractometer using copper anode, e.g., at wavelength alpha1 of 1.5406 Å and wavelength alpha2 of 1.5444 Å;
g. at least five peaks having 2-theta angle values selected from the group consisting of: 5.6, 8.7, 6.1, 9.2, 9.8, 10.7, 10.9, 18.9, 21.8, and 22.0 degrees, wherein the XRPD pattern is measured in a diffractometer using copper anode, e.g., at wavelength alpha1 of 1.5406 Å and wavelength alpha2 of 1.5444 Å; or
h. at least five peaks having 2-theta angle values selected from the group consisting of: 5.7, 11.4, 11.6, 12.5, 18.9, 19.2, 20.2, 20.4, 20.6, and 22.1 degrees, wherein the XRPD pattern is measured in a diffractometer using copper anode, e.g., at wavelength alpha1 of 1.5406 Å and wavelength alpha2 of 1.5444 Å.

5. (canceled)

6. (canceled)

7. The salt according to claim 3, wherein said salt crystal exhibits a Differential Scanning Calorimetry (DSC) pattern comprising:
a. an endothermic peak at about 169° C.-172° C., e.g., at about 170° C.;
b. an endothermic peak at about 140° C. to 142° C., e.g., at about 141° C., and/or between about 190° C. to 192° C., e.g., at about 191° C.;
c. an endothermic peak between about 155° C. and 157° C., e.g., at about 156° C., and/or between about 275° C. and 277° C., e.g., at about 276° C.;
d. an endothermic peak between about 194° C. and 196° C., e.g., at about 195° C., and/or between about 209° C. and 211° C., e.g., at about 210° C.;
e. an endothermic peak between about 158° C. and 161° C., e.g., at about 159° C.;
f. an endothermic peak between about 129° C. and 133° C., e.g., at about 131° C.;
g. an endothermic peak between about 144° C. and 147° C., e.g., at about 145° C.; or
h. an endothermic peak between about 196° C. and 200° C., e.g., at about 198° C.

8. (canceled)

9. The salt according to claim 2, wherein the salt is a malate salt.

10. The salt according to claim 9, wherein said salt crystal exhibits an X-ray powder diffraction pattern comprising at least five peaks having 2-theta angle values selected from the group consisting of: 5.9, 7.2, 12.0, 16.0, 17.7, 17.8, 20.9, 21.2, 21.7, and 21.8 degrees, wherein the XRPD pattern is measured in a diffractometer using copper anode, e.g., at wavelength alpha1 of 1.5406 Å and wavelength alpha2 of 1.5444 Å.

11. (canceled)

12. (canceled)

13. The salt according to claim 9, wherein said salt crystal exhibits a Differential Scanning Calorimetry (DSC) pattern comprising an endothermic peak between about 94° C. and 96° C., e.g., at about 95° C.

14. The salt according to claim 2, wherein the salt is an oxalate salt.

15. The salt according to claim 14, wherein said salt crystal exhibits an X-ray powder diffraction pattern comprising:
a. at least five peaks having 2-theta angle values selected from the group consisting of: 7.1, 8.5, 12.2, 12.3, 16.3, 19.2, 20.7, 22.9, 24.1, and 25.4 degrees, wherein the XRPD pattern is measured in a diffractometer using copper anode, e.g., at wavelength alpha1 of 1.5406 Å and wavelength alpha2 of 1.5444 Å;
b. at least five peaks having 2-theta angle values selected from the group consisting of: 6.5, 6.7, 7.2, 16.3, 16.7, 17.0, 19.5, 20.0, 20.6, and 20.8 degrees, wherein the XRPD pattern is measured in a diffractometer using copper anode, e.g., at wavelength alpha1 of 1.5406 Å and wavelength alpha2 of 1.5444 Å;
c. at least five peaks having 2-theta angle values selected from the group consisting of: 5.3, 6.0, 11.9, 16.6, 17.7, 18.3, 19.6, 20.5, 20.7, and 21.2 degrees, wherein the XRPD pattern is measured in a diffractometer using copper anode, e.g., at wavelength alpha1 of 1.5406 Å and wavelength alpha2 of 1.5444 Å; or
d. at least five peaks having 2-theta angle values selected from the group consisting of: 5.8, 11.6, 12.1, 18.1, 18.5, 20.4, 21.4, 21.9, 27.1, and 23.2 degrees, wherein the XRPD pattern is measured in a diffractometer using copper anode, e.g., at wavelength alpha1 of 1.5406 Å and wavelength alpha2 of 1.5444 Å.

16. (canceled)

17. (canceled)

18. The salt according to claim 14, wherein said salt crystal exhibits a Differential Scanning Calorimetry (DSC) pattern comprising:
a. an endothermic peak between about 218° C. and 220° C., e.g., at about 219° C.;
b. an endothermic peak between about 165° C. and 167° C., e.g., at about 166° C., between about 205° C. and 207° C., e.g., at about 207° C., and/or between about 214° C. and 216° C., e.g., at about 215° C.;
c. an endothermic peak between about 214° C. and 220° C., e.g., at about 214° C., 218° C. or 219° C.; or
d. an endothermic peak between about 125° C. and 128° C., e.g., at about 126° C., and/or between about 138° C. and 148° C., e.g., at about 139° C.

19. The salt according to claim 2, wherein the salt is a tartrate salt.

20. The salt according to claim 19, wherein said salt crystal exhibits an X-ray powder diffraction pattern comprising:
a. at least five peaks having 2-theta angle values selected from the group consisting of: 3.7, 6.0, 6.9, 10.4, 11.6, 15.0, 17.5, 20.3, 20.8, and 21.7 degrees, wherein the XRPD pattern is measured in a diffractometer using copper anode, e.g., at wavelength alpha1 of 1.5406 Å and wavelength alpha2 of 1.5444 Å; or
b. at least five peaks having 2-theta angle values selected from the group consisting of: 5.9, 6.3, 8.0, 10.2, 11.1, 12.2, 12.6, 17.0, 17.4, and 21.5 degrees, wherein the XRPD pattern is measured in a diffractometer using copper anode, e.g., at wavelength alpha1 of 1.5406 Å and wavelength alpha2 of 1.5444 Å.

21. (canceled)

22. (canceled)

23. The salt according to claim 19, wherein said salt crystal exhibits a Differential Scanning Calorimetry (DSC) pattern comprising:
a. an endothermic peak between about 134° C. and 136° C., e.g., at about 135° C.; or
b. an endothermic peak between about 103° C. and 106° C., e.g., at about 104° C., between about 120° C. and 123° C., e.g., at about 121° C., and/or between about 134° C. and 137° C., e.g., at about 136° C.

24. A method for the production of stable acid addition salts of (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one (“Compound A”), e.g., crystallinic acid addition salts, with an acid selected from hydrochloric acid, malic acid, fumaric acid, sulfuric acid, ethane sulfonic acid, galactaric acid, adipic acid, lactic acid, oxalic acid, palmitic acid, 2-oxo-glutaric acid, xinafoic acid, toluene sulfonic acid, tartaric acid, succinic acid, methane sulfonic acid, naphthalene disulfonic acid, ethane disulfonic acid, propionic naphthalene disulfonic acid, caprylic naphthalene disulfonic acid, benzenesulfonic acid, benzoic acid, nicotinic acid, isonicotinic acid, orotic acid, camsylic acid, salicylic acid, aminosalicylic acid, mandelic acid, acetamido-benzoic acid, trifluoroacetic acid, dichloroacetic acid, caproic acid, or lauric acid, the method comprising the steps of reacting Compound A in free base form with the acid in a solvent and isolating the salt obtained, optionally wherein the acid and Compound A in free base form are reacted in a molar ratio of 1:1.

25. The method according to claim 24, further comprising the step of forming a slurry of Compound A with the acid in the solvent at a temperature between about 30° C. to 70° C., optionally wherein the slurry is formed at a temperature of 30° C. to 70° C. for a period of at least 1-3 hours, e.g., at a temperature of about 40° C. to 60° C., e.g., about 45° C. to 65° C., e.g., about 50° C.

26. (canceled)

27. The method according to claim 24, further comprising the step of cooling the solution to a temperature of about −10° C. to about 20° C., e.g., about 0° C. to about 10° C., e.g., about 5° C., optionally wherein the solution is cooled for a period of at least about 5 hours, e.g., for a period of about 5 hours to about 24 hours, e.g., for a period of about 8 hours.

28. (canceled)

29. The method according to claim 24, further comprising the step of drying the solution by evaporation, e.g., wherein the solution is placed under vacuum to evaporate the solvent.

30. The method according to claim 24, wherein the obtained salt is crystalline, and are dissolved in a second solvent and are subjected to one or more cooling cycles.

31. The method according to claim 24, wherein the cooling cycle comprises heating then cooling the solution for at least 2 cycles (e.g., at least 3 cycles, at least 4 cycles, at least 5 cycles).

32. (canceled)
